SEQUENCE LISTING (100 sequences)

SEQ ID NO: 1 - DNA, 2487 bp, Artificial
  Synthetic Construct; SFD2/D4Long Gqi5
atgaagacgatcatcgccctgagctacatcttctgcctggtgttcgccgactacaaggacgatgatgacgccatggatccactgaatctgtcctggtatgatgatgatctggagaggcagaactggagccggcccttcaacgggtcagacgggaaggcggacagaccccactacaactactatgccacactgctcaccctgctcatcgctgtcatcgtcttcggcaacgtgctggtgtgcatggctgtgtcccgcgagaaggcgctgcagaccaccaccaactacctgatcgtcagcctcgcagtggccgacctcctcgtcgccacactggtcatgccttggtttgtatacctggaggtggtaggtgagtggaaattcagcaggattcactgtgacatcttaatgactctggacgtcatgatgtgcacggcgagcatcctgaacttgtgtgccatcagcatcgacaggtacacagctgtggccatgcccatgctgtacaatacgcgctacagctccaagcgccgggtcaccgtcatgatctccatcgtctgggtcctgtccttcaccatctcctgcccactcctcttcggactcaataacgcagaccagaacgagtgcatcattgccaacccggccttcgtggtctactcctccatcgtctccttctacgtgcccttcattgtcaccctgctggtctacatcaagatctacattgtcctccgcagacgccgcaagcgagtcaacaccaaacgcagcagccgagctttcagggcccacctgagggctccactaaagggcaactgtactcaccccgaggacatgaaactctgcaccgttatcatgaagtctaatgggagtttcccagtgaacaggcggagagtggaggctgcccggcgagcccaggagctggagatggagatgctctccagcaccagcccacccgagaggacccggtacagccccatcccacccagccaccaccagctgactctccccgacccgtcccaccatggtctccacagcactcccgacagccccgccaaaccagagaagaatgggcatgccaaagaccaccccaagattgccaagatctttgagatccagaccatgcccaatggcaaaacgcgtacctccctcaagaccatgagccgtaggaagctctcccagcagaaggcaaagaaagctcgtcagatgctcgccattgttctcggcgtgttcatcatctgctggctgcccttcttcatcacacacatcctgaacatacactgtgactgcaacatcccgcctgtcctggttagcgccttcacgtggctgggctatgtcaacagcgccgtgaaccccatcatctacaccaccttcaacattgagttccgcaaggccttcctgaagatcctccactgtttcgaaatgactctggagtccatcatggcgtgctgcctgagcgaggaggccaaggaagcccggcggatcaacgacgagatcgagcggcagctccgcagggacaagcgggacgcccgccgggagctcaagctgctgctgctcgggacaggagagagtggcaagagtacgtttatcaagcagatgagaatcatccatgggtcaggatactctgatgaagataaaaggggcttcaccaagctggtgtatcagaacatcttcacggccatgcaggccatgatcagagccatggacacactcaagatcccatacaagtatgagcacaataaggctcatgcacaattagttcgagaagttgatgtggagaaggtgtctgcttttgagaatccatatgtagatgcaataaagagtttatggaatgatcctggaatccaggaatgctatgatagacgacgagaatatcaattatctgactctaccaaatactatcttaatgacttggaccgcgtagctgaccctgcctacctgcctacgcaacaagatgtgcttagagttcgagtccccaccacagggatcatcgaatacccctttgacttacaaagtgtcattttcagaatggtcgatgtagggggccaaaggtcagagagaagaaaatggatacactgctttgaaaatgtcacctctatcatgtttctagtagcgcttagtgaatatgatcaagttctcgtggagtcagacaatgagaaccgaatggaggaaagcaaggctctctttagaacaattatcacatacccctggttccagaactcctcggttattctgttcttaaacaagaaagatcttctagaggagaaaatcatgtattcccatctagtcgactacttcccagaatatgatggaccccagagagatgcccaggcagcccgagaattcattctgaagatgttcgtggacctgaacccagacagtgacaaaattatctactcccacttcacgtgcgccacagacaccgagaatatccgctttgtctttgctgccgtcaaggacaccatcctccagttgaacctgaaggatattggtctgttctaa

SEQ ID NO: 2 - DNA, 2487 bp, Artificial
  Synthetic Construct; SFD2/D4Long/CAM Gqi5
atgaagacgatcatcgccctgagctacatcttctgcctggtgttcgccgactacaaggacgatgatgacgccatggatccactgaatctgtcctggtatgatgatgatctggagaggcagaactggagccggcccttcaacgggtcagacgggaaggcggacagaccccactacaactactatgccacactgctcaccctgctcatcgctgtcatcgtcttcggcaacgtgctggtgtgcatggctgtgtcccgcgagaaggcgctgcagaccaccaccaactacctgatcgtcagcctcgcagtggccgacctcctcgtcgccacactggtcatgccttggtttgtatacctggaggtggtaggtgagtggaaattcagcaggattcactgtgacatcttaatgactctggacgtcatgatgtgcacggcgagcatcctgaacttgtgtgccatcagcatcgacaggtacacagctgtggccatgcccatgctgtacaatacgcgctacagctccaagcgccgggtcaccgtcatgatctccatcgtctgggtcctgtccttcaccatctcctgcccactcctcttcggactcaataacgcagaccagaacgagtgcatcattgccaacccggccttcgtggtctactcctccatcgtctccttctacgtgcccttcattgtcaccctgctggtctacatcaagatctacattgtcctccgcagacgccgcaagcgagtcaacaccaaacgcagcagccgagctttcagggcccacctgagggctccactaaagggcaactgtactcaccccgaggacatgaaactctgcaccgttatcatgaagtctaatgggagtttcccagtgaacaggcggagagtggaggctgcccggcgagcccaggagctggagatggagatgctctccagcaccagcccacccgagaggacccggtacagccccatcccacccagccaccaccagctgactctccccgacccgtcccaccatggtctccacagcactcccgacagccccgccaaaccagagaagaatgggcatgccaaagaccaccccaagattgccaagatctttgagatccagaccatgcccaatggcaaaacgcgtacctccctcaagaccatgagccgtaggaagctctcccagcagaaggagaagaaagccactcagatgctcgccattgttctcggcgtgttcatcatctgctggctgcccttcttcatcacacacatcctgaacatacactgtgactgcaacatcccgcctgtcctggttagcgccttcacgtggctgggctatgtcaacagcgccgtgaaccccatcatctacaccaccttcaacattgagttccgcaaggccttcctgaagatcctccactgtttcgaaatgactctggagtccatcatggcgtgctgcctgagcgaggaggccaaggaagcccggcggatcaacgacgagatcgagcggcagctccgcagggacaagcgggacgcccgccgggagctcaagctgctgctgctcgggacaggagagagtggcaagagtacgtttatcaagcagatgagaatcatccatgggtcaggatactctgatgaagataaaaggggcttcaccaagctggtgtatcagaacatcttcacggccatgcaggccatgatcagagccatggacacactcaagatcccatacaagtatgagcacaataaggctcatgcacaattagttcgagaagttgatgtggagaaggtgtctgcttttgagaatccatatgtagatgcaataaagagtttatggaatgatcctggaatccaggaatgctatgatagacgacgagaatatcaattatctgactctaccaaatactatcttaatgacttggaccgcgtagctgaccctgcctacctgcctacgcaacaagatgtgcttagagttcgagtccccaccacagggatcatcgaatacccctttgacttacaaagtgtcattttcagaatggtcgatgtagggggccaaaggtcagagagaagaaaatggatacactgctttgaaaatgtcacctctatcatgtttctagtagcgcttagtgaatatgatcaagttctcgtggagtcagacaatgagaaccgaatggaggaaagcaaggctctctttagaacaattatcacatacccctggttccagaactcctcggttattctgttcttaaacaagaaagatcttctagaggagaaaatcatgtattcccatctagtcgactacttcccagaatatgatggaccccagagagatgcccaggcagcccgagaattcattctgaagatgttcgtggacctgaacccagacagtgacaaaattatctactcccacttcacgtgcgccacagacaccgagaatatccgctttgtctttgctgccgtcaaggacaccatcctccagttgaacctgaaggatattggtctgttctaa

SEQ ID NO: 3 - DNA, 2400 bp, Artificial
  Synthetic Construct; SFD2/D4short Gqi5
atgaagacgatcatcgccctgagctacatcttctgcctggtgttcgccgactacaaggacgatgatgacgccatggatccactgaatctgtcctggtatgatgatgatctggagaggcagaactggagccggcccttcaacgggtcagacgggaaggcggacagaccccactacaactactatgccacactgctcaccctgctcatcgctgtcatcgtcttcggcaacgtgctggtgtgcatggctgtgtcccgcgagaaggcgctgcagaccaccaccaactacctgatcgtcagcctcgcagtggccgacctcctcgtcgccacactggtcatgccttggtttgtatacctggaggtggtaggtgagtggaaattcagcaggattcactgtgacatcttaatgactctggacgtcatgatgtgcacggcgagcatcctgaacttgtgtgccatcagcatcgacaggtacacagctgtggccatgcccatgctgtacaatacgcgctacagctccaagcgccgggtcaccgtcatgatctccatcgtctgggtcctgtccttcaccatctcctgcccactcctcttcggactcaataacgcagaccagaacgagtgcatcattgccaacccggccttcgtggtctactcctccatcgtctccttctacgtgcccttcattgtcaccctgctggtctacatcaagatctacattgtcctccgcagacgccgcaagcgagtcaacaccaaacgcagcagccgagctttcagggcccacctgagggctccactaaaggaggctgcccggcgagcccaggagctggagatggagatgctctccagcaccagcccacccgagaggacccggtacagccccatcccacccagccaccaccagctgactctccccgacccgtcccaccatggtctccacagcactcccgacagccccgccaaaccagagaagaatgggcatgccaaagaccaccccaagattgccaagatctttgagatccagaccatgcccaatggcaaaacgcgtacctccctcaagaccatgagccgtaggaagctctcccagcagaaggagaagaaagccactcagatgctcgccattgttctcggcgtgttcatcatctgctggctgcccttcttcatcacacacatcctgaacatacactgtgactgcaacatcccgcctgtcctggttagcgccttcacgtggctgggctatgtcaacagcgccgtgaaccccatcatctacaccaccttcaacattgagttccgcaaggccttcctgaagatcctccactgtttcgaaatgactctggagtccatcatggcgtgctgcctgagcgaggaggccaaggaagcccggcggatcaacgacgagatcgagcggcagctccgcagggacaagcgggacgcccgccgggagctcaagctgctgctgctcgggacaggagagagtggcaagagtacgtttatcaagcagatgagaatcatccatgggtcaggatactctgatgaagataaaaggggcttcaccaagctggtgtatcagaacatcttcacggccatgcaggccatgatcagagccatggacacactcaagatcccatacaagtatgagcacaataaggctcatgcacaattagttcgagaagttgatgtggagaaggtgtctgcttttgagaatccatatgtagatgcaataaagagtttatggaatgatcctggaatccaggaatgctatgatagacgacgagaatatcaattatctgactctaccaaatactatcttaatgacttggaccgcgtagctgaccctgcctacctgcctacgcaacaagatgtgcttagagttcgagtccccaccacagggatcatcgaatacccctttgacttacaaagtgtcattttcagaatggtcgatgtagggggccaaaggtcagagagaagaaaatggatacactgctttgaaaatgtcacctctatcatgtttctagtagcgcttagtgaatatgatcaagttctcgtggagtcagacaatgagaaccgaatggaggaaagcaaggctctctttagaacaattatcacatacccctggttccagaactcctcggttattctgttcttaaacaagaaagatcttctagaggagaaaatcatgtattcccatctagtcgactacttcccagaatatgatggaccccagagagatgcccaggcagcccgagaattcattctgaagatgttcgtggacctgaacccagacagtgacaaaattatctactcccacttcacgtgcgccacagacaccgagaatatccgctttgtctttgctgccgtcaaggacaccatcctccagttgaacctgaaggatattggtctgttctaa

SEQ ID NO: 4 - DNA, 2400 bp, Artificial
  Synthetic Construct; SFD2S AAAA(219-222RRKR) Gqi5
atgaagacgatcatcgccctgagctacatcttctgcctggtgttcgccgactacaaggacgatgatgacgccatggatccactgaatctgtcctggtatgatgatgatctggagaggcagaactggagccggcccttcaacgggtcagacgggaaggcggacagaccccactacaactactatgccacactgctcaccctgctcatcgctgtcatcgtcttcggcaacgtgctggtgtgcatggctgtgtcccgcgagaaggcgctgcagaccaccaccaactacctgatcgtcagcctcgcagtggccgacctcctcgtcgccacactggtcatgccctgggttgtctacctggaggtggtaggtgagtggaaattcagcaggattcactgtgacatcttcgtcactctggacgtcatgatgtgcacggcgagcatcctgaacttgtgtgccatcagcatcgacaggtacacagctgtggccatgcccatgctgtacaatacgcgctacagctccaagcgccgggtcaccgtcatgatctccatcgtctgggtcctgtccttcaccatctcctgcccactcctcttcggactcaataacgcagaccagaacgagtgcatcattgccaacccggccttcgtggtctactcctccatcgtctccttctacgtgcccttcattgtcaccctgctggtctacatcaagatctacattgtcctccgcagagcagcagcagcagtcaacaccaaacgcagcagccgagctttcagggcccacctgagggctccactaaaggaggctgcccggcgagcccaggagctggagatggagatgctctccagcaccagcccacccgagaggacccggtacagccccatcccacccagccaccaccagctgactctccccgacccgtcccaccatggtctccacagcactcccgacagccccgccaaaccagagaagaatgggcatgccaaagaccaccccaagattgccaagatctttgagatccagaccatgcccaatggcaaaacgcgtacctccctcaagaccatgagccgtaggaagctctcccagcagaaggagaagaaagccactcagatgctcgccattgttctcggcgtgttcatcatctgctggctgcccttcttcatcacacacatcctgaacatacactgtgactgcaacatcccgcctgtcctgtacagcgccttcacgtggctgggctatgtcaacagcgccgtgaaccccatcatctacaccaccttcaacattgagttccgcaaggccttcctgaagatcctccactgtttcgaaatgactctggagtccatcatggcgtgctgcctgagcgaggaggccaaggaagcccggcggatcaacgacgagatcgagcggcagctccgcagggacaagcgggacgcccgccgggagctcaagctgctgctgctcgggacaggagagagtggcaagagtacgtttatcaagcagatgagaatcatccatgggtcaggatactctgatgaagataaaaggggcttcaccaagctggtgtatcagaacatcttcacggccatgcaggccatgatcagagccatggacacactcaagatcccatacaagtatgagcacaataaggctcatgcacaattagttcgagaagttgatgtggagaaggtgtctgcttttgagaatccatatgtagatgcaataaagagtttatggaatgatcctggaatccaggaatgctatgatagacgacgagaatatcaattatctgactctaccaaatactatcttaatgacttggaccgcgtagctgaccctgcctacctgcctacgcaacaagatgtgcttagagttcgagtccccaccacagggatcatcgaatacccctttgacttacaaagtgtcattttcagaatggtcgatgtagggggccaaaggtcagagagaagaaaatggatacactgctttgaaaatgtcacctctatcatgtttctagtagcgcttagtgaatatgatcaagttctcgtggagtcagacaatgagaaccgaatggaggaaagcaaggctctctttagaacaattatcacatacccctggttccagaactcctcggttattctgttcttaaacaagaaagatcttctagaggagaaaatcatgtattcccatctagtcgactacttcccagaatatgatggaccccagagagatgcccaggcagcccgagaattcattctgaagatgttcgtggacctgaacccagacagtgacaaaattatctactcccacttcacgtgcgccacagacaccgagaatatccgctttgtctttgctgccgtcaaggacaccatcctccagttgaacctgaaggatattggtctgttctaa

SEQ ID NO: 5 - DNA, 2400 bp, Artificial
  Synthetic Construct; SFD2S AAAA(219-222RRKR)/CAM Gqi5
atgaagacgatcatcgccctgagctacatcttctgcctggtgttcgccgactacaaggacgatgatgacgccatggatccactgaatctgtcctggtatgatgatgatctggagaggcagaactggagccggcccttcaacgggtcagacgggaaggcggacagaccccactacaactactatgccacactgctcaccctgctcatcgctgtcatcgtcttcggcaacgtgctggtgtgcatggctgtgtcccgcgagaaggcgctgcagaccaccaccaactacctgatcgtcagcctcgcagtggccgacctcctcgtcgccacactggtcatgccctgggttgtctacctggaggtggtaggtgagtggaaattcagcaggattcactgtgacatcttcgtcactctggacgtcatgatgtgcacggcgagcatcctgaacttgtgtgccatcagcatcgacaggtacacagctgtggccatgcccatgctgtacaatacgcgctacagctccaagcgccgggtcaccgtcatgatctccatcgtctgggtcctgtccttcaccatctcctgcccactcctcttcggactcaataacgcagaccagaacgagtgcatcattgccaacccggccttcgtggtctactcctccatcgtctccttctacgtgcccttcattgtcaccctgctggtctacatcaagatctacattgtcctccgcagagcagcagcagcagtcaacaccaaacgcagcagccgagctttcagggcccacctgagggctccactaaaggaggctgcccggcgagcccaggagctggagatggagatgctctccagcaccagcccacccgagaggacccggtacagccccatcccacccagccaccaccagctgactctccccgacccgtcccaccatggtctccacagcactcccgacagccccgccaaaccagagaagaatgggcatgccaaagaccaccccaagattgccaagatctttgagatccagaccatgcccaatggcaaaacgcgtacctccctcaagaccatgagccgtaggaagctctcccagcagaaggcaaagaaagctcgtcagatgctcgccattgttctcggcgtgttcatcatctgctggctgcccttcttcatcacacacatcctgaacatacactgtgactgcaacatcccgcctgtcctgtacagcgccttcacgtggctgggctatgtcaacagcgccgtgaaccccatcatctacaccaccttcaacattgagttccgcaaggccttcctgaagatcctccactgtttcgaaatgactctggagtccatcatggcgtgctgcctgagcgaggaggccaaggaagcccggcggatcaacgacgagatcgagcggcagctccgcagggacaagcgggacgcccgccgggagctcaagctgctgctgctcgggacaggagagagtggcaagagtacgtttatcaagcagatgagaatcatccatgggtcaggatactctgatgaagataaaaggggcttcaccaagctggtgtatcagaacatcttcacggccatgcaggccatgatcagagccatggacacactcaagatcccatacaagtatgagcacaataaggctcatgcacaattagttcgagaagttgatgtggagaaggtgtctgcttttgagaatccatatgtagatgcaataaagagtttatggaatgatcctggaatccaggaatgctatgatagacgacgagaatatcaattatctgactctaccaaatactatcttaatgacttggaccgcgtagctgaccctgcctacctgcctacgcaacaagatgtgcttagagttcgagtccccaccacagggatcatcgaatacccctttgacttacaaagtgtcattttcagaatggtcgatgtagggggccaaaggtcagagagaagaaaatggatacactgctttgaaaatgtcacctctatcatgtttctagtagcgcttagtgaatatgatcaagttctcgtggagtcagacaatgagaaccgaatggaggaaagcaaggctctctttagaacaattatcacatacccctggttccagaactcctcggttattctgttcttaaacaagaaagatcttctagaggagaaaatcatgtattcccatctagtcgactacttcccagaatatgatggaccccagagagatgcccaggcagcccgagaattcattctgaagatgttcgtggacctgaacccagacagtgacaaaattatctactcccacttcacgtgcgccacagacaccgagaatatccgctttgtctttgctgccgtcaaggacaccatcctccagttgaacctgaaggatattggtctgttctaa

SEQ ID NO: 6 - DNA, 2400 bp, Artificial
  Synthetic Construct; SFD2S D131A/R132A Gqi5
atgaagacgatcatcgccctgagctacatcttctgcctggtgttcgccgactacaaggacgatgatgacgccatggatccactgaatctgtcctggtatgatgatgatctggagaggcagaactggagccggcccttcaacgggtcagacgggaaggcggacagaccccactacaactactatgccacactgctcaccctgctcatcgctgtcatcgtcttcggcaacgtgctggtgtgcatggctgtgtcccgcgagaaggcgctgcagaccaccaccaactacctgatcgtcagcctcgcagtggccgacctcctcgtcgccacactggtcatgccctgggttgtctacctggaggtggtaggtgagtggaaattcagcaggattcactgtgacatcttcgtcactctggacgtcatgatgtgcacggcgagcatcctgaacttgtgtgccatcagcatcgcagcatacacagctgtggccatgcccatgctgtacaatacgcgctacagctccaagcgccgggtcaccgtcatgatctccatcgtctgggtcctgtccttcaccatctcctgcccactcctcttcggactcaataacgcagaccagaacgagtgcatcattgccaacccggccttcgtggtctactcctccatcgtctccttctacgtgcccttcattgtcaccctgctggtctacatcaagatctacattgtcctccgcagacgccgcaagcgagtcaacaccaaacgcagcagccgagctttcagggcccacctgagggctccactaaaggaggctgcccggcgagcccaggagctggagatggagatgctctccagcaccagcccacccgagaggacccggtacagccccatcccacccagccaccaccagctgactctccccgacccgtcccaccatggtctccacagcactcccgacagccccgccaaaccagagaagaatgggcatgccaaagaccaccccaagattgccaagatctttgagatccagaccatgcccaatggcaaaacgcgtacctccctcaagaccatgagccgtaggaagctctcccagcagaaggagaagaaagccactcagatgctcgccattgttctcggcgtgttcatcatctgctggctgcccttcttcatcacacacatcctgaacatacactgtgactgcaacatcccgcctgtcctgtacagcgccttcacgtggctgggctatgtcaacagcgccgtgaaccccatcatctacaccaccttcaacattgagttccgcaaggccttcctgaagatcctccactgtttcgaaatgactctggagtccatcatggcgtgctgcctgagcgaggaggccaaggaagcccggcggatcaacgacgagatcgagcggcagctccgcagggacaagcgggacgcccgccgggagctcaagctgctgctgctcgggacaggagagagtggcaagagtacgtttatcaagcagatgagaatcatccatgggtcaggatactctgatgaagataaaaggggcttcaccaagctggtgtatcagaacatcttcacggccatgcaggccatgatcagagccatggacacactcaagatcccatacaagtatgagcacaataaggctcatgcacaattagttcgagaagttgatgtggagaaggtgtctgcttttgagaatccatatgtagatgcaataaagagtttatggaatgatcctggaatccaggaatgctatgatagacgacgagaatatcaattatctgactctaccaaatactatcttaatgacttggaccgcgtagctgaccctgcctacctgcctacgcaacaagatgtgcttagagttcgagtccccaccacagggatcatcgaatacccctttgacttacaaagtgtcattttcagaatggtcgatgtagggggccaaaggtcagagagaagaaaatggatacactgctttgaaaatgtcacctctatcatgtttctagtagcgcttagtgaatatgatcaagttctcgtggagtcagacaatgagaaccgaatggaggaaagcaaggctctctttagaacaattatcacatacccctggttccagaactcctcggttattctgttcttaaacaagaaagatcttctagaggagaaaatcatgtattcccatctagtcgactacttcccagaatatgatggaccccagagagatgcccaggcagcccgagaattcattctgaagatgttcgtggacctgaacccagacagtgacaaaattatctactcccacttcacgtgcgccacagacaccgagaatatccgctttgtctttgctgccgtcaaggacaccatcctccagttgaacctgaaggatattggtctgttctaa

SEQ ID NO: 7 - DNA, 2400 bp, Artificial
  Synthetic Construct; SFD2S CAM (E339A/T343R) Gqi5
atgaagacgatcatcgccctgagctacatcttctgcctggtgttcgccgactacaaggacgatgatgacgccatggatccactgaatctgtcctggtatgatgatgatctggagaggcagaactggagccggcccttcaacgggtcagacgggaaggcggacagaccccactacaactactatgccacactgctcaccctgctcatcgctgtcatcgtcttcggcaacgtgctggtgtgcatggctgtgtcccgcgagaaggcgctgcagaccaccaccaactacctgatcgtcagcctcgcagtggccgacctcctcgtcgccacactggtcatgccctgggttgtctacctggaggtggtaggtgagtggaaattcagcaggattcactgtgacatcttcgtcactctggacgtcatgatgtgcacggcgagcatcctgaacttgtgtgccatcagcatcgacaggtacacagctgtggccatgcccatgctgtacaatacgcgctacagctccaagcgccgggtcaccgtcatgatctccatcgtctgggtcctgtccttcaccatctcctgcccactcctcttcggactcaataacgcagaccagaacgagtgcatcattgccaacccggccttcgtggtctactcctccatcgtctccttctacgtgcccttcattgtcaccctgctggtctacatcaagatctacattgtcctccgcagacgccgcaagcgagtcaacaccaaacgcagcagccgagctttcagggcccacctgagggctccactaaaggaggctgcccggcgagcccaggagctggagatggagatgctctccagcaccagcccacccgagaggacccggtacagccccatcccacccagccaccaccagctgactctccccgacccgtcccaccatggtctccacagcactcccgacagccccgccaaaccagagaagaatgggcatgccaaagaccaccccaagattgccaagatctttgagatccagaccatgcccaatggcaaaacgcgtacctccctcaagaccatgagccgtaggaagctctcccagcagaaggcaaagaaagctcgtcagatgctcgccattgttctcggcgtgttcatcatctgctggctgcccttcttcatcacacacatcctgaacatacactgtgactgcaacatcccgcctgtcctgtacagcgccttcacgtggctgggctatgtcaacagcgccgtgaaccccatcatctacaccaccttcaacattgagttccgcaaggccttcctgaagatcctccactgtttcgaaatgactctggagtccatcatggcgtgctgcctgagcgaggaggccaaggaagcccggcggatcaacgacgagatcgagcggcagctccgcagggacaagcgggacgcccgccgggagctcaagctgctgctgctcgggacaggagagagtggcaagagtacgtttatcaagcagatgagaatcatccatgggtcaggatactctgatgaagataaaaggggcttcaccaagctggtgtatcagaacatcttcacggccatgcaggccatgatcagagccatggacacactcaagatcccatacaagtatgagcacaataaggctcatgcacaattagttcgagaagttgatgtggagaaggtgtctgcttttgagaatccatatgtagatgcaataaagagtttatggaatgatcctggaatccaggaatgctatgatagacgacgagaatatcaattatctgactctaccaaatactatcttaatgacttggaccgcgtagctgaccctgcctacctgcctacgcaacaagatgtgcttagagttcgagtccccaccacagggatcatcgaatacccctttgacttacaaagtgtcattttcagaatggtcgatgtagggggccaaaggtcagagagaagaaaatggatacactgctttgaaaatgtcacctctatcatgtttctagtagcgcttagtgaatatgatcaagttctcgtggagtcagacaatgagaaccgaatggaggaaagcaaggctctctttagaacaattatcacatacccctggttccagaactcctcggttattctgttcttaaacaagaaagatcttctagaggagaaaatcatgtattcccatctagtcgactacttcccagaatatgatggaccccagagagatgcccaggcagcccgagaattcattctgaagatgttcgtggacctgaacccagacagtgacaaaattatctactcccacttcacgtgcgccacagacaccgagaatatccgctttgtctttgctgccgtcaaggacaccatcctccagttgaacctgaaggatattggtctgttctaa

SEQ ID NO: 8 - DNA, 2400 bp, Artificial
  Synthetic Construct; SFD2S CAM/N393A Gqi5
atgaagacgatcatcgccctgagctacatcttctgcctggtgttcgccgactacaaggacgatgatgacgccatggatccactgaatctgtcctggtatgatgatgatctggagaggcagaactggagccggcccttcaacgggtcagacgggaaggcggacagaccccactacaactactatgccacactgctcaccctgctcatcgctgtcatcgtcttcggcaacgtgctggtgtgcatggctgtgtcccgcgagaaggcgctgcagaccaccaccaactacctgatcgtcagcctcgcagtggccgacctcctcgtcgccacactggtcatgccctgggttgtctacctggaggtggtaggtgagtggaaattcagcaggattcactgtgacatcttcgtcactctggacgtcatgatgtgcacggcgagcatcctgaacttgtgtgccatcagcatcgacaggtacacagctgtggccatgcccatgctgtacaatacgcgctacagctccaagcgccgggtcaccgtcatgatctccatcgtctgggtcctgtccttcaccatctcctgcccactcctcttcggactcaataacgcagaccagaacgagtgcatcattgccaacccggccttcgtggtctactcctccatcgtctccttctacgtgcccttcattgtcaccctgctggtctacatcaagatctacattgtcctccgcagacgccgcaagcgagtcaacaccaaacgcagcagccgagctttcagggcccacctgagggctccactaaaggaggctgcccggcgagcccaggagctggagatggagatgctctccagcaccagcccacccgagaggacccggtacagccccatcccacccagccaccaccagctgactctccccgacccgtcccaccatggtctccacagcactcccgacagccccgccaaaccagagaagaatgggcatgccaaagaccaccccaagattgccaagatctttgagatccagaccatgcccaatggcaaaacgcgtacctccctcaagaccatgagccgtaggaagctctcccagcagaaggcaaagaaagctcgtcagatgctcgccattgttctcggcgtgttcatcatctgctggctgcccttcttcatcacacacatcctgaacatacactgtgactgcaacatcccgcctgtcctgtacagcgccttcacgtggctgggctatgtcaacagcgccgtggctcccatcatctacaccaccttcaacattgagttccgcaaggccttcctgaagatcctccactgtttcgaaatgactctggagtccatcatggcgtgctgcctgagcgaggaggccaaggaagcccggcggatcaacgacgagatcgagcggcagctccgcagggacaagcgggacgcccgccgggagctcaagctgctgctgctcgggacaggagagagtggcaagagtacgtttatcaagcagatgagaatcatccatgggtcaggatactctgatgaagataaaaggggcttcaccaagctggtgtatcagaacatcttcacggccatgcaggccatgatcagagccatggacacactcaagatcccatacaagtatgagcacaataaggctcatgcacaattagttcgagaagttgatgtggagaaggtgtctgcttttgagaatccatatgtagatgcaataaagagtttatggaatgatcctggaatccaggaatgctatgatagacgacgagaatatcaattatctgactctaccaaatactatcttaatgacttggaccgcgtagctgaccctgcctacctgcctacgcaacaagatgtgcttagagttcgagtccccaccacagggatcatcgaatacccctttgacttacaaagtgtcattttcagaatggtcgatgtagggggccaaaggtcagagagaagaaaatggatacactgctttgaaaatgtcacctctatcatgtttctagtagcgcttagtgaatatgatcaagttctcgtggagtcagacaatgagaaccgaatggaggaaagcaaggctctctttagaacaattatcacatacccctggttccagaactcctcggttattctgttcttaaacaagaaagatcttctagaggagaaaatcatgtattcccatctagtcgactacttcccagaatatgatggaccccagagagatgcccaggcagcccgagaattcattctgaagatgttcgtggacctgaacccagacagtgacaaaattatctactcccacttcacgtgcgccacagacaccgagaatatccgctttgtctttgctgccgtcaaggacaccatcctccagttgaacctgaaggatattggtctgttctaa

SEQ ID NO: 9 - DNA, 2400 bp, Artificial
  Synthetic Construct; SFD2S D114A Gqi5
atgaagacgatcatcgccctgagctacatcttctgcctggtgttcgccgactacaaggacgatgatgacgctgcggatccactgaatctgtcctggtatgatgatgatctggagaggcagaactggagccggcccttcaacgggtcagacgggaaggcggacagaccccactacaactactatgccacactgctcaccctgctcatcgctgtcatcgtcttcggcaacgtgctggtgtgcatggctgtgtcccgcgagaaggcgctgcagaccaccaccaactacctgatcgtcagcctcgcagtggccgacctcctcgtcgccacactggtcatgccctgggttgtctacctggaggtggtaggtgagtggaaattcagcaggattcactgtgacatcttcgtcactctggccgtcatgatgtgcacggcgagcatcctgaacttgtgtgccatcagcatcgacaggtacacagctgtggccatgcccatgctgtacaatacgcgctacagctccaagcgccgggtcaccgtcatgatctccatcgtctgggtcctgtccttcaccatctcctgcccactcctcttcggactcaataacgcagaccagaacgagtgcatcattgccaacccggccttcgtggtctactcctccatcgtctccttctacgtgcccttcattgtcaccctgctggtctacatcaagatctacattgtcctccgcagacgccgcaagcgagtcaacaccaaacgcagcagccgagctttcagggcccacctgagggctccactaaaggaggctgcccggcgagcccaggagctggagatggagatgctctccagcaccagcccacccgagaggacccggtacagccccatcccacccagccaccaccagctgactctccccgacccgtcccaccatggtctccacagcactcccgacagccccgccaaaccagagaagaatgggcatgccaaagaccaccccaagattgccaagatctttgagatccagaccatgcccaatggcaaaacgcgtacctccctcaagaccatgagccgtaggaagctctcccagcagaaggagaagaaagccactcagatgctcgccattgttctcggcgtgttcatcatctgctggctgcccttcttcatcacacacatcctgaacatacactgtgactgcaacatcccgcctgtcctgtacagcgccttcacgtggctgggctatgtcaacagcgccgtgaaccccatcatctacaccaccttcaacattgagttccgcaaggccttcctgaagatcctccactgtttcgaaatgactctggagtccatcatggcgtgctgcctgagcgaggaggccaaggaagcccggcggatcaacgacgagatcgagcggcagctccgcagggacaagcgggacgcccgccgggagctcaagctgctgctgctcgggacaggagagagtggcaagagtacgtttatcaagcagatgagaatcatccatgggtcaggatactctgatgaagataaaaggggcttcaccaagctggtgtatcagaacatcttcacggccatgcaggccatgatcagagccatggacacactcaagatcccatacaagtatgagcacaataaggctcatgcacaattagttcgagaagttgatgtggagaaggtgtctgcttttgagaatccatatgtagatgcaataaagagtttatggaatgatcctggaatccaggaatgctatgatagacgacgagaatatcaattatctgactctaccaaatactatcttaatgacttggaccgcgtagctgaccctgcctacctgcctacgcaacaagatgtgcttagagttcgagtccccaccacagggatcatcgaatacccctttgacttacaaagtgtcattttcagaatggtcgatgtagggggccaaaggtcagagagaagaaaatggatacactgctttgaaaatgtcacctctatcatgtttctagtagcgcttagtgaatatgatcaagttctcgtggagtcagacaatgagaaccgaatggaggaaagcaaggctctctttagaacaattatcacatacccctggttccagaactcctcggttattctgttcttaaacaagaaagatcttctagaggagaaaatcatgtattcccatctagtcgactacttcccagaatatgatggaccccagagagatgcccaggcagcccgagaattcattctgaagatgttcgtggacctgaacccagacagtgacaaaattatctactcccacttcacgtgcgccacagacaccgagaatatccgctttgtctttgctgccgtcaaggacaccatcctccagttgaacctgaaggatattggtctgttctaa

SEQ ID NO: 10 - DNA, 2400 bp, Artificial
  Synthetic Construct; SFD2S D114A/CAM Gqi5
atgaagacgatcatcgccctgagctacatcttctgcctggtgttcgccgactacaaggacgatgatgacgccatggatccactgaatctgtcctggtatgatgatgatctggagaggcagaactggagccggcccttcaacgggtcagacgggaaggcggacagaccccactacaactactatgccacactgctcaccctgctcatcgctgtcatcgtcttcggcaacgtgctggtgtgcatggctgtgtcccgcgagaaggcgctgcagaccaccaccaactacctgatcgtcagcctcgcagtggccgacctcctcgtcgccacactggtcatgccctgggttgtctacctggaggtggtaggtgagtggaaattcagcaggattcactgtgacatcttcgtcactctggccgtcatgatgtgcacggcgagcatcctgaacttgtgtgccatcagcatcgacaggtacacagctgtggccatgcccatgctgtacaatacgcgctacagctccaagcgccgggtcaccgtcatgatctccatcgtctgggtcctgtccttcaccatctcctgcccactcctcttcggactcaataacgcagaccagaacgagtgcatcattgccaacccggccttcgtggtctactcctccatcgtctccttctacgtgcccttcattgtcaccctgctggtctacatcaagatctacattgtcctccgcagacgccgcaagcgagtcaacaccaaacgcagcagccgagctttcagggcccacctgagggctccactaaaggaggctgcccggcgagcccaggagctggagatggagatgctctccagcaccagcccacccgagaggacccggtacagccccatcccacccagccaccaccagctgactctccccgacccgtcccaccatggtctccacagcactcccgacagccccgccaaaccagagaagaatgggcatgccaaagaccaccccaagattgccaagatctttgagatccagaccatgcccaatggcaaaacgcgtacctccctcaagaccatgagccgtaggaagctctcccagcagaaggcaaagaaagctcgtcagatgctcgccattgttctcggcgtgttcatcatctgctggctgcccttcttcatcacacacatcctgaacatacactgtgactgcaacatcccgcctgtcctgtacagcgccttcacgtggctgggctatgtcaacagcgccgtgaaccccatcatctacaccaccttcaacattgagttccgcaaggccttcctgaagatcctccactgtttcgaaatgactctggagtccatcatggcgtgctgcctgagcgaggaggccaaggaagcccggcggatcaacgacgagatcgagcggcagctccgcagggacaagcgggacgcccgccgggagctcaagctgctgctgctcgggacaggagagagtggcaagagtacgtttatcaagcagatgagaatcatccatgggtcaggatactctgatgaagataaaaggggcttcaccaagctggtgtatcagaacatcttcacggccatgcaggccatgatcagagccatggacacactcaagatcccatacaagtatgagcacaataaggctcatgcacaattagttcgagaagttgatgtggagaaggtgtctgcttttgagaatccatatgtagatgcaataaagagtttatggaatgatcctggaatccaggaatgctatgatagacgacgagaatatcaattatctgactctaccaaatactatcttaatgacttggaccgcgtagctgaccctgcctacctgcctacgcaacaagatgtgcttagagttcgagtccccaccacagggatcatcgaatacccctttgacttacaaagtgtcattttcagaatggtcgatgtagggggccaaaggtcagagagaagaaaatggatacactgctttgaaaatgtcacctctatcatgtttctagtagcgcttagtgaatatgatcaagttctcgtggagtcagacaatgagaaccgaatggaggaaagcaaggctctctttagaacaattatcacatacccctggttccagaactcctcggttattctgttcttaaacaagaaagatcttctagaggagaaaatcatgtattcccatctagtcgactacttcccagaatatgatggaccccagagagatgcccaggcagcccgagaattcattctgaagatgttcgtggacctgaacccagacagtgacaaaattatctactcccacttcacgtgcgccacagacaccgagaatatccgctttgtctttgctgccgtcaaggacaccatcctccagttgaacctgaaggatattggtctgttctaa

SEQ ID NO: 11 - DNA, 2400 bp, Artificial
  Synthetic Construct; SFD2S D80A Gqi5
atgaagacgatcatcgccctgagctacatcttctgcctggtgttcgccgactacaaggacgatgatgacgccatggatccactgaatctgtcctggtatgatgatgatctggagaggcagaactggagccggcccttcaacgggtcagacgggaaggcggacagaccccactacaactactatgccacactgctcaccctgctcatcgctgtcatcgtcttcggcaacgtgctggtgtgcatggctgtgtcccgcgagaaggcgctgcagaccaccaccaactacctgatcgtcagcctcgcagtggccgctctcctcgtcgccacactggtcatgccctgggttgtctacctggaggtggtaggtgagtggaaattcagcaggattcactgtgacatcttcgtcactctggacgtcatgatgtgcacggcgagcatcctgaacttgtgtgccatcagcatcgacaggtacacagctgtggccatgcccatgctgtacaatacgcgctacagctccaagcgccgggtcaccgtcatgatctccatcgtctgggtcctgtccttcaccatctcctgcccactcctcttcggactcaataacgcagaccagaacgagtgcatcattgccaacccggccttcgtggtctactcctccatcgtctccttctacgtgcccttcattgtcaccctgctggtctacatcaagatctacattgtcctccgcagacgccgcaagcgagtcaacaccaaacgcagcagccgagctttcagggcccacctgagggctccactaaaggaggctgcccggcgagcccaggagctggagatggagatgctctccagcaccagcccacccgagaggacccggtacagccccatcccacccagccaccaccagctgactctccccgacccgtcccaccatggtctccacagcactcccgacagccccgccaaaccagagaagaatgggcatgccaaagaccaccccaagattgccaagatctttgagatccagaccatgcccaatggcaaaacgcgtacctccctcaagaccatgagccgtaggaagctctcccagcagaaggagaagaaagccactcagatgctcgccattgttctcggcgtgttcatcatctgctggctgcccttcttcatcacacacatcctgaacatacactgtgactgcaacatcccgcctgtcctgtacagcgccttcacgtggctgggctatgtcaacagcgccgtgaaccccatcatctacaccaccttcaacattgagttccgcaaggccttcctgaagatcctccactgtttcgaaatgactctggagtccatcatggcgtgctgcctgagcgaggaggccaaggaagcccggcggatcaacgacgagatcgagcggcagctccgcagggacaagcgggacgcccgccgggagctcaagctgctgctgctcgggacaggagagagtggcaagagtacgtttatcaagcagatgagaatcatccatgggtcaggatactctgatgaagataaaaggggcttcaccaagctggtgtatcagaacatcttcacggccatgcaggccatgatcagagccatggacacactcaagatcccatacaagtatgagcacaataaggctcatgcacaattagttcgagaagttgatgtggagaaggtgtctgcttttgagaatccatatgtagatgcaataaagagtttatggaatgatcctggaatccaggaatgctatgatagacgacgagaatatcaattatctgactctaccaaatactatcttaatgacttggaccgcgtagctgaccctgcctacctgcctacgcaacaagatgtgcttagagttcgagtccccaccacagggatcatcgaatacccctttgacttacaaagtgtcattttcagaatggtcgatgtagggggccaaaggtcagagagaagaaaatggatacactgctttgaaaatgtcacctctatcatgtttctagtagcgcttagtgaatatgatcaagttctcgtggagtcagacaatgagaaccgaatggaggaaagcaaggctctctttagaacaattatcacatacccctggttccagaactcctcggttattctgttcttaaacaagaaagatcttctagaggagaaaatcatgtattcccatctagtcgactacttcccagaatatgatggaccccagagagatgcccaggcagcccgagaattcattctgaagatgttcgtggacctgaacccagacagtgacaaaattatctactcccacttcacgtgcgccacagacaccgagaatatccgctttgtctttgctgccgtcaaggacaccatcctccagttgaacctgaaggatattggtctgttctaa

SEQ ID NO: 12 - DNA, 2400 bp, Artificial
  Synthetic Construct; SFD2S D80A/CAM Gqi5
atgaagacgatcatcgccctgagctacatcttctgcctggtgttcgccgactacaaggacgatgatgacgccatggatccactgaatctgtcctggtatgatgatgatctggagaggcagaactggagccggcccttcaacgggtcagacgggaaggcggacagaccccactacaactactatgccacactgctcaccctgctcatcgctgtcatcgtcttcggcaacgtgctggtgtgcatggctgtgtcccgcgagaaggcgctgcagaccaccaccaactacctgatcgtcagcctcgcagtggccgctctcctcgtcgccacactggtcatgccctgggttgtctacctggaggtggtaggtgagtggaaattcagcaggattcactgtgacatcttcgtcactctggacgtcatgatgtgcacggcgagcatcctgaacttgtgtgccatcagcatcgacaggtacacagctgtggccatgcccatgctgtacaatacgcgctacagctccaagcgccgggtcaccgtcatgatctccatcgtctgggtcctgtccttcaccatctcctgcccactcctcttcggactcaataacgcagaccagaacgagtgcatcattgccaacccggccttcgtggtctactcctccatcgtctccttctacgtgcccttcattgtcaccctgctggtctacatcaagatctacattgtcctccgcagacgccgcaagcgagtcaacaccaaacgcagcagccgagctttcagggcccacctgagggctccactaaaggaggctgcccggcgagcccaggagctggagatggagatgctctccagcaccagcccacccgagaggacccggtacagccccatcccacccagccaccaccagctgactctccccgacccgtcccaccatggtctccacagcactcccgacagccccgccaaaccagagaagaatgggcatgccaaagaccaccccaagattgccaagatctttgagatccagaccatgcccaatggcaaaacgcgtacctccctcaagaccatgagccgtaggaagctctcccagcagaaggcaaagaaagctcgtcagatgctcgccattgttctcggcgtgttcatcatctgctggctgcccttcttcatcacacacatcctgaacatacactgtgactgcaacatcccgcctgtcctgtacagcgccttcacgtggctgggctatgtcaacagcgccgtgaaccccatcatctacaccaccttcaacattgagttccgcaaggccttcctgaagatcctccactgtttcgaaatgactctggagtccatcatggcgtgctgcctgagcgaggaggccaaggaagcccggcggatcaacgacgagatcgagcggcagctccgcagggacaagcgggacgcccgccgggagctcaagctgctgctgctcgggacaggagagagtggcaagagtacgtttatcaagcagatgagaatcatccatgggtcaggatactctgatgaagataaaaggggcttcaccaagctggtgtatcagaacatcttcacggccatgcaggccatgatcagagccatggacacactcaagatcccatacaagtatgagcacaataaggctcatgcacaattagttcgagaagttgatgtggagaaggtgtctgcttttgagaatccatatgtagatgcaataaagagtttatggaatgatcctggaatccaggaatgctatgatagacgacgagaatatcaattatctgactctaccaaatactatcttaatgacttggaccgcgtagctgaccctgcctacctgcctacgcaacaagatgtgcttagagttcgagtccccaccacagggatcatcgaatacccctttgacttacaaagtgtcattttcagaatggtcgatgtagggggccaaaggtcagagagaagaaaatggatacactgctttgaaaatgtcacctctatcatgtttctagtagcgcttagtgaatatgatcaagttctcgtggagtcagacaatgagaaccgaatggaggaaagcaaggctctctttagaacaattatcacatacccctggttccagaactcctcggttattctgttcttaaacaagaaagatcttctagaggagaaaatcatgtattcccatctagtcgactacttcccagaatatgatggaccccagagagatgcccaggcagcccgagaattcattctgaagatgttcgtggacctgaacccagacagtgacaaaattatctactcccacttcacgtgcgccacagacaccgagaatatccgctttgtctttgctgccgtcaaggacaccatcctccagttgaacctgaaggatattggtctgttctaa

SEQ ID NO: 13 - DNA, 2379 bp, Artificial
  Synthetic Construct; SFD2S delta213-219 Gqi5
atgaagacgatcatcgccctgagctacatcttctgcctggtgttcgccgactacaaggacgatgatgacgccatggatccactgaatctgtcctggtatgatgatgatctggagaggcagaactggagccggcccttcaacgggtcagacgggaaggcggacagaccccactacaactactatgccacactgctcaccctgctcatcgctgtcatcgtcttcggcaacgtgctggtgtgcatggctgtgtcccgcgagaaggcgctgcagaccaccaccaactacctgatcgtcagcctcgcagtggccgacctcctcgtcgccacactggtcatgccctgggttgtctacctggaggtggtaggtgagtggaaattcagcaggattcactgtgacatcttcgtcactctggacgtcatgatgtgcacggcgagcatcctgaacttgtgtgccatcagcatcgacaggtacacagctgtggccatgcccatgctgtacaatacgcgctacagctccaagcgccgggtcaccgtcatgatctccatcgtctgggtcctgtccttcaccatctcctgcccactcctcttcggactcaataacgcagaccagaacgagtgcatcattgccaacccggccttcgtggtctactcctccatcgtctccttctacgtgcccttcattgtcaccctgctggtctacatcaaaatacgcaagcgagtcaacaccaaacgcagcagccgagctttcagggcccacctgagggctccactaaaggaggctgcccggcgagcccaggagctggagatggagatgctctccagcaccagcccacccgagaggacccggtacagccccatcccacccagccaccaccagctgactctccccgacccgtcccaccatggtctccacagcactcccgacagccccgccaaaccagagaagaatgggcatgccaaagaccaccccaagattgccaagatctttgagatccagaccatgcccaatggcaaaacgcgtacctccctcaagaccatgagccgtaggaagctctcccagcagaaggagaagaaagccactcagatgctcgccattgttctcggcgtgttcatcatctgctggctgcccttcttcatcacacacatcctgaacatacactgtgactgcaacatcccgcctgtcctgtacagcgccttcacgtggctgggctatgtcaacagcgccgtgaaccccatcatctacaccaccttcaacattgagttccgcaaggccttcctgaagatcctccactgtttcgaaatgactctggagtccatcatggcgtgctgcctgagcgaggaggccaaggaagcccggcggatcaacgacgagatcgagcggcagctccgcagggacaagcgggacgcccgccgggagctcaagctgctgctgctcgggacaggagagagtggcaagagtacgtttatcaagcagatgagaatcatccatgggtcaggatactctgatgaagataaaaggggcttcaccaagctggtgtatcagaacatcttcacggccatgcaggccatgatcagagccatggacacactcaagatcccatacaagtatgagcacaataaggctcatgcacaattagttcgagaagttgatgtggagaaggtgtctgcttttgagaatccatatgtagatgcaataaagagtttatggaatgatcctggaatccaggaatgctatgatagacgacgagaatatcaattatctgactctaccaaatactatcttaatgacttggaccgcgtagctgaccctgcctacctgcctacgcaacaagatgtgcttagagttcgagtccccaccacagggatcatcgaatacccctttgacttacaaagtgtcattttcagaatggtcgatgtagggggccaaaggtcagagagaagaaaatggatacactgctttgaaaatgtcacctctatcatgtttctagtagcgcttagtgaatatgatcaagttctcgtggagtcagacaatgagaaccgaatggaggaaagcaaggctctctttagaacaattatcacatacccctggttccagaactcctcggttattctgttcttaaacaagaaagatcttctagaggagaaaatcatgtattcccatctagtcgactacttcccagaatatgatggaccccagagagatgcccaggcagcccgagaattcattctgaagatgttcgtggacctgaacccagacagtgacaaaattatctactcccacttcacgtgcgccacagacaccgagaatatccgctttgtctttgctgccgtcaaggacaccatcctccagttgaacctgaaggatattggtctgttctaa

SEQ ID NO: 14 - DNA, 2379 bp, Artificial
  Synthetic Construct; SFD2S delta213-219/CAM Gqi5
atgaagacgatcatcgccctgagctacatcttctgcctggtgttcgccgactacaaggacgatgatgacgccatggatccactgaatctgtcctggtatgatgatgatctggagaggcagaactggagccggcccttcaacgggtcagacgggaaggcggacagaccccactacaactactatgccacactgctcaccctgctcatcgctgtcatcgtcttcggcaacgtgctggtgtgcatggctgtgtcccgcgagaaggcgctgcagaccaccaccaactacctgatcgtcagcctcgcagtggccgacctcctcgtcgccacactggtcatgccctgggttgtctacctggaggtggtaggtgagtggaaattcagcaggattcactgtgacatcttcgtcactctggacgtcatgatgtgcacggcgagcatcctgaacttgtgtgccatcagcatcgacaggtacacagctgtggccatgcccatgctgtacaatacgcgctacagctccaagcgccgggtcaccgtcatgatctccatcgtctgggtcctgtccttcaccatctcctgcccactcctcttcggactcaataacgcagaccagaacgagtgcatcattgccaacccggccttcgtggtctactcctccatcgtctccttctacgtgcccttcattgtcaccctgctggtctacatcaaaatacgcaagcgagtcaacaccaaacgcagcagccgagctttcagggcccacctgagggctccactaaaggaggctgcccggcgagcccaggagctggagatggagatgctctccagcaccagcccacccgagaggacccggtacagccccatcccacccagccaccaccagctgactctccccgacccgtcccaccatggtctccacagcactcccgacagccccgccaaaccagagaagaatgggcatgccaaagaccaccccaagattgccaagatctttgagatccagaccatgcccaatggcaaaacgcgtacctccctcaagaccatgagccgtaggaagctctcccagcagaaggcaaagaaagctcgtcagatgctcgccattgttctcggcgtgttcatcatctgctggctgcccttcttcatcacacacatcctgaacatacactgtgactgcaacatcccgcctgtcctgtacagcgccttcacgtggctgggctatgtcaacagcgccgtgaaccccatcatctacaccaccttcaacattgagttccgcaaggccttcctgaagatcctccactgtttcgaaatgactctggagtccatcatggcgtgctgcctgagcgaggaggccaaggaagcccggcggatcaacgacgagatcgagcggcagctccgcagggacaagcgggacgcccgccgggagctcaagctgctgctgctcgggacaggagagagtggcaagagtacgtttatcaagcagatgagaatcatccatgggtcaggatactctgatgaagataaaaggggcttcaccaagctggtgtatcagaacatcttcacggccatgcaggccatgatcagagccatggacacactcaagatcccatacaagtatgagcacaataaggctcatgcacaattagttcgagaagttgatgtggagaaggtgtctgcttttgagaatccatatgtagatgcaataaagagtttatggaatgatcctggaatccaggaatgctatgatagacgacgagaatatcaattatctgactctaccaaatactatcttaatgacttggaccgcgtagctgaccctgcctacctgcctacgcaacaagatgtgcttagagttcgagtccccaccacagggatcatcgaatacccctttgacttacaaagtgtcattttcagaatggtcgatgtagggggccaaaggtcagagagaagaaaatggatacactgctttgaaaatgtcacctctatcatgtttctagtagcgcttagtgaatatgatcaagttctcgtggagtcagacaatgagaaccgaatggaggaaagcaaggctctctttagaacaattatcacatacccctggttccagaactcctcggttattctgttcttaaacaagaaagatcttctagaggagaaaatcatgtattcccatctagtcgactacttcccagaatatgatggaccccagagagatgcccaggcagcccgagaattcattctgaagatgttcgtggacctgaacccagacagtgacaaaattatctactcccacttcacgtgcgccacagacaccgagaatatccgctttgtctttgctgccgtcaaggacaccatcctccagttgaacctgaaggatattggtctgttctaa

SEQ ID NO: 15 - DNA, 2400 bp, Artificial
  Synthetic Construct; SFD2S N393A Gqi5
atgaagacgatcatcgccctgagctacatcttctgcctggtgttcgccgactacaaggacgatgatgacgccatggatccactgaatctgtcctggtatgatgatgatctggagaggcagaactggagccggcccttcaacgggtcagacgggaaggcggacagaccccactacaactactatgccacactgctcaccctgctcatcgctgtcatcgtcttcggcaacgtgctggtgtgcatggctgtgtcccgcgagaaggcgctgcagaccaccaccaactacctgatcgtcagcctcgcagtggccgacctcctcgtcgccacactggtcatgccctgggttgtctacctggaggtggtaggtgagtggaaattcagcaggattcactgtgacatcttcgtcactctggacgtcatgatgtgcacggcgagcatcctgaacttgtgtgccatcagcatcgacaggtacacagctgtggccatgcccatgctgtacaatacgcgctacagctccaagcgccgggtcaccgtcatgatctccatcgtctgggtcctgtccttcaccatctcctgcccactcctcttcggactcaataacgcagaccagaacgagtgcatcattgccaacccggccttcgtggtctactcctccatcgtctccttctacgtgcccttcattgtcaccctgctggtctacatcaagatctacattgtcctccgcagacgccgcaagcgagtcaacaccaaacgcagcagccgagctttcagggcccacctgagggctccactaaaggaggctgcccggcgagcccaggagctggagatggagatgctctccagcaccagcccacccgagaggacccggtacagccccatcccacccagccaccaccagctgactctccccgacccgtcccaccatggtctccacagcactcccgacagccccgccaaaccagagaagaatgggcatgccaaagaccaccccaagattgccaagatctttgagatccagaccatgcccaatggcaaaacgcgtacctccctcaagaccatgagccgtaggaagctctcccagcagaaggagaagaaagccactcagatgctcgccattgttctcggcgtgttcatcatctgctggctgcccttcttcatcacacacatcctgaacatacactgtgactgcaacatcccgcctgtcctgtacagcgccttcacgtggctgggctatgtcaacagcgccgtggctcccatcatctacaccaccttcaacattgagttccgcaaggccttcctgaagatcctccactgtttcgaaatgactctggagtccatcatggcgtgctgcctgagcgaggaggccaaggaagcccggcggatcaacgacgagatcgagcggcagctccgcagggacaagcgggacgcccgccgggagctcaagctgctgctgctcgggacaggagagagtggcaagagtacgtttatcaagcagatgagaatcatccatgggtcaggatactctgatgaagataaaaggggcttcaccaagctggtgtatcagaacatcttcacggccatgcaggccatgatcagagccatggacacactcaagatcccatacaagtatgagcacaataaggctcatgcacaattagttcgagaagttgatgtggagaaggtgtctgcttttgagaatccatatgtagatgcaataaagagtttatggaatgatcctggaatccaggaatgctatgatagacgacgagaatatcaattatctgactctaccaaatactatcttaatgacttggaccgcgtagctgaccctgcctacctgcctacgcaacaagatgtgcttagagttcgagtccccaccacagggatcatcgaatacccctttgacttacaaagtgtcattttcagaatggtcgatgtagggggccaaaggtcagagagaagaaaatggatacactgctttgaaaatgtcacctctatcatgtttctagtagcgcttagtgaatatgatcaagttctcgtggagtcagacaatgagaaccgaatggaggaaagcaaggctctctttagaacaattatcacatacccctggttccagaactcctcggttattctgttcttaaacaagaaagatcttctagaggagaaaatcatgtattcccatctagtcgactacttcccagaatatgatggaccccagagagatgcccaggcagcccgagaattcattctgaagatgttcgtggacctgaacccagacagtgacaaaattatctactcccacttcacgtgcgccacagacaccgagaatatccgctttgtctttgctgccgtcaaggacaccatcctccagttgaacctgaaggatattggtctgttctaa

SEQ ID NO: 16 - DNA, 2400 bp, Artificial
  Synthetic Construct
atgaagacgatcatcgccctgagctacatcttctgcctggtgttcgccgactacaaggacgatgatgacgctgcggatccactgaatctgtcctggtatgatgatgatctggagaggcagaactggagccggcccttcaacgggtcagacgggaaggcggacagaccccactacaactactatgccacactgctcaccctgctcatcgctgtcatcgtcttcggcaacgtgctggtgtgcatggctgtgtcccgcgagaaggcgctgcagaccaccaccaactacctgatcgtcagcctcgcagtggccgacctcctcgtcgccacactggtcatgccctgggttgtctacctggaggtggtaggtgagtggaaattcagcaggattcactgtgacatcttcgtcactctggacgtcatgatgtgcacggcgagcatcctgaacttgtgtgccatcagcatcgacgcttacacagctgtggccatgcccatgctgtacaatacgcgctacagctccaagcgccgggtcaccgtcatgatctccatcgtctgggtcctgtccttcaccatctcctgcccactcctcttcggactcaataacgcagaccagaacgagtgcatcattgccaacccggccttcgtggtctactcctccatcgtctccttctacgtgcccttcattgtcaccctgctggtctacatcaagatctacattgtcctccgcagacgccgcaagcgagtcaacaccaaacgcagcagccgagctttcagggcccacctgagggctccactaaaggaggctgcccggcgagcccaggagctggagatggagatgctctccagcaccagcccacccgagaggacccggtacagccccatcccacccagccaccaccagctgactctccccgacccgtcccaccatggtctccacagcactcccgacagccccgccaaaccagagaagaatgggcatgccaaagaccaccccaagattgccaagatctttgagatccagaccatgcccaatggcaaaacgcgtacctccctcaagaccatgagccgtaggaagctctcccagcagaaggagaagaaagccactcagatgctcgccattgttctcggcgtgttcatcatctgctggctgcccttcttcatcacacacatcctgaacatacactgtgactgcaacatcccgcctgtcctgtacagcgccttcacgtggctgggctatgtcaacagcgccgtgaaccccatcatctacaccaccttcaacattgagttccgcaaggccttcctgaagatcctccactgtttcgaaatgactctggagtccatcatggcgtgctgcctgagcgaggaggccaaggaagcccggcggatcaacgacgagatcgagcggcagctccgcagggacaagcgggacgcccgccgggagctcaagctgctgctgctcgggacaggagagagtggcaagagtacgtttatcaagcagatgagaatcatccatgggtcaggatactctgatgaagataaaaggggcttcaccaagctggtgtatcagaacatcttcacggccatgcaggccatgatcagagccatggacacactcaagatcccatacaagtatgagcacaataaggctcatgcacaattagttcgagaagttgatgtggagaaggtgtctgcttttgagaatccatatgtagatgcaataaagagtttatggaatgatcctggaatccaggaatgctatgatagacgacgagaatatcaattatctgactctaccaaatactatcttaatgacttggaccgcgtagctgaccctgcctacctgcctacgcaacaagatgtgcttagagttcgagtccccaccacagggatcatcgaatacccctttgacttacaaagtgtcattttcagaatggtcgatgtagggggccaaaggtcagagagaagaaaatggatacactgctttgaaaatgtcacctctatcatgtttctagtagcgcttagtgaatatgatcaagttctcgtggagtcagacaatgagaaccgaatggaggaaagcaaggctctctttagaacaattatcacatacccctggttccagaactcctcggttattctgttcttaaacaagaaagatcttctagaggagaaaatcatgtattcccatctagtcgactacttcccagaatatgatggaccccagagagatgcccaggcagcccgagaattcattctgaagatgttcgtggacctgaacccagacagtgacaaaattatctactcccacttcacgtgcgccacagacaccgagaatatccgctttgtctttgctgccgtcaaggacaccatcctccagttgaacctgaaggatattggtctgttctaa

SEQ ID NO: 17 - DNA, 2400 bp, Artificial
  Synthetic Construct; SFD2S R132A/CAM Gqi5
atgaagacgatcatcgccctgagctacatcttctgcctggtgttcgccgactacaaggacgatgatgacgccatggatccactgaatctgtcctggtatgatgatgatctggagaggcagaactggagccggcccttcaacgggtcagacgggaaggcggacagaccccactacaactactatgccacactgctcaccctgctcatcgctgtcatcgtcttcggcaacgtgctggtgtgcatggctgtgtcccgcgagaaggcgctgcagaccaccaccaactacctgatcgtcagcctcgcagtggccgacctcctcgtcgccacactggtcatgccctgggttgtctacctggaggtggtaggtgagtggaaattcagcaggattcactgtgacatcttcgtcactctggacgtcatgatgtgcacggcgagcatcctgaacttgtgtgccatcagcatcgacgcttacacagctgtggccatgcccatgctgtacaatacgcgctacagctccaagcgccgggtcaccgtcatgatctccatcgtctgggtcctgtccttcaccatctcctgcccactcctcttcggactcaataacgcagaccagaacgagtgcatcattgccaacccggccttcgtggtctactcctccatcgtctccttctacgtgcccttcattgtcaccctgctggtctacatcaagatctacattgtcctccgcagacgccgcaagcgagtcaacaccaaacgcagcagccgagctttcagggcccacctgagggctccactaaaggaggctgcccggcgagcccaggagctggagatggagatgctctccagcaccagcccacccgagaggacccggtacagccccatcccacccagccaccaccagctgactctccccgacccgtcccaccatggtctccacagcactcccgacagccccgccaaaccagagaagaatgggcatgccaaagaccaccccaagattgccaagatctttgagatccagaccatgcccaatggcaaaacgcgtacctccctcaagaccatgagccgtaggaagctctcccagcagaaggcaaagaaagctcgtcagatgctcgccattgttctcggcgtgttcatcatctgctggctgcccttcttcatcacacacatcctgaacatacactgtgactgcaacatcccgcctgtcctgtacagcgccttcacgtggctgggctatgtcaacagcgccgtgaaccccatcatctacaccaccttcaacattgagttccgcaaggccttcctgaagatcctccactgtttcgaaatgactctggagtccatcatggcgtgctgcctgagcgaggaggccaaggaagcccggcggatcaacgacgagatcgagcggcagctccgcagggacaagcgggacgcccgccgggagctcaagctgctgctgctcgggacaggagagagtggcaagagtacgtttatcaagcagatgagaatcatccatgggtcaggatactctgatgaagataaaaggggcttcaccaagctggtgtatcagaacatcttcacggccatgcaggccatgatcagagccatggacacactcaagatcccatacaagtatgagcacaataaggctcatgcacaattagttcgagaagttgatgtggagaaggtgtctgcttttgagaatccatatgtagatgcaataaagagtttatggaatgatcctggaatccaggaatgctatgatagacgacgagaatatcaattatctgactctaccaaatactatcttaatgacttggaccgcgtagctgaccctgcctacctgcctacgcaacaagatgtgcttagagttcgagtccccaccacagggatcatcgaatacccctttgacttacaaagtgtcattttcagaatggtcgatgtagggggccaaaggtcagagagaagaaaatggatacactgctttgaaaatgtcacctctatcatgtttctagtagcgcttagtgaatatgatcaagttctcgtggagtcagacaatgagaaccgaatggaggaaagcaaggctctctttagaacaattatcacatacccctggttccagaactcctcggttattctgttcttaaacaagaaagatcttctagaggagaaaatcatgtattcccatctagtcgactacttcccagaatatgatggaccccagagagatgcccaggcagcccgagaattcattctgaagatgttcgtggacctgaacccagacagtgacaaaattatctactcccacttcacgtgcgccacagacaccgagaatatccgctttgtctttgctgccgtcaaggacaccatcctccagttgaacctgaaggatattggtctgttctaa

SEQ ID NO: 18 - DNA, 2400 bp, Artificial
  Synthetic Construct; SFD2S V136D/M140E Gqi5
atgaagacgatcatcgccctgagctacatcttctgcctggtgttcgccgactacaaggacgatgatgacgccatggatccactgaatctgtcctggtatgatgatgatctggagaggcagaactggagccggcccttcaacgggtcagacgggaaggcggacagaccccactacaactactatgccacactgctcaccctgctcatcgctgtcatcgtcttcggcaacgtgctggtgtgcatggctgtgtcccgcgagaaggcgctgcagaccaccaccaactacctgatcgtcagcctcgcagtggccgacctcctcgtcgccacactggtcatgccctgggttgtctacctggaggtggtaggtgagtggaaattcagcaggattcactgtgacatcttcgtcactctggacgtcatgatgtgcacggcgagcatcctgaacttgtgtgccatcagcatcgacaggtacacagctgatgccatgcccgaactgtacaatacgcgctacagctccaagcgccgggtcaccgtcatgatctccatcgtctgggtcctgtccttcaccatctcctgcccactcctcttcggactcaataacgcagaccagaacgagtgcatcattgccaacccggccttcgtggtctactcctccatcgtctccttctacgtgcccttcattgtcaccctgctggtctacatcaagatctacattgtcctccgcagacgccgcaagcgagtcaacaccaaacgcagcagccgagctttcagggcccacctgagggctccactaaaggaggctgcccggcgagcccaggagctggagatggagatgctctccagcaccagcccacccgagaggacccggtacagccccatcccacccagccaccaccagctgactctccccgacccgtcccaccatggtctccacagcactcccgacagccccgccaaaccagagaagaatgggcatgccaaagaccaccccaagattgccaagatctttgagatccagaccatgcccaatggcaaaacgcgtacctccctcaagaccatgagccgtaggaagctctcccagcagaaggagaagaaagccactcagatgctcgccattgttctcggcgtgttcatcatctgctggctgcccttcttcatcacacacatcctgaacatacactgtgactgcaacatcccgcctgtcctgtacagcgccttcacgtggctgggctatgtcaacagcgccgtgaaccccatcatctacaccaccttcaacattgagttccgcaaggccttcctgaagatcctccactgtttcgaaatgactctggagtccatcatggcgtgctgcctgagcgaggaggccaaggaagcccggcggatcaacgacgagatcgagcggcagctccgcagggacaagcgggacgcccgccgggagctcaagctgctgctgctcgggacaggagagagtggcaagagtacgtttatcaagcagatgagaatcatccatgggtcaggatactctgatgaagataaaaggggcttcaccaagctggtgtatcagaacatcttcacggccatgcaggccatgatcagagccatggacacactcaagatcccatacaagtatgagcacaataaggctcatgcacaattagttcgagaagttgatgtggagaaggtgtctgcttttgagaatccatatgtagatgcaataaagagtttatggaatgatcctggaatccaggaatgctatgatagacgacgagaatatcaattatctgactctaccaaatactatcttaatgacttggaccgcgtagctgaccctgcctacctgcctacgcaacaagatgtgcttagagttcgagtccccaccacagggatcatcgaatacccctttgacttacaaagtgtcattttcagaatggtcgatgtagggggccaaaggtcagagagaagaaaatggatacactgctttgaaaatgtcacctctatcatgtttctagtagcgcttagtgaatatgatcaagttctcgtggagtcagacaatgagaaccgaatggaggaaagcaaggctctctttagaacaattatcacatacccctggttccagaactcctcggttattctgttcttaaacaagaaagatcttctagaggagaaaatcatgtattcccatctagtcgactacttcccagaatatgatggaccccagagagatgcccaggcagcccgagaattcattctgaagatgttcgtggacctgaacccagacagtgacaaaattatctactcccacttcacgtgcgccacagacaccgagaatatccgctttgtctttgctgccgtcaaggacaccatcctccagttgaacctgaaggatattggtctgttctaa

SEQ ID NO: 19 - DNA, 2400 bp, Artificial
  Synthetic Construct; SFD2S V136D/M140E/CAM Gqi5
atgaagacgatcatcgccctgagctacatcttctgcctggtgttcgccgactacaaggacgatgatgacgccatggatccactgaatctgtcctggtatgatgatgatctggagaggcagaactggagccggcccttcaacgggtcagacgggaaggcggacagaccccactacaactactatgccacactgctcaccctgctcatcgctgtcatcgtcttcggcaacgtgctggtgtgcatggctgtgtcccgcgagaaggcgctgcagaccaccaccaactacctgatcgtcagcctcgcagtggccgacctcctcgtcgccacactggtcatgccctgggttgtctacctggaggtggtaggtgagtggaaattcagcaggattcactgtgacatcttcgtcactctggacgtcatgatgtgcacggcgagcatcctgaacttgtgtgccatcagcatcgacaggtacacagctgatgccatgcccgaactgtacaatacgcgctacagctccaagcgccgggtcaccgtcatgatctccatcgtctgggtcctgtccttcaccatctcctgcccactcctcttcggactcaataacgcagaccagaacgagtgcatcattgccaacccggccttcgtggtctactcctccatcgtctccttctacgtgcccttcattgtcaccctgctggtctacatcaagatctacattgtcctccgcagacgccgcaagcgagtcaacaccaaacgcagcagccgagctttcagggcccacctgagggctccactaaaggaggctgcccggcgagcccaggagctggagatggagatgctctccagcaccagcccacccgagaggacccggtacagccccatcccacccagccaccaccagctgactctccccgacccgtcccaccatggtctccacagcactcccgacagccccgccaaaccagagaagaatgggcatgccaaagaccaccccaagattgccaagatctttgagatccagaccatgcccaatggcaaaacgcgtacctccctcaagaccatgagccgtaggaagctctcccagcagaaggcaaagaaagctcgtcagatgctcgccattgttctcggcgtgttcatcatctgctggctgcccttcttcatcacacacatcctgaacatacactgtgactgcaacatcccgcctgtcctgtacagcgccttcacgtggctgggctatgtcaacagcgccgtgaaccccatcatctacaccaccttcaacattgagttccgcaaggccttcctgaagatcctccactgtttcgaaatgactctggagtccatcatggcgtgctgcctgagcgaggaggccaaggaagcccggcggatcaacgacgagatcgagcggcagctccgcagggacaagcgggacgcccgccgggagctcaagctgctgctgctcgggacaggagagagtggcaagagtacgtttatcaagcagatgagaatcatccatgggtcaggatactctgatgaagataaaaggggcttcaccaagctggtgtatcagaacatcttcacggccatgcaggccatgatcagagccatggacacactcaagatcccatacaagtatgagcacaataaggctcatgcacaattagttcgagaagttgatgtggagaaggtgtctgcttttgagaatccatatgtagatgcaataaagagtttatggaatgatcctggaatccaggaatgctatgatagacgacgagaatatcaattatctgactctaccaaatactatcttaatgacttggaccgcgtagctgaccctgcctacctgcctacgcaacaagatgtgcttagagttcgagtccccaccacagggatcatcgaatacccctttgacttacaaagtgtcattttcagaatggtcgatgtagggggccaaaggtcagagagaagaaaatggatacactgctttgaaaatgtcacctctatcatgtttctagtagcgcttagtgaatatgatcaagttctcgtggagtcagacaatgagaaccgaatggaggaaagcaaggctctctttagaacaattatcacatacccctggttccagaactcctcggttattctgttcttaaacaagaaagatcttctagaggagaaaatcatgtattcccatctagtcgactacttcccagaatatgatggaccccagagagatgcccaggcagcccgagaattcattctgaagatgttcgtggacctgaacccagacagtgacaaaattatctactcccacttcacgtgcgccacagacaccgagaatatccgctttgtctttgctgccgtcaaggacaccatcctccagttgaacctgaaggatattggtctgttctaa

SEQ ID NO: 20 - DNA, 2400 bp, Artificial
  Synthetic Construct; SFD2SAAAA(IYIV212-215)Gqi5
atgaagacgatcatcgccctgagctacatcttctgcctggtgttcgccgactacaaggacgatgatgacgccatggatccactgaatctgtcctggtatgatgatgatctggagaggcagaactggagccggcccttcaacgggtcagacgggaaggcggacagaccccactacaactactatgccacactgctcaccctgctcatcgctgtcatcgtcttcggcaacgtgctggtgtgcatggctgtgtcccgcgagaaggcgctgcagaccaccaccaactacctgatcgtcagcctcgcagtggccgacctcctcgtcgccacactggtcatgccctgggttgtctacctggaggtggtaggtgagtggaaattcagcaggattcactgtgacatcttcgtcactctggacgtcatgatgtgcacggcgagcatcctgaacttgtgtgccatcagcatcgacaggtacacagctgtggccatgcccatgctgtacaatacgcgctacagctccaagcgccgggtcaccgtcatgatctccatcgtctgggtcctgtccttcaccatctcctgcccactcctcttcggactcaataacgcagaccagaacgagtgcatcattgccaacccggccttcgtggtctactcctccatcgtctccttctacgtgcccttcattgtcaccctgctggtctacatcaaggcagcagcagcactccgcagacgccgcaagcgagtcaacaccaaacgcagcagccgagctttcagggcccacctgagggctccactaaaggaggctgcccggcgagcccaggagctggagatggagatgctctccagcaccagcccacccgagaggacccggtacagccccatcccacccagccaccaccagctgactctccccgacccgtcccaccatggtctccacagcactcccgacagccccgccaaaccagagaagaatgggcatgccaaagaccaccccaagattgccaagatctttgagatccagaccatgcccaatggcaaaacgcgtacctccctcaagaccatgagccgtaggaagctctcccagcagaaggagaagaaagccactcagatgctcgccattgttctcggcgtgttcatcatctgctggctgcccttcttcatcacacacatcctgaacatacactgtgactgcaacatcccgcctgtcctgtacagcgccttcacgtggctgggctatgtcaacagcgccgtgaaccccatcatctacaccaccttcaacattgagttccgcaaggccttcctgaagatcctccactgtttcgaaatgactctggagtccatcatggcgtgctgcctgagcgaggaggccaaggaagcccggcggatcaacgacgagatcgagcggcagctccgcagggacaagcgggacgcccgccgggagctcaagctgctgctgctcgggacaggagagagtggcaagagtacgtttatcaagcagatgagaatcatccatgggtcaggatactctgatgaagataaaaggggcttcaccaagctggtgtatcagaacatcttcacggccatgcaggccatgatcagagccatggacacactcaagatcccatacaagtatgagcacaataaggctcatgcacaattagttcgagaagttgatgtggagaaggtgtctgcttttgagaatccatatgtagatgcaataaagagtttatggaatgatcctggaatccaggaatgctatgatagacgacgagaatatcaattatctgactctaccaaatactatcttaatgacttggaccgcgtagctgaccctgcctacctgcctacgcaacaagatgtgcttagagttcgagtccccaccacagggatcatcgaatacccctttgacttacaaagtgtcattttcagaatggtcgatgtagggggccaaaggtcagagagaagaaaatggatacactgctttgaaaatgtcacctctatcatgtttctagtagcgcttagtgaatatgatcaagttctcgtggagtcagacaatgagaaccgaatggaggaaagcaaggctctctttagaacaattatcacatacccctggttccagaactcctcggttattctgttcttaaacaagaaagatcttctagaggagaaaatcatgtattcccatctagtcgactacttcccagaatatgatggaccccagagagatgcccaggcagcccgagaattcattctgaagatgttcgtggacctgaacccagacagtgacaaaattatctactcccacttcacgtgcgccacagacaccgagaatatccgctttgtctttgctgccgtcaaggacaccatcctccagttgaacctgaaggatattggtctgttctaa

SEQ ID NO: 21 - DNA, 2400 bp, Artificial
  Synthetic Construct; SFD2SWT Gqi5
atgaagacgatcatcgccctgagctacatcttctgcctggtgttcgccgactacaaggacgatgatgacgccatggatccactgaatctgtcctggtatgatgatgatctggagaggcagaactggagccggcccttcaacgggtcagacgggaaggcggacagaccccactacaactactatgccacactgctcaccctgctcatcgctgtcatcgtcttcggcaacgtgctggtgtgcatggctgtgtcccgcgagaaggcgctgcagaccaccaccaactacctgatcgtcagcctcgcagtggccgacctcctcgtcgccacactggtcatgccctgggttgtctacctggaggtggtaggtgagtggaaattcagcaggattcactgtgacatcttcgtcactctggacgtcatgatgtgcacggcgagcatcctgaacttgtgtgccatcagcatcgacaggtacacagctgtggccatgcccatgctgtacaatacgcgctacagctccaagcgccgggtcaccgtcatgatctccatcgtctgggtcctgtccttcaccatctcctgcccactcctcttcggactcaataacgcagaccagaacgagtgcatcattgccaacccggccttcgtggtctactcctccatcgtctccttctacgtgcccttcattgtcaccctgctggtctacatcaagatctacattgtcctccgcagacgccgcaagcgagtcaacaccaaacgcagcagccgagctttcagggcccacctgagggctccactaaaggaggctgcccggcgagcccaggagctggagatggagatgctctccagcaccagcccacccgagaggacccggtacagccccatcccacccagccaccaccagctgactctccccgacccgtcccaccatggtctccacagcactcccgacagccccgccaaaccagagaagaatgggcatgccaaagaccaccccaagattgccaagatctttgagatccagaccatgcccaatggcaaaacgcgtacctccctcaagaccatgagccgtaggaagctctcccagcagaaggagaagaaagccactcagatgctcgccattgttctcggcgtgttcatcatctgctggctgcccttcttcatcacacacatcctgaacatacactgtgactgcaacatcccgcctgtcctgtacagcgccttcacgtggctgggctatgtcaacagcgccgtgaaccccatcatctacaccaccttcaacattgagttccgcaaggccttcctgaagatcctccactgtttcgaaatgactctggagtccatcatggcgtgctgcctgagcgaggaggccaaggaagcccggcggatcaacgacgagatcgagcggcagctccgcagggacaagcgggacgcccgccgggagctcaagctgctgctgctcgggacaggagagagtggcaagagtacgtttatcaagcagatgagaatcatccatgggtcaggatactctgatgaagataaaaggggcttcaccaagctggtgtatcagaacatcttcacggccatgcaggccatgatcagagccatggacacactcaagatcccatacaagtatgagcacaataaggctcatgcacaattagttcgagaagttgatgtggagaaggtgtctgcttttgagaatccatatgtagatgcaataaagagtttatggaatgatcctggaatccaggaatgctatgatagacgacgagaatatcaattatctgactctaccaaatactatcttaatgacttggaccgcgtagctgaccctgcctacctgcctacgcaacaagatgtgcttagagttcgagtccccaccacagggatcatcgaatacccctttgacttacaaagtgtcattttcagaatggtcgatgtagggggccaaaggtcagagagaagaaaatggatacactgctttgaaaatgtcacctctatcatgtttctagtagcgcttagtgaatatgatcaagttctcgtggagtcagacaatgagaaccgaatggaggaaagcaaggctctctttagaacaattatcacatacccctggttccagaactcctcggttattctgttcttaaacaagaaagatcttctagaggagaaaatcatgtattcccatctagtcgactacttcccagaatatgatggaccccagagagatgcccaggcagcccgagaattcattctgaagatgttcgtggacctgaacccagacagtgacaaaattatctactcccacttcacgtgcgccacagacaccgagaatatccgctttgtctttgctgccgtcaaggacaccatcctccagttgaacctgaaggatattggtctgttctaa

SEQ ID NO: 22 - DNA, 1407 bp, Artificial
  Synthetic Construct; smycD2/D4L
atgaagacgatcatcgccctgagctacatcttctgcctggtgttcgccgagcagaagctgatctccgaagaggacctggatccactgaatctgtcctggtatgatgatgatctggagaggcagaactggagccggcccttcaacgggtcagacgggaaggcggacagaccccactacaactactatgccacactgctcaccctgctcatcgctgtcatcgtcttcggcaacgtgctggtgtgcatggctgtgtcccgcgagaaggcgctgcagaccaccaccaactacctgatcgtcagcctcgcagtggccgacctcctcgtcgccacactggtcatgccttggtttgtatacctggaggtggtaggtgagtggaaattcagcaggattcactgtgacatcttaatgactctggacgtcatgatgtgcacggcgagcatcctgaacttgtgtgccatcagcatcgacaggtacacagctgtggccatgcccatgctgtacaatacgcgctacagctccaagcgccgggtcaccgtcatgatctccatcgtctgggtcctgtccttcaccatctcctgcccactcctcttcggactcaataacgcagaccagaacgagtgcatcattgccaacccggccttcgtggtctactcctccatcgtctccttctacgtgcccttcattgtcaccctgctggtctacatcaagatctacattgtcctccgcagacgccgcaagcgagtcaacaccaaacgcagcagccgagctttcagggcccacctgagggctccactaaagggcaactgtactcaccccgaggacatgaaactctgcaccgttatcatgaagtctaatgggagtttcccagtgaacaggcggagagtggaggctgcccggcgagcccaggagctggagatggagatgctctccagcaccagcccacccgagaggacccggtacagccccatcccacccagccaccaccagctgactctccccgacccgtcccaccatggtctccacagcactcccgacagccccgccaaaccagagaagaatgggcatgccaaagaccaccccaagattgccaagatctttgagatccagaccatgcccaatggcaaaacgcgtacctccctcaagaccatgagccgtaggaagctctcccagcagaaggagaagaaagccactcagatgctcgccattgttctcggcgtgttcatcatctgctggctgcccttcttcatcacacacatcctgaacatacactgtgactgcaacatcccgcctgtcctggttagcgccttcacgtggctgggctatgtcaacagcgccgtgaaccccatcatctacaccaccttcaacattgagttccgcaaggccttcctgaagatcctccactgttga

SEQ ID NO: 23 - DNA, 1320 bp, Artificial
  Synthetic Construct; smycD2/D4S
atgaagacgatcatcgccctgagctacatcttctgcctggtgttcgccgagcagaagctgatctccgaagaggacctggatccactgaatctgtcctggtatgatgatgatctggagaggcagaactggagccggcccttcaacgggtcagacgggaaggcggacagaccccactacaactactatgccacactgctcaccctgctcatcgctgtcatcgtcttcggcaacgtgctggtgtgcatggctgtgtcccgcgagaaggcgctgcagaccaccaccaactacctgatcgtcagcctcgcagtggccgacctcctcgtcgccacactggtcatgccttggtttgtatacctggaggtggtaggtgagtggaaattcagcaggattcactgtgacatcttaatgactctggacgtcatgatgtgcacggcgagcatcctgaacttgtgtgccatcagcatcgacaggtacacagctgtggccatgcccatgctgtacaatacgcgctacagctccaagcgccgggtcaccgtcatgatctccatcgtctgggtcctgtccttcaccatctcctgcccactcctcttcggactcaataacgcagaccagaacgagtgcatcattgccaacccggccttcgtggtctactcctccatcgtctccttctacgtgcccttcattgtcaccctgctggtctacatcaagatctacattgtcctccgcagacgccgcaagcgagtcaacaccaaacgcagcagccgagctttcagggcccacctgagggctccactaaaggaggctgcccggcgagcccaggagctggagatggagatgctctccagcaccagcccacccgagaggacccggtacagccccatcccacccagccaccaccagctgactctccccgacccgtcccaccatggtctccacagcactcccgacagccccgccaaaccagagaagaatgggcatgccaaagaccaccccaagattgccaagatctttgagatccagaccatgcccaatggcaaaacgcgtacctccctcaagaccatgagccgtaggaagctctcccagcagaaggagaagaaagccactcagatgctcgccattgttctcggcgtgttcatcatctgctggctgcccttcttcatcacacacatcctgaacatacactgtgactgcaacatcccgcctgtcctggttagcgccttcacgtggctgggctatgtcaacagcgccgtgaaccccatcatctacaccaccttcaacattgagttccgcaaggccttcctgaagatcctccactgttga

SEQ ID NO: 24 - DNA, 1320 bp, Artificial
  Synthetic Construct; smycD2SWT
atgaagacgatcatcgccctgagctacatcttctgcctggtgttcgccgagcagaagctgatctccgaagaggacctggatccactgaatctgtcctggtatgatgatgatctggagaggcagaactggagccggcccttcaacgggtcagacgggaaggcggacagaccccactacaactactatgccacactgctcaccctgctcatcgctgtcatcgtcttcggcaacgtgctggtgtgcatggctgtgtcccgcgagaaggcgctgcagaccaccaccaactacctgatcgtcagcctcgcagtggccgacctcctcgtcgccacactggtcatgccctgggttgtctacctggaggtggtaggtgagtggaaattcagcaggattcactgtgacatcttcgtcactctggacgtcatgatgtgcacggcgagcatcctgaacttgtgtgccatcagcatcgacaggtacacagctgtggccatgcccatgctgtacaatacgcgctacagctccaagcgccgggtcaccgtcatgatctccatcgtctgggtcctgtccttcaccatctcctgcccactcctcttcggactcaataacgcagaccagaacgagtgcatcattgccaacccggccttcgtggtctactcctccatcgtctccttctacgtgcccttcattgtcaccctgctggtctacatcaagatctacattgtcctccgcagacgccgcaagcgagtcaacaccaaacgcagcagccgagctttcagggcccacctgagggctccactaaaggaggctgcccggcgagcccaggagctggagatggagatgctctccagcaccagcccacccgagaggacccggtacagccccatcccacccagccaccaccagctgactctccccgacccgtcccaccatggtctccacagcactcccgacagccccgccaaaccagagaagaatgggcatgccaaagaccaccccaagattgccaagatctttgagatccagaccatgcccaatggcaaaacgcgtacctccctcaagaccatgagccgtaggaagctctcccagcagaaggagaagaaagccactcagatgctcgccattgttctcggcgtgttcatcatctgctggctgcccttcttcatcacacacatcctgaacatacactgtgactgcaacatcccgcctgtcctgtacagcgccttcacgtggctgggctatgtcaacagcgccgtgaaccccatcatctacaccaccttcaacattgagttccgcaaggccttcctgaagatcctccactgttga

SEQ ID NO: 25 - DNA, 1320 bp, Artificial
  Synthetic Construct; smycD2S AAAA(RRKR219-222)
atgaagacgatcatcgccctgagctacatcttctgcctggtgttcgccgagcagaagctgatctccgaagaggacctggatccactgaatctgtcctggtatgatgatgatctggagaggcagaactggagccggcccttcaacgggtcagacgggaaggcggacagaccccactacaactactatgccacactgctcaccctgctcatcgctgtcatcgtcttcggcaacgtgctggtgtgcatggctgtgtcccgcgagaaggcgctgcagaccaccaccaactacctgatcgtcagcctcgcagtggccgacctcctcgtcgccacactggtcatgccctgggttgtctacctggaggtggtaggtgagtggaaattcagcaggattcactgtgacatcttcgtcactctggacgtcatgatgtgcacggcgagcatcctgaacttgtgtgccatcagcatcgacaggtacacagctgtggccatgcccatgctgtacaatacgcgctacagctccaagcgccgggtcaccgtcatgatctccatcgtctgggtcctgtccttcaccatctcctgcccactcctcttcggactcaataacgcagaccagaacgagtgcatcattgccaacccggccttcgtggtctactcctccatcgtctccttctacgtgcccttcattgtcaccctgctggtctacatcaagatctacattgtcctccgcagagcagcagcagcagtcaacaccaaacgcagcagccgagctttcagggcccacctgagggctccactaaaggaggctgcccggcgagcccaggagctggagatggagatgctctccagcaccagcccacccgagaggacccggtacagccccatcccacccagccaccaccagctgactctccccgacccgtcccaccatggtctccacagcactcccgacagccccgccaaaccagagaagaatgggcatgccaaagaccaccccaagattgccaagatctttgagatccagaccatgcccaatggcaaaacgcgtacctccctcaagaccatgagccgtaggaagctctcccagcagaaggagaagaaagccactcagatgctcgccattgttctcggcgtgttcatcatctgctggctgcccttcttcatcacacacatcctgaacatacactgtgactgcaacatcccgcctgtcctgtacagcgccttcacgtggctgggctatgtcaacagcgccgtgaaccccatcatctacaccaccttcaacattgagttccgcaaggccttcctgaagatcctccactgttga

SEQ ID NO: 26 - DNA, 1320 bp, Artificial
  Synthetic Construct; smycD2S D131A/R132A
atgaagacgatcatcgccctgagctacatcttctgcctggtgttcgccgagcagaagctgatctccgaagaggacctggatccactgaatctgtcctggtatgatgatgatctggagaggcagaactggagccggcccttcaacgggtcagacgggaaggcggacagaccccactacaactactatgccacactgctcaccctgctcatcgctgtcatcgtcttcggcaacgtgctggtgtgcatggctgtgtcccgcgagaaggcgctgcagaccaccaccaactacctgatcgtcagcctcgcagtggccgacctcctcgtcgccacactggtcatgccctgggttgtctacctggaggtggtaggtgagtggaaattcagcaggattcactgtgacatcttcgtcactctggacgtcatgatgtgcacggcgagcatcctgaacttgtgtgccatcagcatcgcagcatacacagctgtggccatgcccatgctgtacaatacgcgctacagctccaagcgccgggtcaccgtcatgatctccatcgtctgggtcctgtccttcaccatctcctgcccactcctcttcggactcaataacgcagaccagaacgagtgcatcattgccaacccggccttcgtggtctactcctccatcgtctccttctacgtgcccttcattgtcaccctgctggtctacatcaagatctacattgtcctccgcagacgccgcaagcgagtcaacaccaaacgcagcagccgagctttcagggcccacctgagggctccactaaaggaggctgcccggcgagcccaggagctggagatggagatgctctccagcaccagcccacccgagaggacccggtacagccccatcccacccagccaccaccagctgactctccccgacccgtcccaccatggtctccacagcactcccgacagccccgccaaaccagagaagaatgggcatgccaaagaccaccccaagattgccaagatctttgagatccagaccatgcccaatggcaaaacgcgtacctccctcaagaccatgagccgtaggaagctctcccagcagaaggagaagaaagccactcagatgctcgccattgttctcggcgtgttcatcatctgctggctgcccttcttcatcacacacatcctgaacatacactgtgactgcaacatcccgcctgtcctgtacagcgccttcacgtggctgggctatgtcaacagcgccgtgaaccccatcatctacaccaccttcaacattgagttccgcaaggccttcctgaagatcctccactgttga

SEQ ID NO: 27 - DNA, 1320 bp, Artificial
  Synthetic Construct; smycD2S CAM
atgaagacgatcatcgccctgagctacatcttctgcctggtgttcgccgagcagaagctgatctccgaagaggacctggatccactgaatctgtcctggtatgatgatgatctggagaggcagaactggagccggcccttcaacgggtcagacgggaaggcggacagaccccactacaactactatgccacactgctcaccctgctcatcgctgtcatcgtcttcggcaacgtgctggtgtgcatggctgtgtcccgcgagaaggcgctgcagaccaccaccaactacctgatcgtcagcctcgcagtggccgacctcctcgtcgccacactggtcatgccctgggttgtctacctggaggtggtaggtgagtggaaattcagcaggattcactgtgacatcttcgtcactctggacgtcatgatgtgcacggcgagcatcctgaacttgtgtgccatcagcatcgacaggtacacagctgtggccatgcccatgctgtacaatacgcgctacagctccaagcgccgggtcaccgtcatgatctccatcgtctgggtcctgtccttcaccatctcctgcccactcctcttcggactcaataacgcagaccagaacgagtgcatcattgccaacccggccttcgtggtctactcctccatcgtctccttctacgtgcccttcattgtcaccctgctggtctacatcaagatctacattgtcctccgcagacgccgcaagcgagtcaacaccaaacgcagcagccgagctttcagggcccacctgagggctccactaaaggaggctgcccggcgagcccaggagctggagatggagatgctctccagcaccagcccacccgagaggacccggtacagccccatcccacccagccaccaccagctgactctccccgacccgtcccaccatggtctccacagcactcccgacagccccgccaaaccagagaagaatgggcatgccaaagaccaccccaagattgccaagatctttgagatccagaccatgcccaatggcaaaacgcgtacctccctcaagaccatgagccgtaggaagctctcccagcagaaggcaaagaaagcccgtcagatgctcgccattgttctcggcgtgttcatcatctgctggctgcccttcttcatcacacacatcctgaacatacactgtgactgcaacatcccgcctgtcctgtacagcgccttcacgtggctgggctatgtcaacagcgccgtgaaccccatcatctacaccaccttcaacattgagttccgcaaggccttcctgaagatcctccactgttga

SEQ ID NO: 28 - DNA, 1320 bp, Artificial
  Synthetic Construct; smycD2S D114A
atgaagacgatcatcgccctgagctacatcttctgcctggtgttcgccgagcagaagctgatctccgaagaggacctggatccactgaatctgtcctggtatgatgatgatctggagaggcagaactggagccggcccttcaacgggtcagacgggaaggcggacagaccccactacaactactatgccacactgctcaccctgctcatcgctgtcatcgtcttcggcaacgtgctggtgtgcatggctgtgtcccgcgagaaggcgctgcagaccaccaccaactacctgatcgtcagcctcgcagtggccgacctcctcgtcgccacactggtcatgccctgggttgtctacctggaggtggtaggtgagtggaaattcagcaggattcactgtgacatcttcgtcactctggccgtcatgatgtgcacggcgagcatcctgaacttgtgtgccatcagcatcgacaggtacacagctgtggccatgcccatgctgtacaatacgcgctacagctccaagcgccgggtcaccgtcatgatctccatcgtctgggtcctgtccttcaccatctcctgcccactcctcttcggactcaataacgcagaccagaacgagtgcatcattgccaacccggccttcgtggtctactcctccatcgtctccttctacgtgcccttcattgtcaccctgctggtctacatcaagatctacattgtcctccgcagacgccgcaagcgagtcaacaccaaacgcagcagccgagctttcagggcccacctgagggctccactaaaggaggctgcccggcgagcccaggagctggagatggagatgctctccagcaccagcccacccgagaggacccggtacagccccatcccacccagccaccaccagctgactctccccgacccgtcccaccatggtctccacagcactcccgacagccccgccaaaccagagaagaatgggcatgccaaagaccaccccaagattgccaagatctttgagatccagaccatgcccaatggcaaaacgcgtacctccctcaagaccatgagccgtaggaagctctcccagcagaaggagaagaaagccactcagatgctcgccattgttctcggcgtgttcatcatctgctggctgcccttcttcatcacacacatcctgaacatacactgtgactgcaacatcccgcctgtcctgtacagcgccttcacgtggctgggctatgtcaacagcgccgtgaaccccatcatctacaccaccttcaacattgagttccgcaaggccttcctgaagatcctccactgttga

SEQ ID NO: 29 - DNA, 1320 bp, Artificial
  Synthetic Construct; smycD2S D80A
atgaagacgatcatcgccctgagctacatcttctgcctggtgttcgccgagcagaagctgatctccgaagaggacctggatccactgaatctgtcctggtatgatgatgatctggagaggcagaactggagccggcccttcaacgggtcagacgggaaggcggacagaccccactacaactactatgccacactgctcaccctgctcatcgctgtcatcgtcttcggcaacgtgctggtgtgcatggctgtgtcccgcgagaaggcgctgcagaccaccaccaactacctgatcgtcagcctcgcagtggccgctctcctcgtcgccacactggtcatgccctgggttgtctacctggaggtggtaggtgagtggaaattcagcaggattcactgtgacatcttcgtcactctggacgtcatgatgtgcacggcgagcatcctgaacttgtgtgccatcagcatcgacaggtacacagctgtggccatgcccatgctgtacaatacgcgctacagctccaagcgccgggtcaccgtcatgatctccatcgtctgggtcctgtccttcaccatctcctgcccactcctcttcggactcaataacgcagaccagaacgagtgcatcattgccaacccggccttcgtggtctactcctccatcgtctccttctacgtgcccttcattgtcaccctgctggtctacatcaagatctacattgtcctccgcagacgccgcaagcgagtcaacaccaaacgcagcagccgagctttcagggcccacctgagggctccactaaaggaggctgcccggcgagcccaggagctggagatggagatgctctccagcaccagcccacccgagaggacccggtacagccccatcccacccagccaccaccagctgactctccccgacccgtcccaccatggtctccacagcactcccgacagccccgccaaaccagagaagaatgggcatgccaaagaccaccccaagattgccaagatctttgagatccagaccatgcccaatggcaaaacgcgtacctccctcaagaccatgagccgtaggaagctctcccagcagaaggagaagaaagccactcagatgctcgccattgttctcggcgtgttcatcatctgctggctgcccttcttcatcacacacatcctgaacatacactgtgactgcaacatcccgcctgtcctgtacagcgccttcacgtggctgggctatgtcaacagcgccgtgaaccccatcatctacaccaccttcaacattgagttccgcaaggccttcctgaagatcctccactgctga

SEQ ID NO: 30 - DNA, 1299 bp, Artificial
  Synthetic Construct; smycD2S delta213-219
atgaagacgatcatcgccctgagctacatcttctgcctggtgttcgccgagcagaagctgatctccgaagaggacctggatccactgaatctgtcctggtatgatgatgatctggagaggcagaactggagccggcccttcaacgggtcagacgggaaggcggacagaccccactacaactactatgccacactgctcaccctgctcatcgctgtcatcgtcttcggcaacgtgctggtgtgcatggctgtgtcccgcgagaaggcgctgcagaccaccaccaactacctgatcgtcagcctcgcagtggccgacctcctcgtcgccacactggtcatgccctgggttgtctacctggaggtggtaggtgagtggaaattcagcaggattcactgtgacatcttcgtcactctggacgtcatgatgtgcacggcgagcatcctgaacttgtgtgccatcagcatcgacaggtacacagctgtggccatgcccatgctgtacaatacgcgctacagctccaagcgccgggtcaccgtcatgatctccatcgtctgggtcctgtccttcaccatctcctgcccactcctcttcggactcaataacgcagaccagaacgagtgcatcattgccaacccggccttcgtggtctactcctccatcgtctccttctacgtgcccttcattgtcaccctgctggtctacatcaaaatacgcaagcgagtcaacaccaaacgcagcagccgagctttcagggcccacctgagggctccactaaaggaggctgcccggcgagcccaggagctggagatggagatgctctccagcaccagcccacccgagaggacccggtacagccccatcccacccagccaccaccagctgactctccccgacccgtcccaccatggtctccacagcactcccgacagccccgccaaaccagagaagaatgggcatgccaaagaccaccccaagattgccaagatctttgagatccagaccatgcccaatggcaaaacgcgtacctccctcaagaccatgagccgtaggaagctctcccagcagaaggagaagaaagccactcagatgctcgccattgttctcggcgtgttcatcatctgctggctgcccttcttcatcacacacatcctgaacatacactgtgactgcaacatcccgcctgtcctgtacagcgccttcacgtggctgggctatgtcaacagcgccgtgaaccccatcatctacaccaccttcaacattgagttccgcaaggccttcctgaagatcctccactgttga

SEQ ID NO: 31 - DNA, 1320 bp, Artificial
  Synthetic Construct; smycD2S N393A
atgaagacgatcatcgccctgagctacatcttctgcctggtgttcgccgagcagaagctgatctccgaagaggacctggatccactgaatctgtcctggtatgatgatgatctggagaggcagaactggagccggcccttcaacgggtcagacgggaaggcggacagaccccactacaactactatgccacactgctcaccctgctcatcgctgtcatcgtcttcggcaacgtgctggtgtgcatggctgtgtcccgcgagaaggcgctgcagaccaccaccaactacctgatcgtcagcctcgcagtggccgacctcctcgtcgccacactggtcatgccctgggttgtctacctggaggtggtaggtgagtggaaattcagcaggattcactgtgacatcttcgtcactctggacgtcatgatgtgcacggcgagcatcctgaacttgtgtgccatcagcatcgacaggtacacagctgtggccatgcccatgctgtacaatacgcgctacagctccaagcgccgggtcaccgtcatgatctccatcgtctgggtcctgtccttcaccatctcctgcccactcctcttcggactcaataacgcagaccagaacgagtgcatcattgccaacccggccttcgtggtctactcctccatcgtctccttctacgtgcccttcattgtcaccctgctggtctacatcaagatctacattgtcctccgcagacgccgcaagcgagtcaacaccaaacgcagcagccgagctttcagggcccacctgagggctccactaaaggaggctgcccggcgagcccaggagctggagatggagatgctctccagcaccagcccacccgagaggacccggtacagccccatcccacccagccaccaccagctgactctccccgacccgtcccaccatggtctccacagcactcccgacagccccgccaaaccagagaagaatgggcatgccaaagaccaccccaagattgccaagatctttgagatccagaccatgcccaatggcaaaacgcgtacctccctcaagaccatgagccgtaggaagctctcccagcagaaggagaagaaagccactcagatgctcgccattgttctcggcgtgttcatcatctgctggctgcccttcttcatcacacacatcctgaacatacactgtgactgcaacatcccgcctgtcctgtacagcgccttcacgtggctgggctatgtcaacagcgccgtggctcccatcatctacaccaccttcaacattgagttccgcaaggccttcctgaagatcctccactgttga

SEQ ID NO: 32 - DNA, 1320 bp, Artificial
  Synthetic Construct; smycD2S R132A
atgaagacgatcatcgccctgagctacatcttctgcctggtgttcgccgagcagaagctgatctccgaagaggacctggatccactgaatctgtcctggtatgatgatgatctggagaggcagaactggagccggcccttcaacgggtcagacgggaaggcggacagaccccactacaactactatgccacactgctcaccctgctcatcgctgtcatcgtcttcggcaacgtgctggtgtgcatggctgtgtcccgcgagaaggcgctgcagaccaccaccaactacctgatcgtcagcctcgcagtggccgacctcctcgtcgccacactggtcatgccctgggttgtctacctggaggtggtaggtgagtggaaattcagcaggattcactgtgacatcttcgtcactctggacgtcatgatgtgcacggcgagcatcctgaacttgtgtgccatcagcatcgacgcttacacagctgtggccatgcccatgctgtacaatacgcgctacagctccaagcgccgggtcaccgtcatgatctccatcgtctgggtcctgtccttcaccatctcctgcccactcctcttcggactcaataacgcagaccagaacgagtgcatcattgccaacccggccttcgtggtctactcctccatcgtctccttctacgtgcccttcattgtcaccctgctggtctacatcaagatctacattgtcctccgcagacgccgcaagcgagtcaacaccaaacgcagcagccgagctttcagggcccacctgagggctccactaaaggaggctgcccggcgagcccaggagctggagatggagatgctctccagcaccagcccacccgagaggacccggtacagccccatcccacccagccaccaccagctgactctccccgacccgtcccaccatggtctccacagcactcccgacagccccgccaaaccagagaagaatgggcatgccaaagaccaccccaagattgccaagatctttgagatccagaccatgcccaatggcaaaacgcgtacctccctcaagaccatgagccgtaggaagctctcccagcagaaggagaagaaagccactcagatgctcgccattgttctcggcgtgttcatcatctgctggctgcccttcttcatcacacacatcctgaacatacactgtgactgcaacatcccgcctgtcctgtacagcgccttcacgtggctgggctatgtcaacagcgccgtgaaccccatcatctacaccaccttcaacattgagttccgcaaggccttcctgaagatcctccactgttga

SEQ ID NO: 33 - DNA, 1320 bp, Artificial
  Synthetic Construct; smycD2S V136D/M140E
atgaagacgatcatcgccctgagctacatcttctgcctggtgttcgccgagcagaagctgatctccgaagaggacctggatccactgaatctgtcctggtatgatgatgatctggagaggcagaactggagccggcccttcaacgggtcagacgggaaggcggacagaccccactacaactactatgccacactgctcaccctgctcatcgctgtcatcgtcttcggcaacgtgctggtgtgcatggctgtgtcccgcgagaaggcgctgcagaccaccaccaactacctgatcgtcagcctcgcagtggccgacctcctcgtcgccacactggtcatgccctgggttgtctacctggaggtggtaggtgagtggaaattcagcaggattcactgtgacatcttcgtcactctggacgtcatgatgtgcacggcgagcatcctgaacttgtgtgccatcagcatcgacaggtacacagctgatgccatgcccgaactgtacaatacgcgctacagctccaagcgccgggtcaccgtcatgatctccatcgtctgggtcctgtccttcaccatctcctgcccactcctcttcggactcaataacgcagaccagaacgagtgcatcattgccaacccggccttcgtggtctactcctccatcgtctccttctacgtgcccttcattgtcaccctgctggtctacatcaagatctacattgtcctccgcagacgccgcaagcgagtcaacaccaaacgcagcagccgagctttcagggcccacctgagggctccactaaaggaggctgcccggcgagcccaggagctggagatggagatgctctccagcaccagcccacccgagaggacccggtacagccccatcccacccagccaccaccagctgactctccccgacccgtcccaccatggtctccacagcactcccgacagccccgccaaaccagagaagaatgggcatgccaaagaccaccccaagattgccaagatctttgagatccagaccatgcccaatggcaaaacgcgtacctccctcaagaccatgagccgtaggaagctctcccagcagaaggagaagaaagccactcagatgctcgccattgttctcggcgtgttcatcatctgctggctgcccttcttcatcacacacatcctgaacatacactgtgactgcaacatcccgcctgtcctgtacagcgccttcacgtggctgggctatgtcaacagcgccgtgaaccccatcatctacaccaccttcaacattgagttccgcaaggccttcctgaagatcctccactgttga

SEQ ID NO: 34 - DNA, 1320 bp, Artificial
  Synthetic Construct; smycD2S CAM/N393A
atgaagacgatcatcgccctgagctacatcttctgcctggtgttcgccgagcagaagctgatctccgaagaggacctggatccactgaatctgtcctggtatgatgatgatctggagaggcagaactggagccggcccttcaacgggtcagacgggaaggcggacagaccccactacaactactatgccacactgctcaccctgctcatcgctgtcatcgtcttcggcaacgtgctggtgtgcatggctgtgtcccgcgagaaggcgctgcagaccaccaccaactacctgatcgtcagcctcgcagtggccgacctcctcgtcgccacactggtcatgccctgggttgtctacctggaggtggtaggtgagtggaaattcagcaggattcactgtgacatcttcgtcactctggacgtcatgatgtgcacggcgagcatcctgaacttgtgtgccatcagcatcgacaggtacacagctgtggccatgcccatgctgtacaatacgcgctacagctccaagcgccgggtcaccgtcatgatctccatcgtctgggtcctgtccttcaccatctcctgcccactcctcttcggactcaataacgcagaccagaacgagtgcatcattgccaacccggccttcgtggtctactcctccatcgtctccttctacgtgcccttcattgtcaccctgctggtctacatcaagatctacattgtcctccgcagacgccgcaagcgagtcaacaccaaacgcagcagccgagctttcagggcccacctgagggctccactaaaggaggctgcccggcgagcccaggagctggagatggagatgctctccagcaccagcccacccgagaggacccggtacagccccatcccacccagccaccaccagctgactctccccgacccgtcccaccatggtctccacagcactcccgacagccccgccaaaccagagaagaatgggcatgccaaagaccaccccaagattgccaagatctttgagatccagaccatgcccaatggcaaaacgcgtacctccctcaagaccatgagccgtaggaagctctcccagcagaaggcaaagaaagcccgtcagatgctcgccattgttctcggcgtgttcatcatctgctggctgcccttcttcatcacacacatcctgaacatacactgtgactgcaacatcccgcctgtcctgtacagcgccttcacgtggctgggctatgtcaacagcgccgtggctcccatcatctacaccaccttcaacattgagttccgcaaggccttcctgaagatcctccactgttga

SEQ ID NO: 35 - DNA, 1320 bp, Artificial
  Synthetic Construct; smycD2S D114A/CAM
atgaagacgatcatcgccctgagctacatcttctgcctggtgttcgccgagcagaagctgatctccgaagaggacctggatccactgaatctgtcctggtatgatgatgatctggagaggcagaactggagccggcccttcaacgggtcagacgggaaggcggacagaccccactacaactactatgccacactgctcaccctgctcatcgctgtcatcgtcttcggcaacgtgctggtgtgcatggctgtgtcccgcgagaaggcgctgcagaccaccaccaactacctgatcgtcagcctcgcagtggccgacctcctcgtcgccacactggtcatgccctgggttgtctacctggaggtggtaggtgagtggaaattcagcaggattcactgtgacatcttcgtcactctggccgtcatgatgtgcacggcgagcatcctgaacttgtgtgccatcagcatcgacaggtacacagctgtggccatgcccatgctgtacaatacgcgctacagctccaagcgccgggtcaccgtcatgatctccatcgtctgggtcctgtccttcaccatctcctgcccactcctcttcggactcaataacgcagaccagaacgagtgcatcattgccaacccggccttcgtggtctactcctccatcgtctccttctacgtgcccttcattgtcaccctgctggtctacatcaagatctacattgtcctccgcagacgccgcaagcgagtcaacaccaaacgcagcagccgagctttcagggcccacctgagggctccactaaaggaggctgcccggcgagcccaggagctggagatggagatgctctccagcaccagcccacccgagaggacccggtacagccccatcccacccagccaccaccagctgactctccccgacccgtcccaccatggtctccacagcactcccgacagccccgccaaaccagagaagaatgggcatgccaaagaccaccccaagattgccaagatctttgagatccagaccatgcccaatggcaaaacgcgtacctccctcaagaccatgagccgtaggaagctctcccagcagaaggcaaagaaagcccgtcagatgctcgccattgttctcggcgtgttcatcatctgctggctgcccttcttcatcacacacatcctgaacatacactgtgactgcaacatcccgcctgtcctgtacagcgccttcacgtggctgggctatgtcaacagcgccgtgaaccccatcatctacaccaccttcaacattgagttccgcaaggccttcctgaagatcctccactgttga

SEQ ID NO: 36 - DNA, 1320 bp, Artificial
  Synthetic Construct; smycD2S D114A/D131N
atgaagacgatcatcgccctgagctacatcttctgcctggtgttcgccgagcagaagctgatctccgaagaggacctggatccactgaatctgtcctggtatgatgatgatctggagaggcagaactggagccggcccttcaacgggtcagacgggaaggcggacagaccccactacaactactatgccacactgctcaccctgctcatcgctgtcatcgtcttcggcaacgtgctggtgtgcatggctgtgtcccgcgagaaggcgctgcagaccaccaccaactacctgatcgtcagcctcgcagtggccgacctcctcgtcgccacactggtcatgccctgggttgtctacctggaggtggtaggtgagtggaaattcagcaggattcactgtgacatcttcgtcactctggccgtcatgatgtgcacggcgagcatcctgaacttgtgtgccatcagcatcaacaggtacacagctgtggccatgcccatgctgtacaatacgcgctacagctccaagcgccgggtcaccgtcatgatctccatcgtctgggtcctgtccttcaccatctcctgcccactcctcttcggactcaataacgcagaccagaacgagtgcatcattgccaacccggccttcgtggtctactcctccatcgtctccttctacgtgcccttcattgtcaccctgctggtctacatcaagatctacattgtcctccgcagacgccgcaagcgagtcaacaccaaacgcagcagccgagctttcagggcccacctgagggctccactaaaggaggctgcccggcgagcccaggagctggagatggagatgctctccagcaccagcccacccgagaggacccggtacagccccatcccacccagccaccaccagctgactctccccgacccgtcccaccatggtctccacagcactcccgacagccccgccaaaccagagaagaatgggcatgccaaagaccaccccaagattgccaagatctttgagatccagaccatgcccaatggcaaaacgcgtacctccctcaagaccatgagccgtaggaagctctcccagcagaaggagaagaaagccactcagatgctcgccattgttctcggcgtgttcatcatctgctggctgcccttcttcatcacacacatcctgaacatacactgtgactgcaacatcccgcctgtcctgtacagcgccttcacgtggctgggctatgtcaacagcgccgtgaaccccatcatctacaccaccttcaacattgagttccgcaaggccttcctgaagatcctccactgttga

SEQ ID NO: 37 - DNA, 1320 bp, Artificial
  Synthetic Construct; smycD2S D114A/R132A
atgaagacgatcatcgccctgagctacatcttctgcctggtgttcgccgagcagaagctgatctccgaagaggacctggatccactgaatctgtcctggtatgatgatgatctggagaggcagaactggagccggcccttcaacgggtcagacgggaaggcggacagaccccactacaactactatgccacactgctcaccctgctcatcgctgtcatcgtcttcggcaacgtgctggtgtgcatggctgtgtcccgcgagaaggcgctgcagaccaccaccaactacctgatcgtcagcctcgcagtggccgacctcctcgtcgccacactggtcatgccctgggttgtctacctggaggtggtaggtgagtggaaattcagcaggattcactgtgacatcttcgtcactctggccgtcatgatgtgcacggcgagcatcctgaacttgtgtgccatcagcatcgacgcttacacagctgtggccatgcccatgctgtacaatacgcgctacagctccaagcgccgggtcaccgtcatgatctccatcgtctgggtcctgtccttcaccatctcctgcccactcctcttcggactcaataacgcagaccagaacgagtgcatcattgccaacccggccttcgtggtctactcctccatcgtctccttctacgtgcccttcattgtcaccctgctggtctacatcaagatctacattgtcctccgcagacgccgcaagcgagtcaacaccaaacgcagcagccgagctttcagggcccacctgagggctccactaaaggaggctgcccggcgagcccaggagctggagatggagatgctctccagcaccagcccacccgagaggacccggtacagccccatcccacccagccaccaccagctgactctccccgacccgtcccaccatggtctccacagcactcccgacagccccgccaaaccagagaagaatgggcatgccaaagaccaccccaagattgccaagatctttgagatccagaccatgcccaatggcaaaacgcgtacctccctcaagaccatgagccgtaggaagctctcccagcagaaggagaagaaagccactcagatgctcgccattgttctcggcgtgttcatcatctgctggctgcccttcttcatcacacacatcctgaacatacactgtgactgcaacatcccgcctgtcctgtacagcgccttcacgtggctgggctatgtcaacagcgccgtgaaccccatcatctacaccaccttcaacattgagttccgcaaggccttcctgaagatcctccactgttga

SEQ ID NO: 38 - DNA, 1320 bp, Artificial
  Synthetic Construct; smycD2S D114A/V136D/M140E
atgaagacgatcatcgccctgagctacatcttctgcctggtgttcgccgagcagaagctgatctccgaagaggacctggatccactgaatctgtcctggtatgatgatgatctggagaggcagaactggagccggcccttcaacgggtcagacgggaaggcggacagaccccactacaactactatgccacactgctcaccctgctcatcgctgtcatcgtcttcggcaacgtgctggtgtgcatggctgtgtcccgcgagaaggcgctgcagaccaccaccaactacctgatcgtcagcctcgcagtggccgacctcctcgtcgccacactggtcatgccctgggttgtctacctggaggtggtaggtgagtggaaattcagcaggattcactgtgacatcttcgtcactctggccgtcatgatgtgcacggcgagcatcctgaacttgtgtgccatcagcatcgacaggtacacagctgatgccatgcccgaactgtacaatacgcgctacagctccaagcgccgggtcaccgtcatgatctccatcgtctgggtcctgtccttcaccatctcctgcccactcctcttcggactcaataacgcagaccagaacgagtgcatcattgccaacccggccttcgtggtctactcctccatcgtctccttctacgtgcccttcattgtcaccctgctggtctacatcaagatctacattgtcctccgcagacgccgcaagcgagtcaacaccaaacgcagcagccgagctttcagggcccacctgagggctccactaaaggaggctgcccggcgagcccaggagctggagatggagatgctctccagcaccagcccacccgagaggacccggtacagccccatcccacccagccaccaccagctgactctccccgacccgtcccaccatggtctccacagcactcccgacagccccgccaaaccagagaagaatgggcatgccaaagaccaccccaagattgccaagatctttgagatccagaccatgcccaatggcaaaacgcgtacctccctcaagaccatgagccgtaggaagctctcccagcagaaggagaagaaagccactcagatgctcgccattgttctcggcgtgttcatcatctgctggctgcccttcttcatcacacacatcctgaacatacactgtgactgcaacatcccgcctgtcctgtacagcgccttcacgtggctgggctatgtcaacagcgccgtgaaccccatcatctacaccaccttcaacattgagttccgcaaggccttcctgaagatcctccactgttga

SEQ ID NO: 39 - DNA, 1320 bp, Artificial
  Synthetic Construct; smycD2S D131N
atgaagacgatcatcgccctgagctacatcttctgcctggtgttcgccgagcagaagctgatctccgaagaggacctggatccactgaatctgtcctggtatgatgatgatctggagaggcagaactggagccggcccttcaacgggtcagacgggaaggcggacagaccccactacaactactatgccacactgctcaccctgctcatcgctgtcatcgtcttcggcaacgtgctggtgtgcatggctgtgtcccgcgagaaggcgctgcagaccaccaccaactacctgatcgtcagcctcgcagtggccgacctcctcgtcgccacactggtcatgccctgggttgtctacctggaggtggtaggtgagtggaaattcagcaggattcactgtgacatcttcgtcactctggacgtcatgatgtgcacggcgagcatcctgaacttgtgtgccatcagcatcaacaggtacacagctgtggccatgcccatgctgtacaatacgcgctacagctccaagcgccgggtcaccgtcatgatctccatcgtctgggtcctgtccttcaccatctcctgcccactcctcttcggactcaataacgcagaccagaacgagtgcatcattgccaacccggccttcgtggtctactcctccatcgtctccttctacgtgcccttcattgtcaccctgctggtctacatcaagatctacattgtcctccgcagacgccgcaagcgagtcaacaccaaacgcagcagccgagctttcagggcccacctgagggctccactaaaggaggctgcccggcgagcccaggagctggagatggagatgctctccagcaccagcccacccgagaggacccggtacagccccatcccacccagccaccaccagctgactctccccgacccgtcccaccatggtctccacagcactcccgacagccccgccaaaccagagaagaatgggcatgccaaagaccaccccaagattgccaagatctttgagatccagaccatgcccaatggcaaaacgcgtacctccctcaagaccatgagccgtaggaagctctcccagcagaaggagaagaaagccactcagatgctcgccattgttctcggcgtgttcatcatctgctggctgcccttcttcatcacacacatcctgaacatacactgtgactgcaacatcccgcctgtcctgtacagcgccttcacgtggctgggctatgtcaacagcgccgtgaaccccatcatctacaccaccttcaacattgagttccgcaaggccttcctgaagatcctccactgttga

SEQ ID NO: 40 - DNA, 1320 bp, Artificial
  Synthetic Construct; smycD2S Y397F (Y7.53F)
atgaagacgatcatcgccctgagctacatcttctgcctggtgttcgccgagcagaagctgatctccgaagaggacctggatccactgaatctgtcctggtatgatgatgatctggagaggcagaactggagccggcccttcaacgggtcagacgggaaggcggacagaccccactacaactactatgccacactgctcaccctgctcatcgctgtcatcgtcttcggcaacgtgctggtgtgcatggctgtgtcccgcgagaaggcgctgcagaccaccaccaactacctgatcgtcagcctcgcagtggccgacctcctcgtcgccacactggtcatgccctgggttgtctacctggaggtggtaggtgagtggaaattcagcaggattcactgtgacatcttcgtcactctggacgtcatgatgtgcacggcgagcatcctgaacttgtgtgccatcagcatcgacaggtacacagctgtggccatgcccatgctgtacaatacgcgctacagctccaagcgccgggtcaccgtcatgatctccatcgtctgggtcctgtccttcaccatctcctgcccactcctcttcggactcaataacgcagaccagaacgagtgcatcattgccaacccggccttcgtggtctactcctccatcgtctccttctacgtgcccttcattgtcaccctgctggtctacatcaagatctacattgtcctccgcagacgccgcaagcgagtcaacaccaaacgcagcagccgagctttcagggcccacctgagggctccactaaaggaggctgcccggcgagcccaggagctggagatggagatgctctccagcaccagcccacccgagaggacccggtacagccccatcccacccagccaccaccagctgactctccccgacccgtcccaccatggtctccacagcactcccgacagccccgccaaaccagagaagaatgggcatgccaaagaccaccccaagattgccaagatctttgagatccagaccatgcccaatggcaaaacgcgtacctccctcaagaccatgagccgtaggaagctctcccagcagaaggagaagaaagccactcagatgctcgccattgttctcggcgtgttcatcatctgctggctgcccttcttcatcacacacatcctgaacatacactgtgactgcaacatcccgcctgtcctgtacagcgccttcacgtggctgggctatgtcaacagcgccgtgaaccccatcatcttcaccaccttcaacattgagttccgcaaggccttcctgaagatcctccactgttga

SEQ ID NO: 41 - DNA, 1425 bp, Artificial
  Synthetic Construct; 3HA-human D1
atgtacccatacgatgttccagattacgcttacccatacgatgttccagattacgcttacccatacgatgttccagattacgctgataggactctgaacacctctgccatggacgggactgggctggtggtggagagggacttctctgttcgtatcctcactgcctgtttcctgtcgctgctcatcctgtccacgctcctggggaacacgctggtctgtgctgccgttatcaggttccgacacctgcggtccaaggtgaccaacttctttgtcatctccttggctgtgtcagatctcttggtggccgtcctggtcatgccctggaaggcagtggctgagattgctggcttctggccctttgggtccttctgtaacatctgggtggcctttgacatcatgtgctccactgcatccatcctcaacctctgtgtgatcagcgtggacaggtattgggctatctccagccctttccggtatgagagaaagatgacccccaaggcagccttcatcctgatcagtgtggcatggaccttgtctgtactcatctccttcatcccagtgcagctcagctggcacaaggcaaaacccacaagcccctctgatggaaatgccacttccctggctgagaccatagacaactgtgactccagcctcagcaggacatatgccatctcatcctctgtaataagcttttacatccctgtggccatcatgattgtcacctacaccaggatctacaggattgctcagaaacaaatacggcgcattgcggccttggagagggcagcagtccacgccaagaattgccagaccaccacaggtaatggaaagcctgtcgaatgttctcaaccggaaagttcttttaagatgtccttcaaaagagaaactaaagtcctgaagactctgtcggtgatcatgggtgtgtttgtgtgctgttggctacctttcttcatcttgaactgcattttgcccttctgtgggtctggggagacgcagcccttctgcattgattccaacacctttgacgtgtttgtgtggtttgggtgggctaattcatccttgaaccccatcatttatgcctttaatgctgattttcggaaggcattttcaaccctcttaggatgctacagactttgccctgcgacgaataatgccatagagacggtgagtatcaataacaatggggccgcgatgttttccagccatcatgagccacgaggctccatctccaaggagtgcaatctggtttacctgatcccacatgctgtgggctcctctgaggacctgaaaaaggaggaggcagctggcatcgccagacccttggagaagctgtccccagccctatcggtcatattggactatgacactgacgtctctctggagaagatccaacccatcacacaaaacggtcagcacccaacctga

SEQ ID NO: 42 - DNA, 2526 bp, Artificial
  Synthetic Construct; 3HAD1-linker-Gqi5
atgtacccatacgatgttccagattacgcttacccatacgatgttccagattacgcttacccatacgatgttccagattacgctgataggactctgaacacctctgccatggacgggactgggctggtggtggagagggacttctctgttcgtatcctcactgcctgtttcctgtcgctgctcatcctgtccacgctcctggggaacacgctggtctgtgctgccgttatcaggttccgacacctgcggtccaaggtgaccaacttctttgtcatctccttggctgtgtcagatctcttggtggccgtcctggtcatgccctggaaggcagtggctgagattgctggcttctggccctttgggtccttctgtaacatctgggtggcctttgacatcatgtgctccactgcatccatcctcaacctctgtgtgatcagcgtggacaggtattgggctatctccagccctttccggtatgagagaaagatgacccccaaggcagccttcatcctgatcagtgtggcatggaccttgtctgtactcatctccttcatcccagtgcagctcagctggcacaaggcaaaacccacaagcccctctgatggaaatgccacttccctggctgagaccatagacaactgtgactccagcctcagcaggacatatgccatctcatcctctgtaataagcttttacatccctgtggccatcatgattgtcacctacaccaggatctacaggattgctcagaaacaaatacggcgcattgcggccttggagagggcagcagtccacgccaagaattgccagaccaccacaggtaatggaaagcctgtcgaatgttctcaaccggaaagttcttttaagatgtccttcaaaagagaaactaaagtcctgaagactctgtcggtgatcatgggtgtgtttgtgtgctgttggctacctttcttcatcttgaactgcattttgcccttctgtgggtctggggagacgcagcccttctgcattgattccaacacctttgacgtgtttgtgtggtttgggtgggctaattcatccttgaaccccatcatttatgcctttaatgctgattttcggaaggcattttcaaccctcttaggatgctacagactttgccctgcgacgaataatgccatagagacggtgagtatcaataacaatggggccgcgatgttttccagccatcatgagccacgaggctccatctccaaggagtgcaatctggtttacctgatcccacatgctgtgggctcctctgaggacctgaaaaaggaggaggcagctggcatcgccagacccttggagaagctgtccccagccctatcggtcatattggactatgacactgacgtctctctggagaagatccaacccatcacacaaaacggtcagcacccaaccttcgaaagacctgcagacggtagaatgactctggagtccatcatggcgtgctgcctgagcgaggaggccaaggaagcccggcggatcaacgacgagatcgagcggcagctccgcagggacaagcgggacgcccgccgggagctcaagctgctgctgctcgggacaggagagagtggcaagagtacgtttatcaagcagatgagaatcatccatgggtcaggatactctgatgaagataaaaggggcttcaccaagctggtgtatcagaacatcttcacggccatgcaggccatgatcagagccatggacacactcaagatcccatacaagtatgagcacaataaggctcatgcacaattagttcgagaagttgatgtggagaaggtgtctgcttttgagaatccatatgtagatgcaataaagagtttatggaatgatcctggaatccaggaatgctatgatagacgacgagaatatcaattatctgactctaccaaatactatcttaatgacttggaccgcgtagctgaccctgcctacctgcctacgcaacaagatgtgcttagagttcgagtccccaccacagggatcatcgaatacccctttgacttacaaagtgtcattttcagaatggtcgatgtagggggccaaaggtcagagagaagaaaatggatacactgctttgaaaatgtcacctctatcatgtttctagtagcgcttagtgaatatgatcaagttctcgtggagtcagacaatgagaaccgaatggaggaaagcaaggctctctttagaacaattatcacatacccctggttccagaactcctcggttattctgttcttaaacaagaaagatcttctagaggagaaaatcatgtattcccatctagtcgactacttcccagaatatgatggaccccagagagatgcccaggcagcccgagaattcattctgaagatgttcgtggacctgaacccagacagtgacaaaattatctactcccacttcacgtgcgccacagacaccgagaatatccgctttgtctttgctgccgtcaaggacaccatcctccagttgaacctgaaggatattggtctgttctaa

SEQ ID NO: 43 - DNA, 1257 bp, Artificial
  Synthetic Construct; 3HA-human 5HT1b
atgtacccatacgatgttccagattacgcttacccatacgatgttccagattacgcttacccatacgatgttccagattacgctgatgaggaaccgggtgctcagtgcgctccaccgccgcccgcgggctccgagacctgggttcctcaagccaacttatcctctgctccctcccaaaactgcagcgccaaggactacatttaccaggactccatctccctaccctggaaagtactgctggttatgctattggcgctcatcaccttggccaccacgctctccaatgcctttgtgattgccacagtgtaccggacccggaaactgcacaccccggctaactacctgatcgcctctctggcggtcaccgacctgcttgtgtccatcctggtgatgcccatcagcaccatgtacactgtcaccggccgctggacactgggccaggtggtctgtgacttctggctgtcgtcggacatcacttgttgcactgcctccatcctgcacctctgtgtcatcgccctggaccgctactgggccatcacggacgccgtggagtactcagctaaaaggactcccaagagggcggcggtcatgatcgcgctggtgtgggtcttctccatctctatctcgctgccgcccttcttctggcgtcaggctaaggccgaagaggaggtgtcggaatgcgtggtgaacaccgaccacatcctctacacggtctactccacggtgggtgctttctacttccccaccctgctcctcatcgccctctatggccgcatctacgtagaagcccgctcccggattttgaaacagacgcccaacaggaccggcaagcgcttgacccgagcccagctgataaccgactcccccgggtccacgtcctcggtcacctctattaactcgcgggttcccgacgtgcccagcgaatccggatctcctgtgtatgtgaaccaagtcaaagtgcgagtctccgacgccctgctggaaaagaagaaactcatggccgctagggagcgcaaagccaccaagaccctagggatcattttgggagcctttattgtgtgttggctacccttcttcatcatctccctagtgatgcctatctgcaaagatgcctgctggttccacctagccatctttgacttcttcacatggctgggctatctcaactccctcatcaaccccataatctataccatgtccaatgaggactttaaacaagcattccataaactgatacgttttaagtgcacaagttga

SEQ ID NO: 44 - DNA, 1326 bp, Artificial
  Synthetic Construct; 3HA-human A2a
atgtacccatacgatgttccagattacgcttacccatacgatgttccagattacgcttacccatacgatgttccagattacgctgatatccccatcatgggctcctcggtgtacatcacggtggagctggccattgctgtgctggccatcctgggcaatgtgctggtgtgctgggccgtgtggctcaacagcaacctgcagaacgtcaccaactactttgtggtgtcactggcggcggccgacatcgcagtgggtgtgctcgccatcccctttgccatcaccatcagcaccgggttctgcgctgcctgccacggctgcctcttcattgcctgcttcgtcctggtcctcacgcagagctccatcttcagtctcctggccatcgccattgaccgctacattgccatccgcatcccgctccggtacaatggcttggtgaccggcacgagggctaagggcatcattgccatctgctgggtgctgtcgtttgccatcggcctgactcccatgctaggttggaacaactgcggtcagccaaaggagggcaagaaccactcccagggctgcggggagggccaagtggcctgtctctttgaggatgtggtccccatgaactacatggtgtacttcaacttctttgcctgtgtgctggtgcccctgctgctcatgctgggtgtctatttgcggatcttcctggcggcgcgacgacagctgaagcagatggagagccagcctctgccgggggagcgggcacggtccacactgcagaaggaggtccatgctgccaagtcactggccatcattgtggggctctttgccctctgctggctgcccctacacatcatcaactgcttcactttcttctgccccgactgcagccacgcccctctctggctcatgtacctggccatcgtcctctcccacaccaattcggttgtgaatcccttcatctacgcctaccgtatccgcgagttccgccagaccttccgcaagatcattcgcagccacgtcctgaggcagcaagaacctttcaaggcagctggcaccagtgcccgggtcttggcagctcatggcagtgacggagagcaggtcagcctccgtctcaacggccacccgccaggagtgtgggccaacggcagtgctccccaccctgagcggaggcccaatggctacgccctggggctggtgagtggagggagtgcccaagagtcccaggggaacacgggcctcccagacgtggagctccttagccatgagctcaagggagtgtgcccagagccccctggcctagatgaccccctggcccaggatggagcaggagtgtcctga

SEQ ID NO: 45 - DNA, 1503 bp, Artificial
  Synthetic Construct; 3HA-human CB1
atgtacccatacgatgttccagattacgcttacccatacgatgttccagattacgcttacccatacgatgttccagattacgctgataagtcgatcctagatggccttgcagataccaccttccgcaccatcaccactgacctcctgtacgtgggctcaaatgacattcagtacgaagacatcaaaggtgacatggcatccaaattagggtacttcccacagaaattccctttaacttcctttaggggaagtcccttccaagagaagatgactgcgggagacaacccccagctagtcccagcagaccaggtgaacattacagaattttacaacaagtctctctcgtccttcaaggagaatgaggagaacatccagtgtggggagaacttcatggacatagagtgtttcatggtcctgaaccccagccagcagctggccattgcagtcctgtccctcacgctgggcaccttcacggtcctggagaacctcctggtgctgtgcgtcatcctccactcccgcagcctccgctgcaggccttcctaccacttcatcggcagcctggcggtggcagacctcctggggagtgtcatttttgtctacagcttcattgacttccacgtgttccaccgcaaagatagccgcaacgtgtttctgttcaaactgggtggggtcacggcctccttcactgcctccgtgggcagcctgttcctcacagccatcgacaggtacatatccattcacaggcccctggcctataagaggattgtcaccaggcccaaggccgtggtggcgttttgcctgatgtggaccatagccattgtgatcgccgtgctgcctctcctgggctggaactgcgagaaactgcaatctgtttgctcagacattttcccacacattgatgaaacctacctgatgttctggatcggggtcaccagcgtactgcttctgttcatcgtgtatgcgtacatgtatattctctggaaggctcacagccacgccgtccgcatgattcagcgtggcacccagaagagcatcatcatccacacgtctgaggatgggaaggtacaggtgacccggccagaccaagcccgcatggacattaggttagccaagaccctggtcctgatcctggtggtgttgatcatctgctggggccctctgcttgcaatcatggtgtatgatgtctttgggaagatgaacaagctcattaagacggtgtttgcattctgcagtatgctctgcctgctgaactccaccgtgaaccccatcatctatgctctgaggagtaaggacctgcgacacgctttccggagcatgtttccctcttgtgaaggcactgcgcagcctctggataacagcatgggggactcggactgcctgcacaaacacgcaaacaatgcagccagtgttcacagggccgcagaaagctgcatcaagagcacggtcaagattgccaaggtaaccatgtctgtgtccacagacacgtctgcagaggctctgtga

SEQ ID NO: 46 - DNA, 3600 bp, Rattus rattus
atggtccggctcctcttgattttcttcccaatgatctttttggagatgtccattttgcccaggatgcctgacagaaaagtattgctggcaggtgcctcgtcccagcgctccgtggcgagaatggacggagatgtcatcatcggagccctcttctcagtccatcaccagcctccagccgagaaggtacccgaaaggaagtgtggggagatcagggaacagtatggtatccagagggtggaggccatgttccacacgttggataagattaacgcggacccggtgctcctgcccaacatcactctgggcagtgagatccgggactcctgctggcactcttcagtggctctcgaacagagcatcgaattcatcagagactccctgatttccatccgagatgagaaggatgggctgaaccgatgcctgcctgatggccagaccctgccccctggcaggactaagaagcctattgctggagtgatcggccctggctccagctctgtggccattcaagtccagaatcttctccagctgttcgacatcccacagatcgcctattctgccacaagcatagacctgagtgacaaaactttgtacaaatacttcctgagggtggtcccttctgacactttgcaggcaagggcgatgctcgacatagtcaagcgttacaactggacctatgtctcagcagtccacacagaagggaattacggcgagagtggaatggatgctttcaaagaactggctgcccaggaaggcctctgcatcgcacactcggacaaaatctacagcaatgctggcgagaagagctttgaccggctcctgcgtaaactccgggagcggcttcccaaggccagggttgtggtctgcttctgcgagggcatgacagtgcggggcttactgagtgccatgcgccgcctgggcgtcgtgggcgagttctcactcattggaagtgatggatgggcagacagagatgaagtcatcgaaggctatgaggtggaagccaacggagggatcacaataaagcttcagtctccagaggtcaggtcatttgatgactacttcctgaagctgaggctggacaccaacacaaggaatccttggttccctgagttctggcaacatcgcttccagtgtcgcctacctggacacctcttggaaaaccccaactttaagaaagtgtgcacaggaaatgaaagcttggaagaaaactatgtccaggacagcaaaatgggatttgtcatcaatgccatctatgccatggcacatgggctgcagaacatgcaccatgctctgtgtcccggccatgtgggcctgtgtgatgctatgaaacccattgatggcaggaagctcctggatttcctcatcaaatcctcttttgtcggagtgtctggagaggaggtgtggttcgatgagaagggggatgctcccggaaggtatgacattatgaatctgcagtacacagaagctaatcgctatgactatgtccacgtggggacctggcatgaaggagtgctgaatattgatgattacaaaatccagatgaacaaaagcggaatggtacgatctgtgtgcagtgagccttgcttaaagggtcagattaaggtcatacggaaaggagaagtgagctgctgctggatctgcacggcctgcaaagagaatgagtttgtgcaggacgagttcacctgcagagcctgtgacctggggtggtggcccaacgcagagctcacaggctgtgagcccattcctgtccgttatcttgagtggagtgacatagaatctatcatagccatcgccttttcttgcctgggcatcctcgtgacgctgtttgtcaccctcatcttcgttctgtaccgggacacacccgtggtcaaatcctccagtagggagctctgctatatcattctggctggtattttcctcggctatgtgtgccctttcaccctcatcgccaaacctactaccacatcctgctacctccagcgcctcctagttggcctctcttctgccatgtgctactctgctttagtgaccaaaaccaatcgtattgcacgcatcctggctggcagcaagaagaagatctgcacccggaagcccagattcatgagcgcttgggcccaagtgatcatagcctccattctgattagtgtacagctaacactagtggtgaccttgatcatcatggagcctcccatgcccattttgtcctacccgagtatcaaggaagtctaccttatctgcaataccagcaacctgggtgtagtggcccctgtgggttacaatggactcctcatcatgagctgtacctactatgccttcaagacccgcaacgtgccggccaacttcaatgaggctaaatacatcgccttcaccatgtacactacctgcatcatctggctggctttcgttcccatttactttgggagcaactacaagatcatcactacctgcttcgcggtgagcctcagtgtgacggtggccctggggtgcatgtttactccgaagatgtacatcatcattgccaaacctgagaggaacgtccgcagtgccttcacgacctctgatgttgtccgcatgcacgtcggtgatggcaaactgccgtgccgctccaacaccttcctcaacattttccggagaaagaagcccggggcagggaatgccaattctaacggcaagtctgtgtcatggtctgaaccaggtggaagacaggcgcccaagggacagcacgtgtggcagcgcctctctgtgcacgtgaagaccaacgagacggcctgtaaccaaacagccgtaatcaaacccctcactaaaagttaccaaggctctggcaagagcctgaccttttcagatgccagcaccaagaccctttacaatgtggaagaagaggacaataccccttctgctcacttcagccctcccagcagcccttctatggtggtgcaccgacgcgggccacccgtggccaccacaccacctctgccaccccatctgaccgcagaagagacccccctgttcctggctgattccgtcatccccaagggcttgcctcctcctctcccgcagcagcagccacagcagccgccccctcagcagcccccgcagcagcccaagtccctgatggaccagctgcaaggcgtagtcaccaacttcggttcggggattccagatttccatgcggtgctggcaggcccggggacaccaggaaacagcctgcgctctctgtacccgcccccgcctccgccgcaacacctgcagatgctgcccctgcacctgagcaccttccaggaggagtccatctcccctcctggggaggacatcgatgatgacagtgagagattcaagctcctgcaggagttcgtgtacgagcgcgaagggaacaccgaagaagatgaattggaagaggaggaggacctgcccacagccagcaagctgacccctgaggattctcctgccctgacgcctccttctcctttccgagattccgtggcctctggcagctcagtgcccagttcccccgtatctgagtcggtcctctgcacccctccaaatgtaacctacgcctctgtcattctgagggactacaagcaaagctcttccaccctgtag

SEQ ID NO: 47 - DNA, 3516 bp, Rattus rattus
atggtccttctgttgatcctgtcagtcctacttctgaaagaagatgtacgagggagtgcacagtccagtgagaggagggtggtggctcacatgccaggtgacatcattattggagctctcttctctgtccaccaccaaccaactgtggacaaagttcatgagaggaagtgtggtgcagttcgtgagcaatatgggattcagagagtggaggccatgctgcataccttggaaaggatcaactcagatcccacactcttgcccaacatcacacttggctgtgagatcagagattcctgctggcattccgctgtggccctagaacaaagcattgagtttataagggattccctcatctcttcagaagaggaggaaggtttggtacgctgtgtagatggatcttcttccttccgctccaagaaacccatagtgggagtcattgggcctggctcgagttctgtggccattcaagtccagaacttactccagcttttcaacataccacagattgcttactctgcaaccagcatggatttgagtgacaagactctattcaagtacttcatgagggttgtaccttccgatgcccagcaagctcgagccatggtagacatagtgaagagatacaactggacttatgtctcagctgtgcacacagaaggcaactatggagaaagtgggatggaggctttcaaagatatgtcagcgaaggaagggatttgcatcgcccactcttacaaaatttacagcaatgctggggaacagagctttgacaagctgttgaaaaaactcagaagtcatttacctaaagcccgggtggtagcttgcttctgtgaaggcatgacagttcgaggtctgctcatggccatgagacgcttaggtctagcaggggagtttctacttctgggcagtgatggctgggccgacaggtatgacgtgacagatggatatcagcgagaagctgtcggtggaattacaatcaagcttcagtctcctgatgtcaagtggtttgatgattattatctgaaactccggccagaaacaaacctcagaaacccttggtttcaagaattttggcagcatcgttttcagtgccggctggaagggtttgcacaggagaacagcaagtacaacaagacttgcaacagttctctgactctgagaacacatcatgttcaagattccaaaatgggatttgtgataaatgcaatctattctatggcttatggactccacaatatgcagatgtccctgtgtccaggctatgcaggcctctgtgatgcgatgaagccaattgatggacggaaacttttggattccctgatgaaaaccaattttactggagtttctggagatatgatcctgtttgatgaaaatggagactctccaggaaggtatgaaataatgaatttcaaggaaatgggaaaagattattttgattacatcaatgttggaagttgggacaatggggaattaaaaatggatgatgacgaagtgtggtccaagaaaaataacatcatcagatctgtgtgcagtgaaccatgtgagaaaggccaaataaaggtgatccggaagggagaagtcagctgttgttggacctgcacaccttgtaaagagaatgagtatgtgtttgatgagtacacctgcaaggcatgccaactgggctcctggcccactgacgacctgacaggctgtgacttgatcccagtccagtatcttcgctggggtgaccctgagccgattgcagctgtggtgtttgcctgcctcggtctgctagccaccttattcgttactgtaatcttcatcatttatcgggacactccggtggtcaagtcctccagcagggagctctgctacattatccttgctggcatctgcctgggatacttatgtaccttttgcctcattgcaaagcccaagcagatttactgctatcttcagagaattggcattggactctctccagccatgagctactcagcccttgtaaccaagaccaaccgtattgcaaggattctagctggaagcaagaagaaaatctgtaccaagaagcccagattcatgagcgcctgtgctcagttagtgatcgctttcattctcatctgtattcagctgggcattatcgtggccctctttatcatggagcctccggatataatgcatgactatccaagcatccgagaagtctacttgatttgtaacaccaccaacctaggggttgttactcctcttggatacaatggattattgattttgagttgcacgttctatgcgtttaagaccagaaatgttccagccaactttaatgaggccaaatatattgctttcaccatgtacacaacctgcatcatatggctggccttcgtgcctatctactttggcagcaactacaaaatcatcaccatgtgtttttcagtcagcctcagtgccacagtggccctgggttgcatgtttgtcccgaaggtgtacatcatcctagccaaaccggagagaaatgtgcgcagcgccttcacaacctctacagtggtacgcatgcatgtaggagacggcaaatcatcgtccgctgccagcagatccagcagcctagtcaacctgtggaagaggaggggctcctctggggaaaccctaagctccaacggaaaatctgtgacttgggcccagaatgagaagagtacccgggggcaacatttgtggcagcgactgtctgtccacatcaacaagaaggagaaccccaaccaaacggcggtcatcaaaccctttcccaagagcacagagaaccgggggcctggtgcagcggcaggtggtggctcgggtcccggtgtagctggtgctggtaacgcaggatgcacagcaacaggtggcccggagccaccggacgccggtcccaaagcgctttatgatgtggcggaggcagaggagagcttccccgcggctgccaggccgcgctcgccatcgcccatcagcacgttgagccacctcgcaggctcggcgggccgcacagatgatgatgcgccgtcgctgcactctgagacagctgctcgcagcagctcgtcccagggctcgctcatggagcagatcagcagcgtagtgacgcgcttcacagccaacatctccgagctcaactccatgatgttgtccaccgcggccacgccggggccccctggtactccaatctgctcctcctacctgatccccaaagagattcagctgcccacgaccatgacgaccttcgcagagatccagccgctgccggccatcgaggtgaccggaggagctcagggggcgacaggcgtatcacctgcccaggagacgcccacaggagctgaatccgccccgggcaaaccagatctggaggagcttgtggccctcactccaccatcgcccttcagggactcggtggactcggggagcaccaccccaaactctccagtctcagaatcggccctctgcatcccatcctctcccaaatatgacactctcatcatcagagattacacgcagagttcttcatcgttgtga

SEQ ID NO: 48 - DNA, 1275 bp, Artificial
  Synthetic Construct; SF-human D3
atgaagacgatcatcgccctgagctacatcttctgcctggtgttcgccgactacaaggacgatgatgacgccatggcatctctgagtcagctgagtagccacctgaactacacctgtggggcagagaactccacaggtgccagccaggcccgcccacatgcctactatgccctctcctactgcgcgctcatcctggccatcgtcttcggcaatggcctggtgtgcatggctgtgctgaaggagcgggccctgcagactaccaccaactacttagtagtgagcctggctgtggcagacttgctggtggccaccttggtgatgccctgggtggtatacctggaggtgacaggtggagtctggaatttcagccgcatttgctgtgatgtttttgtcaccctggatgtcatgatgtgtacagccagcatccttaatctctgtgccatcagcatagacaggtacactgcagtggtcatgcccgttcactaccagcatggcacgggacagagctcctgtcggcgcgtggccctcatgatcacggccgtctgggtactggcctttgctgtgtcctgccctcttctgtttggctttaataccacaggggaccccactgtctgctccatctccaaccctgattttgtcatctactcttcagtggtgtccttctacctgccctttggagtgactgtccttgtctatgccagaatctatgtggtgctgaaacaaaggagacggaaaaggatcctcactcgacagaacagtcagtgcaacagtgtcaggcctggcttcccccaacaaaccctctctcctgacccggcacatctggagctgaagcgttactacagcatctgccaggacactgccttgggtggaccaggcttccaagaaagaggaggagagttgaaaagagaggagaagactcggaattccctgagtcccaccatagcgcccaagctcagcttagaagttcgaaaactcagcaatggcagattatcgacatctttgaagctggggcccctgcaacctcggggagtgccacttcgggagaagaaggcaacccaaatggtggccattgtgcttggggccttcattgtctgctggctgcccttcttcttgacccatgttctcaatacccactgccagacatgccacgtgtccccagagctttacagtgccacgacatggctgggctacgtgaatagcgccctcaaccctgtgatctataccaccttcaatatcgagttccggaaagccttcctcaagatcctgtcttgctga

SEQ ID NO: 49 - DNA, 2358 bp, Artificial
  Synthetic Construct; SFD3Gqi5
atgaagacgatcatcgccctgagctacatcttctgcctggtgttcgccgactacaaggacgatgatgacgccatggcatctctgagtcagctgagtagccacctgaactacacctgtggggcagagaactccacaggtgccagccaggcccgcccacatgcctactatgccctctcctactgcgcgctcatcctggccatcgtcttcggcaatggcctggtgtgcatggctgtgctgaaggagcgggccctgcagactaccaccaactacttagtagtgagcctggctgtggcagacttgctggtggccaccttggtgatgccctgggtggtatacctggaggtgacaggtggagtctggaatttcagccgcatttgctgtgatgtttttgtcaccctggatgtcatgatgtgtacagccagcatccttaatctctgtgccatcagcatagacaggtacactgcagtggtcatgcccgttcactaccagcatggcacgggacagagctcctgtcggcgcgtggccctcatgatcacggccgtctgggtactggcctttgctgtgtcctgccctcttctgtttggctttaataccacaggggaccccactgtctgctccatctccaaccctgattttgtcatctactcttcagtggtgtccttctacctgccctttggagtgactgtccttgtctatgccagaatctatgtggtgctgaaacaaaggagacggaaaaggatcctcactcgacagaacagtcagtgcaacagtgtcaggcctggcttcccccaacaaaccctctctcctgacccggcacatctggagctgaagcgttactacagcatctgccaggacactgccttgggtggaccaggcttccaagaaagaggaggagagttgaaaagagaggagaagactcggaattccctgagtcccaccatagcgcccaagctcagcttagaagttcgaaaactcagcaatggcagattatcgacatctttgaagctggggcccctgcaacctcggggagtgccacttcgggagaagaaggcaacccaaatggtggccattgtgcttggggccttcattgtctgctggctgcccttcttcttgacccatgttctcaatacccactgccagacatgccacgtgtccccagagctttacagtgccacgacatggctgggctacgtgaatagcgccctcaaccctgtgatctataccaccttcaatatcgagttccggaaagccttcctcaagatcctgtcttgcttcgaaatgactctggagtccatcatggcgtgctgcctgagcgaggaggccaaggaagcccggcggatcaacgacgagatcgagcggcagctccgcagggacaagcgggacgcccgccgggagctcaagctgctgctgctcgggacaggagagagtggcaagagtacgtttatcaagcagatgagaatcatccatgggtcaggatactctgatgaagataaaaggggcttcaccaagctggtgtatcagaacatcttcacggccatgcaggccatgatcagagccatggacacactcaagatcccatacaagtatgagcacaataaggctcatgcacaattagttcgagaagttgatgtggagaaggtgtctgcttttgagaatccatatgtagatgcaataaagagtttatggaatgatcctggaatccaggaatgctatgatagacgacgagaatatcaattatctgactctaccaaatactatcttaatgacttggaccgcgtagctgaccctgcctacctgcctacgcaacaagatgtgcttagagttcgagtccccaccacagggatcatcgaatacccctttgacttacaaagtgtcattttcagaatggtcgatgtagggggccaaaggtcagagagaagaaaatggatacactgctttgaaaatgtcacctctatcatgtttctagtagcgcttagtgaatatgatcaagttctcgtggagtcagacaatgagaaccgaatggaggaaagcaaggctctctttagaacaattatcacatacccctggttccagaactcctcggttattctgttcttaaacaagaaagatcttctagaggagaaaatcatgtattcccatctagtcgactacttcccagaatatgatggaccccagagagatgcccaggcagcccgagaattcattctgaagatgttcgtggacctgaacccagacagtgacaaaattatctactcccacttcacgtgcgccacagacaccgagaatatccgctttgtctttgctgccgtcaaggacaccatcctccagttgaacctgaaggatattggtctgttctaa

SEQ ID NO: 50 - DNA, 2376 bp, Artificial
  Synthetic Construct; SFD3-linker-Gqi5
atgaagacgatcatcgccctgagctacatcttctgcctggtgttcgccgactacaaggacgatgatgacgccatggcatctctgagtcagctgagtagccacctgaactacacctgtggggcagagaactccacaggtgccagccaggcccgcccacatgcctactatgccctctcctactgcgcgctcatcctggccatcgtcttcggcaatggcctggtgtgcatggctgtgctgaaggagcgggccctgcagactaccaccaactacttagtagtgagcctggctgtggcagacttgctggtggccaccttggtgatgccctgggtggtatacctggaggtgacaggtggagtctggaatttcagccgcatttgctgtgatgtttttgtcaccctggatgtcatgatgtgtacagccagcatccttaatctctgtgccatcagcatagacaggtacactgcagtggtcatgcccgttcactaccagcatggcacgggacagagctcctgtcggcgcgtggccctcatgatcacggccgtctgggtactggcctttgctgtgtcctgccctcttctgtttggctttaataccacaggggaccccactgtctgctccatctccaaccctgattttgtcatctactcttcagtggtgtccttctacctgccctttggagtgactgtccttgtctatgccagaatctatgtggtgctgaaacaaaggagacggaaaaggatcctcactcgacagaacagtcagtgcaacagtgtcaggcctggcttcccccaacaaaccctctctcctgacccggcacatctggagctgaagcgttactacagcatctgccaggacactgccttgggtggaccaggcttccaagaaagaggaggagagttgaaaagagaggagaagactcggaattccctgagtcccaccatagcgcccaagctcagcttagaagttcgaaaactcagcaatggcagattatcgacatctttgaagctggggcccctgcaacctcggggagtgccacttcgggagaagaaggcaacccaaatggtggccattgtgcttggggccttcattgtctgctggctgcccttcttcttgacccatgttctcaatacccactgccagacatgccacgtgtccccagagctttacagtgccacgacatggctgggctacgtgaatagcgccctcaaccctgtgatctataccaccttcaatatcgagttccggaaagccttcctcaagatcctgtcttgcttcgaaagacctgcagacggtagaatgactctggagtccatcatggcgtgctgcctgagcgaggaggccaaggaagcccggcggatcaacgacgagatcgagcggcagctccgcagggacaagcgggacgcccgccgggagctcaagctgctgctgctcgggacaggagagagtggcaagagtacgtttatcaagcagatgagaatcatccatgggtcaggatactctgatgaagataaaaggggcttcaccaagctggtgtatcagaacatcttcacggccatgcaggccatgatcagagccatggacacactcaagatcccatacaagtatgagcacaataaggctcatgcacaattagttcgagaagttgatgtggagaaggtgtctgcttttgagaatccatatgtagatgcaataaagagtttatggaatgatcctggaatccaggaatgctatgatagacgacgagaatatcaattatctgactctaccaaatactatcttaatgacttggaccgcgtagctgaccctgcctacctgcctacgcaacaagatgtgcttagagttcgagtccccaccacagggatcatcgaatacccctttgacttacaaagtgtcattttcagaatggtcgatgtagggggccaaaggtcagagagaagaaaatggatacactgctttgaaaatgtcacctctatcatgtttctagtagcgcttagtgaatatgatcaagttctcgtggagtcagacaatgagaaccgaatggaggaaagcaaggctctctttagaacaattatcacatacccctggttccagaactcctcggttattctgttcttaaacaagaaagatcttctagaggagaaaatcatgtattcccatctagtcgactacttcccagaatatgatggaccccagagagatgcccaggcagcccgagaattcattctgaagatgttcgtggacctgaacccagacagtgacaaaattatctactcccacttcacgtgcgccacagacaccgagaatatccgctttgtctttgctgccgtcaaggacaccatcctccagttgaacctgaaggatattggtctgttctaa

SEQ ID NO: 51 - DNA, 1167 bp, Artificial
  Synthetic Construct; SF-human SSTR5
atgaagacgatcatcgccctgagctacatcttctgcctggtgttcgccgactacaaggacgatgatgacgccatggagcccctgttcccagcctccacgcccagctggaacgcctcctccccgggggctgcctctggaggcggtgacaacaggacgctggtggggccggcgccctcggcaggggcccgggcggtgctggtgcccgtgctgtacctgctggtgtgtgcggccgggctgggcgggaacacgctggtcatctacgtggtgctgcgcttcgccaagatgaagaccgtcaccaacatctacattctcaacctggcagtggccgacgtcctgtacatgctggggctgcctttcctggccacgcagaacgccgcgtccttctggcccttcggccccgtcctgtgccgcctggtcatgacgctggacggcgtcaaccagttcaccagtgtcttctgcctgacagtcatgagcgtggaccgctacctggcagtggtgcacccgctgagctcggcccgctggcgccgcccgcgtgtggccaagctggcgagcgccgcggcctgggtcctgtctctgtgcatgtcgctgccgctcctggtgttcgcggacgtgcaggagggcggtacctgcaacgccagctggccggagcccgtggggctgtggggcgccgtcttcatcatctacacggccgtgctgggcttcttcgcgccgctgctggtcatctgcctgtgctacctgctcatcgtggtgaaggtgagggcggcgggcgtgcgcgtgggctgcgtgcggcggcgctcggagcggaaggtgacgcgcatggtgttggtggtggtgctggtgtttgcgggatgttggctgcccttcttcaccgtcaacatcgtcaacctggccgtggcgctgccccaggagcccgcctccgccggcctctacttcttcgtggtcatcctctcctacgccaacagctgtgccaaccccgtcctctacggcttcctctctgacaacttccgccagagcttccagaaggttctgtgcctccgcaagggctctggtgccaaggacgctgacgccacggagccgcgtccagacaggatccggcagcagcaggaggccacgccgcccgcgcaccgcgccgcagccaacgggcttatgcagaccagcaagctgtga

SEQ ID NO: 52 - DNA, 2250 bp, Artificial
  Synthetic Construct; SF-human SSTR5Gqi5
atgaagacgatcatcgccctgagctacatcttctgcctggtgttcgccgactacaaggacgatgatgacgccatggagcccctgttcccagcctccacgcccagctggaacgcctcctccccgggggctgcctctggaggcggtgacaacaggacgctggtggggccggcgccctcggcaggggcccgggcggtgctggtgcccgtgctgtacctgctggtgtgtgcggccgggctgggcgggaacacgctggtcatctacgtggtgctgcgcttcgccaagatgaagaccgtcaccaacatctacattctcaacctggcagtggccgacgtcctgtacatgctggggctgcctttcctggccacgcagaacgccgcgtccttctggcccttcggccccgtcctgtgccgcctggtcatgacgctggacggcgtcaaccagttcaccagtgtcttctgcctgacagtcatgagcgtggaccgctacctggcagtggtgcacccgctgagctcggcccgctggcgccgcccgcgtgtggccaagctggcgagcgccgcggcctgggtcctgtctctgtgcatgtcgctgccgctcctggtgttcgcggacgtgcaggagggcggtacctgcaacgccagctggccggagcccgtggggctgtggggcgccgtcttcatcatctacacggccgtgctgggcttcttcgcgccgctgctggtcatctgcctgtgctacctgctcatcgtggtgaaggtgagggcggcgggcgtgcgcgtgggctgcgtgcggcggcgctcggagcggaaggtgacgcgcatggtgttggtggtggtgctggtgtttgcgggatgttggctgcccttcttcaccgtcaacatcgtcaacctggccgtggcgctgccccaggagcccgcctccgccggcctctacttcttcgtggtcatcctctcctacgccaacagctgtgccaaccccgtcctctacggcttcctctctgacaacttccgccagagcttccagaaggttctgtgcctccgcaagggctctggtgccaaggacgctgacgccacggagccgcgtccagacaggatccggcagcagcaggaggccacgccgcccgcgcaccgcgccgcagccaacgggcttatgcagaccagcaagctgttcgaaatgactctggagtccatcatggcgtgctgcctgagcgaggaggccaaggaagcccggcggatcaacgacgagatcgagcggcagctccgcagggacaagcgggacgcccgccgggagctcaagctgctgctgctcgggacaggagagagtggcaagagtacgtttatcaagcagatgagaatcatccatgggtcaggatactctgatgaagataaaaggggcttcaccaagctggtgtatcagaacatcttcacggccatgcaggccatgatcagagccatggacacactcaagatcccatacaagtatgagcacaataaggctcatgcacaattagttcgagaagttgatgtggagaaggtgtctgcttttgagaatccatatgtagatgcaataaagagtttatggaatgatcctggaatccaggaatgctatgatagacgacgagaatatcaattatctgactctaccaaatactatcttaatgacttggaccgcgtagctgaccctgcctacctgcctacgcaacaagatgtgcttagagttcgagtccccaccacagggatcatcgaatacccctttgacttacaaagtgtcattttcagaatggtcgatgtagggggccaaaggtcagagagaagaaaatggatacactgctttgaaaatgtcacctctatcatgtttctagtagcgcttagtgaatatgatcaagttctcgtggagtcagacaatgagaaccgaatggaggaaagcaaggctctctttagaacaattatcacatacccctggttccagaactcctcggttattctgttcttaaacaagaaagatcttctagaggagaaaatcatgtattcccatctagtcgactacttcccagaatatgatggaccccagagagatgcccaggcagcccgagaattcattctgaagatgttcgtggacctgaacccagacagtgacaaaattatctactcccacttcacgtgcgccacagacaccgagaatatccgctttgtctttgctgccgtcaaggacaccatcctccagttgaacctgaaggatattggtctgttctaa

SEQ ID NO: 53 - DNA, 1176 bp, Artificial
  Synthetic Construct; smyc-human SSTR5
atgaagacgatcatcgccctgagctacatcttctgcctggtattcgccgaacaaaagctcatttccgaagaggatcttgccatggagcccctgttcccagcctccacgcccagctggaacgcctcctccccgggggctgcctctggaggcggtgacaacaggacgctggtggggccggcgccctcggcaggggcccgggcggtgctggtgcccgtgctgtacctgctggtgtgtgcggccgggctgggcgggaacacgctggtcatctacgtggtgctgcgcttcgccaagatgaagaccgtcaccaacatctacattctcaacctggcagtggccgacgtcctgtacatgctggggctgcctttcctggccacgcagaacgccgcgtccttctggcccttcggccccgtcctgtgccgcctggtcatgacgctggacggcgtcaaccagttcaccagtgtcttctgcctgacagtcatgagcgtggaccgctacctggcagtggtgcacccgctgagctcggcccgctggcgccgcccgcgtgtggccaagctggcgagcgccgcggcctgggtcctgtctctgtgcatgtcgctgccgctcctggtgttcgcggacgtgcaggagggcggtacctgcaacgccagctggccggagcccgtggggctgtggggcgccgtcttcatcatctacacggccgtgctgggcttcttcgcgccgctgctggtcatctgcctgtgctacctgctcatcgtggtgaaggtgagggcggcgggcgtgcgcgtgggctgcgtgcggcggcgctcggagcggaaggtgacgcgcatggtgttggtggtggtgctggtgtttgcgggatgttggctgcccttcttcaccgtcaacatcgtcaacctggccgtggcgctgccccaggagcccgcctccgccggcctctacttcttcgtggtcatcctctcctacgccaacagctgtgccaaccccgtcctctacggcttcctctctgacaacttccgccagagcttccagaaggttctgtgcctccgcaagggctctggtgccaaggacgctgacgccacggagccgcgtccagacaggatccggcagcagcaggaggccacgccgcccgcgcaccgcgccgcagccaacgggcttatgcagaccagcaagctgtga

SEQ ID NO: 54 - DNA, 2625 bp, Artificial
  Synthetic Construct; 3HA-M4-linker-Gqi5
atgtacccatacgatgttccagattacgcttacccatacgatgttccagattacgcttacccatacgatgttccagattacgctgatgccaacttcacacctgtcaatggcagctcgggcaatcagtccgtgcgcctggtcacgtcatcatcccacaatcgctatgagacggtggaaatggtcttcattgccacagtgacaggctccctgagcctggtgactgtcgtgggcaacatcctggtgatgctgtccatcaaggtcaacaggcagctgcagacagtcaacaactacttcctcttcagcctggcgtgtgctgatctcatcataggcgccttctccatgaacctctacaccgtgtacatcatcaagggctactggcccctgggcgccgtggtctgcgacctgtggctggccctggactacgtggtgagcaacgcctccgtcatgaaccttctcatcatcagctttgaccgctacttctgcgtcaccaagcctctcacctaccctgcccggcgcaccaccaagatggcaggcctcatgattgctgctgcctgggtactgtccttcgtgctctgggcgcctgccatcttgttctggcagtttgtggtgggtaagcggacggtgcccgacaaccagtgcttcatccagttcctgtccaacccagcagtgacctttggcacagccattgctgccttctacctgcctgtggtcatcatgacggtgctgtacatccacatctccctggccagtcgcagccgagtccacaagcaccggcccgagggcccgaaggagaagaaagccaagacgctggccttcctcaagagcccactaatgaagcagagcgtcaagaagcccccgcccggggaggccgcccgggaggagctgcgcaatggcaagctggaggaggcccccccgccagcgctgccaccgccaccgcgccccgtggctgataaggacacttccaatgagtccagctcaggcagtgccacccagaacaccaaggaacgcccagccacagagctgtccaccacagaggccaccacgcccgccatgcccgcccctcccctgcagccgcgggccctcaacccagcctccagatggtccaagatccagattgtgacgaagcagacaggcaatgagtgtgtgacagccattgagattgtgcctgccacgccggctggcatgcgccctgcggccaacgtggcccgcaagttcgccagcatcgctcgcaaccaggtgcgcaagaagcggcagatggcggcccgggagcgcaaagtgacacgaacgatctttgccattctgctagccttcatcctcacctggacgccctacaacgtcatggtcctggtgaacaccttctgccagagctgcatccctgacacggtgtggtccattggctactggctctgctacgtcaacagcaccatcaaccctgcctgctatgctctgtgcaacgccacctttaaaaagaccttccggcacctgctgctgtgccagtatcggaacatcggcactgccaggttcgaaagacctgcagacggtagaatgactctggagtccatcatggcgtgctgcctgagcgaggaggccaaggaagcccggcggatcaacgacgagatcgagcggcagctccgcagggacaagcgggacgcccgccgggagctcaagctgctgctgctcgggacaggagagagtggcaagagtacgtttatcaagcagatgagaatcatccatgggtcaggatactctgatgaagataaaaggggcttcaccaagctggtgtatcagaacatcttcacggccatgcaggccatgatcagagccatggacacactcaagatcccatacaagtatgagcacaataaggctcatgcacaattagttcgagaagttgatgtggagaaggtgtctgcttttgagaatccatatgtagatgcaataaagagtttatggaatgatcctggaatccaggaatgctatgatagacgacgagaatatcaattatctgactctaccaaatactatcttaatgacttggaccgcgtagctgaccctgcctacctgcctacgcaacaagatgtgcttagagttcgagtccccaccacagggatcatcgaatacccctttgacttacaaagtgtcattttcagaatggtcgatgtagggggccaaaggtcagagagaagaaaatggatacactgctttgaaaatgtcacctctatcatgtttctagtagcgcttagtgaatatgatcaagttctcgtggagtcagacaatgagaaccgaatggaggaaagcaaggctctctttagaacaattatcacatacccctggttccagaactcctcggttattctgttcttaaacaagaaagatcttctagaggagaaaatcatgtattcccatctagtcgactacttcccagaatatgatggaccccagagagatgcccaggcagcccgagaattcattctgaagatgttcgtggacctgaacccagacagtgacaaaattatctactcccacttcacgtgcgccacagacaccgagaatatccgctttgtctttgctgccgtcaaggacaccatcctccagttgaacctgaaggatattggtctgttctaa

SEQ ID NO: 55 - DNA, 2607 bp, Artificial
  Synthetic Construct; 3HA-M4Gqi5a
atgtacccatacgatgttccagattacgcttacccatacgatgttccagattacgcttacccatacgatgttccagattacgctgatgccaacttcacacctgtcaatggcagctcgggcaatcagtccgtgcgcctggtcacgtcatcatcccacaatcgctatgagacggtggaaatggtcttcattgccacagtgacaggctccctgagcctggtgactgtcgtgggcaacatcctggtgatgctgtccatcaaggtcaacaggcagctgcagacagtcaacaactacttcctcttcagcctggcgtgtgctgatctcatcataggcgccttctccatgaacctctacaccgtgtacatcatcaagggctactggcccctgggcgccgtggtctgcgacctgtggctggccctggactacgtggtgagcaacgcctccgtcatgaaccttctcatcatcagctttgaccgctacttctgcgtcaccaagcctctcacctaccctgcccggcgcaccaccaagatggcaggcctcatgattgctgctgcctgggtactgtccttcgtgctctgggcgcctgccatcttgttctggcagtttgtggtgggtaagcggacggtgcccgacaaccagtgcttcatccagttcctgtccaacccagcagtgacctttggcacagccattgctgccttctacctgcctgtggtcatcatgacggtgctgtacatccacatctccctggccagtcgcagccgagtccacaagcaccggcccgagggcccgaaggagaagaaagccaagacgctggccttcctcaagagcccactaatgaagcagagcgtcaagaagcccccgcccggggaggccgcccgggaggagctgcgcaatggcaagctggaggaggcccccccgccagcgctgccaccgccaccgcgccccgtggctgataaggacacttccaatgagtccagctcaggcagtgccacccagaacaccaaggaacgcccagccacagagctgtccaccacagaggccaccacgcccgccatgcccgcccctcccctgcagccgcgggccctcaacccagcctccagatggtccaagatccagattgtgacgaagcagacaggcaatgagtgtgtgacagccattgagattgtgcctgccacgccggctggcatgcgccctgcggccaacgtggcccgcaagttcgccagcatcgctcgcaaccaggtgcgcaagaagcggcagatggcggcccgggagcgcaaagtgacacgaacgatctttgccattctgctagccttcatcctcacctggacgccctacaacgtcatggtcctggtgaacaccttctgccagagctgcatccctgacacggtgtggtccattggctactggctctgctacgtcaacagcaccatcaaccctgcctgctatgctctgtgcaacgccacctttaaaaagaccttccggcacctgctgctgtgccagtatcggaacatcggcactgccaggttcgaaatgactctggagtccatcatggcgtgctgcctgagcgaggaggccaaggaagcccggcggatcaacgacgagatcgagcggcagctccgcagggacaagcgggacgcccgccgggagctcaagctgctgctgctcgggacaggagagagtggcaagagtacgtttatcaagcagatgagaatcatccatgggtcaggatactctgatgaagataaaaggggcttcaccaagctggtgtatcagaacatcttcacggccatgcaggccatgatcagagccatggacacactcaagatcccatacaagtatgagcacaataaggctcatgcacaattagttcgagaagttgatgtggagaaggtgtctgcttttgagaatccatatgtagatgcaataaagagtttatggaatgatcctggaatccaggaatgctatgatagacgacgagaatatcaattatctgactctaccaaatactatcttaatgacttggaccgcgtagctgaccctgcctacctgcctacgcaacaagatgtgcttagagttcgagtccccaccacagggatcatcgaatacccctttgacttacaaagtgtcattttcagaatggtcgatgtagggggccaaaggtcagagagaagaaaatggatacactgctttgaaaatgtcacctctatcatgtttctagtagcgcttagtgaatatgatcaagttctcgtggagtcagacaatgagaaccgaatggaggaaagcaaggctctctttagaacaattatcacatacccctggttccagaactcctcggttattctgttcttaaacaagaaagatcttctagaggagaaaatcatgtattcccatctagtcgactacttcccagaatatgatggaccccagagagatgcccaggcagcccgagaattcattctgaagatgttcgtggacctgaacccagacagtgacaaaattatctactcccacttcacgtgcgccacagacaccgagaatatccgctttgtctttgctgccgtcaaggacaccatcctccagttgaacctgaaggatattggtctgttctaa

SEQ ID NO: 56 - DNA, 1059 bp, Homo sapiens
atggattatcaagtgtcaagtccaatctatgacatcaattattatacatcggagccctgccaaaaaatcaatgtgaagcaaatcgcagcccgcctcctgcctccgctctactcactggtgttcatctttggttttgtgggcaacatgctggtcatcctcatcctgataaactgcaaaaggctgaagagcatgactgacatctacctgctcaacctggccatctctgacctgtttttccttcttactgtccccttctgggctcactatgctgccgcccagtgggactttggaaatacaatgtgtcaactcttgacagggctctattttataggcttcttctctggaatcttcttcatcatcctcctgacaatcgataggtacctggctgtcgtccatgctgtgtttgctttaaaagccaggacggtcacctttggggtggtgacaagtgtgatcacttgggtggtggctgtgtttgcgtctctcccaggaatcatctttaccagatctcaaaaagaaggtcttcattacacctgcagctctcattttccatacagtcagtatcaattctggaagaatttccagacattaaagatagtcatcttggggctggtcctgccgctgcttgtcatggtcatctgctactcgggaatcctaaaaactctgcttcggtgtcgaaatgagaagaagaggcacagggctgtgaggcttatcttcaccatcatgattgtttattttctcttctgggctccctacaacattgtccttctcctgaacaccttccaggaattctttggcctgaataattgcagtagctctaacaggttggaccaagctatgcaggtgacagagactcttgggatgacgcactgctgcatcaaccccatcatctatgcctttgtcggggagaagttcagaaactacctcttagtcttcttccaaaagcacattgccaaacgcttctgcaaatgctgttctattttccagcaagaggctcccgagcgagcaagctcagtttacacccgatccactggggagcaggaaatatctgtgggcttgtga

SEQ ID NO: 57 - DNA, 2142 bp, Artificial
  Synthetic Construct; CCR5 Gqi5
atggattatcaagtgtcaagtccaatctatgacatcaattattatacatcggagccctgccaaaaaatcaatgtgaagcaaatcgcagcccgcctcctgcctccgctctactcactggtgttcatctttggttttgtgggcaacatgctggtcatcctcatcctgataaactgcaaaaggctgaagagcatgactgacatctacctgctcaacctggccatctctgacctgtttttccttcttactgtccccttctgggctcactatgctgccgcccagtgggactttggaaatacaatgtgtcaactcttgacagggctctattttataggcttcttctctggaatcttcttcatcatcctcctgacaatcgataggtacctggctgtcgtccatgctgtgtttgctttaaaagccaggacggtcacctttggggtggtgacaagtgtgatcacttgggtggtggctgtgtttgcgtctctcccaggaatcatctttaccagatctcaaaaagaaggtcttcattacacctgcagctctcattttccatacagtcagtatcaattctggaagaatttccagacattaaagatagtcatcttggggctggtcctgccgctgcttgtcatggtcatctgctactcgggaatcctaaaaactctgcttcggtgtcgaaatgagaagaagaggcacagggctgtgaggcttatcttcaccatcatgattgtttattttctcttctgggctccctacaacattgtccttctcctgaacaccttccaggaattctttggcctgaataattgcagtagctctaacaggttggaccaagctatgcaggtgacagagactcttgggatgacgcactgctgcatcaaccccatcatctatgcctttgtcggggagaagttcagaaactacctcttagtcttcttccaaaagcacattgccaaacgcttctgcaaatgctgttctattttccagcaagaggctcccgagcgagcaagctcagtttacacccgatccactggggagcaggaaatatctgtgggcttgttcgaaatgactctggagtccatcatggcgtgctgcctgagcgaggaggccaaggaagcccggcggatcaacgacgagatcgagcggcagctccgcagggacaagcgggacgcccgccgggagctcaagctgctgctgctcgggacaggagagagtggcaagagtacgtttatcaagcagatgagaatcatccatgggtcaggatactctgatgaagataaaaggggcttcaccaagctggtgtatcagaacatcttcacggccatgcaggccatgatcagagccatggacacactcaagatcccatacaagtatgagcacaataaggctcatgcacaattagttcgagaagttgatgtggagaaggtgtctgcttttgagaatccatatgtagatgcaataaagagtttatggaatgatcctggaatccaggaatgctatgatagacgacgagaatatcaattatctgactctaccaaatactatcttaatgacttggaccgcgtagctgaccctgcctacctgcctacgcaacaagatgtgcttagagttcgagtccccaccacagggatcatcgaatacccctttgacttacaaagtgtcattttcagaatggtcgatgtagggggccaaaggtcagagagaagaaaatggatacactgctttgaaaatgtcacctctatcatgtttctagtagcgcttagtgaatatgatcaagttctcgtggagtcagacaatgagaaccgaatggaggaaagcaaggctctctttagaacaattatcacatacccctggttccagaactcctcggttattctgttcttaaacaagaaagatcttctagaggagaaaatcatgtattcccatctagtcgactacttcccagaatatgatggaccccagagagatgcccaggcagcccgagaattcattctgaagatgttcgtggacctgaacccagacagtgacaaaattatctactcccacttcacgtgcgccacagacaccgagaatatccgctttgtctttgctgccgtcaaggacaccatcctccagttgaacctgaaggatattggtctgttctaa

SEQ ID NO: 58 - DNA, 1200 bp, Artificial
  Synthetic Construct; smycDOR
atgaagacgatcatcgccctgagctacatcttctgcctggtattcgccgaacaaaagctcatttccgaagaggatcttgccatggagctggtgccctctgcccgtgcggagctgcagtcctcgcccctcgtcaacctctcggacgcctttcccagcgccttccccagcgcgggcgccaatgcgtcggggtcgccgggagcccgtagtgcctcgtccctcgccctagccatcgccatcaccgcgctctactcggctgtgtgcgcagtggggcttctgggcaacgtgctcgtcatgtttggcatcgtccggtacaccaaattgaagaccgccaccaacatctacatcttcaatctggctttggctgatgcgctggccaccagcacgctgcccttccagagcgccaagtacttgatggaaacgtggccgtttggcgagctgctgtgcaaggctgtgctctccattgactactacaacatgttcactagcatcttcaccctcaccatgatgagcgtggaccgctacattgctgtctgccatcctgtcaaagccctggacttccggacaccagccaaggccaagctgatcaatatatgcatctgggtcttggcttcaggtgtcggggtccccatcatggtcatggcagtgacccaaccccgggatggtgcagtggtatgcatgctccagttccccagtcccagctggtactgggacactgtgaccaagatctgcgtgttcctctttgccttcgtggtgccgatcctcatcatcacggtgtgctatggcctcatgctactgcgcctgcgcagcgtgcgtctgctgtccggttccaaggagaaggaccgcagcctgcggcgcatcacgcgcatggtgctggtggtggtgggcgccttcgtggtgtgctgggcgcccatccacatcttcgtcatcgtctggacgctggtggacatcaatcggcgcgacccacttgtggtggccgcactgcacctgtgcattgcgctgggctacgccaacagcagcctcaacccggttctctacgccttcctggacgagaacttcaagcgctgcttccgccagctctgtcgcacgccctgcggccgccaagaacccggcagtctccgtcgtccccgccaggccaccacgcgtgagcgtgtcactgcctgcaccccctccgacggcccgggcggtggcgctgccgcctga

SEQ ID NO: 59 - DNA, 3378 bp, Artificial
  Synthetic Construct; TSHr Gqi5
atgaggccggcggacttgctgcagctggtgctgctgctcgacctgcccagggacctgggcggaatggggtgttcgtctccaccctgcgagtgccatcaggaggaggacttcagagtcacctgcaaggatattcaacgcatccccagcttaccgcccagtacgcagactctgaagcttattgagactcacctgagaactattccaagtcatgcattttctaatctgcccaatatttccagaatctacgtatctatagatctgactctgcagcagctggaatcacactccttctacaatttgagtaaagtgactcacatagaaattcggaataccaggaacttaacttacatagaccctgatgccctcaaagagctccccctcctaaagttccttggcattttcaacactggacttaaaatgttccctgacctgaccaaagtttattccactgatatattctttatacttgaaattacagacaacccttacatgacgtcaatccctgtgaatgcttttcagggactatgcaatgaaaccttgacactgaagctgtacaacaatggctttacttcagtccaaggatatgctttcaatgggacaaagctggatgctgtttacctaaacaagaataaatacctgacagttattgacaaagatgcatttggaggagtatacagtggaccaagcttgctggacgtgtctcaaaccagtgtcactgcccttccatccaaaggcctggagcacctgaaggaactgatagcaagaaacacctggactcttaagaaacttccactttccttgagtttccttcacctcacacgggctgacctttcttacccaagccactgctgtgcttttaagaatcagaagaaaatcagaggaatccttgagtccttgatgtgtaatgagagcagtatgcagagcttgcgccagagaaaatctgtgaatgccttgaatagccccctccaccaggaatatgaagagaatctgggtgacagcattgttgggtacaaggaaaagtccaagttccaggatactcataacaacgctcattattacgtcttctttgaagaacaagaggatgagatcattggttttggccaggagctcaaaaacccccaggaagagactctacaagcttttgacagccattatgactacaccatatgtggggacagtgaagacatggtgtgtacccccaagtccgatgagttcaacccgtgtgaagacataatgggctacaagttcctgagaattgtggtgtggttcgttagtctgctggctctcctgggcaatgtctttgtcctgcttattctcctcaccagccactacaaactgaacgtcccccgctttctcatgtgcaacctggcctttgcggatttctgcatggggatgtacctgctcctcatcgcctctgtagacctctacactcactctgagtactacaaccatgccatcgactggcagacaggccctgggtgcaacacggctggtttcttcactgtctttgcaagcgagttatcggtgtatacgctgacggtcatcaccctggagcgctggtatgccatcaccttcgccatgcgcctggaccggaagatccgcctcaggcacgcatgtgccatcatggttgggggctgggtttgctgcttccttctcgccctgcttcctttggtgggaataagtagctatgccaaagtcagtatctgcctgcccatggacaccgagacccctcttgctctggcatatattgtttttgttctgacgctcaacatagttgccttcgtcatcgtctgctgctgttatgtgaagatctacatcacagtccgaaatccgcagtacaacccaggggacaaagataccaaaattgccaagaggatggctgtgttgatcttcaccgacttcatatgcatggccccaatctcattctatgctctgtcagcaattctgaacaagcctctcatcactgttagcaactccaaaatcttgctggtactcttctatccacttaactcctgtgccaatccattcctctatgctattttcaccaaggccttccagagggatgtgttcatcctactcagcaagtttggcatctgtaaacgccaggctcaggcataccgggggcagagggttcctccaaagaacagcactgatattcaggttcaaaaggttacccacgacatgaggcagggtctccacaacatggaagatgtctatgaactgattgaaaactcccatctaaccccaaagaagcaaggccaaatctcagaagagtatatgcaaacggttttgttcgaaatgactctggagtccatcatggcgtgctgcctgagcgaggaggccaaggaagcccggcggatcaacgacgagatcgagcggcagctccgcagggacaagcgggacgcccgccgggagctcaagctgctgctgctcgggacaggagagagtggcaagagtacgtttatcaagcagatgagaatcatccatgggtcaggatactctgatgaagataaaaggggcttcaccaagctggtgtatcagaacatcttcacggccatgcaggccatgatcagagccatggacacactcaagatcccatacaagtatgagcacaataaggctcatgcacaattagttcgagaagttgatgtggagaaggtgtctgcttttgagaatccatatgtagatgcaataaagagtttatggaatgatcctggaatccaggaatgctatgatagacgacgagaatatcaattatctgactctaccaaatactatcttaatgacttggaccgcgtagctgaccctgcctacctgcctacgcaacaagatgtgcttagagttcgagtccccaccacagggatcatcgaatacccctttgacttacaaagtgtcattttcagaatggtcgatgtagggggccaaaggtcagagagaagaaaatggatacactgctttgaaaatgtcacctctatcatgtttctagtagcgcttagtgaatatgatcaagttctcgtggagtcagacaatgagaaccgaatggaggaaagcaaggctctctttagaacaattatcacatacccctggttccagaactcctcggttattctgttcttaaacaagaaagatcttctagaggagaaaatcatgtattcccatctagtcgactacttcccagaatatgatggaccccagagagatgcccaggcagcccgagaattcattctgaagatgttcgtggacctgaacccagacagtgacaaaattatctactcccacttcacgtgcgccacagacaccgagaatatccgctttgtctttgctgccgtcaaggacaccatcctccagttgaacctgaaggatattggtctgttctaa

SEQ ID NO: 60 - DNA, 1245 bp, Homo sapiens
atggatccactgaatctgtcctggtatgatgatgatctggagaggcagaactggagccggcccttcaacgggtcagacgggaaggcggacagaccccactacaactactatgccacactgctcaccctgctcatcgctgtcatcgtcttcggcaacgtgctggtgtgcatggctgtgtcccgcgagaaggcgctgcagaccaccaccaactacctgatcgtcagcctcgcagtggccgacctcctcgtcgccacactggtcatgccctgggttgtctacctggaggtggtaggtgagtggaaattcagcaggattcactgtgacatcttcgtcactctggacgtcatgatgtgcacggcgagcatcctgaacttgtgtgccatcagcatcgacaggtacacagctgtggccatgcccatgctgtacaatacgcgctacagctccaagcgccgggtcaccgtcatgatctccatcgtctgggtcctgtccttcaccatctcctgcccactcctcttcggactcaataacgcagaccagaacgagtgcatcattgccaacccggccttcgtggtctactcctccatcgtctccttctacgtgcccttcattgtcaccctgctggtctacatcaagatctacattgtcctccgcagacgccgcaagcgagtcaacaccaaacgcagcagccgagctttcagggcccacctgagggctccactaaaggaggctgcccggcgagcccaggagctggagatggagatgctctccagcaccagcccacccgagaggacccggtacagccccatcccacccagccaccaccagctgactctccccgacccgtcccaccatggtctccacagcactcccgacagccccgccaaaccagagaagaatgggcatgccaaagaccaccccaagattgccaagatctttgagatccagaccatgcccaatggcaaaacgcgtacctccctcaagaccatgagccgtaggaagctctcccagcagaaggagaagaaagccactcagatgctcgccattgttctcggcgtgttcatcatctgctggctgcccttcttcatcacacacatcctgaacatacactgtgactgcaacatcccgcctgtcctgtacagcgccttcacgtggctgggctatgtcaacagcgccgtgaaccccatcatctacaccaccttcaacattgagttccgcaaggccttcctgaagatcctccactgctga

SEQ ID NO: 61 - protein, 414 aa, Homo sapiens
MDPLNLSWYDDDLERQNWSRPFNGSDGKADRPHYNYYATLLTLLIAVIVFGNVLVCMAVSREKALQTTTNYLIVSLAVADLLVATLVMPWVVYLEVVGEWKFSRIHCDIFVTLDVMMCTASILNLCAISIDRYTAVAMPMLYNTRYSSKRRVTVMISIVWVLSFTISCPLLFGLNNADQNECIIANPAFVVYSSIVSFYVPFIVTLLVYIKIYIVLRRRRKRVNTKRSSRAFRAHLRAPLKEAARRAQELEMEMLSSTSPPERTRYSPIPPSHHQLTLPDPSHHGLHSTPDSPAKPEKNGHAKDHPKIAKIFEIQTMPNGKTRTSLKTMSRRKLSQQKEKKATQMLAIVLGVFIICWLPFFITHILNIHCDCNIPPVLYSAFTWLGYVNSAVNPIIYTTFNIEFRKAFLKILHC

SEQ ID NO: 62 - DNA, 1317 bp, Artificial
  Synthetic construct; hD2 with signal sequence and FLAG tag
atgaagacgatcatcgccctgagctacatcttctgcctggtattcgccgactacaaggacgatgatgacgccatggatccactgaatctgtcctggtatgatgatgatctggagaggcagaactggagccggcccttcaacgggtcagacgggaaggcggacagaccccactacaactactatgccacactgctcaccctgctcatcgctgtcatcgtcttcggcaacgtgctggtgtgcatggctgtgtcccgcgagaaggcgctgcagaccaccaccaactacctgatcgtcagcctcgcagtggccgacctcctcgtcgccacactggtcatgccctgggttgtctacctggaggtggtaggtgagtggaaattcagcaggattcactgtgacatcttcgtcactctggacgtcatgatgtgcacggcgagcatcctgaacttgtgtgccatcagcatcgacaggtacacagctgtggccatgcccatgctgtacaatacgcgctacagctccaagcgccgggtcaccgtcatgatctccatcgtctgggtcctgtccttcaccatctcctgcccactcctcttcggactcaataacgcagaccagaacgagtgcatcattgccaacccggccttcgtggtctactcctccatcgtctccttctacgtgcccttcattgtcaccctgctggtctacatcaagatctacattgtcctccgcagacgccgcaagcgagtcaacaccaaacgcagcagccgagctttcagggcccacctgagggctccactaaaggaggctgcccggcgagcccaggagctggagatggagatgctctccagcaccagcccacccgagaggacccggtacagccccatcccacccagccaccaccagctgactctccccgacccgtcccaccatggtctccacagcactcccgacagccccgccaaaccagagaagaatgggcatgccaaagaccaccccaagattgccaagatctttgagatccagaccatgcccaatggcaaaacgcgtacctccctcaagaccatgagccgtaggaagctctcccagcagaaggagaagaaagccactcagatgctcgccattgttctcggcgtgttcatcatctgctggctgcccttcttcatcacacacatcctgaacatacactgtgactgcaacatcccgcctgtcctgtacagcgccttcacgtggctgggctatgtcaacagcgccgtgaaccccatcatctacaccaccttcaacattgagttccgcaaggccttcctgaagatcctccactgctga

SEQ ID NO: 63 - protein, 438 aa, Artificial
  Synthetic Construct; hD2 with signal and FLAG
MKTIIALSYIFCLVFADYKDDDDAMDPLNLSWYDDDLERQNWSRPFNGSDGKADRPHYNYYATLLTLLIAVIVFGNVLVCMAVSREKALQTTTNYLIVSLAVADLLVATLVMPWVVYLEVVGEWKFSRIHCDIFVTLDVMMCTASILNLCAISIDRYTAVAMPMLYNTRYSSKRRVTVMISIVWVLSFTISCPLLFGLNNADQNECIIANPAFVVYSSIVSFYVPFIVTLLVYIKIYIVLRRRRKRVNTKRSSRAFRAHLRAPLKEAARRAQELEMEMLSSTSPPERTRYSPIPPSHHQLTLPDPSHHGLHSTPDSPAKPEKNGHAKDHPKIAKIFEIQTMPNGKTRTSLKTMSRRKLSQQKEKKATQMLAIVLGVFIICWLPFFITHILNIHCDCNIPPVLYSAFTWLGYVNSAVNPIIYTTFNIEFRKAFLKILHC

SEQ ID NO: 64 - protein, 799 aa, Artificial
  Synthetic Construct; SFD2sWTGqi5
MKTIIALSYIFCLVFADYKDDDDAMDPLNLSWYDDDLERQNWSRPFNGSDGKADRPHYNYYATLLTLLIAVIVFGNVLVCMAVSREKALQTTTNYLIVSLAVADLLVATLVMPWVVYLEVVGEWKFSRIHCDIFVTLDVMMCTASILNLCAISIDRYTAVAMPMLYNTRYSSKRRVTVMISIVWVLSFTISCPLLFGLNNADQNECIIANPAFVVYSSIVSFYVPFIVTLLVYIKIYIVLRRRRKRVNTKRSSRAFRAHLRAPLKEAARRAQELEMEMLSSTSPPERTRYSPIPPSHHQLTLPDPSHHGLHSTPDSPAKPEKNGHAKDHPKIAKIFEIQTMPNGKTRTSLKTMSRRKLSQQKEKKATQMLAIVLGVFIICWLPFFITHILNIHCDCNIPPVLYSAFTWLGYVNSAVNPIIYTTFNIEFRKAFLKILHCFEMTLESIMACCLSEEAKEARRINDEIERQLRRDKRDARRELKLLLLGTGESGKSTFIKQMRIIHGSGYSDEDKRGFTKLVYQNIFTAMQAMIRAMDTLKIPYKYEHNKAHAQLVREVDVEKVSAFENPYVDAIKSLWNDPGIQECYDRRREYQLSDSTKYYLNDLDRVADPAYLPTQQDVLRVRVPTTGIIEYPFDLQSVIFRMVDVGGQRSERRKWIHCFENVTSIMFLVALSEYDQVLVESDNENRMEESKALFRTIITYPWFQNSSVILFLNKKDLLEEKIMYSHLVDYFPEYDGPQRDAQAAREFILKMFVDLNPDSDKIIYSHFTCATDTENIRFVFAAVKDTILQLNLKDIGLF

SEQ ID NO: 65 - protein, 439 aa, Artificial
  Synthetic construct; sMycD2sWT
MKTIIALSYIFCLVFAEQKLISEEDLDPLNLSWYDDDLERQNWSRPFNGSDGKADRPHYNYYATLLTLLIAVIVFGNVLVCMAVSREKALQTTTNYLIVSLAVADLLVATLVMPWVVYLEVVGEWKFSRIHCDIFVTLDVMMCTASILNLCAISIDRYTAVAMPMLYNTRYSSKRRVTVMISIVWVLSFTISCPLLFGLNNADQNECIIANPAFVVYSSIVSFYVPFIVTLLVYIKIYIVLRRRRKRVNTKRSSRAFRAHLRAPLKEAARRAQELEMEMLSSTSPPERTRYSPIPPSHHQLTLPDPSHHGLHSTPDSPAKPEKNGHAKDHPKIAKIFEIQTMPNGKTRTSLKTMSRRKLSQQKEKKATQMLAIVLGVFIICWLPFFITHILNIHCDCNIPPVLYSAFTWLGYVNSAVNPIIYTTFNIEFRKAFLKILHC

SEQ ID NO: 66 - protein, 443 aa, Homo sapiens
MDPLNLSWYDDDLERQNWSRPFNGSDGKADRPHYNYYATLLTLLIAVIVFGNVLVCMAVSREKALQTTTNYLIVSLAVADLLVATLVMPWVVYLEVVGEWKFSRIHCDIFVTLDVMMCTASILNLCAISIDRYTAVAMPMLYNTRYSSKRRVTVMISIVWVLSFTISCPLLFGLNNADQNECIIANPAFVVYSSIVSFYVPFIVTLLVYIKIYIVLRRRRKRVNTKRSSRAFRAHLRAPLKGNCTHPEDMKLCTVIMKSNGSFPVNRRRVEAARRAQELEMEMLSSTSPPERTRYSPIPPSHHQLTLPDPSHHGLHSTPDSPAKPEKNGHAKDHPKIAKIFEIQTMPNGKTRTSLKTMSRRKLSQQKEKKATQMLAIVLGVFIICWLPFFITHILNIHCDCNIPPVLYSAFTWLGYVNSAVNPIIYTTFNIEFRKAFLKILHC

SEQ ID NO: 67 - DNA, 1080 bp, Artificial
  Synthetic Construct; Gqi5
atgactctggagtccatcatggcgtgctgcctgagcgaggaggccaaggaagcccggcggatcaacgacgagatcgagcggcagctccgcagggacaagcgggacgcccgccgggagctcaagctgctgctgctcgggacaggagagagtggcaagagtacgtttatcaagcagatgagaatcatccatgggtcaggatactctgatgaagataaaaggggcttcaccaagctggtgtatcagaacatcttcacggccatgcaggccatgatcagagccatggacacactcaagatcccatacaagtatgagcacaataaggctcatgcacaattagttcgagaagttgatgtggagaaggtgtctgcttttgagaatccatatgtagatgcaataaagagtttatggaatgatcctggaatccaggaatgctatgatagacgacgagaatatcaattatctgactctaccaaatactatcttaatgacttggaccgcgtagctgaccctgcctacctgcctacgcaacaagatgtgcttagagttcgagtccccaccacagggatcatcgaatacccctttgacttacaaagtgtcattttcagaatggtcgatgtagggggccaaaggtcagagagaagaaaatggatacactgctttgaaaatgtcacctctatcatgtttctagtagcgcttagtgaatatgatcaagttctcgtggagtcagacaatgagaaccgaatggaggaaagcaaggctctctttagaacaattatcacatacccctggttccagaactcctcggttattctgttcttaaacaagaaagatcttctagaggagaaaatcatgtattcccatctagtcgactacttcccagaatatgatggaccccagagagatgcccaggcagcccgagaattcattctgaagatgttcgtggacctgaacccagacagtgacaaaattatctactcccacttcacgtgcgccacagacaccgagaatatccgctttgtctttgctgccgtcaaggacaccatcctccagttgaacctgaaggatattggtctgttctaa

SEQ ID NO: 68 - protein, 359 aa, Artificial
  Synthetic construct, Gqi5
MTLESIMACCLSEEAKEARRINDEIERQLRRDKRDARRELKLLLLGTGESGKSTFIKQMRIIHGSGYSDEDKRGFTKLVYQNIFTAMQAMIRAMDTLKIPYKYEHNKAHAQLVREVDVEKVSAFENPYVDAIKSLWNDPGIQECYDRRREYQLSDSTKYYLNDLDRVADPAYLPTQQDVLRVRVPTTGIIEYPFDLQSVIFRMVDVGGQRSERRKWIHCFENVTSIMFLVALSEYDQVLVESDNENRMEESKALFRTIITYPWFQNSSVILFLNKKDLLEEKIMYSHLVDYFPEYDGPQRDAQAAREFILKMFVDLNPDSDKIIYSHFTCATDTENIRFVFAAVKDTILQLNLKDIGLF

SEQ ID NO: 69 - DNA, 2418 bp, Artificial
  Synthetic construct; flag-tagged D2s-linker-Gqi5
atgaagacgatcatcgccctgagctacatcttctgcctggtattcgccgactacaaggacgatgatgacgccatggatccactgaatctgtcctggtatgatgatgatctggagaggcagaactggagccggcccttcaacgggtcagacgggaaggcggacagaccccactacaactactatgccacactgctcaccctgctcatcgctgtcatcgtcttcggcaacgtgctggtgtgcatggctgtgtcccgcgagaaggcgctgcagaccaccaccaactacctgatcgtcagcctcgcagtggccgacctcctcgtcgccacactggtcatgccctgggttgtctacctggaggtggtaggtgagtggaaattcagcaggattcactgtgacatcttcgtcactctggacgtcatgatgtgcacggcgagcatcctgaacttgtgtgccatcagcatcgacaggtacacagctgtggccatgcccatgctgtacaatacgcgctacagctccaagcgccgggtcaccgtcatgatctccatcgtctgggtcctgtccttcaccatctcctgcccactcctcttcggactcaataacgcagaccagaacgagtgcatcattgccaacccggccttcgtggtctactcctccatcgtctccttctacgtgcccttcattgtcaccctgctggtctacatcaagatctacattgtcctccgcagacgccgcaagcgagtcaacaccaaacgcagcagccgagctttcagggcccacctgagggctccactaaaggaggctgcccggcgagcccaggagctggagatggagatgctctccagcaccagcccacccgagaggacccggtacagccccatcccacccagccaccaccagctgactctccccgacccgtcccaccatggtctccacagcactcccgacagccccgccaaaccagagaagaatgggcatgccaaagaccaccccaagattgccaagatctttgagatccagaccatgcccaatggcaaaacgcgtacctccctcaagaccatgagccgtaggaagctctcccagcagaaggagaagaaagccactcagatgctcgccattgttctcggcgtgttcatcatctgctggctgcccttcttcatcacacacatcctgaacatacactgtgactgcaacatcccgcctgtcctgtacagcgccttcacgtggctgggctatgtcaacagcgccgtgaaccccatcatctacaccaccttcaacattgagttccgcaaggccttcctgaagatcctccactgtttcgaaagacctgcagacggtagaatgactctggagtccatcatggcgtgctgcctgagcgaggaggccaaggaagcccggcggatcaacgacgagatcgagcggcagctccgcagggacaagcgggacgcccgccgggagctcaagctgctgctgctcgggacaggagagagtggcaagagtacgtttatcaagcagatgagaatcatccatgggtcaggatactctgatgaagataaaaggggcttcaccaagctggtgtatcagaacatcttcacggccatgcaggccatgatcagagccatggacacactcaagatcccatacaagtatgagcacaataaggctcatgcacaattagttcgagaagttgatgtggagaaggtgtctgcttttgagaatccatatgtagatgcaataaagagtttatggaatgatcctggaatccaggaatgctatgatagacgacgagaatatcaattatctgactctaccaaatactatcttaatgacttggaccgcgtagctgaccctgcctacctgcctacgcaacaagatgtgcttagagttcgagtccccaccacagggatcatcgaatacccctttgacttacaaagtgtcattttcagaatggtcgatgtagggggccaaaggtcagagagaagaaaatggatacactgctttgaaaatgtcacctctatcatgtttctagtagcgcttagtgaatatgatcaagttctcgtggagtcagacaatgagaaccgaatggaggaaagcaaggctctctttagaacaattatcacatacccctggttccagaactcctcggttattctgttcttaaacaagaaagatcttctagaggagaaaatcatgtattcccatctagtcgactacttcccagaatatgatggaccccagagagatgcccaggcagcccgagaattcattctgaagatgttcgtggacctgaacccagacagtgacaaaattatctactcccacttcacgtgcgccacagacaccgagaatatccgctttgtctttgctgccgtcaaggacaccatcctccagttgaacctgaaggatattggtctgttctaa

SEQ ID NO: 70 - protein, 805 aa, Artificial
  Synthetic construct; D2s-linker-Gqi5 G208A
MKTIIALSYIFCLVFADYKDDDDAMDPLNLSWYDDDLERQNWSRPFNGSDGKADRPHYNYYATLLTLLIAVIVFGNVLVCMAVSREKALQTTTNYLIVSLAVADLLVATLVMPWVVYLEVVGEWKFSRIHCDIFVTLDVMMCTASILNLCAISIDRYTAVAMPMLYNTRYSSKRRVTVMISIVWVLSFTISCPLLFGLNNADQNECIIANPAFVVYSSIVSFYVPFIVTLLVYIKIYIVLRRRRKRVNTKRSSRAFRAHLRAPLKEAARRAQELEMEMLSSTSPPERTRYSPIPPSHHQLTLPDPSHHGLHSTPDSPAKPEKNGHAKDHPKIAKIFEIQTMPNGKTRTSLKTMSRRKLSQQKEKKATQMLAIVLGVFIICWLPFFITHILNIHCDCNIPPVLYSAFTWLGYVNSAVNPIIYTTFNIEFRKAFLKILHCFERPADGRMTLESIMACCLSEEAKEARRINDEIERQLRRDKRDARRELKLLLLGTGESGKSTFIKQMRIIHGSGYSDEDKRGFTKLVYQNIFTAMQAMIRAMDTLKIPYKYEHNKAHAQLVREVDVEKVSAFENPYVDAIKSLWNDPGIQECYDRRREYQLSDSTKYYLNDLDRVADPAYLPTQQDVLRVRVPTTGIIEYPFDLQSVIFRMVDVGAQRSERRKWIHCFENVTSIMFLVALSEYDQVLVESDNENRMEESKALFRTIITYPWFQNSSVILFLNKKDLLEEKIMYSHLVDYFPEYDGPQRDAQAAREFILKMFVDLNPDSDKIIYSHFTCATDTENIRFVFAAVKDTILQLNLKDIGLF

SEQ ID NO: 71 - DNA, 2284 bp, Artificial
  Synthetic construct; Smyc-DOR-Gqi5
catgaagacgatcatcgccctgagctacatcttctgcctggtattcgccgaacaaaagctcatttccgaagaggatcttgccatggagctggtgccctctgcccgtgcggagctgcagtcctcgcccctcgtcaacctctcggacgcctttcccagcgccttccccagcgcgggcgccaatgcgtcggggtcgccgggagcccgtagtgcctcgtccctcgccctagccatcgccatcaccgcgctctactcggctgtgtgcgcagtggggcttctgggcaacgtgctcgtcatgtttggcatcgtccggtacaccaaattgaagaccgccaccaacatctacatcttcaatctggctttggctgatgcgctggccaccagcacgctgcccttccagagcgccaagtacttgatggaaacgtggccgtttggcgagctgctgtgcaaggctgtgctctccattgactactacaacatgttcactagcatcttcaccctcaccatgatgagcgtggaccgctacattgctgtctgccatcctgtcaaagccctggacttccggacaccagccaaggccaagctgatcaatatatgcatctgggtcttggcttcaggtgtcggggtccccatcatggtcatggcagtgacccaaccccgggatggtgcagtggtatgcatgctccagttccccagtcccagctggtactgggacactgtgaccaagatctgcgtgttcctctttgccttcgtggtgccgatcctcatcatcacggtgtgctatggcctcatgctactgcgcctgcgcagcgtgcgtctgctgtccggttccaaggagaaggaccgcagcctgcggcgcatcacgcgcatggtgctggtggtggtgggcgccttcgtggtgtgctgggcgcccatccacatcttcgtcatcgtctggacgctggtggacatcaatcggcgcgacccacttgtggtggccgcactgcacctgtgcattgcgctgggctacgccaacagcagcctcaacccggttctctacgccttcctggacgagaacttcaagcgctgcttccgccagctctgtcgcacgccctgcggccgccaagaacccggcagtctccgtcgtccccgccaggccaccacgcgtgagcgtgtcactgcctgcaccccctccgacggcccgggcggtggcgctgccgccttcgaaatgactctggagtccatcatggcgtgctgcctgagcgaggaggccaaggaagcccggcggatcaacgacgagatcgagcggcagctccgcagggacaagcgggacgcccgccgggagctcaagctgctgctgctcgggacaggagagagtggcaagagtacgtttatcaagcagatgagaatcatccatgggtcaggatactctgatgaagataaaaggggcttcaccaagctggtgtatcagaacatcttcacggccatgcaggccatgatcagagccatggacacactcaagatcccatacaagtatgagcacaataaggctcatgcacaattagttcgagaagttgatgtggagaaggtgtctgcttttgagaatccatatgtagatgcaataaagagtttatggaatgatcctggaatccaggaatgctatgatagacgacgagaatatcaattatctgactctaccaaatactatcttaatgacttggaccgcgtagctgaccctgcctacctgcctacgcaacaagatgtgcttagagttcgagtccccaccacagggatcatcgaatacccctttgacttacaaagtgtcattttcagaatggtcgatgtagggggccaaaggtcagagagaagaaaatggatacactgctttgaaaatgtcacctctatcatgtttctagtagcgcttagtgaatatgatcaagttctcgtggagtcagacaatgagaaccgaatggaggaaagcaaggctctctttagaacaattatcacatacccctggttccagaactcctcggttattctgttcttaaacaagaaagatcttctagaggagaaaatcatgtattcccatctagtcgactacttcccagaatatgatggaccccagagagatgcccaggcagcccgagaattcattctgaagatgttcgtggacctgaacccagacagtgacaaaattatctactcccacttcacgtgcgccacagacaccgagaatatccgctttgtctttgctgccgtcaaggacaccatcctccagttgaacctgaaggatattggtctgttctaa

SEQ ID NO: 72 - protein, 760 aa, Artificial
  synthetic construct; myc-tagged DOR-Gqi5
MKTIIALSYIFCLVFAEQKLISEEDLAMELVPSARAELQSSPLVNLSDAFPSAFPSAGANASGSPGARSASSLALAIAITALYSAVCAVGLLGNVLVMFGIVRYTKLKTATNIYIFNLALADALATSTLPFQSAKYLMETWPFGELLCKAVLSIDYYNMFTSIFTLTMMSVDRYIAVCHPVKALDFRTPAKAKLINICIWVLASGVGVPIMVMAVTQPRDGAVVCMLQFPSPSWYWDTVTKICVFLFAFVVPILIITVCYGLMLLRLRSVRLLSGSKEKDRSLRRITRMVLVVVGAFVVCWAPIHIFVIVWTLVDINRRDPLVVAALHLCIALGYANSSLNPVLYAFLDENFKRCFRQLCRTPCGRQEPGSLRRPRQATTRERVTACTPSDGPGGGAAAFEMTLESIMACCLSEEAKEARRINDEIERQLRRDKRDARRELKLLLLGTGESGKSTFIKQMRIIHGSGYSDEDKRGFTKLVYQNIFTAMQAMIRAMDTLKIPYKYEHNKAHAQLVREVDVEKVSAFENPYVDAIKSLWNDPGIQECYDRRREYQLSDSTKYYLNDLDRVADPAYLPTQQDVLRVRVPTTGIIEYPFDLQSVIFRMVDVGGQRSERRKWIHCFENVTSIMFLVALSEYDQVLVESDNENRMEESKALFRTIITYPWFQNSSVILFLNKKDLLEEKIMYSHLVDYFPEYDGPQRDAQAAREFILKMFVDLNPDSDKIIYSHFTCATDTENIRFVFAAVKDTILQLNLKDIGLF

SEQ ID NO: 73 - protein, 805 aa, Artificial
  Synthetic construct; flag-tagged D2s-linker-Gqi5
MKTIIALSYIFCLVFADYKDDDDAMDPLNLSWYDDDLERQNWSRPFNGSDGKADRPHYNYYATLLTLLIAVIVFGNVLVCMAVSREKALQTTTNYLIVSLAVADLLVATLVMPWVVYLEVVGEWKFSRIHCDIFVTLDVMMCTASILNLCAISIDRYTAVAMPMLYNTRYSSKRRVTVMISIVWVLSFTISCPLLFGLNNADQNECIIANPAFVVYSSIVSFYVPFIVTLLVYIKIYIVLRRRRKRVNTKRSSRAFRAHLRAPLKEAARRAQELEMEMLSSTSPPERTRYSPIPPSHHQLTLPDPSHHGLHSTPDSPAKPEKNGHAKDHPKIAKIFEIQTMPNGKTRTSLKTMSRRKLSQQKEKKATQMLAIVLGVFIICWLPFFITHILNIHCDCNIPPVLYSAFTWLGYVNSAVNPIIYTTFNIEFRKAFLKILHCFERPADGRMTLESIMACCLSEEAKEARRINDEIERQLRRDKRDARRELKLLLLGTGESGKSTFIKQMRIIHGSGYSDEDKRGFTKLVYQNIFTAMQAMIRAMDTLKIPYKYEHNKAHAQLVREVDVEKVSAFENPYVDAIKSLWNDPGIQECYDRRREYQLSDSTKYYLNDLDRVADPAYLPTQQDVLRVRVPTTGIIEYPFDLQSVIFRMVDVGGQRSERRKWIHCFENVTSIMFLVALSEYDQVLVESDNENRMEESKALFRTIITYPWFQNSSVILFLNKKDLLEEKIMYSHLVDYFPEYDGPQRDAQAAREFILKMFVDLNPDSDKIIYSHFTCATDTENIRFVFAAVKDTILQLNLKDIGLF

SEQ ID NO: 74 - DNA, 24 bp, Artificial
  Synthetic construct; DNA encoding linker
ttcgaaagacctgcagacggtaga

SEQ ID NO: 75 - protein, 8 aa, Artificial
  Synthetic construct; linker
FERPADGR

SEQ ID NO: 76 - protein, 46 aa, Artificial
  Synthetic construct; DRD2 human
HCDCNIPPVLYSAFTWLGYVNSAVNPIIYTTFNIEFRKAFLKILHC

SEQ ID NO: 77 - protein, 60 aa, Artificial
  DRD1 human
CIDSNTFDVFVWFGWANSSLNPIIYAFNADFRKAFSTLLGCYRLCPATNNAIETVSINNN

SEQ ID NO: 78 - protein, 46 aa, artificial
  Synthetic construct; DRD3 human
CQTCHVSPELYSATTWLGYVNSALNPVIYTTFNIEFRKAFLKILSC

SEQ ID NO: 79 - protein, 48 aa, Artificial
  Synthetic construct; DRD4 human
CPACSVPPRLVSAVTWLGYVNSALNPVIYTVFNAEFRNVFRKALRACC

SEQ ID NO: 80 - protein, 64 aa, Artificial
  synthetic construct; DRD5 human
AGFPCVSETTFDVFVWFGWANSSLNPVIYAFNADFQKVFAQLLGCSHFCSRTPVETVNISNELI

SEQ ID NO: 81 - protein, 61 aa, Artificial
  Synthetic construct; ADA1A human
FPDFKPSETVFKIVFWLGYLNSCINPIIYPCSSQEFKKAFQNVLRIQCLCRKQSSKHALGY

SEQ ID NO: 82 - protein, 50 aa, Artificial
  synthetic construct; ADA2A human
CSVPRTLFKFFFWFGYCNSSLNPVIYTIFNHDFRRAFKKILCRGDRKRIV

SEQ ID NO: 83 - protein, 65 aa, Artificial
  Synthetic construct; ADRB1 human
FHRELVPDRLFVFFNWLGYANSAFNPIIYCRSPDFRKAFQRLLCCARRAARRRHATHGDRPRASG

SEQ ID NO: 84 - protein, 62 aa, Artificial
  Synthetic construct; ADRB2 human
IQDNLIRKEVYILLNWIGYVNSGFNPLIYCRSPDFRIAFQELLCLRRSSLKAYGNGYSSNGN

SEQ ID NO: 85 - protein, 55 aa, Artificial
  synthetic construct; acm4 human
FCQSCIPDTVWSIGYWLCYVNSTINPACYALCNATFKKTFRHLLLCQYRNIGTAR

SEQ ID NO: 86 - protein, 48 aa, Artificial
  Synthetic construct; 5HT5A_HUMAN
LCSCDIPAIWKSIFLWLGYSNSFFNPLIYTAFNKNYNSAFKNFFSRQH

SEQ ID NO: 87 - protein, 65 aa, Artificial
  Synthetic Construct; CCR5_HUMAN
CSSSNRLDQAMQVTETLGMTHCCINPIIYAFVGEKFRNYLLVFFQKHIAKRFCKCCSIFQQEAPE

SEQ ID NO: 88 - protein, 65 aa, Artificial
  synthetic construct; oprd human
DRRDPLVVAALHLCIALGYANSSLNPVLYAFLDENFKRCFRQLCRKPCGRPDPSSFSRAREATAR

SEQ ID NO: 89 - protein, 65 aa, Artificial
  Synthetic construct; OPRK human
TSHSTAALSSYYFCIALGYTNSSLNPILYAFLDENFKRCFRDFCFPLKMRMERQSTSRVRNTVQD

SEQ ID NO: 90 - protein, 65 aa, Artificial
  Synthetic construct; OPRM_HUMAN
TIPETTFQTVSWHFCIALGYTNSCLNPVLYAFLDENFKRCFREFCIPTSSNIEQQNSTRIRQNTR

SEQ ID NO: 91 - protein, 63 aa, Artificial
  Synthetic Construct; OPSD human
HQGSNFGPIFMTIPAFFAKSAAIYNPVIYIMMNKQFRNCMLTTICCGKNPLGDDEASATVSKT

SEQ ID NO: 92 - protein, 65 aa, Artificial
  Synthetic construct; CNR1 human
GKMNKLIKTVFAFCSMLCLLNSTVNPIIYALRSKDLRHAFRSMFPSCEGTAQPLDNSMGDSDCLH

SEQ ID NO: 93 - protein, 65 aa, Artificial
  Synthetic construct; AA2AR
PDCSHAPLWLMYLAIVLSHTNSVVNPFIYAYRIREFRQTFRKIIRSHVLRQQEPFKAAGTSARVL

SEQ ID NO: 94 - protein, 65 aa, Artificial
  Synthetic construct; TSHR_HUMAN
NKPLITVSNSKILLVLFYPLNSCANPFLYAIFTKAFQRDVFILLSKFGICKRQAQAYRGQRVPPK

SEQ ID NO: 95 - DNA, 6248 bp, Artificial
  Synthetic Construct
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttggtaccgagctcggatcgatatcatgaagacgatcatcgccctgagctacatcttctgcctggtattcgccgaacaaaagctcatttccgaagaggatcttgccatggagcccctgttcccagcctccacgcccagctggaacgcctcctccccgggggctgcctctggaggcggtgacaacaggacgctggtggggccggcgccctcggcaggggcccgggcggtgctggtgcccgtgctgtacctgctggtgtgtgcggccgggctgggcgggaacacgctggtcatctacgtggtgctgcgcttcgccaagatgaagaccgtcaccaacatctacattctcaacctggcagtggccgacgtcctgtacatgctggggctgcctttcctggccacgcagaacgccgcgtccttctggcccttcggccccgtcctgtgccgcctggtcatgacgctggacggcgtcaaccagttcaccagtgtcttctgcctgacagtcatgagcgtggaccgctacctggcagtggtgcacccgctgagctcggcccgctggcgccgcccgcgtgtggccaagctggcgagcgccgcggcctgggtcctgtctctgtgcatgtcgctgccgctcctggtgttcgcggacgtgcaggagggcggtacctgcaacgccagctggccggagcccgtggggctgtggggcgccgtcttcatcatctacacggccgtgctgggcttcttcgcgccgctgctggtcatctgcctgtgctacctgctcatcgtggtgaaggtgagggcggcgggcgtgcgcgtgggctgcgtgcggcggcgctcggagcggaaggtgacgcgcatggtgttggtggtggtgctggtgtttgcgggatgttggctgcccttcttcaccgtcaacatcgtcaacctggccgtggcgctgccccaggagcccgcctccgccggcctctacttcttcgtggtcatcctctcctacgccaacagctgtgccaaccccgtcctctacggcttcctctctgacaacttccgccagagcttccagaaggttctgtgcctccgcaagggctctggtgccaaggacgctgacgccacggagccgcgtccagacaggatccggcagcagcaggaggccacgccgcccgcgcaccgcgccgcagccaacgggcttatgcagaccagcaagctgtgagccagtgtgctggaattaattcgctgtctgcgagggccagctgttggggtgagtactccctctcaaaagcgggcatgacttctgcgctaagattgtcagtttccaaaaacgaggaggatttgatattcacctggcccgcggtgatgcctttgagggtggccgcgtccatctggtcagaaaagacaatctttttgttgtcaagcttgaggtgtggcaggcttgagatctggccatacacttgagtgacaatgacatccactttgcctttctctccacaggtgtccactcccaggtccaactgcaggtcgagcatgcatctagggcggccaattccgcccctctccctcccccccccctaacgttactggccgaagccgcttggaataaggccggtgtgcgtttgtctatatgtgattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataagcttgccacaacccacaaggagacgaccttccatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgagtgcccgaaggaccgcgcgacctggtgcatgacccgcaagcccggtgcctgacgcccgccccacgacccgcagcgcccgaccgaaaggagcgcacgaccccatggctccgaccgaagccgacccgggcggccccgccgaccccgcacccgcccccgaggcccaccgactctagataactgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcgtcgagtgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 96 - DNA, 1095 bp, Homo sapiens
atggagcccctgttcccagcctccacgcccagctggaacgcctcctccccgggggctgcctctggaggcggtgacaacaggacgctggtggggccggcgccctcggcaggggcccgggcggtgctggtgcccgtgctgtacctgctggtgtgtgcggccgggctgggcgggaacacgctggtcatctacgtggtgctgcgcttcgccaagatgaagaccgtcaccaacatctacattctcaacctggcagtggccgacgtcctgtacatgctggggctgcctttcctggccacgcagaacgccgcgtccttctggcccttcggccccgtcctgtgccgcctggtcatgacgctggacggcgtcaaccagttcaccagtgtcttctgcctgacagtcatgagcgtggaccgctacctggcagtggtgcacccgctgagctcggcccgctggcgccgcccgcgtgtggccaagctggcgagcgccgcggcctgggtcctgtctctgtgcatgtcgctgccgctcctggtgttcgcggacgtgcaggagggcggtacctgcaacgccagctggccggagcccgtggggctgtggggcgccgtcttcatcatctacacggccgtgctgggcttcttcgcgccgctgctggtcatctgcctgtgctacctgctcatcgtggtgaaggtgagggcggcgggcgtgcgcgtgggctgcgtgcggcggcgctcggagcggaaggtgacgcgcatggtgttggtggtggtgctggtgtttgcgggatgttggctgcccttcttcaccgtcaacatcgtcaacctggccgtggcgctgccccaggagcccgcctccgccggcctctacttcttcgtggtcatcctctcctacgccaacagctgtgccaaccccgtcctctacggcttcctctctgacaacttccgccagagcttccagaaggttctgtgcctccgcaagggctctggtgccaaggacgctgacgccacggagccgcgtccagacaggatccggcagcagcaggaggccacgccgcccgcgcaccgcgccgcagccaacgggcttatgcagaccagcaagctgtga

SEQ ID NO: 97 - protein, 347 aa, Homo sapiens
MEPLFPASTPSWNASSPGAASGGGDNRTLVGPAPSAGARAVLVPVLYLLVCAAGLGGNTLVIYVVLRFAKMKTVTNIYILNLAVADVLYMLGLPFLATQNAASFWPFGPVLCRLVMTLDGVNQFTSVFCLTVMSVDRYLAVVHPLSSARWRRPRVAKLASAAAWVLSLCMSLPLLVFADVQEGGTCNASWPEPVGLWGAVFIIYTAVLGFFAPLLVICLCYLLIVVKVRAAGVRVGCVRRRSERKVTRMVLVVVLVFAGCWLPFFTVNIVNLAVALPQEPASAGLYFFVVILSYANSCANPVLYGFLSDNFRQSFQKVLCLRKGSGAKDADATEPRPDRIRQQQEAT

SEQ ID NO: 98 - protein, 14 aa, Artificial
  synthetic construct
LKILHCFEMTLESI

SEQ ID NO: 99 - protein, 20 aa, Artificial
  synthetic construct
LKILHCFERPADGRMTLESI

SEQ ID NO: 100 - DNA, 19 bp, Artificial
  synthetic construct; siRNA for Gq/11 knockdown
gatgttcgtggacctgaac

(19) United States
(12) Patent Application Publication
(10) Pub. No.: US 2011/0160081 A1
(43) Pub. Date: Jun. 30, 2011

(54) FUNCTIONAL COMPLEMENTATION ASSAY FOR DEFINED GPCR OLIGOMERS

(76) Inventors: Jonathan A. Javitch, New Rochelle, NY (US); Yang Han, New York, NY (US)

(21) Appl. No.: 13/000,613

(22) PCT Filed: Jul. 1, 2009
(86) PCT No.: PCT/US09/03933

§ 371 Date: Mar. 10, 2011

Related U.S. Application Data
(60) Provisional application No. 61/133,714, filed on Jul. 1, 2008.

(51) Int. Cl. C40B 30/04; C07K 14/00; C12P 21/00; C12Q 1/68; G01N 33/53

(52) U.S. Cl. 506/9; 530/350; 435/69.1; 435/6.1; 435/7.2

(57) ABSTRACT

The present invention is directed to, inter alia, a biological reagent that includes a complex having a first GPCR and a second GPCR linked to a G-protein, the linkage between the second GPCR and the G-protein being of a length, which pre-vents productive interaction between the G-protein and the second GPCR, wherein the first GPCR and the second GPCR linked to the G-protein alone are incapable of producing a signal when presented with a ligand. The invention also provides methods of producing such a biological reagent, as well as methods of determining oligomeric GPCR interactions, methods of identifying compounds that have an effect on GPCR oligomers, methods of identifying a compound capable of interacting with GPCR oligomers, methods of identifying a compound having the ability to modulate binding between a GPCR oligomer and its ligand, and methods for evaluating differential G-protein coupling.

### CROSS-REFERENCE TO RELATED APPLICATIONS

The present application claims benefit to U.S. provisional patent application No. 61/133,714 filed on Jul. 1, 2008, the entire contents of which is incorporated by reference in its entirety as if recited in full herein.

### FEDERALLY SPONSORED RESEARCH OR DEVELOPMENT

This invention was made with government support under Grant Numbers RO1 MH54137, DA022413, and DA012923 awarded by the National Institutes of Health. The government has certain rights in the invention.

### FIELD OF INVENTION

The present invention relates to, inter alia, a biological reagent comprising a complex of G protein-coupled receptors (GPCR), methods of producing the same, methods for determining oligomeric GPCR interactions, and methods for identifying compounds that interact with GPCR oligomers, e.g., dimers.

### BACKGROUND

G protein-coupled receptors comprise a diverse, well-studied system for transducing signals from the extracellular milieu to a variety of intracellular signaling molecules (1). Although GPCRs have been recently considered to be oligomers such as dimers in the plasma membrane (2), understanding of the structural details and functional role of this spatial organization is still limited (3). Most importantly, it has not been established whether activation of Class A rhodopsin-like GPCRs is affected by such an organization in a particular quaternary structure. Recently both rhodopsin (4) and the β₂-adrenergic receptor (B2AR) (5) have been shown to signal efficiently to G proteins when reconstituted into lipid nanodiscs containing only a single receptor. Thus, after solubilization and reconstitution, these GPCRs can function alone. However, such studies cannot clarify whether these receptors do function alone in vivo, and this question still needs to be addressed directly through an exploration of their native organization.

The Class C heterodimeric GABA_{B} receptor has been shown to function as a dimer through a “transactivation” mechanism in which agonist binding to one protomer signals through the second protomer to G protein (6). A clever adaptation of the endoplasmic reticulum (ER) retention signal from the GABA_{B} receptor has enabled controlled cell surface expression and study of signaling by defined metabotropic glutamate receptor (mGluR) “hetero”-dimers (7), which have been shown to signal through both trans- and cis-activation (7). Such an approach to engineered ER retention signals has not yet been successful in Class A receptors, but Class A glycoprotein hormone receptors with large N-terminal binding sites also appear to be capable of both trans- as well as cis-activation (8).

The native functional signaling unit in other Class A rhodopsin-like receptors remains unclear. A number of studies have shown that coexpression of two different Class A GPCRs can lead to signaling properties that differ from their properties when expressed alone (9, 10). This could result from downstream signaling crosstalk or from a heteromeric signaling unit, which would require communication between the protomers. A conformational change at the dimer interface has been associated with activation (13). In addition, agonist binding to a single protomer of the rhodopsin-like leukotriene B₄ receptor BLT1 induced asymmetric conformational changes within the dimer, consistent with transfer of information between the protomers (113). In contrast to the GABA_{B} receptor and TSH and LH receptors, the data for BLT1 support the existence of cis- but not trans-activation.

This proposed asymmetric nature of the signaling unit might account in full or in part for the negative cooperativity that has been observed for ligand binding in class A GPCRs. For example, in cells expressing chemokine receptor heterodimers, a selective ligand for protomer 1 can lead to dissociation of a ligand prebound to protomer 2 (96), consistent with transmittal of an altered conformation across the dimer interface.

Receptor-G protein fusion constructs, in which the C-terminus of a GPCR is fused to the N-terminus of a Ga protein, have been widely used to explore receptor signaling (14-20). Coexpression of such GPCR-G protein fusions with a second GPCR has been used to study heterodimer signaling; in such a scenario the unfused GPCR can activate the G protein fused to a coexpressed GPCR (16-20). However, coexpression of GPCRs is likely to lead to a combination of different signaling units consisting of both homodimers and heterodimers, which makes it difficult to study the functional interactions between two receptors in a defined heteromeric signaling unit. Indeed, it has been shown that tethered G proteins fused to a single membrane-spanning segment can be activated efficiently by a coexpressed GPCR (16, 21), suggesting that a GPCR-G protein fusion construct also might provide G protein for activation by another receptor without actually participating in the relevant dimeric signaling unit. The long GPCR cytoplasmic tails and flexible linkers to which G proteins have been fused are likely to lead to promiscuous interactions that exacerbate this problem. Indeed, the tether attaching the B2AR to fused G proteins can be dramatically shortened with preserved function (22), but whether the G protein in this case is activated by its own receptor or another receptor is not known.

The catecholamine dopamine plays a major role in the regulation of cognitive, emotional and behavioral functions, and abnormalities in its regulation have been implicated in a number of psychiatric and neurological disorders. Dopamine acts through D2-like (D2, D3, D4) and D1-like (D1, D5) receptors, which are members of the seven transmembrane segment GPCR superfamily. Many drugs used to treat psychiatric disorders, including schizophrenia, attention-deficit hyperactivity disorder (ADHD), and depression, target dopamine receptors, either directly or indirectly. That dopamine receptors may exist and function in complex with other GPCRs opens new pharmacological possibilities that will be best exploited if based on a clear understanding of the mechanistic basis of this signaling crosstalk.

What is most physiologically relevant is understanding the role of the oligomeric, e.g., dimeric organization of GPCRs in signaling (1, 3). Indeed, one of the great challenges in GPCR biology today is the weak mechanistic link between the physical interaction of receptors in the membrane and signaling crosstalk of presumed heterodimers or hetero-oligomers. There is a great deal of evidence from many laboratories that many GPCRs interact as heterodimers (61, 62). A number of findings, now support the existence of higher order homo-oligomers (63-66). This raises the possibility that GPCR heteromers may interact not as heterodimers per se but rather as higher order hetero-oligomers composed of homodimer subunits.

A large number of studies have demonstrated signaling cross-talk between coexpressed GPCRs (67). In almost all cases, however, the mechanistic link between heteromerization and signaling is tenuous. Although activation of two co-expressed receptors may be essential, signaling crosstalk could nonetheless take place downstream of parallel homomeric receptor-mediated G protein activation and in such a case would not be a direct result of heteromeric signaling. Such a downstream crosstalk mechanism, while often ignored, is very difficult to rule out. One example of this complexity is a recent study of a presumed dopamine D1-D2 receptor heterodimer that has been carried out both in heterologous cells (68) and in the brain (69). These receptors appear to be co-expressed in some neurons in vivo (69). In heterologous cells, they have been inferred to physically interact based on fluorescence resonance energy transfer (FRET) (70, 71) as well as co-internalization (72, 73) and co-retention of mutants (74). Activating both dopamine D1 receptor (D1R) and dopamine D2 receptors (D2R) leads to altered signaling and recruitment of Gq-mediated signaling (68, 69), whereas D1R signaling is normally Gs/olf mediated and D2R signaling is normally Go/i mediated. These findings are intriguing and open exciting avenues of drug design targeted at selective heteromers (75). In this study, however, D1R-mediated Gq signaling was observed in the brain (76, 77), but in other studies, it has been shown to be insensitive to D2R blockade (78), suggesting a role for other cellular factors in the coupling of D1R to the Gq pathway. That D2R signaling appears to be essential in one case and not in the other suggests a complex interaction of signaling mechanisms. Evidence for a priming effect for D1R-mediated Gq signaling is an example of such a potential mechanism (79, 80).

D2R has also been reported to interact with the dopamine D3 receptor (D3R), and coexpression of the D2 and D3 receptors has been reported to modulate the function of both receptors (81-83). More recently the D2R has been shown to modulate and to physically associate with the dopamine transporter as well (84, 85).

In addition to its reported interactions with receptors from the dopamine subfamily, there is a substantial literature on heteromerization of D2R with multiple other Family A receptors. There is evidence for direct physical interaction between D2R and the somatostatin subtype 5 receptor (SSTR5) (86), D2R and adenosine A2A receptor (87, 88), and D2R and CB1 cannabinoid receptor (89). In each of these cases, changes in signaling were observed upon receptor coexpression, with either altered D2R pharmacology by the partner protomer and/or an alteration in the properties of the partner in response to drugs acting at the D2R. In the case of the D2R-CB1 heteromer, dual-agonist mediated activation of Gs was reported, although neither receptor alone is able to activate this Ga subunit (89). These results are intriguing and suggest the possibility of an untapped level of pharmacological diversity for new compound development, as well as a host of potential roles for in vivo signaling specificity for these putative heteromers. However, in none of these studies is it possible to rule out downstream signaling crosstalk and thus to establish incontrovertibly that direct signaling by the D2R heteromer is responsible for the crosstalk.

Such a mechanistic interrogation of heteromeric signaling in Family A GPCRs has been difficult. As mentioned above, mechanistic understanding of the functional role of GPCR dimerization is more advanced in the Family C receptors, due, in part, to the availability of a clever adaptation of the endoplasmic reticulum (ER) retention signal from the GABAB receptor to enable controlled cell surface expression and signaling by defined metabotropic glutamate receptor (mGluR) heterodimers (6). These studies have shown evidence for asymmetric activation of the heterodimer (11, 90). Furthermore, one agonist can activate the dimer, but two agonists are required for full activation (91). In addition, within the same Family C, T1R3 taste receptors are known to form functional heterodimers with either T1R1 or T1R2 in order to respond to a large panel of ligands and to trigger umami and sweet taste sensations respectively (92).

Unfortunately, related approaches with ER retention signals have been unsuccessful in Family A receptors, and it has not been possible to differentiate clearly the role of each subunit in homomeric and heteromeric signaling with coexpressed receptors. However, multiple lines of study do suggest interaction between Family A receptors in a heteromeric functional unit. Thus, for example, ligand binding dissociation kinetics measurements have recently been linked to the GPCR dimerization process (93). In chemokine receptor heteromers, a CCR2 specific drug accelerates the dissociation of a CCR5 or CXCR4 selective drug when the receptors are coexpressed in heterologous cells and in native lymphocytes (94-96). Moreover, although it remains to be proven conclusively, it seems reasonable to infer that bivalent drugs engaging two different receptors, i.e. heteromer-selective compounds, might act simultaneously on two protomers in a heteromer and thereby directly activate downstream heteromer-specific signaling machinery (97-99) raising the possibility of their selective therapeutic potential (100). Although there is evidence of G protein signaling by coexpressed nonfunctional receptor chimeras, this was proposed to occur by transmembrane domain swapping (101), which is unlikely to be universal (102), and researchers have been unable to generate such functional recovery in adrenergic or dopaminergic receptors. Curiously, coexpression of two loss of function glycoprotein hormone receptors (receptors with either agonist binding or the ability to activate G proteins compromised) (103-105) led to function, but among Family A receptors such rescue seems to be limited to glycoprotein hormone receptors, which have very large extracellular N-terminal binding sites. This is similar to the transactivation seen in the Family C GABAB receptor, in which agonist binding to one protomer signals to G protein through the second protomer (6).

Another example of the potential complexity of receptor interactions is the relationship between the delta opiate receptor (DOR) and the D2R. Although there is substantial evidence for synergy and modulation of signaling in vivo by co-application of selective DOR and D2R ligands (106-108), to the inventor's knowledge, there has been no proposal of direct interaction of these receptors. Curiously, D2R and DOR co-exist in vivo in the striatum in the terminals of dopaminergic neurons, in the terminals of corticostriatal neurons, and in post-synaptic medium spiny neurons (109-110).

### SUMMARY OF THE INVENTION

In view of the foregoing, there is a need for compositions and methods for evaluating, inter alia, GPCR oligomeric, particularly dimeric, signaling via the oligomer. The present invention is directed to meeting this and other needs.

One embodiment of the present invention is a biological reagent. This biological reagent comprises a complex having (a) a first G-protein coupled receptor (GPCR); and (b) a second GPCR linked to a G-protein, the linkage between the second GPCR and the G-protein being of a length, which prevents productive interaction between the G-protein and the second GPCR, wherein the first GPCR and the second GPCR linked to the G-protein alone are incapable of producing a signal when presented with a ligand.

Another embodiment of the present invention is a method of producing a biological reagent. This method comprises the steps of: (a) expressing a first nucleic acid in a cell, the nucleic acid encoding a first GPCR; (b) expressing a second nucleic acid in the cell, the second nucleic acid encoding a fusion protein comprising a second GPCR fused to a G-protein, the G-protein being fused to the second GPCR in such a manner so that it cannot participate in a productive interaction with the second GPCR; and (c) allowing the expressed proteins from steps (a) and (b) to assemble into a complex in the cell membrane, wherein the expressed proteins from steps (a) and (b) alone are incapable of producing a signal when presented with a ligand.

An additional embodiment of the present invention is a method of determining whether a first and second GPCR have affinity for each other such that they form a functional GPCR oligomer. This method comprises (a) producing or providing a first nucleic acid construct encoding a first GPCR; (b) producing or providing a second nucleic acid construct encoding a second GPCR and its associated G-protein as a fusion protein, the G-protein being fused to the second GPCR in such a manner so that it cannot participate in a productive interaction with the second GPCR, wherein the first GPCR and the second GPCR and its associated G-protein alone are incapable of producing a signal when presented with a ligand; (c) co-expressing the first and second nucleic acid constructs in a cell; and (d) determining the presence of a complex comprising the first and second GPCRs.

A further embodiment of the present invention is a method of determining an effect a compound has on a GPCR oligomer. This method comprises (a) contacting a compound with a first cell expressing a GPCR oligomer having (i) a first GPCR; and (ii) a second GPCR fused to a G-protein, wherein the G-protein is fused to the second GPCR in such a manner so that it cannot participate in a productive interaction with the second GPCR, and the first GPCR and the second GPCR fused to the G-protein alone are incapable of producing a signal when presented with a ligand; (b) detecting the presence of a cellular signal resulting from contact between the compound and the GPCR oligomer; and (c) determining an effect the compound has on the GPCR oligomer.

An additional embodiment of the present invention is a method of identifying a compound capable of interacting with a GPCR oligomer. This method comprises (a) providing a cell expressing a biological reagent according to the present invention; (b) contacting the biological reagent with the compound; and (c) determining whether the compound interacts with the GPCR oligomer.

Yet another embodiment of the present invention is a method of identifying a compound having the ability to modulate binding between a GPCR oligomer and its ligand. This method comprises (a) providing a cell expressing a GPCR oligomer comprising: (i) a first GPCR; and (ii) a second GPCR linked to a G-protein, the linkage between the second GPCR and the G-protein being of a length, which prevents productive interaction between the G-protein and the second GPCR, wherein the first GPCR and the second GPCR linked to the G-protein alone are incapable of producing a signal when presented with a ligand; (b) contacting the cell with a test compound in the presence of the ligand; and (c) comparing the ability of the ligand to bind to the GPCR oligomer with the ability of the ligand to bind to the GPCR oligomer under comparable conditions but in the absence of the compound.

A further embodiment of the present invention is a method for evaluating differential G-protein coupling. This method comprises:
(a) providing a first cell expressing a first GPCR oligomer comprising:
   (i) a first wild type GPCR;
   (ii) a second wild type GPCR linked to a G-protein, the linkage between the second GPCR and the G-protein being of a length, which prevents productive interaction between the G-protein and the second GPCR, wherein the first GPCR and the second GPCR linked to the G-protein alone are incapable of producing a signal when presented with a ligand;
(b) providing a second cell expressing a second GPCR oligomer comprising:
   (i) the first GPCR comprising a mutation;
   (ii) the second GPCR linked to a G-protein, the linkage between the second GPCR and the G-protein being of a length, which prevents productive interaction between the G-protein and the second GPCR, wherein the mutant first GPCR and the second GPCR linked to the G-protein alone are incapable of producing a signal when presented with a ligand;
(c) providing a third cell expressing a third GPCR oligomer comprising:
   (i) the first GPCR;
   (ii) the second GPCR, which comprises a mutation and is linked to a G-protein, the linkage between the second mutant GPCR and the G-protein being of a length, which prevents productive interaction between the G-protein and the second mutant GPCR, wherein the first GPCR and the second mutant GPCR linked to the G-protein alone are incapable of producing a signal when presented with a ligand;
(d) contacting the first, second and third cells with a compound capable of binding to a ligand binding site present on the first and/or the second GPCR;
(e) repeating steps (a) to (d) with a different G-protein; and
(f) evaluating differential G-protein coupling.

Another embodiment of the present invention is a method of identifying a compound having the ability to modulate the activity of a GPCR oligomer. This method comprises:
(a) providing a cell expressing a GPCR oligomer comprising:
   (i) a first GPCR; and
   (ii) a second GPCR linked to a G-protein, the linkage between the second GPCR and the G-protein being of a length, which prevents productive interaction between the G-protein and the second GPCR, wherein the first GPCR and the second GPCR linked to the G-protein alone are incapable of producing a signal when presented with a ligand;
(b) contacting the cell with a test compound in the presence of a ligand of the first GPCR or of the second GPCR; and
(c) comparing the activity of the GPCR oligomer with the activity of the GPCR oligomer under comparable conditions but in the absence of the compound.

A further embodiment of the present invention is a method for evaluating differential effects of a compound on the activity of a GPCR oligomer. This method comprises:
(a) providing a first cell expressing a first GPCR oligomer comprising:
   (i) a first GPCR;
   (ii) a second GPCR linked to a G-protein, the linkage between the second GPCR and the G-protein being of a length, which prevents productive interaction between the G-protein and the second GPCR, wherein the first GPCR and second GPCR linked to the G-protein alone are incapable of producing a signal when presented with a first ligand;
(b) providing a second cell expressing a second GPCR oligomer comprising:
   (i) a third GPCR;
   (ii) a fourth GPCR linked to a G-protein, the linkage between the fourth GPCR and the G-protein being of a length, which prevents productive interaction between the G-protein and the fourth GPCR, wherein the third GPCR and the fourth GPCR linked to the G-protein alone are incapable of producing a signal when presented with a second ligand;
(c) contacting the first and second cells with a compound capable of binding to the first, the second, the third, and/or the fourth GPCR; and
(d) evaluating the differential activity, if any, of each GPCR oligomer under comparable conditions but in the absence of the compound.

### BRIEF DESCRIPTION OF THE FIGURES

The following drawings form part of the present specification and are included to further demonstrate certain aspects of the present invention. The invention may be better understood by reference to one or more of these drawings in combination with the detailed description of specific embodiments presented herein.

FIG. 1 demonstrates the functional complementation of two “non-functional receptors.” An aequorin assay that couples Gq (or Gqi5) activation to a luminescence readout was used. FIG. 1A shows that the agonist quinpirole did not lead to D2R-induced Gq activation. D2R when coexpressed with free Gqi5 (FIG. 1B) or D2R fused with Gqi5 via an eight amino acid linker (D2-linker-Gqi5) (FIG. 1C) led to quinpirole-induced luminescence. FIG. 1C also shows that a nonfunctional Ga deficient fusion construct, D2-linker-Gqi5_{G208A} failed to produce luminescence. FIG. 1D shows that free Gqi5 rescued the function of D2-linker-Gqi5_{G208A}. FIG. 1E shows that free Gqi5 failed to rescue the function of D2R-Gqi5, another fusion protein in which D2R is linked to Gqi5 by a two amino acid linker. D2R-Gqi5, unlike D2R-linker-Gqi5, did not signal when expressed alone. FIG. 1F shows that coexpressing D2R with D2R-Gqi5 (12 hour tetracycline induction) restored signaling, despite the inability of either construct to signal in this assay when expressed alone. Activation data represent luminescence relative to that seen with 0.1% triton treatment. The mean±standard error of mean (SEM) of at least 3 experiments, each conducted in triplicate, are shown. The symbols used are explained in detail in FIG. 8.

FIG. 2 shows the characterization of D2R mutants. FIG. 2A is a schematic representation showing the positions of the mutations in the D2R. FIG. 2B shows that D2/D4 is activated by quinpirole, albeit with a lower potency and efficacy when compared with WT D2R. D2/D4 is a D2 mutant with 4 amino acids substituted from the D4 receptor (V91^{2.61}F/F110^{3.29}L/V111^{3.28}M/Y408^{7.35}V), making it 1000-times more sensitive to a D4-selective inhibitor (see FIG. 10). FIG. 2C shows that all the other mutants were non-functional. Activation data were normalized as in FIG. 1. The mean±SEM of at least 3 experiments, each conducted in triplicate, are shown.

FIG. 3 shows asymmetric contributions of the protomers to signaling. FIG. 3A and FIG. 3B show that when all mutants (as protomer A) were coexpressed with WT D2R-Gqi5 (as protomer B), only WT and D2/D4 were able to signal (FIG. 3A). FIG. 3B shows that none of the other mutants were able to restore signaling when coexpressed with WT D2R-Gqi5. FIGS. 3C-3E show that the results differed when WT D2R (as protomer A) was coexpressed with the various mutant-Gqi5 constructs (as protomer B). In particular, FIG. 3C shows that D2/D4-Gqi5 (▾) restored the ability of unfused WT D2R to signal. FIG. 3D shows that D114^{3.32}A-Gqi5 (▴) deletion 213-219-Gqi5 (•), and D80^{2.50}A-Gqi5 (▴) also restored the ability of unfused WT D2R to signal. FIG. 3E shows that coexpressing R132^{3.50}A-Gqi5 (▾), V136^{3.54}D/M140^{3.58}E-Gqi5 (♦), or N393^{7.49}A-Gqi5 (▪) with WT D2R failed to rescue signaling. Note that D114A-Gqi5 (▴) and D2/D4-Gqi5 (▾) (as shown in FIG. 3D) showed a higher maximal activation than WT. Activation data represent relative luminescence when compared to WT D2R coexpressed with WT D2R-Gqi5 after normalizing for surface expression of the Gqi5 fusion construct (see Example 1). The mean±SEM of at least 3 experiments, each conducted in triplicate, are shown.

FIG. 4 shows that the second protomer allosterically modulates signaling. Shown are effects on signaling with the D2/D4 construct expressed either as protomer A (FIG. 4A), or as protomer B (D2/D4-Gqi5) (FIG. 4B). FIG. 4A shows that the D4-selective antagonist L745,870 (1 μM) totally blocked signaling of the D2/D4 construct expressed as protomer A with WT-Gqi5. In contrast, FIG. 4B shows that L745,870 increased maximal activation for WT D2R coexpressed with D2/D4-Gqi5 to 156.7±7.3% (n=9) (p<0.01*** by Student's t-test) of that observed for D2R coexpressed with WT D2R-Gqi5 (see FIG. 3A). FIG. 4C shows that coexpression of a constitutively active mutant (FIG. 14) that was unable to bind ligand (D114^{3.32}A/CAM-Gqi5), to enhance the fraction of protomer B in an active conformation, led to 49.6±8.4% (n=9) (p<0.01*** by Student's t-test) of maximal activity (♦) when compared to WT D2R coexpressed with D114A-Gqi5 (⋄). Activation data were normalized to surface expression as disclosed in Example 1. The mean±SEM of at least 3 experiments, each conducted in triplicate, are shown.

FIG. 5 shows a computational model of the complex between the rhodopsin dimer and heterotrimeric Gₜ. FIG. 5A shows a structural representation of the nonameric oligomer array. The dashed box identifies the TM4 dimer contained in Model 2. FIG. 5B shows a structural representation of the complex formed between transducin and the nonameric oligomer array. The optimal representative structure (defined in Example 1) is shown for Model 2. FIG. 5C shows a close-up view of the interaction between specific residues of Gα (CPK representation) and the IL3 and IL2 loops of protomer A and B. FIG. 5D shows side view of the complex showing G_{tα} (red), G_{tβ} (wheat), G_{tγ} (orange), protomer A (green), protomer B (light blue), IL2 of protomer A (magenta), IL2 of protomer B (blue), and IL3 of protomer B (cyan). Other views of the model complex are shown in FIG. 16.

FIG. 6 shows a cartoon of different D2R dimer activation states, with activation data for these states, from the perspective of agonist-mediated activation of protomer A. Bound agonist is represented by a black square. Activation is represented by a trapezoid with a bold base. Extent of activation is indicated by increasingly bold trapezoid boundaries. The inverse agonist bound state is represented by an inverted trapezoid. In configuration (1), neither protomer is activated. In configuration (2), protomer A binds agonist and protomer B is constitutively active (or in the case of a heterodimer, is occupied by protomer B′s agonist). In configuration (3), protomer A binds agonist, whereas protomer B cannot bind (or in a heterodimer, is not agonist-bound). Note that although protomer B is not activated by ligand, it can isomerize to the active state, which would result in configuration (2). In configuration (4), protomer A binds agonist, whereas protomer B is stabilized in the inactive state by inverse agonist. Experimentally determined maximal activation representing these idealized conformations: configuration (1) no ligand, configuration (2) WT D2R coexpressed with D114A/CAM-Gqi5, configuration (3) WT D2R coexpressed with D114A-Gqi5, configuration (4) WT D2R coexpressed with D2/D4-Gqi5 in the presence of the selective D4 antagonist, L745,870. Activation data are normalized to that of WT D2R coexpressed with WT D2R-Gqi5, which is indicated by a dotted line to indicate the potential range of enhanced and reduced signaling achievable by modulation of the “heterodimer” partner.

FIG. 7 show activation of endogenous receptors and stably transfected D2R coexpressed with free Gqi5 or with D2R-Gqi5 in the presence or absence of pertussis toxin. FIG. 7A shows activation of Gq coupled endogenous muscarinic (ACH) and purinergic (ATP) receptors. FIG. 7B shows activation of D2R coexpressed with free Gqi5 in the presence (▴) or absence of pertussis toxin (PTX) (Δ). FIG. 7C shows activation of D2R-Gqi5 coexpressed with D2R in the presence (▪) or absence of PTX (□). The mean±SEM of 3 experiments, each conducted in triplicate, are shown.

FIG. 8 shows symbols used in the drawings of the present invention. Schematic illustration of the sequences of the linker regions of D2R-Gqi5 (SEQ ID NO: 98) and D2R-linker-Gqi5 (SEQ ID NO: 99) are shown at the bottom of FIG. 8.

FIG. 9 shows cell surface expression of D2R VVT and mutants. Myc-D2R mutants and Flag-D2R-Gqi5 (FIG. 9A) or Myc-D2R and Flag-D2R mutant-Gqi5 (FIG. 9B) were detected by fluorescence activated cell sorting (FACS). The mean±SEM of 3 experiments, each conducted in triplicate, are shown.

FIG. 10 shows inhibition of quinpirole-induced activation by the D4-selective antagonist L745,870 in cells in which D2R wild type (▪) or the D2/D4 mutant (▾) were coexpressed with free Gqi5. Due to the much lower EC₅₀ of the D2/D4 mutant for quinpirole, 10 nM and 100 μM quinpirole were used with WT and the D2/D4 mutant, respectively, in order to achieve similar extents of activation. The mean±SEM of 3 experiments, each conducted in triplicate, are shown.

FIG. 11 shows the activation of D2R mutant-linker-Gqi5, in which D2R mutants are linked to Gqi5 via an eight amino acid linker. All mutants including D1143.32A-linker-Gqi5 (▴), deletion 213-219-linker-Gqi5 (•), D802.50A-linker-Gqi5 (▴), R1323.50A-linker-Gqi5 (▾), V1363.54D/M1403.58E-linker-Gqi5 (♦), or N3937.49A-linker-Gqi5 (▪) failed to signal. The mean±SEM of 3 experiments, each conducted in triplicate, are shown.

FIG. 12 shows the relationship between surface expression and activation. Surface expression was determined by FACS (see Example 1 below) for Flag-D2R-Gqi5 (FIG. 12A), stably transfected in pcDNA5/FRT/TO (Invitrogen) and with its expression controlled by varying the length of time after tetracycline induction from 3 hours to 24 hours and for Myc-D2R (FIG. 12B) stably transfected in pIRESpuro3 vector (BD Life Sciences) and expressed constitutively. FIG. 12C shows that cells stably transfected with Flag-D2R-Gqi5 in pcDNA5/FRT/TO were induced by tetracycline for 3 to 24 hours. Specific binding of [³H]N-methylspiperone (0.6 nM) was determined at each time point by subtracting nonspecific binding in the presence of 1 μM sulpiride from the total binding. This concentration was chosen based on FIG. 12D to estimate the Bmax of binding, which was converted to sites/cell based on the specific activity of the ligand, the efficiency of the scintillation counter, and cell counting. FIG. 12D shows the results of binding assays. Flag-D2R-Gqi5 (in pcDNA5/FRT/TO) or Myc-D2R (in pIRESpuro3) were separately stably transfected in Flp-In T-Rex cells. After 24 hours of induction with tetracycline, cells expressing Flag-D2R-Gqi5 were harvested for saturation binding assays. Cells continuously expressing Myc-D2R were harvested for saturation binding assays when suitable confluence was achieved. Saturation binding assays were performed as described in Example 1. FIG. 12E shows linear correlation between surface receptor expression determined by FACS (see FIG. 12A) and by ligand binding (FIG. 12C). FIG. 12F shows the maximal responses of D2R coexpressed with D2-Gqi5 after different periods of tetracycline induction. FIG. 12G shows a plot of these maximal responses against the surface expression level of D2-Gqi5. The standard curve was fit by nonlinear regression with the equation Y=Bmax*X/(K_{d}+X) using GraphPad Prism 4.0, and was used to normalize activation according to surface expression as described in Example 1. The mean±SEM of 3 experiments, each conducted in triplicate, are shown.

FIG. 13 shows that IL2 mutants interact with the WT receptor. FIG. 13A shows the results of titration Bioluminescence Resonance Energy Transfer (BRET) experiments. Increasing amounts of D2-Venus were coexpressed with constant amounts of either WT or mutant D2-RLuc8 in HEK 293T cells. 48 h post-transfection BRET was performed (117). BRET signals were plotted against the relative expression levels of each tagged receptor. Results were analyzed by non-linear regression assuming a model with one site binding (GraphPad Prism 4.0) on a pooled data set from 2 independent experiments. HEK 293T cells transiently coexpressing WT or mutant D2R split RLuc8 (FIG. 13B) or Venus (FIG. 13C) were harvested 48 h post-transfection, washed with PBS, centrifuged and resuspended in PBS. Fluorescence was recorded for 1s using 500 nm excitation and 540 nm emission filters (Polarstar, BMG Labtech GmbH, Durham, N.C.). Unfiltered luminescence was recorded for 1s (Gain 3900). Background was determined with cells expressing only one of the receptor probes and the signal to noise ratio was plotted for cells showing comparable cell surface level of expression for each protomer determined by FACS analysis. The graph is representative of 3 independent experiments performed with triplicate samples.

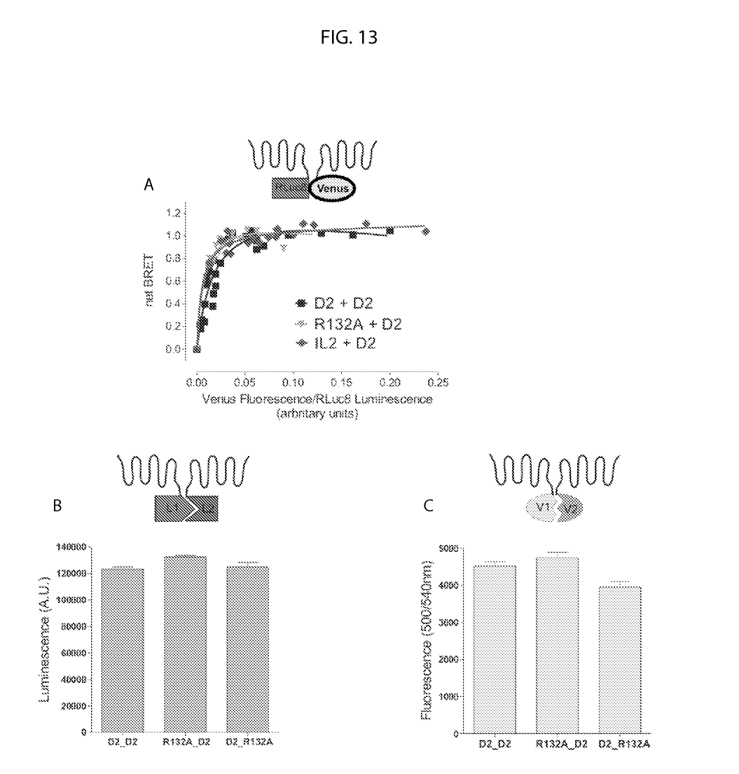

FIG. 14 shows that D2R E339A/T343R is constitutively active. FIG. 14A shows that the inhibitory potency of quinpirole in competition with [³H]N-methylspiperone binding is greatly increased in D2R E339A/T343R compared to WT, consistent with its constitutive activation. Dissociation constants (Ki) of quinpirole binding were 22.45±4.0 μM and 0.913±0.19 μM for WT and the E339A/T343R mutant, respectively. FIG. 14B shows that comparable cell surface expression of coexpressed Flag-D114A-Gqi5 or Flag-D114A/CAM-Gqi5 with Myc-D2R was shown by FACS (see Example 1). The mean±SEM of 3 experiments, each conducted in triplicate, are shown.

FIG. 15 shows a structural representation of the dimer interfaces. FIGS. 15A and 15B show the TM4 dimer interface; FIGS. 15C and 15D show the TM4,5 interface; and FIGS. 15E and 15F show the TM1 dimer interface. The paired panels FIGS. 15A and 15B, FIGS. 15C and 15D, FIGS. 15E and 15F, present top and lateral views of the dimers, respectively. FIGS. 15G and 15H show the nonameric oligomer array, with the various interfaces termed TM1 dimer, TM4,5 dimer, and TM4 dimer indicated by a solid ellipse, a dashed ellipse, and a dashed box, respectively.

FIG. 16 shows a model of the functional complex between the rhodopsin dimer and heterotrimeric Gₜ for the optimal representative of Model 2 (different views of the same construct as FIG. 5). Close-up view of the interaction between specific residues of Ga (CPK representation) and the IL3 and IL2 loops of protomers A and B is shown.

FIG. 17A shows that the co-expression of N393A-Gqi5 with VVT D2R (hollow square) was basically non-functional. However, when co-expressing N393A/CAM-Gqi5 with WT, the function increased by 3-fold compared to co-expressing N393A-Gqi5 with WT D2R (filled square). FIG. 17B shows cell surface expression of Flag tagged D2R mutants-Gqi5 and Myc tagged WT, as detected by FACS.

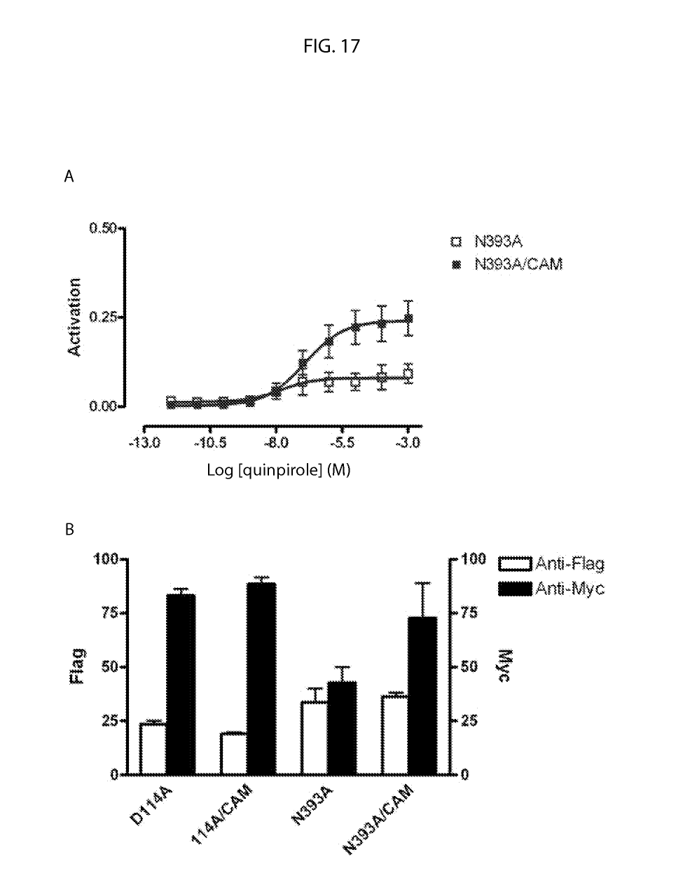

FIG. 18 shows a model of the complex formed between the rhodopsin dimer and heterotrimeric G-protein. FIG. 18A shows a close up view of the interaction between specific residues of Ga (red, CPK representation) and the IL3 (cyan) and IL2 (magenta and blue) loops of protomers A and B. FIG. 18B shows a side view of the complex showing Gα (red), Gβ (wheat), Gγ (orange), protomer A (green), protomer B (light blue), protomer A IL2 (magenta), protomer B IL2 (blue), and IL3 (cyan). Note that the IL2 loops of both protomers are in the vicinity of the red Ga residues, but IL3 of protomer B (lower left) is too far to make any contact.

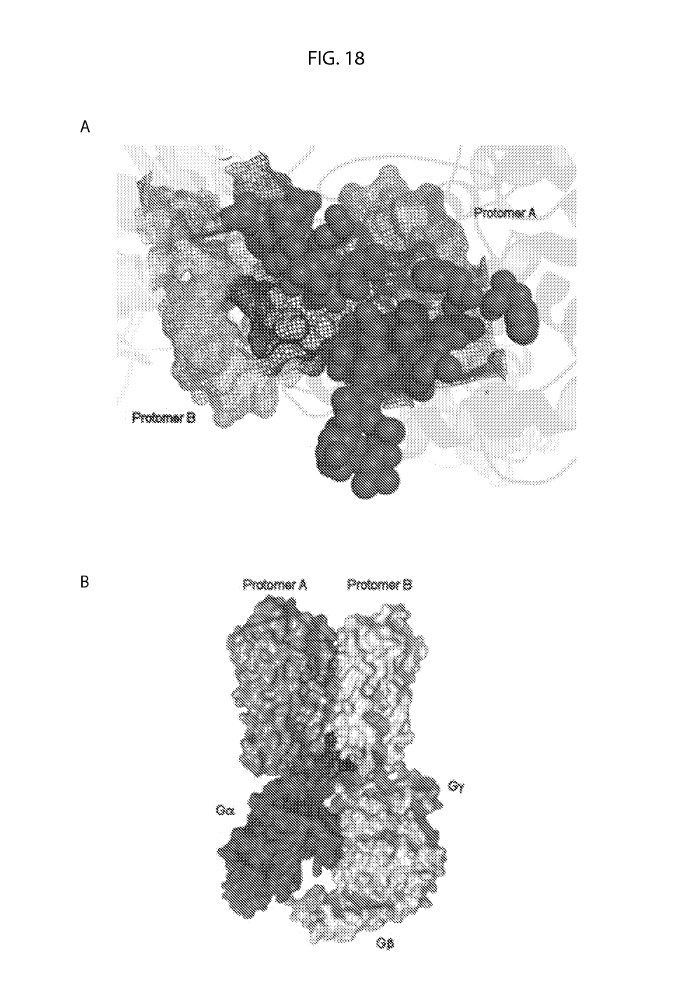

FIG. 19 shows an exemplary flow chart outlining a functional complementation assay for oligomeric signaling according to the present invention.

FIG. 20A is a cartoon showing the co-expression of D2R and delta opiate receptor (DOR) in the CNS in multiple locations in the striatal complex. FIG. 20B shows that a DOR specific agonist increases D2R agonist potency. In this system, an aequorin assay was performed for cells stably coexpressing D2R and DOR-Gqi5 with increasing concentrations of quinpirole in the absence (black squares) or presence (triangles) of 5 nM DPDPE.

FIG. 21 shows a sequence alignment of the C-terminal and of the H8 domain of a representative number of Class A GPCRs. The SEQ ID NOs. of the sequences are as indicated in the figure.

FIG. 22 shows a sequence alignment of the long isoform (“D2_long” SEQ ID NO. 66) and the short isoform (“D2_short” SEQ ID NO. 61) of the human dopamine 2 receptor.

FIG. 23 shows allosteric modulation of signaling of SSTR5 by D2R. FIG. 23A shows that activated SSTR5, which couples to endogenous Gi did not result in luminescence. FIG. 23B shows that coexpressing SSTR5 with D2R-Gqi5 rescued the luminescence readout caused by activating SSTR5 (♦). This activation was blunted by coadministration of 1 μM of the D2R agonist quinpirole (▴), and enhanced by coadministrating 1 μM of the D2R inverse agonist sulpiride (▾). FIG. 23C shows that quinpirole and sulpiride were without effect when SSTR5 was coexpressed with the D2R mutant D114A-Gqi5, which is deficient in ligand binding. FIG. 23D shows the percentage enhancement of activation at each somatostatin concentration in the presence of sulpiride versus that of quinpirole.

### DETAILED DESCRIPTION

One embodiment of the present invention is a biological reagent. This biological reagent comprises a complex having (a) a first G-protein coupled receptor (GPCR); and (b) a second GPCR linked to a G-protein. In this embodiment, the linkage between the second GPCR and the G-protein is of a length, which prevents productive interaction between the G-protein and the second GPCR, and the first GPCR and the second GPCR linked to the G-protein alone are incapable of producing a signal when presented with a ligand.

As used herein, a “complex” means an association comprised of two or more polypeptides, such as e.g., GPCRs, which are in close spatial proximity to each other.

As used herein, “G-protein coupled receptor” or “GPCR” means a 7-transmembrane spanning receptor that upon sensing the appropriate molecule, activates signal transduction pathways and, ultimately, cellular responses, via a guanine nucleotide-binding protein (G-protein). “G-protein” means the α subunit of a heterotrimeric protein that binds guanosine diphosphate (GDP) in its inactive state and binds guanosine triphosphate (GTP) upon activation, and in turn, triggers the signal transduction pathway. The other two subunits of the heterotrimeric protein are β and γ. Exemplary signal transduction pathways include the adenylyl cyclase pathway, the phospholipase C, the Na⁺/H⁺ exchanger pathway, changes in inositol 1, 4, 5 triphosphate level or calcium level.

GPCRs and G-proteins of the invention may be wild type proteins, mutant proteins, chimeric proteins, or chimeric proteins which include further mutations. G-proteins may be divided into four subfamilies: the Gs subfamily, the Gi/o subfamily, the Gq/11 subfamily, and the G12/13 subfamily. As used herein, a “Gq/11 subfamily protein” means a G-protein that, upon activation, is able to activate phospholipase C. Non-limiting examples of Gq/11 subfamily proteins according to the present invention include Gq, G₁₁, G₁₄, and G_{15/16}. A “Gi/o subfamily protein” means a G-protein that, upon activation, is able to inhibit adenylyl cyclase and regulate ion channels. Non-limiting examples of Gi/o subfamily proteins according to the present invention include Gᵢ₁, Gᵢ₂, Gᵢ₃, Gₒ₁, Gₒ₂, Gₒ₃, G_{z}, Gₜ₁, Gₜ₂, and G_{gust}. A “Gs subfamily protein” means a G-protein that, upon activation, is able to stimulate adenylyl cyclase. Non-limiting examples of Gs subfamily proteins according to the present invention include Gₛ and G_{olf}. A “G12/13 subfamily protein” means a G-protein that, upon activation, is able to activate the Na⁺/H⁺ exchanger pathway. Non-limiting examples of G12/13 subfamily proteins according to the present invention include G₁₂ and G₁₃.

Preferably, the first and/or second GPCRs are class A GPCRs. As used herein, “class A GPCRs” mean GPCRs whose sequences are most similar to rhodopsin. They include, for example, 5-Hydroxytryptamine 1A (5HT1A) receptor, 5-Hydroxytryptamine 1B (5HT1B) receptor, 5-Hydroxytryptamine 1D (5HT1D) receptor, 5-Hydroxytryptamine 2A (5HT2A) receptor, 5-Hydroxytryptamine 2C (5HT2C) receptor, 5-Hydroxytryptamine 4 (5HT4) receptor, 5-Hydroxytryptamine 5A (5HT5A) receptor, 5-Hydroxytryptamine 6 (5HT6) receptor, α1A adrenergic receptor, α1b adrenergic receptor, α2a adrenergic receptor, α2b adrenergic receptor, β1 adrenergic receptor, β2 adrenergic receptor, β3 adrenergic receptor, A1 adenosine receptor, A2 adenosine receptor, A3 adenosine receptor, muscarinic acetylcholine 1 (M1) receptor, muscarinic acetylcholine 2 (M2) receptor, muscarinic acetylcholine 3 (M3) receptor, muscarinic acetylcholine 4 (M4) receptor, Melanocortin2 receptor, angiotensin AT1A receptor, angiotensin AT1B receptor, B2 bradykinin receptor, CXCR3, CXCR4, D1 dopamine receptor, D2 dopamine receptor (D2R), D3 dopamine receptor, D4 dopamine receptor, follicle-stimulating hormone receptor (FSHR), gonadotropin-releasing hormone receptor (GRHR), histamine H1 receptor, histamine H2 receptor, lutropin-choriogonadotropic hormone receptor (LSHR), δ opioid receptor 1; κ opioid receptor 1, μ opioid receptor 1, rhodopsin, Oxytocin receptor, P2U purinoreceptor 1, Prostaglandin D2 receptor, Prostaglandin E2 receptor (EP1 subtype), Somatostatin receptor 2, Somatostatin receptor 5 (SSTR5), thyrotropin releasing hormone (TRH) receptor, Vasopressin 1A receptor, chemokine (C-C motif) receptor 5 (CCR5), and cannabinoid receptor 1. More preferably, the GPCR is a D2R.

The first and the second GPCR may be of the same type. For example, both the first and the second GPCR may be D2R. They may also be different. For example, the first GPCR may be a SSTR5, and the second GPCR may be a D2R.

As used herein, “link” or “linked” means to form a connection, for example, by covalent bonding; and “linkage” refers to such a connection. The connection or linkage may be comprised of amino acids, as in the case of fusion proteins, or comprised of chemically modified bonds. “Productive interaction” means actions that result in the triggering of the appropriate signal transduction pathway. “Signal” means any detectable response, for example, changes in cellular levels of certain chemicals, e.g., Ca²⁺, or proteins. “Ligand” means a molecule that binds to a GPCR. Such a molecule may be a full or partial agonist, antagonist, inverse agonist, or inverse antagonist.

In one aspect of this embodiment, the complex is present in a cell membrane. Preferably, the cell membrane is part of an intact cell.

In another aspect of this embodiment, the second GPCR and the G-protein are linked as a fusion protein. As used herein, a “fusion protein” means a polypeptide in which two or more proteins, whether wild-type, mutated, or truncated, are joined together. The joining may occur via, for example, molecular genetic techniques, wherein the polynucleotide sequences of the proteins are fused by polymerase chain reaction or by restriction sites, as disclosed herein.

Preferably, the second GPCR is linked directly to a G-protein. As used herein, “linked directly” means having no exogenous intervening amino acids between the two proteins being linked such that the end of one protein being linked is immediately followed by the beginning of the other protein.

In the present invention, the second GPCR may be linked to the G-protein through a linker. As used herein, “linker” means one or more exogenous amino acids between the two proteins being linked or having a chemical bond between the two proteins being linked other than a peptide bond. In the present invention, any amino acid or amino acid derivative or non-peptide bond, which is sufficient to link, e.g., a GPCR to a G-protein may be used so long as the linkage between the GPCR and the G-protein is of a length which prevents productive interaction between GPCR and the G-protein fused to it. Preferably, the linker is from 1 to 3 amino acids in length, such as 2 amino acids in length. In the present invention, when a range is recited, all members of the range, including the end points, are intended.

In anther aspect of this embodiment, the first GPCR and/or the second GPCR are Gi/o-coupled GPCRs. As used herein, a “Gi/o-coupled GPCR” means a GPCR that is able to have productive interactions with a Gi/o subfamily protein. Representative, non-limiting examples of Gi/o-coupled GPCRs according to the present invention include 5HT1A receptor, 5HT1B receptor, 5HT1D receptor, 5-5HT5A receptor, α2a adrenergic receptor, α2b adrenergic receptor, A1 adenosine receptor, A3 adenosine receptor, M2 receptor, M4 receptor, CXCR3, CXCR4, D2R, D3 dopamine receptor, D4 dopamine receptor, FSHR, LSHR, δ opioid receptor 1, κ opioid receptor 1, μ opioid receptor 1, Oxytocin receptor, Somatostatin receptor 2, SSTR5, CCR5, and cannabinoid receptor 1.

In an additional aspect of this embodiment, the first GPCR and/or the second GPCR are Gq/11-coupled GPCRs. In the present invention, a “Gq/11-coupled GPCR” means a GPCR that is able to have productive interactions with a Gq/11 subfamily protein. Representative, non-limiting examples of Gq/11-coupled GPCRs according to the present invention include 5HT2A receptor, 5HT2C receptor, α1A adrenergic receptor, α1b adrenergic receptor, M1 receptor, M3 receptor, dopamine D1 receptor, D2R, angiotensin AT1A receptor, angiotensin AT1B receptor, B2 bradykinin receptor, histamine H1 receptor, GRHR, P2U purinoreceptor 1, Prostaglandin E2 receptor (EP1 subtype), TRH receptor, and Vasopressin receptor.

In a further aspect of this embodiment, the G-protein is a Gqi. As used herein, a “Gqi” means a protein that shares sequence similarities with both Gq/11 and Gi/o subfamily proteins such that the Gqi is activated by a Gi/o-coupled GPCR and activates the Gq/11 signal transduction pathway (e.g., activation of phospholipase C and regulation of ion channels). An example of a Gqi according to the present invention is Gqi5, which is a polypeptide consisting of the amino acid sequence of Gq, except that the last 5 amino acids of G_{q} are replaced by the last 5 amino acids of Gᵢ₁, and that the fourth Cys from the C-terminus of Gᵢ₁ is changed to Ile, which makes Gqi5 pertussis toxin (PTX) resistant.

In another aspect of this embodiment, the G-protein is a Gq/11 subfamily protein.

In an additional aspect of this embodiment, the second GPCR comprises a cysteine amino acid toward the terminal end of domain H8, which cysteine is palmitylated. As used herein, “domain H8” refers to helix 8 of the second GPCR, an amphiphilic short helix, which follows transmembrane helix 7 of the second GPCR (111). “Palmitylated” means the addition of a palmityl group to e.g., a cysteine residue (112). For example, in the sequence of the human wild type D2R, short isoform (SEQ ID NO: 61), this palmitylated cysteine towards the terminal end of domain H8 is the last residue (amino acid number 414). Preferably, the G-protein is fused directly to the cysteine amino acid toward the terminal end of H8, which preferably is palmitylated.

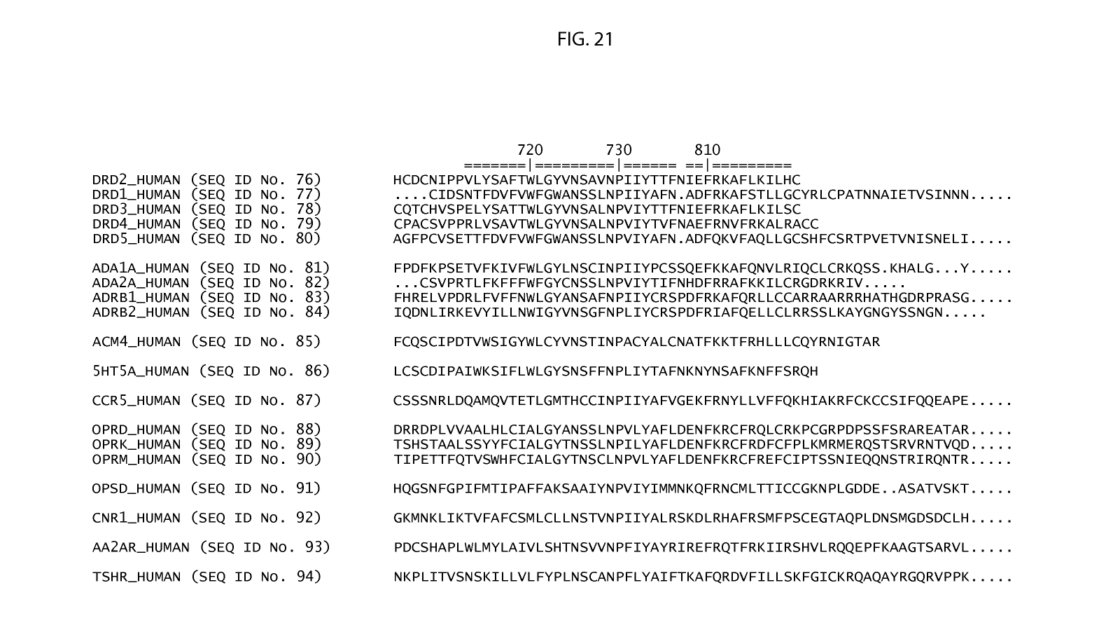

The inventors have shown that the palmitylated cysteine towards the terminal end of H8 is highly conserved among members of the Class A Family of GPCRs. (See e.g., sequence alignment in FIG. 21). Among the 1,184 class A receptors examined, 973 have at least one cysteine that corresponds to positions 410-418 of SEQ ID NO: 61. (Data not shown.) The following table shows the distribution of cysteines at positions that correspond to positions 410-418 of SEQ ID NO: 61.

TABLE 1
Cysteine distribution in Class A GPCRs

| Position | cysteine count |
|---|---|
| 410 | 14 |
| 411 | 44 |
| 412 | 84 |
| 413 | 310 |
| 414 | 505 |
| 415 | 198 |
| 416 | 144 |
| 417 | 111 |
| 418 | 80 |

In another aspect, the G-protein is fused to an amino acid that corresponds to a position selected from the group consisting of position 410, 411, 412, 413, 414, 415, 416, 417, and 418 of the human wild type D2R, short isoform (SEQ ID NO: 61) and isoforms, homologs, and orthologs thereof. As used herein, “isoform” means an alternative form of a protein resulting from differential transcription of the relevant gene either from an alternative promoter or an alternate splicing site. “Homolog” means a gene related to a second gene by descent from a common ancestral DNA sequence. “Ortholog” means a gene in a different species that evolved from a common ancestral gene by speciation.

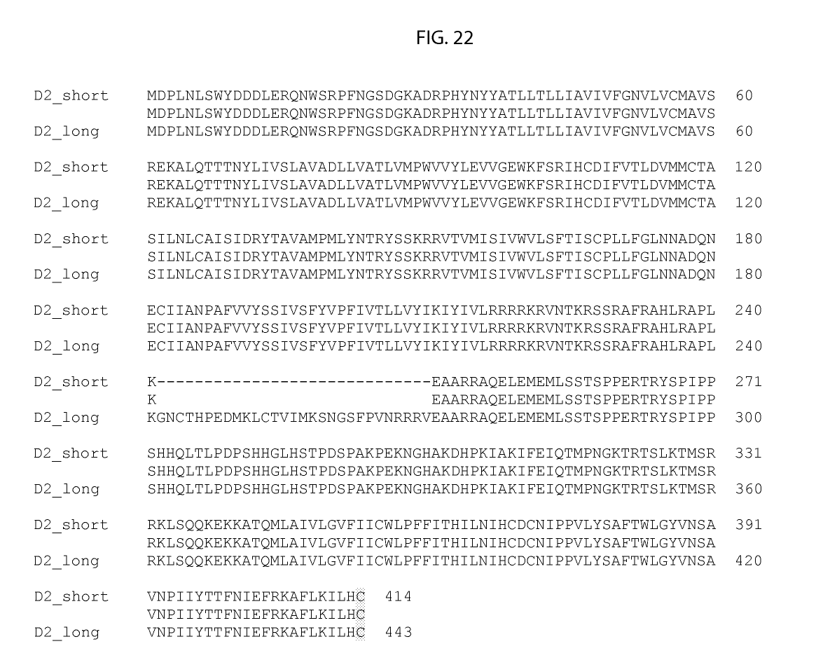

“Corresponds,” with reference to this embodiment, means consistent with, as done by sequence alignment. Multiple sequence alignment methods including pair-wise sequence alignment methods, may be used to determine the position in a GPCR that corresponds to the positions listed above. FIG. 22 shows a comparison between two isoforms of D2R, as performed by BLAST. As shown in the sequence comparison, the amino acid that corresponds to position 414 of the D2 short isoform is cysteine 443 of the D2 long isoform. Another example of such a multiple sequence alignment is shown in FIG. 21. The position that corresponds to position 414 of the D2 short isoform for human CCR5, for example, is a lysine residue (“K”). Methods of sequence alignment are well-known. Many sequence alignment softwares are available. These programs include, e.g., BLAST, ClustalW, SEQALN, DNA Baser, MEME/MAST, BLOCKS, and eMOTIF.

Preferably, the G-protein is fused to an amino acid that corresponds to a position selected from the group consisting of position 413, 414, 415, 416, and 417 of SEQ ID NO: 61 and isoforms, homologs, and orthologs thereof, and more preferably, an amino acid that corresponds to position 414 of SEQ ID NO: 61 and isoforms, homologs, and orthologs thereof. Most preferably, the amino acid is cysteine, and if the amino acid is not cysteine, then the amino acid is modified, using well known procedures, to be cysteine prior to fusion of the G-protein.

In an additional aspect of the embodiment, the first GPCR comprises a mutation. In another aspect, the second GPCR comprises a mutation. In a further aspect, both the first and second GPCRs comprise a mutation. In addition, the G-protein coupled to the second GPCR may be mutated with respect to a wild type form. As used herein, “mutation” means an alteration of the wild type gene, including but not limited to, addition, deletion, or substitution of at least one amino acid. Preferably, the mutation is from 1 to 3 single amino acid substitutions. Also preferably, the mutation creates a mutant D2R. This mutant D2R may be SFD80AGqi5 (SEQ ID NO: 11), SFD80A/CAMGqi5 (SEQ ID NO: 12), sMycD80A (SEQ ID NO: 29), SFD114AGqi5 (SEQ ID NO: 9), SFD114A/CAMGqi5 (SEQ ID NO: 10), sMycD114A (SEQ ID NO: 28), SFR132AGqi5 (SEQ ID NO: 16), SF132A/CAMGqi5 (SEQ ID NO: 17), sMycR132A (SEQ ID NO: 32), SFV136DM140EGqi5 (SEQ ID NO: 18), SFV136DM140E/CAMGqi5 (SEQ ID NO: 19), sMycV136DM140E (SEQ ID NO: 33), SFA213-219Gqi5 (SEQ ID NO: 13), SFA 213-219/CAMGqi5 (SEQ ID NO: 14), sMycA 213-219 (SEQ ID NO: 30), SFAAAA(219-222RRKR) Gqi5 (SEQ ID NO: 4), SFD2S AAAA(219-222RRKR)/CAMGqi5 (SEQ ID NO: 5), sMycAAAA(219-222RRKR) (SEQ ID NO: 25), SFAAAA(IYIV212-215)Gqi5 (SEQ ID NO: 20), sMycAAAA(IVIY212-215), SFN393AGqi5 (SEQ ID NO: 15), SFN393A/CAMGqi5 (SEQ ID NO: 8), sMycN393A (SEQ ID NO: 31), SFD24LGqi5 (SEQ ID NO: 1), SFD24L/CAMGqi5 (SEQ ID NO: 2), sMycD24L (SEQ ID NO: 22), SFCAMGqi5 (SEQ ID NO: 7), sMycD24 short (SEQ ID NO: 23), SFD131A/R132A Gqi5 (SEQ ID NO: 6), sMycCAM (SEQ ID NO: 27), SFD2/D4short Gqi5 (SEQ ID NO: 3), sMycD131N (SEQ ID NO: 39), sMycD131A/R132A (SEQ ID NO: 26), sMycD2S D114A/CAM (SEQ ID NO: 35), sMycD2S D114A/D131N (SEQ ID NO: 36), sMycD2S D114A/R132A (SEQ ID NO: 37), sMycD2S D114AN136D/M140E (SEQ ID NO: 38), or sMycD2S Y397F (Y7.53F) (SEQ ID NO: 40).

In yet another aspect of the embodiment, the complex is capable of producing a signal when presented with a ligand.

Another embodiment of the present invention is a method of producing a biological reagent. This method comprises the steps of: (a) expressing a first nucleic acid in a cell, the nucleic acid encoding a first GPCR; (b) expressing a second nucleic acid in the cell, the second nucleic acid encoding a fusion protein comprising a second GPCR fused to a G-protein, the G-protein being fused to the second GPCR in such a manner so that it cannot participate in a productive interaction with the second GPCR; and (c) allowing the expressed proteins from steps (a) and (b) to assemble into a complex in the cell membrane, wherein the expressed proteins from steps (a) and (b) alone are incapable of producing a signal when presented with a ligand.

In one aspect of this embodiment, the method further comprises, prior to step (a), producing a construct comprising the first nucleic acid encoding the first GPCR and the second nucleic acid encoding the fusion protein of the second GPCR and the G-protein, the G-protein being fused to the second GPCR.

As used herein, a “nucleic acid construct” or “construct” means an artificially constructed segment of nucleic acid that is intended to be introduced into a target tissue or cell, via, e.g., transformation or transfection. It may comprise a DNA sequence encoding a protein of interest, that has been subcloned into a vector, and promoters for expression in the organism. An example of such a construct is set forth in more detail in the Examples below.

In another aspect of this embodiment, the method further comprises, prior to step (a): (i) producing a first construct comprising the first nucleic acid encoding the first GPCR; and (ii) producing a second construct comprising the second nucleic acid encoding the fusion protein of the second GPCR and the G-protein.

In a further aspect of this embodiment, the method further comprises isolating a part of the cell membrane comprising the complex. Isolation of the cell membrane may be accomplished as disclosed in the Examples or by any suitable method known in the art.

An additional embodiment of the present invention is a method of determining whether a first and second GPCR have affinity for each other such that they form, or are capable of forming, a functional GPCR oligomer. This method comprises (a) producing or providing a first nucleic acid construct encoding a first GPCR; (b) producing or providing a second nucleic acid construct encoding a second GPCR and its associated G-protein as a fusion protein, the G-protein being fused to the second GPCR in such a manner so that it cannot participate in a productive interaction with the second GPCR, wherein the first GPCR and the second GPCR and its associated G-protein alone are incapable of producing a signal when presented with a ligand; (c) co-expressing the first and second nucleic acid constructs in a cell; and (d) determining the presence of a complex comprising the first and second GPCRs.

As used herein, “functional” means capable of triggering the appropriate signal transduction pathway upon suitable stimulation. An “oligomer” means dimer, trimer, or an organization of molecules involving even greater numbers of members. In the present embodiment, a dimer—either homodimer or heterodimer—is preferred.

In one aspect of this embodiment, the presence of a complex is determined by contacting the cell with a ligand that binds the first GPCR and determining whether the G-protein is activated. As used herein, an “activated” G-protein is capable of triggering a signaling pathway, resulting in measurable and/or observable changes in levels of molecules, such as calcium levels.

In another aspect, the cell expresses aequorin (AEQ). As used herein, “aequorin” means a photoprotein which emits light upon calcium binding. Such AEQ-expressing cells are described in more detail in the Examples.

Although the present invention is described with reference to AEQ cells, other cell-based systems using different read-outs are contemplated. Especially preferred systems are those that are adapted to high throughput screening (HTS), which, as used herein, defines a process in which large numbers of compounds are tested rapidly and in parallel for binding activity or biological activity against target molecules. In certain embodiments, “large numbers of compounds” may be, for example, more than 100 or more than 300 or more than 500 or more than 1,000 compounds. Preferably, the process is an automated process. HTS is a known method of screening to those skilled in the art.

A further embodiment of the present invention is a method of determining an effect a compound has on a GPCR oligomer. This method comprises (a) contacting a compound with a first cell expressing a GPCR oligomer having (i) a first GPCR; and (ii) a second GPCR fused to a G-protein, wherein the G-protein is fused to the second GPCR in such a manner so that it cannot participate in a productive interaction with the second GPCR, and the first GPCR and the second GPCR fused to the G-protein alone are incapable of producing a signal when presented with a ligand; (b) detecting the presence of a cellular signal resulting from contact between the compound and the GPCR oligomer; and (c) determining an effect the compound has on the GPCR oligomer.

In one aspect of this embodiment, the method further comprises comparing the effect with that resulting from contact between the compound and a mutant of the first GPCR and/or with that resulting from contact between the compound and a mutant of the second GPCR and/or G-protein. Preferably, this method is a HTS.

An additional embodiment of the present invention is a method of identifying a compound capable of interacting with a GPCR oligomer. This method comprises (a) providing a cell expressing a biological reagent according to the present invention; (b) contacting the biological reagent with the compound; and (c) determining whether the compound interacts with the GPCR oligomer.

In one aspect of this embodiment, interaction between the compound and the GPCR oligomer is determined by detecting a change in a cellular signal resulting from the interaction. Preferably, the cellular signal is selected from the group consisting of Ca²⁺ flux, cAMP levels, inositol 1,4,5 triphosphate levels, protein kinase C activation, and MAP kinase activation.

In another aspect of this embodiment, the cellular signal is determined using a reporter assay. As used herein, “a reporter assay” is a means of detection using a reagent system that detects a change in a cellular signal. Detection of the change may be accomplished through any conventional methodology, including, e.g., radioactive, fluorescent, luminescent, chromogenic, or enzymatic means. For example, one reporter assay, as described herein, utilizes aequorin, which emits blue light upon binding to calcium and thus reflects changes in levels of calcium.

In an additional aspect of this embodiment, the cell further comprises a plasmid encoding apoaequorin and the cellular signal is determined by a change in the luminescence of the cell. Preferably, the cell is a Flp-in T-rex 293 cell.

In a further aspect of this embodiment, the compound interacts with the GPCR oligomer as an agonist, antagonist, inverse agonist, or an inverse antagonist. As used herein, an “agonist” means a substance that binds to a receptor and triggers a response in the cell. An “antagonist” means a substance that does not trigger response itself upon binding to a receptor, but blocks or dampens agonist-mediated responses. An “inverse agonist” is a substance which binds to the same receptor binding-site as an agonist for that receptor and reverses constitutive activity of the receptor. An “inverse antagonist” is a substance which reverses the inverse agonist's activity and restores the receptor's activity.

In yet another aspect of this embodiment, the first GPCR has a modified amino acid sequence compared to the wild-type GPCR sequence so as to render it non-functional. As used herein, “non-functional” means incapable of triggering, or triggering at a substantially reduced rate compared to the wild type GPCR, the appropriate signal transduction pathway upon suitable stimulation. Such modification may be, e.g., a deletion, substitution, or addition of one or more amino acids.

In an additional aspect of this embodiment, the second GPCR is a human D2 receptor (hD2) and the first GPCR is selected from the group consisting of hD1, hD3, hCCR5, hSSTR5, hDOR, hTSHR, hGluR1, hGluR5, hCB1, hA2a, hM4, and h5HT1b. In the present invention, a letter preceding a receptor name refers to its species of origin. Thus, hD2 receptor refers to the human D2 receptor.

In a further aspect of this embodiment, the second GPCR is a mutant D2R as disclosed previously herein.

In another aspect of this embodiment, one of the GPCRs is selected from the group consisting of 3HA-human D1 (SEQ ID NO: 41), 3HAD1-linker-Gqi5 (SEQ ID NO: 42), 3HA-human 5HT1b (SEQ ID NO: 43), 3HA-human A2a (SEQ ID NO: 44), 3HA-human CB1 (SEQ ID NO: 45), mGluR1a (rat) (SEQ ID NO: 46), mGluR5a (rat) (SEQ ID NO: 47), SF-human D3 (SEQ ID NO: 48), SFD3Gqi5 (SEQ ID NO: 49), SFD3-linker-Gqi5 (SEQ ID NO: 50), SF-human SSTR5 (SEQ ID NO: 51), smyc-human SSTR5 (SEQ ID NO: 53), 3HA-M4-linker-Gqi5 (SEQ ID NO: 54), 3HA-M4Gqi5a (SEQ ID NO: 55), human CCR5 (SEQ ID NO: 56), CCR5 Gqi5, (SEQ ID NO: 57), smycDOR (SEQ ID NO: 58), and TSHr Gqi5 (SEQ ID NO: 59).

In an additional aspect of this embodiment, the first GPCR is a wild type D2R and the second GPCR fused to a G protein is D2-Gqi5. Preferably, this method is adapted to be a HTS as set forth previously.

Yet another embodiment of the present invention is a method of identifying a compound having the ability to modulate binding between a GPCR oligomer and its ligand. This method comprises (a) providing a cell expressing a GPCR oligomer comprising: (i) a first GPCR; and (ii) a second GPCR linked to a G-protein, the linkage between the second GPCR and the G-protein being of a length, which prevents productive interaction between the G-protein and the second GPCR, wherein the first GPCR and the second GPCR linked to the G-protein alone are incapable of producing a signal when presented with a ligand; (b) contacting the cell with a test compound in the presence of the ligand; and (c) comparing the ability of the ligand to bind to the GPCR oligomer with the ability of the ligand to bind to the GPCR oligomer under comparable conditions but in the absence of the compound.

As used herein, the ability to “modulate binding” means the ability to change (i.e., increase or decrease) the affinity, in this case, between the GPCR oligomer and its ligand.

In one aspect of this embodiment, the compound is a protein or a peptide. Preferably, the protein is a third GPCR.

In another aspect of this embodiment, the ligand binds to a new or altered ligand binding site determined to be present on the oligomer.

In an additional aspect of this embodiment, the first GPCR, the second GPCR, and/or the G-protein has a modified amino acid sequence compared to a wild-type sequence.

A further embodiment of the present invention is a method for evaluating differential G-protein coupling. This method comprises:
(a) providing a first cell expressing a first GPCR oligomer comprising:
   (i) a first wild type GPCR;
   (ii) a second wild type GPCR linked to a G-protein, the linkage between the second GPCR and the G-protein being of a length, which prevents productive interaction between the G-protein and the second GPCR, wherein the first GPCR and the second GPCR linked to the G-protein alone are incapable of producing a signal when presented with a ligand;
(b) providing a second cell expressing a second GPCR oligomer comprising:
   (i) the first GPCR comprising a mutation;
   (ii) the second GPCR linked to a G-protein, the linkage between the second GPCR and the G-protein being of a length, which prevents productive interaction between the G-protein and the second GPCR, wherein the mutant first GPCR and the second GPCR linked to the G-protein alone are incapable of producing a signal when presented with a ligand;
(c) providing a third cell expressing a third GPCR oligomer comprising:
   (i) the first GPCR;
   (ii) the second GPCR, which comprises a mutation and is linked to a G-protein, the linkage between the second mutant GPCR and the G-protein being of a length, which prevents productive interaction between the G-protein and the second mutant GPCR, wherein the first GPCR and the second mutant GPCR linked to the G-protein alone are incapable of producing a signal when presented with a ligand;
(d) contacting the first, second, and third cells with a compound capable of binding to the ligand binding site present on the first and/or the second GPCR;
(e) repeating steps (a) to (d) with a different G-protein; and
(f) evaluating differential G-protein coupling.

In one aspect of this embodiment, the G-protein is Gqi. In another aspect, the G-protein modulates an intracellular signal selected from the group consisting of Ca²⁺ level, cAMP level, cGMP level, inositol 1, 4, 5 triphosphate level, diacylglycerol level, protein kinase C activity, and MAP kinase activity. In a further aspect, the first, second, and third cell each express aequorin and the evaluation step comprises detecting luminescence. In this and other aspects of the present invention, endogenous G-proteins may optionally be inactivated with, e.g., PTX or siRNA prior to contacting the cells with a ligand.

Another embodiment of the present invention is a method of identifying a compound having the ability to modulate the activity of a GPCR oligomer. This method comprises:
(a) providing a cell expressing a GPCR oligomer comprising:
   (i) a first GPCR; and
   (ii) a second GPCR linked to a G-protein, the linkage between the second GPCR and the G-protein being of a length, which prevents productive interaction between the G-protein and the second GPCR, wherein the first GPCR and the second GPCR linked to the G-protein alone are incapable of producing a signal when presented with a ligand;
(b) contacting the cell with a test compound in the presence of a ligand of the first GPCR or of the second GPCR; and
(c) comparing the activity of the GPCR oligomer with the activity of the GPCR oligomer under comparable conditions but in the absence of the compound.

As used herein, the “activity of a GPCR oligomer” means the amount of productive interactions between the GPCR oligomer and a G-protein. In this context, the ability to “modulate” the activity of a GPCR oligomer means the ability to change (i.e., increase or decrease) the amount of productive interactions between the GPCR oligomer and a G-protein. The amount of productive interaction between a GPCR oligomer and a G-protein may be determined, e.g., by detecting a change in a cellular signal resulting from the interaction, such as Ca²⁺ flux, cAMP levels, inositol 1,4,5 triphosphate levels, protein kinase C activation, and MAP kinase activation. Cellular signals may be determined by a reporter assay, such as, e.g., those disclosed herein. Suitable cells for use in this method include Flp-in T-rex 293 cells. Preferably, the cell expresses aequorin. This method may be adapted to be a high throughput screen. The compound may interact with the GPCR oligomer as an agonist, antagonist, inverse agonist, or an inverse antagonist. Furthermore, the compound may bind to the ligand binding site of the GPCRs or to an allosteric site.

In one aspect of this embodiment, the compound binds to the second GPCR but not the first GPCR. Alternatively, the compound binds to the first GPCR but not the second GPCR.

In another aspect of this embodiment, the second GPCR is D2R. In a further aspect of this embodiment, the first GPCR is selected from the group consisting of D2R, SSTR5, and DOR.

A further embodiment of the present invention is a method for evaluating differential effects of a compound on the activity of a GPCR oligomer.

This method comprises:
(a) providing a first cell expressing a first GPCR oligomer comprising:
   (i) a first GPCR;
   (ii) a second GPCR linked to a G-protein, the linkage between the second GPCR and the G-protein being of a length, which prevents productive interaction between the G-protein and the second GPCR, wherein the first GPCR and second GPCR linked to the G-protein alone are incapable of producing a signal when presented with a first ligand;
(b) providing a second cell expressing a second GPCR oligomer comprising:
   (i) a third GPCR;
   (ii) a fourth GPCR linked to a G-protein, the linkage between the fourth GPCR and the G-protein being of a length, which prevents productive interaction between the G-protein and the fourth GPCR, wherein the third GPCR and the fourth GPCR linked to the G-protein alone are incapable of producing a signal when presented with a second ligand;
(c) contacting the first and second cells with a compound capable of binding to the first, the second, the third, and/or the fourth GPCR; and
(d) evaluating the differential activity, if any, of each GPCR oligomer under comparable conditions but in the absence of the compound.

In this embodiment, the activity of the GPCR oligomers may be determined by any means disclosed herein, such as, e.g., a change in a cellular signal resulting from the activity of GPCR oligomers or using any other suitable readout. Preferably, the first and the second cell each express aequorin and the evaluation step comprises detecting luminescence. This method may be adapted to be a high throughput screen. The compound may interact with the GPCR oligomer as an agonist, antagonist, inverse agonist, or an inverse antagonist. Furthermore, the compound may bind to the ligand binding site of the GPCRs or to an allosteric site.

In one aspect of this embodiment, the first, the second, and the fourth GPCRs are the same. In one preferred embodiment, the first, the second, and the fourth GPCRs are D2R. In another preferred embodiment, the third GPCR is SSTR5.

The following examples are provided to further illustrate the compositions and methods of the present invention. These examples are illustrative only and are not intended to limit the scope of the invention in any way.

### EXAMPLES

### Example 1

### Materials

The D2R agonist quinpirole hydrochloride and the D4R antagonist L745,870 (3-(4-[4-Chlorophenyl]piperazin-1-yl)-methyl-1H-pyrrolo[2,3-b]pyridine trihydrochloride) were from Sigma-Aldrich (St. Louis, Mo.).

### DNA Constructs

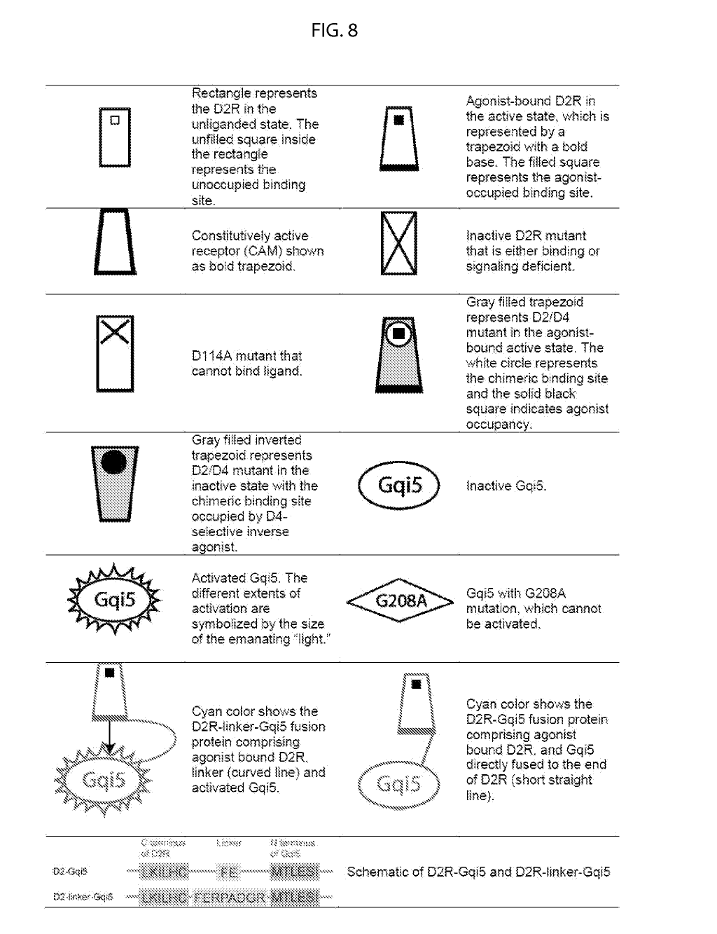

Expression plasmids expressing signal peptide flag-tagged short isoform of D2R wild type (114) and mutant receptors were created using standard molecular biology procedures, as described below. Receptor constructs were fused directly through their C-terminus, or through an 8 amino acid linker (FERPADGR, SEQ ID NO: 75), to a PTX-resistant Gqi5. (FIG. 8).

The stop codons in the signal peptide flag-tagged D2R short isoform (D2s) wild type (SEQ ID NO: 62) (115) and mutant receptors were removed by PCR, and the sequence TTCGAA was inserted in place of the D2R stop codon to create a BstBI site. Gaqi5 (referred to as Gqi5) (SEQ ID NO: 67) was constructed by replacing the last 5 amino acids of Gaq with those of Gail, except that the fourth residue from the C-terminus was mutated from Cys to Ile. This mutation rendered Gai pertussis-toxin resistant (17). The sequence TTCGAA was also inserted immediately priority to the start codon of Gqi5. D2R-Gqi5 (schematic illustration shown in FIG. 8) was made by subcloning the two fragments using the BstBI site. D2R-linker-Gqi5 (schematic illustration shown in FIG. 8) (the flag tagged version is shown in SEQ ID NO: 69) was made using polymerase chain reaction (PCR) by inserting the additional sequence TTCGAAAGACCTGCAGACGGTAGA (SEQ ID NO: 74), which encodes FERPADGR (SEQ ID NO: 75) as a linker, between the last amino acid of D2R and the start codon of Gqi5. For D2R-linker-Gqi5G208A (SEQ ID NO: 70), G208A was mutated by PCR, which resulted in a nonfunctional Gα (28). Flag-tagged D2R, D2R-Gqi5 and D2R-linker-Gqi5 (SEQ ID NOs: 62, 64, and 69, respectively) were subcloned into pcDNA5/FRT/TO vector (Invitrogen), respectively, according to the manufacturer's instructions. cDNA encoding Myc-tagged D2R and Gqi5 (SEQ ID NO: 24 and 67, respectively) were subcloned into the pIRESpuro3 vector (Clontech). Plasmids encoding apoaequorin were obtained from Vincent J. Dupriez (Euroscreen S. A., Brussels, Belgium), and an apoaequorin sequence was subcloned into the pCIN4 plasmid (115) from BD Biosciences Clonetech (Palo Alto, Calif.) using e.g., the manufacturer's instructions to create pCIN4AEQ.

Sequences not provided herein are obtainable from publicly available sources, such as the National Center for Biotechnology Information (NCBI).

### Cell Culture And Transfection

Flp-in T-rex 293 cells (Invitrogen, Carlsbad, Calif.) were maintained in DMEM medium (GIBCO, Carlsbad, Calif.) supplemented with 10% (v/v) FBS (Gemini, W. Sacramento, Calif.) and 2 mM L-glutamine (Invitrogen). Cells were transfected with Lipofectamine 2000 (Invitrogen) according to manufacturer's protocol. pCIN4AEQ was transfected into Flp-in T-rex 293 cells (Invitrogen), followed by G418 (Mediatech Inc., Manassas, Va.) selecting. Single colonies were isolated, and a clone was identified in which acetylcholine-induced activation of endogenous muscarinic M1 receptors (which couple to endogenous Gq) resulted in robust luminescence in the presence of coelenterazine h (Byosinth AG, Switzerland) (see Aequorin Assay section below).

This parental aequorin cell line was transfected with unfused Myc-tagged D2R (SEQ ID NO: 24) in pIRESpuro3 followed by puromycin (Sigma-Aldrich) selection. After selection, cells were transfected with Flag-tagged D2R-Gqi5 fusion (SEQ ID NO: 64) in pcDNA5/FRT/TO, followed by hygromycin b (Mediatech) selection. Stable coexpression of unfused D2R (SEQ ID NO: 62) with unfused Gqi5 (SEQ ID NO: 67) was achieved by the same strategy. In the Examples, when pertussis toxin treatment is notes, cells were treated with 100 ng/ml pertussis toxin (Sigma-Aldrich) 16-24 hours prior to harvest.

### Aequorin Assay

A functional assay based on luminescence of mitochondrial aequorin following intracellular Ca²⁺ release was performed (23, 24). Cells were seeded in a 15 cm plate, and grown in antibiotics-free medium for about 48 hours until mid-log phase. Tetracycline (1 μg/ml) was added to the medium for 3-24 hours prior to harvest to induce the expression of D2R in a FRT/TO vector (Invitrogen), e.g., pcDNA5/FRT/TO. Cells were dissociated, and then pelleted by centrifuge at 0.6×g for 3 minutes. After washing once with DMEM-F12 medium (Invitrogen, supplemented with 0.1% BSA), cells were resuspended in the same medium to the final concentration of 5×10⁶ cells/ml in the presence of 5 μM coelenterazine h (Biosynth AG). The cell solution was further diluted 10-fold after 4 hours of rotating at room temperature in the dark, followed by one hour incubation under the same conditions. A dose-dependent response was measured by injecting 50 μL cell solution into wells containing 50 μL of different concentrations of an appropriate agonist, such as quinpirole (a D2/D3 receptor agonist), in a 96-well plate. Luminescence signals from the first 15 seconds after injection were read by a POLARstar optima reader (BMG Labtech GmbH, Durham, N.C.). Total response was determined by the signal of injecting 50 μL cell solution into 50 μL assay medium containing 0.1% triton, which raises the Ca⁺⁺ concentration directly by membrane permeabilization.

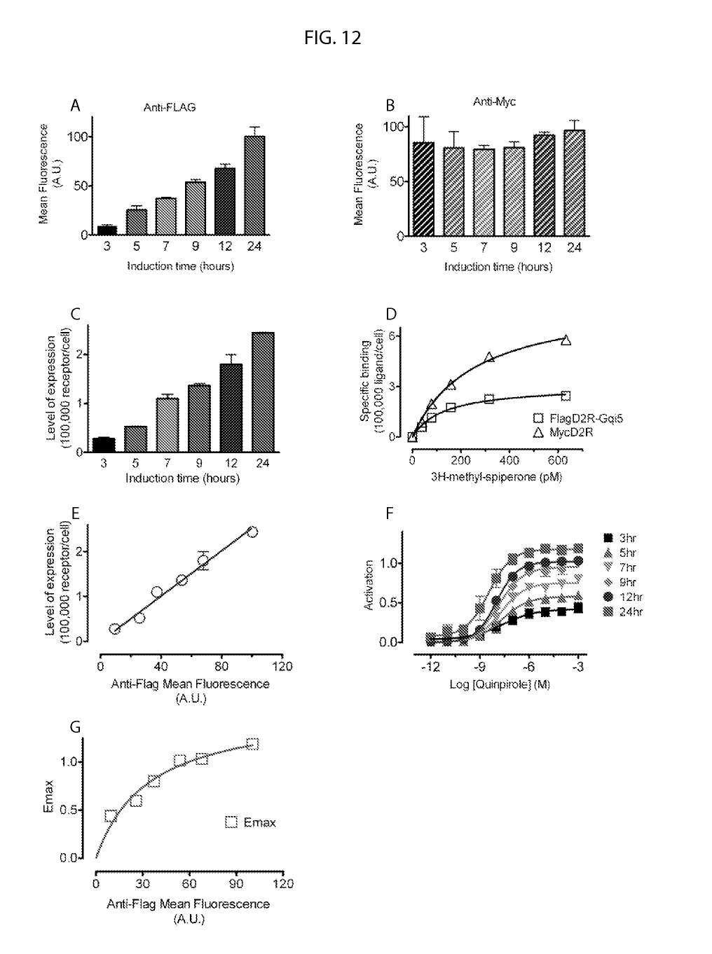

The signals were further normalized according to Flag tagged D2R expression level. To normalize for different levels of surface expression levels of the Flag-D2R-Gqi5 mutant constructs, the Emax at each expression level (FIG. 12F) was plotted as a function of different levels of expression of Flag-tagged wt D2R-Gqi5, the expression of which was controlled by varying the time after tetracycline induction (FIG. 12A). The level of Myc-D2R remained essentially unchanged (FIG. 12B). The standard curve was fit to a 1 site rectangular hyperbola using nonlinear regression in GraphPad Prism 4.0 (GraphPad Software Inc., La Jolla, Calif.) (FIG. 12G). The luminescence response of the various Flag-D2R-Gqi5 constructs was normalized using this standard curve to account for the effects of different expression levels, with activation of 1 defined as that observed after 12 hours of tetracycline induction of WT D2R-Gqi5. The Flag detection was approximately 5-fold more sensitive than that of Myc; thus, the excess of Myc-tagged protomer A, which cannot signal on its own, ensures that normalization based on surface expression of the Flag-tagged Gqi5-fused protomer B accurately reflects the productive signaling entities, each of which must contain a protomer A and a protomer B.

### Cell Surface Expression Assay

Cells that co-expressed Flag tagged D2R Gqi5 fusion and Myc tagged un-fused D2R were induced by 1 μg/ml tetracycline for different amounts of time. An aliquot of the cell solution used for the aequorin assay was used to determine receptor cell surface expression as described in Costagliola et al. (116). Cells were incubated with M2 monoclonal anti-Flag antibody (Sigma) or anti-Myc monoclonal antibody (gift from Cornell) for 30 minutes, followed by another 30 minutes incubation with R-phycoerythrin goat anti mouse IgG (Invitrogen). Cell solutions were diluted to a suitable concentration for FACS assay using Guava Easycyte (Guava technologies, Hayward, Calif.). The surface expression of D2R or D2R-Gqi5 fusion were detected with whole cells without permeation, which could exclude intracellular immature receptors readings.

### Saturation and Competition Binding

Cells expressing Flag-D2R-Gqi5 were harvested after induction by tetracycline for varying times from 3 to 24 hours. Cells continuously expressing Myc-D2R were harvested when confluence was suitable. Binding studies were carried out with [³H]N-methylspiperone (PerkinElmer Life Sciences, Waltham, Mass.) using 1 μM sulpiride (Sigma-Aldrich) to define nonspecific binding, as described previously (19). Cells coexpressing D2R or D2R E339A/T343R with free Gqi5 were induced for 20 hours prior to competition binding assay. Intact cells were harvested for binding, and [³H]N-methylspiperone binding was performed as described previously (19).

### Model Construction

In the absence of experimentally determined structures of dopamine receptor and Gqi5, the templates for the oligomeric constructs were based on a complex between a heterotrimeric G-protein and rhodopsin. The bovine Gtα subunit was built by homology modeling with MODELLER software (42) from the crystal structure of the complex of a Gtα/Giα chimera and the Gtβγ subunits (PDBID: 1GOT) (41). As the very important C-terminal residues of Gtα (residues 340-350) were missing from the resulting complex, the “activated peptide” of Gt (PDBID: 1 LVZ) was grafted to this structure (43). For this purpose it was necessary to overlap the region Ile340-Glu342 and mutate Ser347 back to Cys. The 1GOT structure of the heterotrimeric Gtαβγ was used to model the Gγβ subunit. As the last residues of Gtγ were missing, the same approach as described above was used to complete the structure: the Gtγ (60-71) farnesyl dodecapeptide (PDBID: 1MF6) (44) in complex with an activated rhodopsin was grafted to the Gt modeled by overlapping Asp60-Asn62. Energy minimization of the Gαβγ was then performed using the AMBER force field (45).

Inspection of the first crystal structure of a heterotrimeric G protein had indicated that the surface area of a GPCR monomer was probably too small to interact simultaneously with both α- and β/γ-subunits of a G protein, leading to the suggestion that the signaling unit could be a dimer. To enable the simultaneous probing of many possible dimer arrangements, an oligomer composed of nine rhodopsin monomers was constructed. The rhodopsin monomers were in the activated form obtained by inclusion of all constrains reported for rhodopsin as reported by Niv et al. (46). Three dimeric interfaces were analyzed: Model 1, in which the dimers have a TM4,5 interface; Model 2, with a symmetric TM4 interface (see Guo et al. (39) for structural details of the interfaces); and Model 3, in which the dimers have a TM1 interface (117).

### G-Protein-Rhodopsin Docking

The docking software used was HADDOCK (High Ambiguity Driven protein-protein DOCKing) (47, 48), which produced one of the best results in the CAPRI (Critical Assessment of PRediction of Interactions) contest and is well characterized in the literature. The docking process for the three models was driven by ambiguous interaction restraints (AIRs) (47) to both monomers. The constraints, which were established from literature-derived experimental data for the binding complex, are presented in Table 2. Notably, the docking protocol of Gt to such models using this set of constraints was verified by the full agreement with the complex obtained for the recent structure of opsin (118) representing a putative activated form of the protein (see below). To select a second protomer for the complex, another docking run was made with restraints only to the central rhodopsin, allowing transducin to explore freely different orientations with respect to the rhodopsin oligomer, and therefore, for the calculation of the relative probabilities of TM1,1 dimers compared to TM4,5 dimer interfaces.

TABLE 2
Residues at the Transducin - Rhodopsin interface

| Protein | Residues and Positions | Citation |
|---|---|---|
| Gtα | V214, R309, D311, V312, K313, F330, F332, | (49, 50) |
| | D337, I338, I340, K341, N343, L344, G348, | |
| | L349, F350 | |
| Gtα | C347 | (51) |
| Gtγ | N62A, P63A, F64A | (44) |
| Gtα | L19-R28 | (40, 52) |
| RHO | Y136 (3.51), V137 (3.52), V138 (3.53), V139 | (53) |
| | (3.54) | |
| RHO | C140 (3.55), K141 (3.66), R147 (3.72), F148 | (54) |
| | (3.73) | |
| RHO | T229 (5.64), V230 (5.65), A233 (5.68), A234 | (54-56) |
| | (5.69), S240 (6.23), T242 (6.25), T243 (6.26), | |
| | Q244 (6.27) | |
| RHO | E247 (6.30), K248 (6.31), E249 (6.32) | (53) |
| RHO | N310 (7.57), K311 (7.58), Q312 (7.59) | (53, 57-58) |

Application of the experimentally-derived constraints took advantage of the distinction made by the HADDOCK algorithm between “active” and “passive” residues. The “active” residues are those considered to be involved in the interaction between the two molecules (Table 3) and to be solvent accessible (either main chain or side chain relative accessibility should be typically >40-50%, which is calculated with the software NACCESS (59)). The “passive” residues are all solvent accessible surface neighbors of active residues. An AIR, the maximum distance between any atom of an active residue of one molecule to any atom of an active or passive residue of the second molecule has a maximum value of 3 Å, as the effective distance d_{eff} will always be shorter than the shortest distance entering the sum: d_{eff}=[Sum(1/r⁶)]^{1/6} In Table 3 below, the distance (in Å) between the Cα of specific residues of rhodopsin and transducin for the two studied models is shown in bold italics. Numbers in brackets are the average distances from the docking solutions from which the optimal representatives were chosen as Model 1 (TM4, 5 dimer) and Model 2 (TM4 dimer) as described above.

TABLE 3
Literature-derived constraints used in the construction
of the models for rhodopsin-transducin complexes

| Set | RHO | Transducin | Model 1 | Model 2 |
|---|---|---|---|---|
| 1 | R135A | F350 α | [symbol] <26> | [symbol] <25> |
| | V137A | F350 α | [symbol] <29> | [symbol] <29> |
| | S144A | D337 α | [symbol] <33> | [symbol] <30> |
| | N145A | F350 α | [symbol] <29> | [symbol] <21> |
| | T229A | F350 α | [symbol] <29> | [symbol] <21> |
| | S240A | E342 α | [symbol] <34> | [symbol] <20> |
| | | N343 α | [symbol] <33> | [symbol] <18> |
| | | L344 α | [symbol] <33> | [symbol] <19> |
| | | N345 α | [symbol] <33> | [symbol] <18> |
| | | K313 α | [symbol] <38> | [symbol] <26> |
| | E247A | F350 α | [symbol] <39> | [symbol] <21> |
| | E249A | L344 α | [symbol] <46> | [symbol] <18> |
| 2 | K141A | R28 α | [symbol] <26> | [symbol] <20> |
| | S240A | K28 α | [symbol] <32> | [symbol] <17> |
| | K248A | L19 α | [symbol] <41> | [symbol] <24> |
| 3 | R147 | A23 α | [symbol] <23> | [symbol] <16> |
| | S240B | L19 α | [symbol] <19> | [symbol] <17> |
| | | A23 α | [symbol] <16> | [symbol] <12> |
| | C316B | P63 γ | [symbol] <41> | [symbol] <36> |

By application of the docking protocol consisting of randomization of orientations and rigid body energy minimization, 1000 different conformations were generated. These structures were ranked according to their average interaction energies (sum of E_{elec}, E_{vdw}, E_{AIR}). All structures were screened using eighteen restraints given in Table 3. These represent the information extracted from the experimental data and translated into Cα-Cα intermolecular constraints. Set 1 refers to the interactions of the C-terminus of Gα with protomer A (e.g., GPCR1), set 2 to the interactions between the N-terminus of Gα and protomer A, and set 3 to the interactions between the N-terminus of Gα and protomer B (e.g., GPCR2). Sets 1 and 2 in Table 3 contain interactions that could occur simultaneously, whereas set 3 refers to another group of interesting interactions that do not occur simultaneously in the same protomer, for steric reasons. Set 1 was used to filter the most reliable solution because it includes restraints between residues for which experimental support comes from several different sources. In contrast, the interactions of rhodopsin with the N-terminus of Ga are not as well defined. Experimental studies only demonstrate that a broad region of the N-terminus, residues 19 to 28, is involved in the binding interface (40, 53). Kisselev et al. (44) showed that Gγ(50-71), especially Phe64, interacts with the C316 of rhodopsin. Nevertheless, analysis of the rates of Meta II decay led to the proposition that rhodopsin presents two distinct signaling states, one bound to Gtα and one bound to Gtβγ (61).

With this in mind, Gγ was positioned near the binding interface, although not necessarily in direct interaction with rhodopsin. Only Cα-Cα distances<20 Å were interpreted as direct rhodopsin-Gt interactions. A cutoff of 50% fulfillment of the of the interaction criteria was used for accepting valid constructs. The relative probabilities of such valid G protein complexes with the various model dimers (TM4; TM4,5; TM1) were calculated from the corresponding percentages of acceptable complexes found in the resulting set of 1000 structures retrieved from the docking procedure. The construct fulfilling the largest, number of experimentally derived constraints and with the N-terminal helix of Ga parallel to the cytoplasmic face of the rhodopsin dimer, was chosen as the “optimal representative structure” for each model.

The crystal structure of opsin (Ops*) in complex with the GαCT (340-350) segment published recently (27) provided an opportunity for validation of the docking procedure disclosed herein for a cognate monomer in a proposed activated form. Using the computational protocol disclosed herein, the component structures were docked and scored according to the interaction criteria described above. The structures chosen based on these criteria present Root Mean Square Deviation (RMSD) values lower than 2.5 Å in comparison to the crystallographic complex (PDBID: 3DQB) (27), positioning the GαCT ligand in exactly the same binding crevice as observed in the crystal structure. These results confirm the applicability of the procedure and the scoring criteria used to dock the Gt protein.

### Example 2

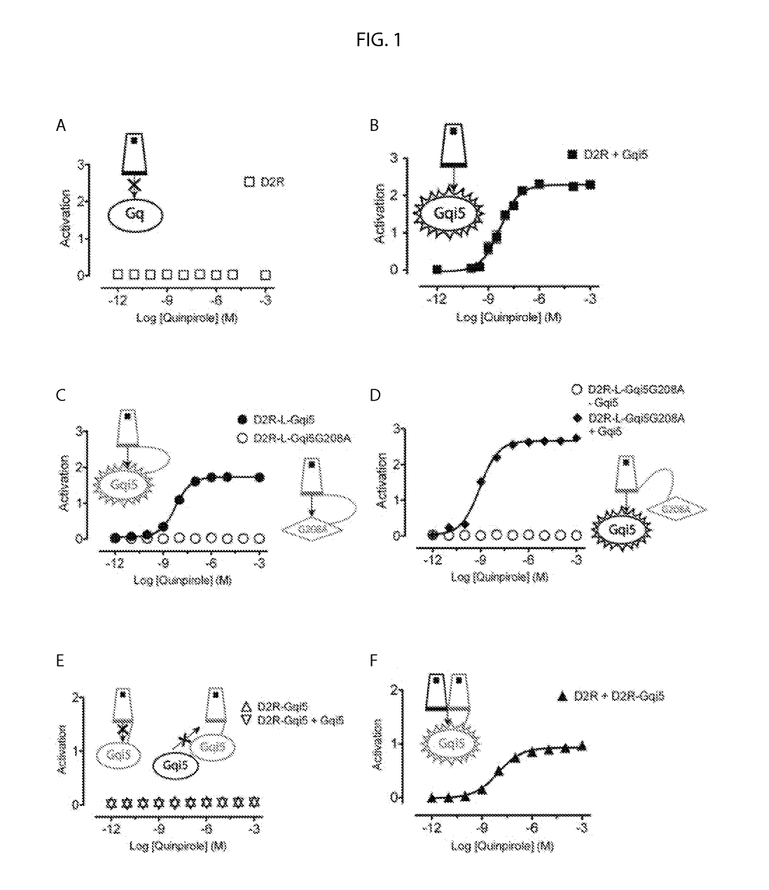

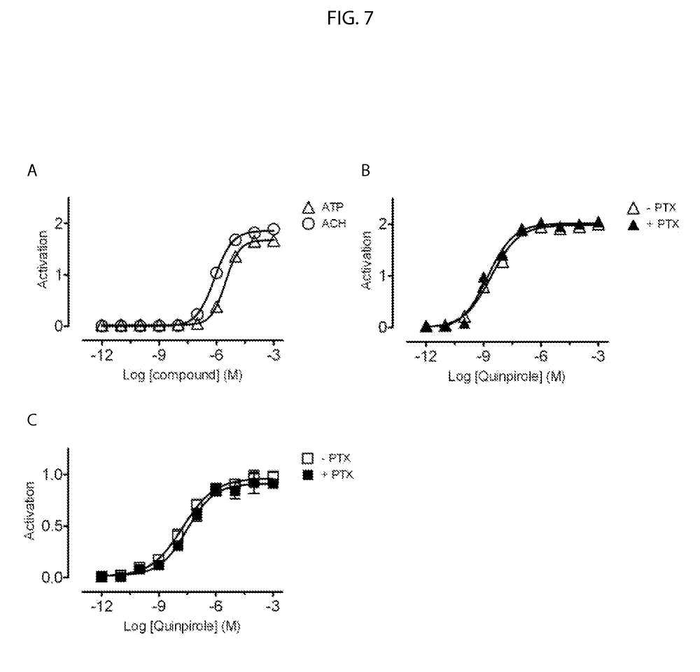

To enable isolation of the signaling of the D2R from endogenous G proteins, and to control each of the components of the signaling complex, Flp-In 1-Rex-293 cells were engineered to stably express aequorin (AEQ cells) (see Example 1). Aequorin produces luminescence in a calcium-dependent manner in the presence of the substrate coelenterazine (23, 24), and it has been used to create a sensitive luminescence readout for GPCR-mediated PLC activation (25). In these cells, endogenous muscarinic or purinergic receptors signaled robustly via endogenous Gq, resulting in strong agonist-induced (ACH and ATP, respectively) luminescence signals (FIG. 7A). In contrast, when D2R was stably expressed in AEQ cells, treatment with the agonist quinpirole did not lead to luminescence, consistent with a lack of D2R coupling to Gq (FIG. 1A, and see FIG. 8 for an explanation of the symbols used in the figures).

To couple D2R activation to a luminescence readout in these cells, a chimeric pertussis toxin-resistant (26) Gq that could signal from Gi-coupled receptors (27) was expressed (See Example 1). D2R signaled robustly when stably co-expressed with this free chimeric Gqi5 or when fused at its C-terminus to Gqi5 through an 8 amino acid linker (D2-linker-Gqi5) (SEQ ID NO: 69) (FIGS. 1B and 1C). The increase in luminescence was unaffected by PTX (FIG. 7B), whereas a mutation in Gqi5 (Gqi5_{G208A}) that prevents GTP-induced Ga activation (28) prevented the luminescence response to D2R activation (FIG. 1C). No quinpirole response was seen when free Gqi5 was expressed without D2R (data not shown), consistent both with the absence of endogenous D2R in these cells and with the lack of other targets for quinpirole-mediated signaling.

Curiously, expression of free Gqi5 (SEQ ID NO: 67) fully rescued the function of the D2-linker-Gqi5_{G208A} (SEQ ID NO: 71) (FIG. 1D), indicating that the linker afforded sufficient flexibility and mobility for the nonfunctional G protein to swing away, permitting a free functional Gqi5 to interact and to restore agonist-mediated signaling. Therefore, this construct cannot be used to monitor functional coupling of two defined protomers (e.g., GPCR1 and GPCR2), because the flexibility of the linker might allow D2-linker-Gqi5 to provide the Ga to another protomer (or even to another dimer of protomers) without the actual participation of the fused receptor in the signaling unit. Consistent with such promiscuous interactions, in preliminary experiments, a number of different inactivating mutations in the G protein-fused GPCR protomer (termed Protomer B, e.g., GPCR2) failed to impair signaling by the unfused “Protomer A”, which suggested that it could provide G protein without participating in the relevant signaling unit.

### Example 3

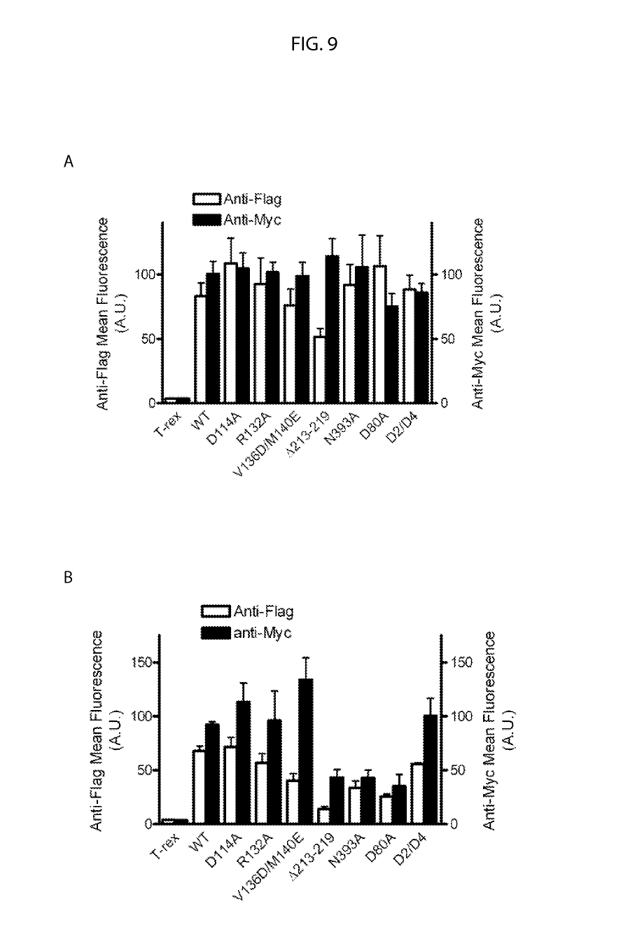

To address this problem, another construct was developed. In this construct (D2-Gqi5), Gqi5 was fused more directly to the short cytoplasmic tail of the D2R, via a two amino acid linker. This construct was expressed at the plasma membrane (FIGS. 9A and 9B) and bound ligand (data not shown), but agonist treatment failed to produce luminescence (FIG. 1E). Although this might have resulted from misfolding of the fused Ga in this construct, it is hypothesized instead that the lack of signaling resulted from the inability of the tethered Ga to be appropriately positioned for a productive interaction with the cytoplasmic loops of the receptor to which it was fused, or with a second protomer. Indeed, in contrast to the D2-linker-Gqi5, D2-Gqi5 signaling was not rescued by free Gqi5 (FIG. 1E), most likely because the tethered Ga sterically blocks free Gi5 from making a productive interaction with the cytoplasmic loops of the fused receptor.

Remarkably, however, co-expression in the AEQ cells of D2R and D2-Gqi5, each of which are completely incapable of signaling in assays when expressed alone, led to robust agonist-mediated receptor activation (FIG. 1F), indicating that when activated the fused Gqi5 is fully capable of interacting with PLC (phospholipase C). That this effect was mediated solely by the fused Gqi5 and not by endogenous Gi/o was supported by the lack of effect of pertussis toxin treatment on activation (FIG. 7C). Because the complementation assay disclosed herein is able to reconstitute a signaling unit from two nonfunctioning protomers, there exists a unique opportunity to manipulate each protomer independently and to determine its role in signaling. The extremely close proximity between these protomers and the inability of protomer B to signal to its own fused G protein or to a neighboring fused G protein indicates that only one G protein serves this signaling unit of two GPCRs. In the simplest scenario this unit is composed of protomer A (e.g., GPCR1), protomer B (e.g., GPCR2), and the G protein fused to protomer B (FIG. 1F), although a higher order complex cannot be ruled out. For simplicity, this signaling unit may be referred to as a “dimer” herein. The inferences regarding this signaling unit are highly consistent with the results from the computational modeling studies disclosed herein, as they indicate that the relatively large size of the G protein heterotrimer would require an overlap of its extensive interface with the cytoplasmic surfaces of at least two neighboring GPCR protomers.

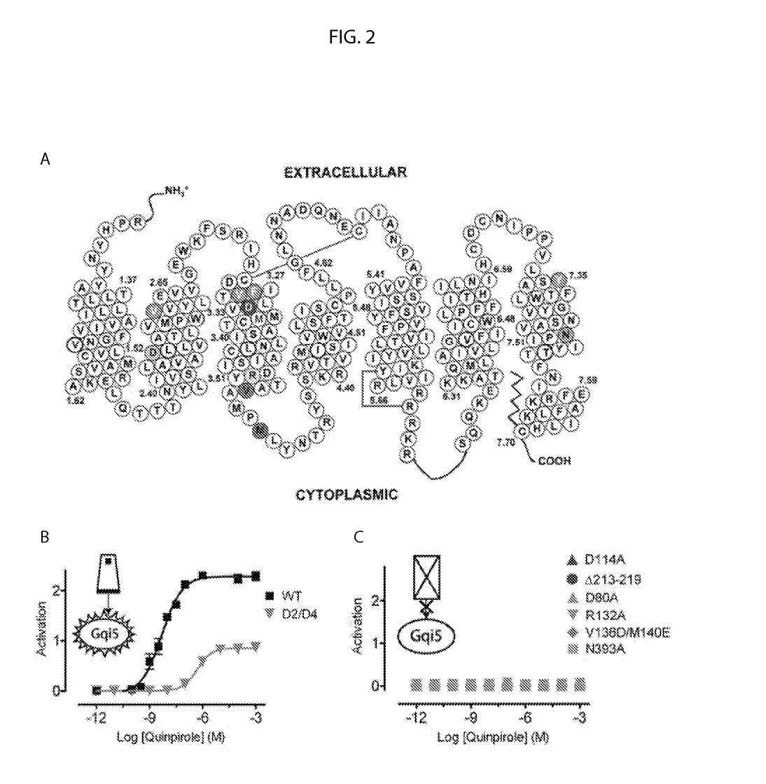

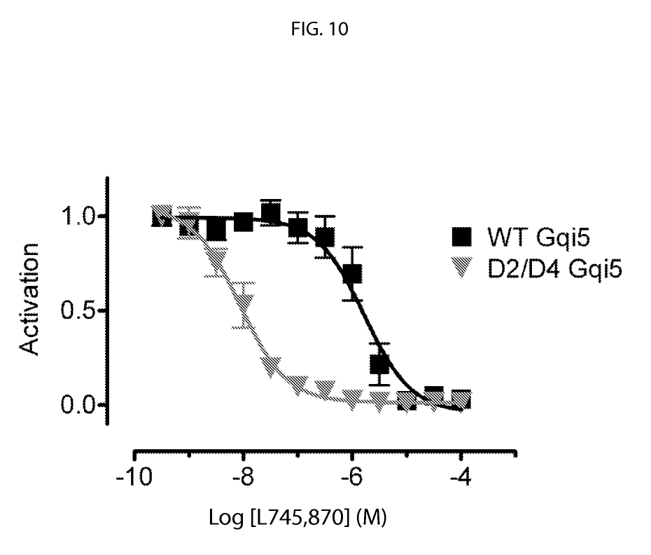

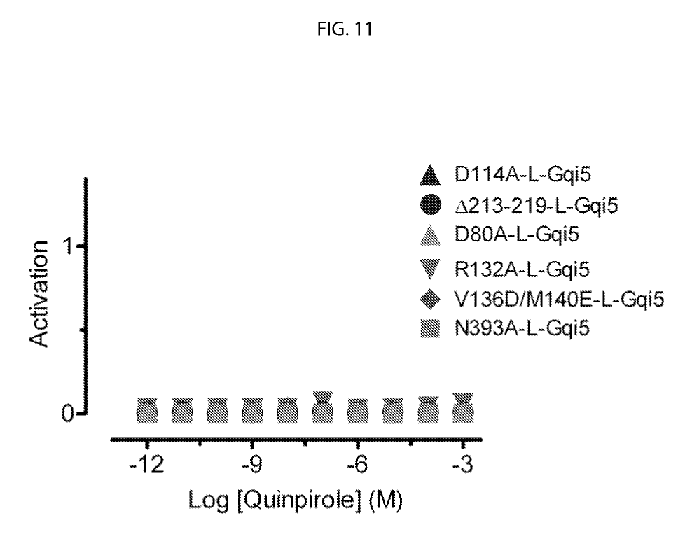

In order to manipulate experimentally the function of each protomer, a panel of D2R mutants predicted to be binding and activation-deficient based on findings in the literature for related Class A GPCRs was created and characterized (FIG. 2A). These include D114^{3.32}A (29) (myc-tagged version shown in SEQ ID NO: 28), which does not bind agonist or antagonist, as well as R132^{3.53}A (30) (myc-tagged version shown in SEQ ID NO: 32), and V136^{3.54}D/M140^{3.58}E in IL2 (31) (myc-tagged version shown in SEQ ID NO: 33), deletion of amino acids 213-219 in IL3 (32) (myc-tagged version shown in SEQ ID NO: 30), and D80^{2.50}A (33) (myc-tagged version shown in SEQ ID NO: 29), and N393^{7.49}A (34) (myc-tagged version shown in SEQ ID NO: 31) in the membrane-spanning segments, all of which were expected to disrupt agonist-mediated G protein activation. A D2R mutant V91^{2.61}F/F110^{3.29}L/V111^{3.28}M/Y408^{7.35}V (termed D2/D4) (FIG. 2A) (myc-tagged version shown in SEQ ID NO: 23) was also expressed. D2/D4, unlike WT D2R (SEQ ID NO: 60), is potently inhibited by the selective D4 antagonist L745,870 (35) (FIG. 10). Each of these constructs expressed at the plasma membrane (FIG. 9), and, except for D114^{3.32}A, each bound to the antagonist ³H-N-methylspiperone (data not shown). In contrast to the robust activation of wild type D2R, a reduction in potency and a large decrease in maximal activation by quinpirole was observed in D2/D4 when expressed with free Gqi5 (FIG. 2B). As anticipated, none of these mutants led to agonist-mediated luminescence when placed into the unfused D2R construct and coepxressed with free Gqi5 (FIG. 2C) or when the mutations were placed in the D2-linker-Gqi5 construct and expressed alone (FIG. 11).

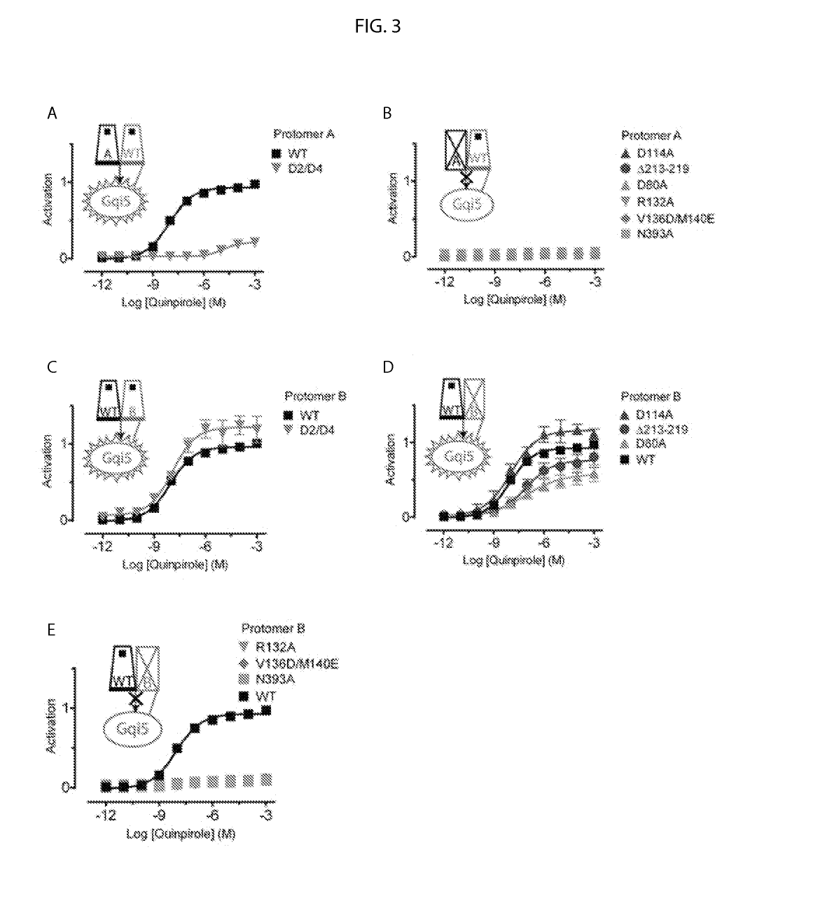

When D2/D4 was expressed as protomer A with WT D2R-Gqi5 as protomer B, a reduction in potency and a large decrease in maximal activation by quinpirole was observed (FIG. 3A), similar to its signaling properties when expressed with free Gqi5 (FIG. 2B) or fused to Gqi5 via an eight amino acid linker (data not shown). These and all subsequent activation data were normalized for surface expression of protomer B; see Example 1, FIG. 9 and FIG. 12.

Interestingly, in the functional complementation assay, the presence of any of the nonbinding or nonsignaling receptor mutants as protomer A completely prevented activation, despite the presence of WT D2R-Gqi5 in protomer B (FIG. 3B). Thus, protomer A, which must interact with the Ga provided by protomer B, appears to play a dominant role in the activation process, as any nonbinding or nonsignaling receptor construct in protomer A led to a loss of activation. This also precluded the possibility of receptor transactivation in this system, because no signaling was observed if protomer A was not intact, whether protomer B was WT D2R-Gqi5 or a nonsignaling or nonbinding receptor fused to Gqi5 (data not shown). The absence of trans-activation was not a result of the present functional complementation system or lack of sufficient mobility of fused G protein.

In contrast, robust agonist-mediated activation was observed with WT D2R as protomer A and D114A-Gqi5 (SEQ ID NO: 9) (FIG. 3C) or D2/D4-Gqi5 (SEQ ID NO: 3) (FIG. 3D) as protomer B. These data suggest that agonist (e.g., quinpirole) binding to protomer A is sufficient for normal activation (see below) and imply an asymmetric organization of the complex of two GPCR protomers with G protein.

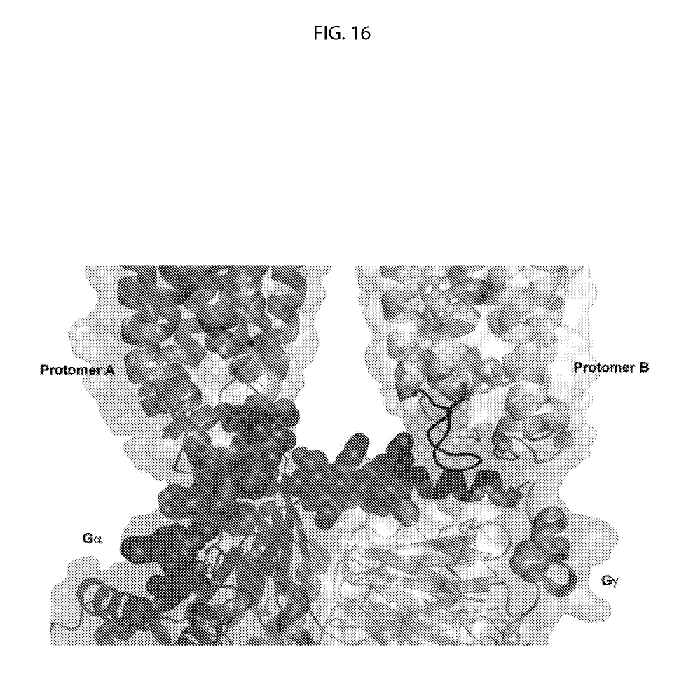

To explore further the precise arrangement of the signaling unit, mutations were introduced into IL2, which is known to play an important role in coupling to G-proteins. When R132^{3.50}A-Gqi5 (SEQ ID NO: 16) or V136^{3.54}D/M140^{3.58}E-Gqi5 (SEQ ID NO: 18) was expressed as protomer B with wild type D2R as protomer A, no activation was observed. Consistently, the docking studies suggested a critical role for IL2 from both protomers in activating a single G protein (see below). In contrast, these docking studies suggested that only IL3 from protomer A but not that from protomer B is positioned where it can contact the docked G protein. Indeed, experimental results show that the IL3 deletion construct completely wiped out activation when placed in protomer A (FIG. 3B) but did not prevent signaling when coexpressed as protomer B (fused to Gqi5) along with WT D2 as protomer A (FIG. 3D). These data support a mechanism in which two GPCRs activate a single G protein through interactions that involve IL2 from both protomers whereas IL3 from only one protomer is essential for signaling. Note that the failure of R132^{3.50}A-Gqi5 or V136^{3.54}D/M140³⁵⁸E-Gqi5 to function with WT is not due to an inability of these protomers to interact, because efficient bioluminescence resonance energy transfer as well as bimolecular luminescence and fluorescence complementation (117) was observed between these mutants and WT D2R (FIG. 16).

To study the nature of the conformational changes that take place in the transmembrane domains of the dimeric receptor unit, inactivating mutations within the membrane-spanning region was also examined. The transduction-uncoupling mutants D80^{2.50}A (myc-tagged version shown in SEQ ID NO: 29), and N^{7.49}393A (myc-tagged version shown in SEQ ID NO: 31), revealed additional differences in the roles of protomers A and B. When either of these mutations was placed in protomer A, signaling was abolished, consistent with the dominant role of this protomer (FIG. 3B). In contrast, when placed in protomer B, D80^{2.50}A-Gqi5 (myc-tagged version shown in SEQ ID NO: 29) signaled when coexpressed with WT D2R as protomer A (FIG. 3D), whereas N393⁷⁴⁹A-Gqi5 (myc-tagged version shown in SEQ ID NO: 31) did not (FIG. 3E). These results suggest that the nature of the conformational changes in protomer B during activation differs from those in protomer A.

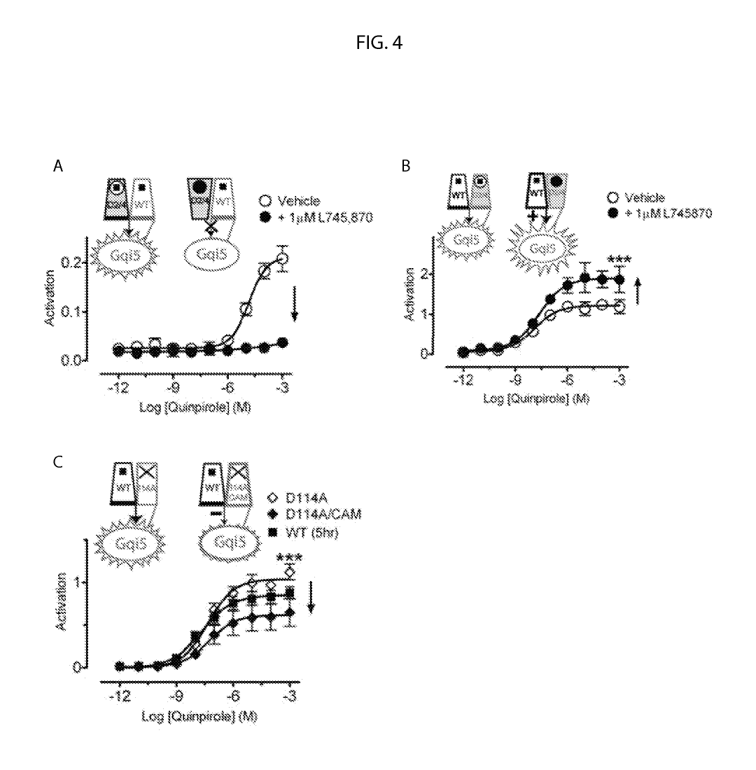

As shown in FIG. 3D agonist, e.g., quinpirole, binding to only protomer A produced full activation, as coexpressed D2R and D114A-Gqi5 were robustly activated by quinpirole. In addition, it appeared that binding of a second agonist to protomer B partially inhibited signaling, as coexpression of D2R and D2-Gqi5 led to lower maximal activation than did D2R coexpressed with D114A-Gqi5 (FIG. 3D). This hypothesis was tested using the D2/D4 chimeric receptor (myc-tagged version shown in SEQ ID NO: 23) (35). As predicted, when D2/D4 was expressed as protomer A with D2R-Gqi5 as protomer B, quinpirole's ability to bind and activate was blocked by the D4-selective antagonist L745,870, reflecting the primacy of protomer A (FIG. 4A). In contrast, coexpressing D2/D4-Gqi5 as protomer B with D2R as protomer A led to receptor activation that was greater than that seen with WT D2R and D2R-Gqi5 (FIG. 3C) and was enhanced in the presence of L745,870, which blocks quinpirole binding to protomer B but not protomer A (FIG. 4B).

These data are consistent with the hypothesis that agonist binding to a single protomer maximally activates a signaling unit of two Class A GPCRs and a single G protein, whereas agonist binding to the second protomer inhibits functional response. This presumably reflects the same mechanism by which agonist binding to and activation of the second protomer inhibits signaling. In contrast, findings in the mGluR suggest that although one agonist can activate the dimeric signaling unit, two agonists are required for full activation (38).

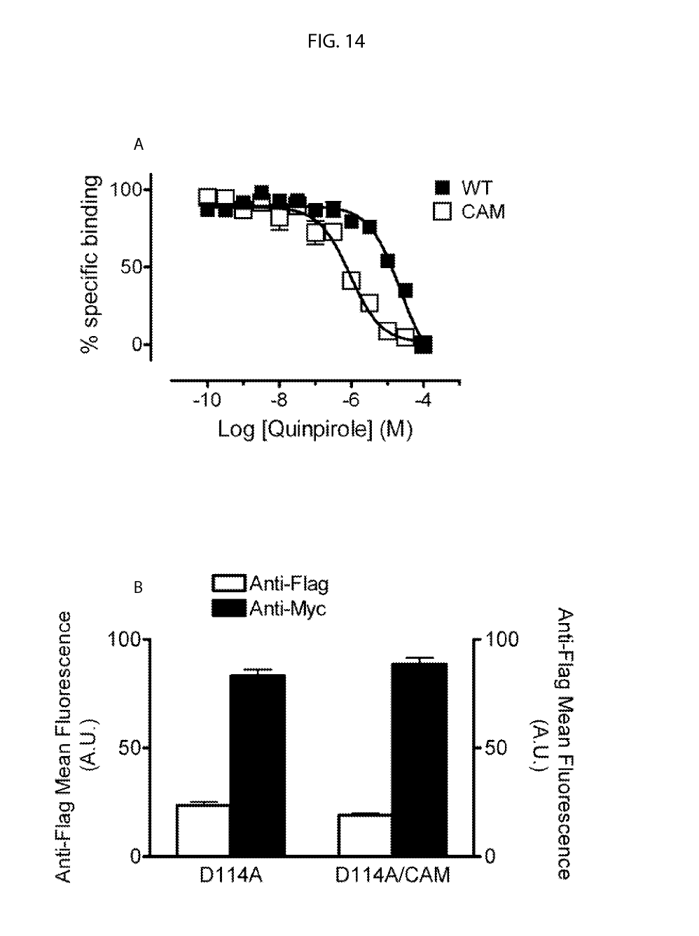

It is the active conformation of the second protomer that inhibits signaling, and not agonist binding per se. This is evidenced by the finding that activating protomer B by constitutively activating mutations (FIGS. 14A and 14B) in a nonbinding receptor (D114^{3.32}A/E339^{6.30}A/T343^{6.34}R-Gq5) (36, 37) (SEQ ID NO: 10) substantially reduced the signaling efficacy of a WT protomer A (FIG. 4C). Thus, activation of the second protomer, either by ligand binding or by its inherent constitutive activity, inhibits signaling by its partner.

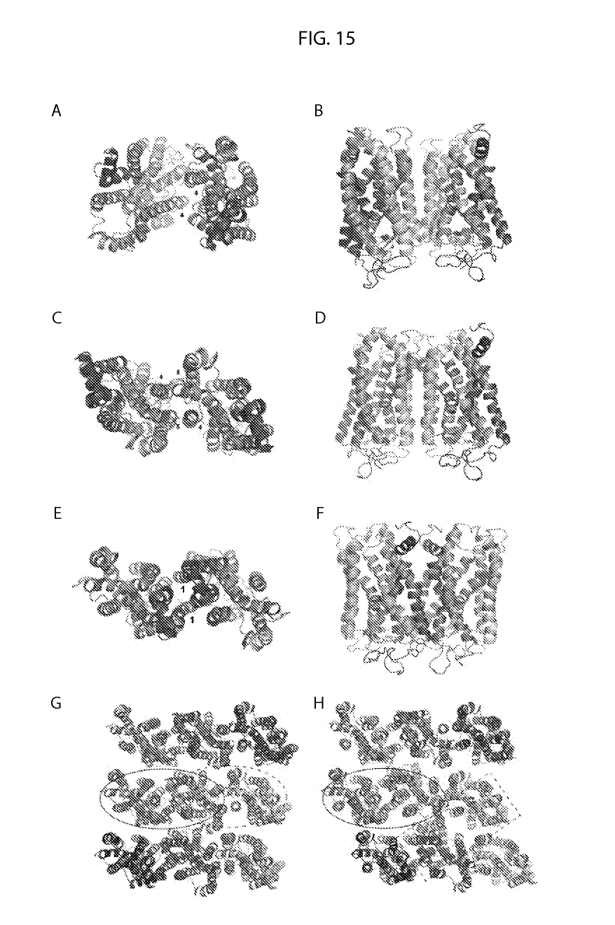

To develop a structural context for this study, independent computational studies that combine molecular modeling with the available experimental data about the modes of interaction of the component GPCRs and G protein in the complexes (but without direct reference to the new findings) were carried out. Because detailed structural information about the D2R is not available, bovine rhodopsin was used as a model for the study. The bovine rhodopsin offers both a known structural template for GPCRs and experimental data about interaction with G protein to guide a protein-protein docking. This experimental data from cross-linking, alanine scanning mutagenesis and other structural and functional studies of the GPCR-G protein interface allowed the identification of several amino acid residues that could be involved in complex formation between both the α- and the βγ-subunits of the G-protein with the respective receptor. The data, derived from the literature, were used not only as constraints to guide transducin docking to a variety of dimer models of rhodopsin (FIG. 15), but also to screen for the best oligomerization solution as detailed in Example 1.

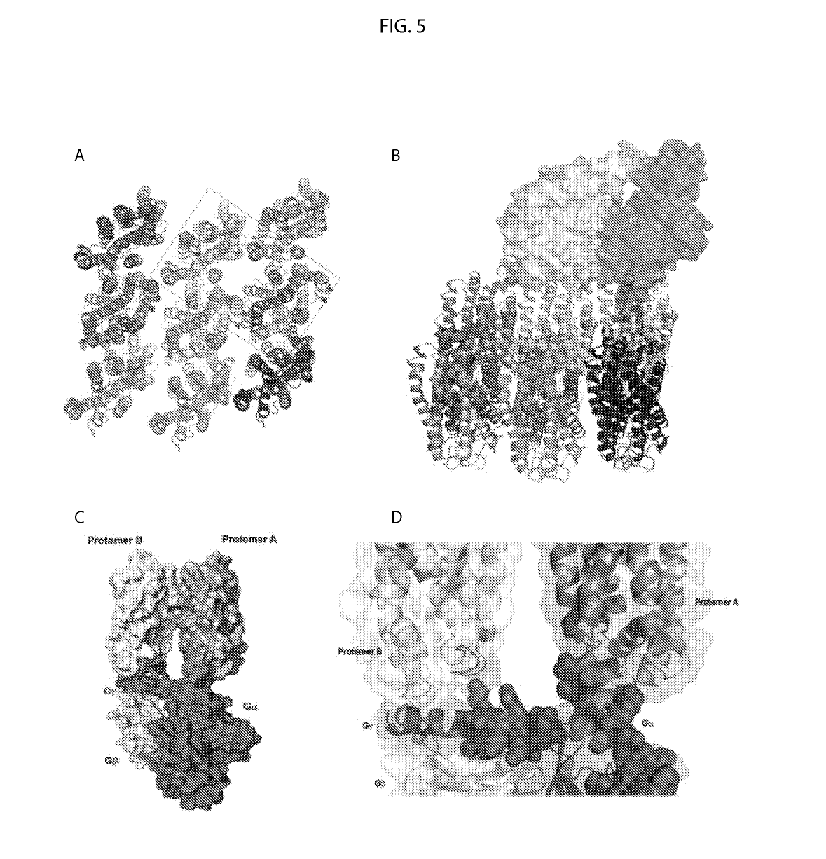

Both TM41/TM5 and TM1 have been implicated in D2R oligomerization (13, 39, 117). In order to discriminate between a functional dimer with an interface involving TM4 and TM5 (TM4,5 dimer) from one with a TM1 interface (TM1 dimer), the transducin molecular model was docked to a rhodopsin nonamer (FIG. 5A; and FIG. 15) subject to specific constraints for the interaction between Gₜ and the central rhodopsin (Table 2). Thus, transducin was free to rotate in any direction and select any one of the dimeric forms in the array. The Gₜ could select a second monomer from the oligomeric structure in which the GPCR interface corresponds to either a TM4, 5 interface, or a TM1 interface dimer. The probability for Gₜ selecting either dimer interface was compared in a scan for optimal interaction carried out on the oligomeric structure shown in (FIG. 5A) and (FIG. 15). The complexes resulting from this scan were considered acceptable (and counted) only if the underlying structural models satisfied at least 50% of the experimentally-based constraints (set 1 in Table 3). As shown in Table 4 below, a substantial fraction of TM4, 5 dimers (21.1%) satisfied this cutoff, but no complex with a TM1 dimer met the filtering criteria.

TABLE 4
Percentage of 1,000 analyzed structures that fit
the filtering criteria in a TM4,5 or TM1 dimer

| | |
|---|---|
| TM4,5 | 21.1% |
| TM1 | 0% |

A possible mode of oligomer reorganization associated with function had been suggested based on crosslinking studies in D2R (39) and rhodopsin (40). To evaluate the functional impact of such a reorganization, Gt was docked to the TM4, 5 and TM4 dimer alternatives (FIG. 15). The Cα-Cα distances for specific interactions between rhodopsin and transducin in the optimal representative structures of the 1000 structures obtained for each alternative in this dimer docking procedure (see Example 1 for details) are summarized in Table 3. For these optimal structures, the sets of Cα-Cα distances are very similar, but the frequency of appearance of optimally positioned complexes is much higher for Model 2 (TM4 dimer; 76.1%) than for Model 1 (TM4, 5 dimer; 23.8%). This is evident from the average values of the distances (Table 3), which are mostly larger in Model 1 than in Model 2 constructs. Thus, Model 2 is considered the better representation of the GPCR dimer complex with the G protein in the context of the oligomeric arrangement. This is consistent with the proposed transition from a TM4, 5 interface to a TM4 interface upon activation, suggested by crosslinking results for the D2R (39), and indicates the relation between optimal G protein binding to the dimer and an activated state.

Notably, in the optimal G protein—dimer complex the cytoplasmic ends of TM3 and IL2 from both protomers interact with the docked G protein. This is shown in Model 2 (FIG. 5), but holds as well for Model 1. In contrast, only IL3 from protomer A, but not from protomer B, contacts the docked Gα, consistent with the experimental results showing that an inactivating IL3 mutation is tolerated in protomer B but not in protomer A.

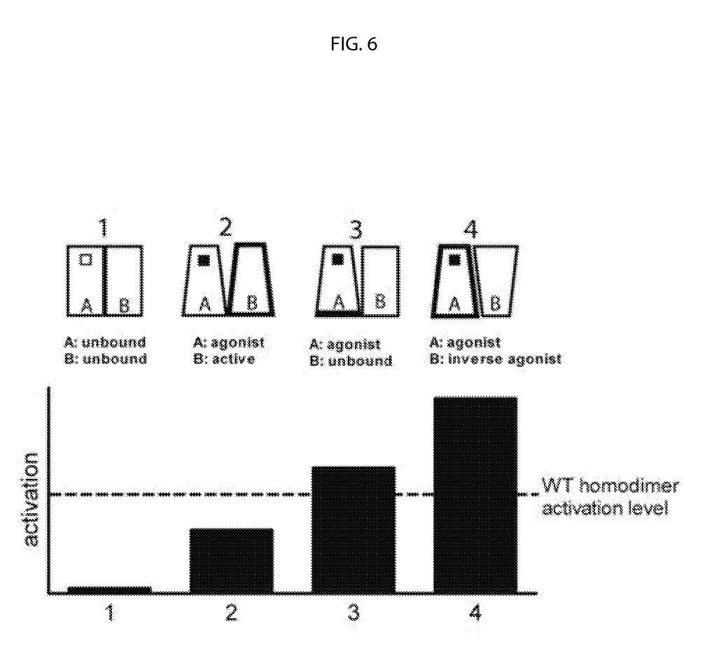

Thus, agonist binding to a single protomer maximally activates a signaling unit comprising two Class A GPCRs and a single G protein. Whereas activation of the second protomer inhibits the functional response, inverse agonist binding to the second protomer enhances signaling (FIG. 6). These results are consistent with studies in the Class C mGluR using allosteric modulators that act within the transmembrane region to show that the inactive state of a protomer caused by inverse agonist binding results in more efficient activation of the adjacent protomer (90, 119). These findings are more difficult to reconcile with other findings in the mGluR showing that although one agonist can activate the dimeric signaling unit, two agonists are required for full activation (91), suggesting differences in the mechanisms of these receptors, which have very different agonist binding sites. These findings are, however, fully consistent with the proposed function of GABA_{B} receptors, in which only R1 binds GABA (120) with efficient signaling by the complex. A similar scenario also seems likely for rhodopsin's ability to respond to single photons, which requires robust activation by a single protomer in a dimeric unit. Indeed in this case, the strong inverse agonist 11-cis-retinal (7) in the binding pocket of second protomer would in fact optimize signaling, just as what was observed in configuration 4 in (FIG. 6). These findings also suggest that optimal signaling in a heteromeric GPCR would result from co-stimulation with an agonist to one protomer and an inverse agonist to the other.

The data and models disclosed herein suggest that the way in which the two protomers contribute to the activated complex with the G protein is not symmetrical, and that activation requires different conformational changes in each protomer. Existing evidence for ligand-induced conformational changes in a second non-binding protomer (11, 12) is consistent with the proposal of conformational changes in both protomers. It has been previously demonstrated an activation-related conformational change at the TM4 dimer interface (39) that also would be consistent with movement of either one or both TM4s. The present finding that transduction-deficient mutants in different TMs differentially affect the ability of protomer B to rescue function is consonant with the importance of conformational changes in this protomer. Interestingly, the apparent negative cooperativity of ligand binding observed in a number of class A GPCRs (93) may well relate to this proposed asymmetry of the signaling unit. For example, in cells expressing chemokine receptor heterodimers, a selective ligand for one protomer leads to dissociation of ligand bound to the other protomer (96), consistent with transmission of an altered conformation across the dimer interface, and with a decreased propensity for simultaneous agonist binding to both protomers.

In summary, the functional complementation assay disclosed herein allows for control of the signaling unit of the human dopamine D2 receptor (D2R) and thus for exploring the individual contributions of each GPCR protomer to G protein signaling. Although a single B2AR or rhodopsin molecule can efficiently activate G protein when reconstituted into a nanodisc, a second protomer is present in vivo and profoundly modulates G protein activation of the first protomer, as shown in the functional complementation studies disclosed herein. Importantly, the studies herein showed that this allosteric modulation of signaling results from a direct interaction of the receptor dimer with the G protein, rather than from a downstream effect. This is likely to explain many of the surprising observations concerning the mutual modulation of heteromeric receptor oligomers by ligand binding to one protomer or the other. Moreover, the studies demonstrate that the constitutive activity of a protomer will modulate the activity of the dimeric signaling unit in which it participates. Thus, inverse agonists at one protomer in a heterodimer are likely to be allosteric potentiators of the signaling of its heterodimer partner, whereas agonists of one protomer will be allosteric inhibitors of the second protomer, offering a mechanistic explanation for the often befuddling observations regarding pharmacological effects of ligands acting on heterodimers. Moreover, the model disclosed herein suggests that modulators might be found that are specific for heterodimers and not homodimers, but heretofore it has not been possible to screen for such compounds without the interference of homodimer-mediated signaling. Indeed, it is possible that findings of functional selectivity, that is, different agonists for a given receptor having different effects on different downstream effectors, might reflect differential pharmacological effects on different heteromeric species (121). The novel methodology disclosed herein makes it possible to identify signaling from a defined heterodimer, and thus to identify modulators of heterodimer function. The modulatory mechanism characterized herein and the approach that made this possible offer a new understanding of GPCR signaling in units composed of at least two GPCRs. Applied to specific systems, the approach will make it possible to understand the effects of drugs that target each protomer of such a signaling unit, either identical or different.

### Example 4

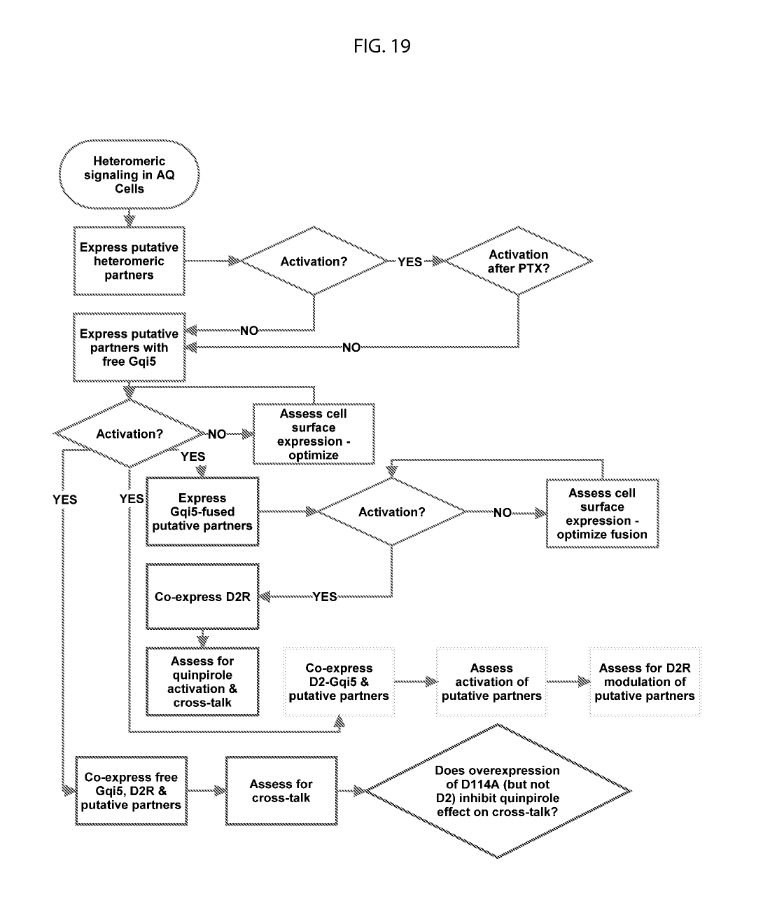

With reference to FIG. 19, a representative complementation assay according to the present invention may be carried out as follows. All of the receptors hypothesized to heteromerize with D2R activate Gi/o, with the exception of D1R. The first step is to establish that they do not couple to endogenous Gq and that they signal to free Gqi5 by creating stable lines of each of the putative heteromeric receptor partners, A2A, CB1, D3, SSTR5, and DOR in cells expressing aequorin (AQ cells) and in AQ cells expressing free Gqi5. If there is a lack of response in the former and an appropriate agonist response in the latter, then experiments may proceed (FIG. 19). Response in the AQ cells without Gqi5 might indicate an indirect effect of Gi/o on PLC, which will be tested by PTX treatment. If PTX eliminates the signal without Gqi5 but not with an engineered PTX-resistant Gqi5, PTX treatment may be used to prevent endogenous Gi/o from interfering with the assay. The following conditions will also be established: that the D2R selective agonist quinpirole does not activate any of the receptors directly and that the prototypical agonists for the heteromeric partners are without effect on D2R when it is expressed alone with free Gqi5.

Next, fusion constructs will be created in which Gqi5 is placed in frame at the C-terminal end of each putative heteromeric partner and will create a stable line for each in AQ cells (FIG. 19). Surface expression of the receptor constructs will be confirmed and quantitated by FACS analysis. These cell lines will be tested for function of the prototypical heteromeric partner agonists. Because each of the putative heteromeric partners has a substantially longer C-terminal tail than the D2R, each of the Gqi5-fusions is expected to function without adding linking amino acids. The C-terminal tail of a putative heteromeric partner may also be truncated at or about the highly conserved cysteine residue that corresponds to residue 414 of the short isoform of human wild type D2R (SEQ ID NO: 61) (including truncations between about −1 and +3 of the amino acid position corresponding to residue 414) before fusing such heteromeric partner to Gqi5.

These lines then will be stably expressed with D2R, and, after confirming surface expression of both constructs, whether quinpirole can signal via the Gqi5 fused to the putative heteromeric partner receptor will be determined (FIG. 19). Whether the D2-agonist, quinpirole, and the inverse agonist, sulpiride, alter the potency and efficacy of signaling via the partner receptors will be tested, and whether an agonist targeting the partner receptor at a low or even subthreshold concentration can alter quinpirole's potency and efficacy will also be tested (FIG. 19, red).

In parallel, each of the putative heteromeric partners as protomer A will be stably coexpressed together with D2-Gqi5 as protomer B (FIG. 19, yellow). Protomer A may be any GPCR, including but not limited to human D1, human D3, human CCR5, human SSTR5, human DOR, mouse DOR, human TSHR, rat GluR1, rat GluR5, human CB1, human A2a, human M4, and human 5HT1b.

Since D2-Gqi5 cannot function on its own but may be activated by a D2R in extremely close proximity, activation of any of the partner receptors by their prototypical agonists will be indicative of signaling through the Gqi5 attached to the D2R and thus signaling through a presumed signaling unit, be it a heterodimer or a higher order complex. Modulatory effects of the D2-agonist, quinpirole, and the inverse agonist, sulpiride, will be tested on the potency and efficacy of signaling via the heteromeric partners (FIG. 19), although it could be inferred that quinpirole alone will be without effect because the D2-Gqi5 cannot signal itself without a second D2R protomer capable of interacting with the Gqi5.

In all cases, cell surface expression will be monitored by FACS analysis against N-terminal epitopes to insure that the receptors express at the cell surface at comparable levels, and expression will be adjusted when necessary by varying the time after addition of tetracycline. These findings will also be validated in the absence of a fused G protein (FIG. 19). Overexpression of D114A can be used to evaluate the mechanism of signaling, but now in the context of heteromeric signaling. It is expected that D114A will effectively inhibit heteromeric signal crosstalk by competing with WT D2R for the heterodimer interface, generating a heteromer in which quinpirole cannot bind and therefore cannot regulate the heteromeric partner. In contrast, as discussed above, D114A should have no or a much smaller inhibitory effect on D2R signaling, and might even boost signaling of the homodimer. The D114A lentivirus may also be used to test this hypothesis. These cellular systems will provide an important validation of this methodology, which can later be applied in vivo to probe signaling mechanisms by disrupting heteromeric (D2R-DOR) while preserving D2R and DOR homomeric signaling.

### Example 5

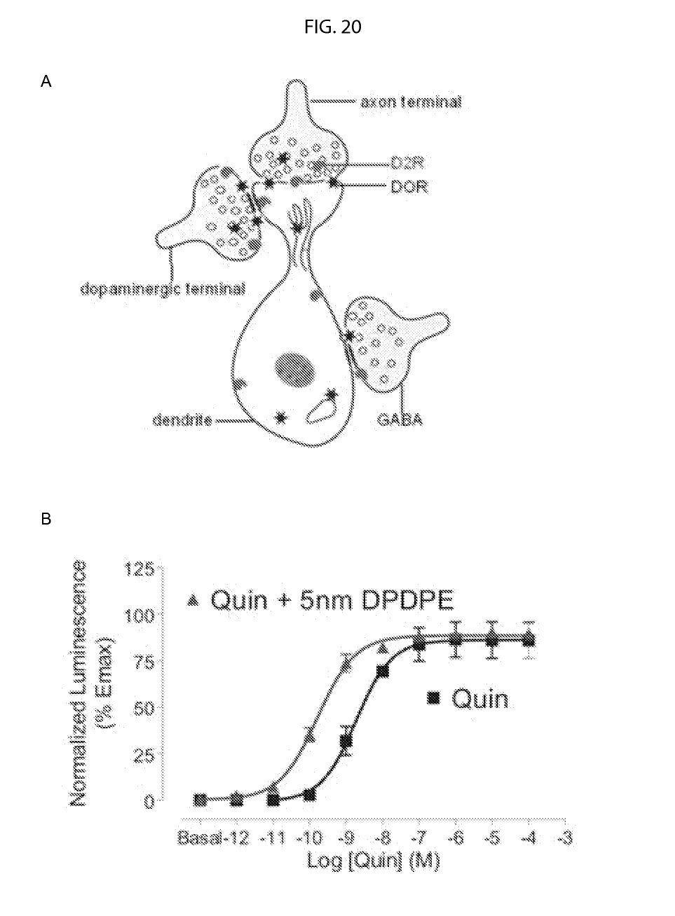

The methodology set forth in Example 4 was carried out with DOR-Gqi5 (SEQ ID NO: 71), at which the delta opiate receptor (DOR) specific agonist DPDPE signals effectively and quinpirole is without effect. Coexpression of D2R leads to robust quinpirole-activation of the Gqi5 fused to mouse DOR. At 5 nM DPDPE, a concentration that produces about 10% activation of DOR-Gqi5, the potency of quinpirole was enhanced 10-fold (FIG. 20B), and this effect was blocked completely by 100 nM naloxone (data not shown). Thus, consistent with previous findings (106), these results demonstrate signaling crosstalk between DOR and D2R.

### Example 6

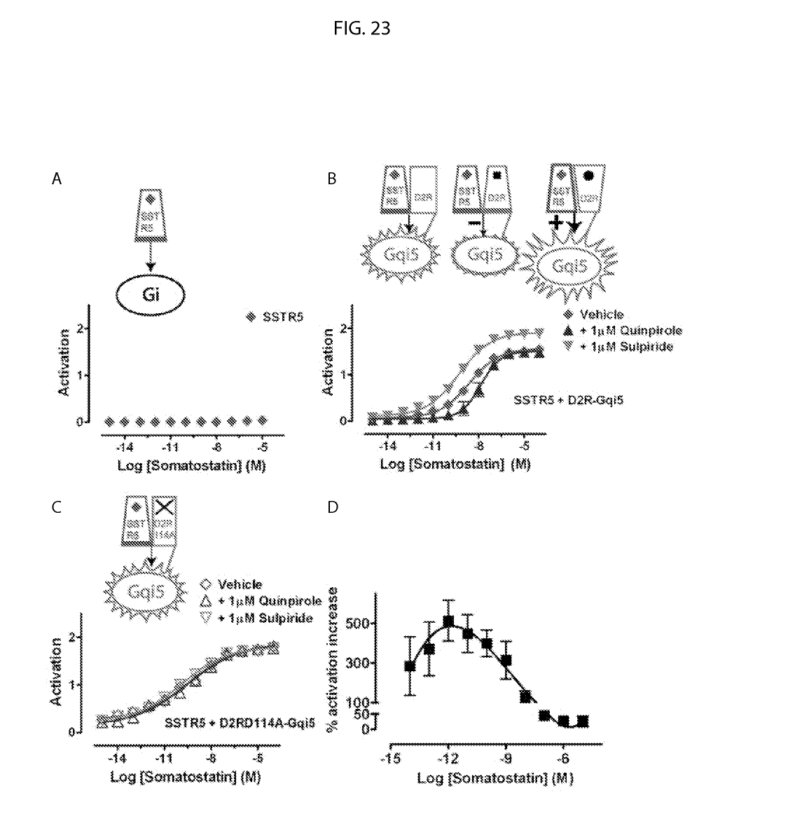

The interactions between somatostatin receptor 5 (SSTR5) and D2R were also examined. Despite being unable to signal directly, D2-Gqi5 (SEQ ID NO. 21) is able to provide G protein to SSTR5 (SEQ ID NO: 97, DNA encoding the myc-tagged version of SSTR5 inserted into the pIRESpuro3 vector is shown in SEQ ID NO: 95), thereby enabling activation by somatostatin (SST). A nonbinding mutant D2R (D114A-Gqi5) (SEQ ID NO. 9) enabled SST activation that was unaffected by dopamine agonist and antagonist. In contrast, WT D2R-Gqi5 enabled SST activation that was subject to profound modulation by dopamine agonists and antagonists. (FIG. 23). The potency of somatostatin was enhanced about 6-fold by the D2R inverse agonist sulpiride relative to the agonist quinpirole, allowing for a previously unimagined complexity of physiological and pharmacological interactions.

These findings suggest that a D2R inverse agonist used as an antipsychotic medication not only blocks D2R signaling but also has the potential to profoundly enhance signaling by D2R heteromeric partners. Moreover enhanced D2R signaling, found for example in the striatum in schizophrenia, is expected to be associated with a reduction in heteromeric partner signaling. This greatly complicates the interpretation of pharmacological data, as different heteromeric partners may play greater or lesser roles in different regions, blurring a simple distinction between “on target” and “off target” effects. Importantly, these findings and the associated methodology allow for screening of compounds that are heteromer selective. Such compounds will exert interprotomer modulation in a particular heteromer but not in a homomer, allowing an unprecedented level of fine tuning of the system.

As preliminary proof of principle, a novel allosteric inhibitor of D2R was studied. This compound binds to an allosteric site in the extracellular loops and leads to a maximal inhibition of D2R signaling of only about 40% (data not shown). Surprisingly, a full shift in the SST curve in the presence of this compound was observed (data not shown), just like what was observed for sulpiride and clozapine, suggesting that a drug with reduced efficacy to inhibit homomeric D2R signaling can still maximally enhance SSTR5 signaling, and suggesting that specificity is possible. An assay may be performed in order to create a platform that can be used to screen for heteromer selective ligands, for example, by screening for compounds that modulate SST signaling in SSTR5-D2 heteromers but not in either homomer.

### Example 7

A stably expressed Gq siRNA cell line will be constructed based on the above-described Flp-in T-Rex 293 (Invitrogen) AEQ cell line in order to knock down endogenous Gq/11. The target sequence for Gq/11 silencing is 5′-GATGTTCGTGGACCTGAAC-3′ (SEQ ID NO: 100) (122, 123). This mature siRNA sequence will be constructed in a pLemiR™ lentiviral vector (Open Biosystem, Huntsville, Ala.), which can be stably expressed in mammalian cells by selecting with puromycin after transfection. Response to activation of endogenous muscarinic receptors will be screened with the goal of achieving sufficient knock down of endogenous Gq/11 to ablate signaling. This will allow for screening for Gq/11-coupled GPCRs co-expressed with GPCR-Gq/11 fusion in this system by a modified FRT/TO vector, which expresses two GPCRs simultaneously. By this means, the interaction between protomers comprised of a Gq/11-coupled GPCR and a GPCR-Gq/11 chimera may be investigated without influence of endogenous Gq/11. Based this special system, the only functional unit will be a heterodimer consisting of an unfused GPCR and a GPCR-Gq/11 chimera. Note that the Gq/11 fusions will be constructed with an altered DNA coding sequence to express a normal protein that is insensitive to the siRNA.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

### CITATIONS

All patents, patent applications, and documents cited within this application, including those set forth below, are hereby incorporated by reference as if recited in full herein.
1. Bartfai, T. et al. The state of GPCR research in 2004. Nature Reviews Drug Discovery 3, 574-626 (2004).
2. Chabre, M., Cone, R. & Saibil, H. Biophysics—Is rhodopsin dimeric in native rods? Nature 426, 30-31 (2003).
3. Pin, J. P. et al. International Union of Basic and Clinical Pharmacology. LXVII. Recommendations for the Recognition and Nomenclature of G Protein-Coupled Receptor Heteromultimers. Pharmacol Rev 59, 5-13 (2007).
4. Bayburt, T. H., Leitz, A. J., Xie, G., Oprian, D. D. & Sligar, S. G. Transducin Activation by Nanoscale Lipid Bilayers Containing One and Two Rhodopsins. J. Biol. Chem. 282, 14875-14881 (2007).
5. Whorton, M. R. et al. A monomeric G protein-coupled receptor isolated in a high-density lipoprotein particle efficiently activates its G protein. PNAS 104, 7682-7687 (2007).
6. Pin, J. P., Galvez, T. & Prezeau, L. Evolution, structure, and activation mechanism of family 3/C G-protein-coupled receptors. Pharmacology & Therapeutics 98, 325-354 (2003).
7. Brock, C. et al. Activation of a Dimeric Metabotropic Glutamate Receptor by Intersubunit Rearrangement. J. Biol. Chem. 282, 33000-33008 (2007).
8. Ji, I. H., Lee, C., Song, Y. S., Conn, P. M. & Ji, T. H. Cis- and trans-activation of hormone receptors: the LH receptor. Mol Endocrinol 16, 1299-1308 (2002).
9. George, S. R. et al. Oligomerization of mu- and delta-opioid receptors—Generation of novel functional properties. J. Biol. Chem. 275, 26128-26135 (2000).
10. George, S. R. & O'Dowd, B. F. A novel dopamine receptor signaling unit in brain: heterooligomers of D1 and D2 dopamine receptors. Scientific World Journal. 7, 58-63 (2007).
11. Damian, M., Martin, A., Mesnier, D., Pin, J. P. & Baneres, J. L. Asymmetric conformational changes in a GPCR dimer controlled by G-proteins. Embo Journal 25, 5693-5702 (2006).
12. Mesnier, D. & Baneres, J. L. Cooperative Conformational Changes in a G-protein-coupled Receptor Dimer, the Leukotriene B4 Receptor BLT1. J. Biol. Chem. 279, 49664-49670 (2004).
13. Guo, W., Shi, L. & Javitch, J. A. The Fourth Transmembrane Segment Forms the Interface of the Dopamine D2 Receptor Homodimer. J. Biol. Chem. 278, 4385-4388 (2003).
14. Hildebrandt, J. D. Bring Your Own G Protein. Mol Pharmacol 69, 1079-1082 (2006).
15. Seifert, R., Wenzel-Seifert, K. & Kobilka, B. K. GPCR-G[alpha] fusion proteins: molecular analysis of receptor-G-protein coupling. Trends in Pharmacological Sciences 20, 383-389 (1999).
16. Molinari, P. et al. Promiscuous Coupling at Receptor-Galpha Fusion Proteins. THE RECEPTOR OF ONE COVALENT COMPLEX INTERACTS WITH THE alpha-SUBUNIT OF ANOTHER. J. Biol. Chem. 278, 15778-15788 (2003).
17. Carrillo, J. J., Pediani, J. & Milligan, G. Dimers of Class A G Protein-coupled Receptors Function via Agonist-mediated Trans-activation of Associated G Proteins. J. Biol. Chem. 278, 42578-42587 (2003).
18. Snook, L. A., Milligan, G., Kieffer, B. L. & Massotte, D. {micro}-{delta} Opioid Receptor Functional Interaction: Insight Using Receptor-G Protein Fusions. J Pharmacol Exp Ther 318, 683-690 (2006).
19. Pascal, G. & Milligan, G. Functional Complementation and the Analysis of Opioid Receptor Homodimerization. Mol Pharmacol 68, 905-915 (2005).
20. Snook, L. A., Milligan, G., Kieffer, B. L. & Massotte, D. Co-expression of mu and delta opioid receptors as receptor-G protein fusions enhances both mu and delta signaling via distinct mechanisms. J Neurochem 105, 865-873 (2008).
21. Lee, T. W., Seifert, R., Guan, X. & Kobilka, B. K. Restricting the Mobility of Gs alpha: Impact on Receptor and Effector Coupling. Biochemistry 38, 13801-13809 (1999).
22. Wenzel-Seifert, K., Lee, T. W., Seifert, R. & Kobilka, B. K. Restricting mobility of Gsalpha relative to the beta2-adrenoceptor enhances adenylate cyclase activity by reducing Gsalpha GTPase activity. Biochem. J. 334, 519-524 (1998).
23. Brini, M. et al. Transfected Aequorin in the Measurement of Cytosolic Ca2+ Concentration ([Ca2+]c). J. Biol. Chem. 270, 9896-9903 (1995).
24. Rizzuto, R., Simpson, A. W. M., Brini, M. & Pozzan, T. Rapid changes of mitochondrial Ca2+ revealed by specifically targeted recombinant aequorin. Nature 358, 325-327 (1992).
25. Blanpain, C. et al. Extracellular Cysteines of CCR5 Are Required for Chemokine Binding, but Dispensable for HIV-1 Coreceptor Activity. J. Biol. Chem. 274, 18902-18908 (1999).
26. Wise, A. et al. A Cysteine-3 to Serine Mutation of the G-Protein Gi1 alpha Abrogates Functional Activation by the alpha 2A-Adrenoceptor but Not Interactions with the beta gamma Complex. Biochemistry 36, 10620-10629 (1997).
27. Conklin, B. R., Farfel, Z., Lustig, K. D., Julius, D. & Bourne, H. R. Substitution of three amino acids switches receptor specificity of Gq[alpha] to that of Gi[alpha]. Nature 363, 274-276 (1993).
28. Stanners, J., Kabouridis, P. S., McGuire, K. L. & Tsoukas, C. D. Interaction between G Proteins and Tyrosine Kinases upon T Cell ReceptorbulletCD3-mediated Signaling. J. Biol. Chem. 270, 30635-30642 (1995).
29. Xu, W. et al. Functional role of the spatial proximity of Asp114(2.50) in TMH 2 and Asn332(7.49) in TMH 7 of the mu opioid receptor. FEBS Lett. 447, 318-324 (1999).
30. Ballesteros, J. A., Shi, L. & Javitch, J. A. Structural mimicry in G protein-coupled receptors: Implications of the high-resolution structure of rhodopsin for structure-function analysis of rhodopsin-like receptors. Mol Pharmacol 60, 1-19 (2001).
31. Visiers, I., Hassan, S. A. & Weinstein, H. Differences in conformational properties of the second intracellular loop (IL2) in 5HT2C receptors modified by RNA editing can account for G protein coupling efficiency. Protein Eng. 14, 409-414 (2001).
32. Strader, C. D. et al. Mutations that uncouple the beta-adrenergic receptor from Gs and increase agonist affinity [published erratum appears in J Biol Chem 1988 Feb. 25; 263(6):3050]. J. Biol. Chem. 262, 16439-16443 (1987).
33. Neve, K. A. et al. Modeling and mutational analysis of a putative sodium-binding pocket on the dopamine D-2 receptor. Mol Pharmacol 60, 373-381 (2001).
34. Urizar, E. et al. An Activation Switch in the Rhodopsin Family of G Protein-coupled Receptors: The thyrotropin receptor. J. Biol. Chem. 280, 17135-17141 (2005).
35. Simpson, M. M. et al. Dopamine D4/D2 Receptor Selectivity Is Determined by A Divergent Aromatic Microdomain Contained within the Second, Third, and Seventh Membrane-Spanning Segments. Mol Pharmacol 56, 1116-1126 (1999).
36. Wilson, J., Lin, H., Fu, D., Javitch, J. A. & Strange, P. G. Mechanisms of inverse agonism of antipsychotic drugs at the D2 dopamine receptor: use of a mutant D2 dopamine receptor that adopts the activated conformation. J Neurochem 77, 493-504 (2001).
37. Ballesteros, J. A. et al. Activation of the beta(2)-adrenergic receptor involves disruption of an ionic lock between the cytoplasmic ends of transmembrane segments 3 and 6. J. Biol. Chem. 276, 29171-29177 (2001).
38. Kniazeff, J. et al. Closed state of both binding domains of homodimeric mGlu receptors is required for full activity. Nature Structural & Molecular Biology 11, 706-713 (2004).
39. Guo, W., Shi, L., Filizola, M., Weinstein, H. & Javitch, J. A. From The Cover: Crosstalk in G protein-coupled receptors: Changes at the transmembrane homodimer interface determine activation. PNAS 102, 17495-17500 (2005).
40. Itoh, Y., Cai, K. & Khorana, H. G. Mapping of contact sites in complex formation between light-activated rhodopsin and transducin by covalent crosslinking: Use of a chemically preactivated reagent. PNAS 98, 4883-4887 (2001).
41. Lambright, D. G., Sondek, J., Bohm, A., Skiba, N. P., Hamm, H. E., Sigler, P. B. The 2.0 a crystal structure of a heterotrimeric G protein. Nature 379, 311-319 (1996).
42. Martin-Renom, M. A., Stuart, A. C., Fiser, A., Sanchez, R., Melo, F., Sali, A. Comparative protein structure modeling of genes and genomes. Annu Rev Biophys Biomol Struct 29, 291-325 (2000).
43. Koenig, B. W., Kontaxis, G., Mitchell, D. C., Louis, J. M., Litman, B. J., Bax, A. Structure and orientation of a G protein fragment in the receptor bound state from residual dipolar couplings. J. Mol. Biol. 322, 441-461 (2002).
44. Kisselev, O. G., Downs, M. A. Rhodopsin-interacting surface of the transducin gamma subunit. Biochemistry, 45, 9386-9392 (2006).
45. Case, D. A., Cheatham, T. E., Darden, T., Gohlke, H., Luo, R., Merz, K. M., Onufriev, A., Simmerling, C., Wang, B., Woods, R. J. The Amber biomolecular simulation programs. J. Comput. Chem. 2005, 26, 1668-1688 (2005).
46. Niv, M. Y., Skrabanek, L., Filizola, M., Weinstein, H. Modeling activated states of GPCRs: the rhodopsin template. J. Comput. Aided Mol. Des. 20, 437-448 (2006).
47. van Dijk, A. D., de Vries, S. J., Dominguez, C., Chen, H., Zhou, H. X., Bonvin, A. M. Data-driven: HADDOCK's adventures in CAPRI. Proteins. 60, 232-238 (2005).
48. van Dijk, M., van Dijk, A. D., Hsu, V., Boelens, R., Bonvin, A. M. Information-driven protein-DNA docking using HADDOCK: it is a matter of flexibility. Nucleic Acids Res. 34, 3317-3325 (2006).
49. Osawa, S., Weiss, E. R. The effect of carboxyl-terminal mutagenesis of Gt alpha on rhodopsin and guanine nucleotide binding. J. Biol. Chem. 270, 31052-31058 (1995).
50. Onrust, R., Herzmark, P., Chi, P., Garcia, P. D., Lichtarge, O, Kingsley, C., Bourne, H R. Receptor and beta gamma binding sites in the alpha subunit of the retinal G protein transducin. Science. 275, 381-384 (1997).
51. Arimoto, R., Kisselev O. G., Makara G. M., Marshall G. R. Rhodopsin-transducin interface; studies with conformationally constrained peptides. Biophys. J. 81, 3285-3293 (2001).
52. Cai, K., Itoh, Y., Khorana, H. G. Mapping of contact sites in complex formation between transducin and light-activated rhodopsin by covalent crosslinking: use of a photoactivatable reagent. Proc. Natl. Acad. Sci. USA. 98, 4877-4882 (2001).
53. Acharya, S., Saad, Y., Karnik, S. S. Transducin-alpha C-terminal peptide binding site consists of C-D and E-F loops of rhodopsin. J. Biol. Chem. 272, 6519-6524 (1997).
54. Natochin, M., Gasimov, K. G., Moussaif, M., Artemyev, N. O. Rhodopsin determinants for transducin activation; a gain-of-function approach. J. Biol. Chem. 278, 37574-37581 (2003).
55. Yang K., Farrens, D. L., Hubbell, W. L., Khorana, H. G. Structure and function in. Single cysteine substitution mutants in the cytoplasmic interhelical E-F loop region show position-specific effects in transducin activation. Biochemistry, 35, 12464-12469 (1996).
56. Janz. J. M., Farrens, D. L. Rhodopsin activation exposes a key hydrophobic binding site for the transducin alpha-subunit C terminus. J. Biol. Chem. 279, 29767-29773 (2004).
57. Marin, E. P., Krishna, A. G., Zvyaga, T. A., lsele, J., Siebert, F., Sakmar, T. P. The terminus of the fourth cytoplasmic loop of rhodopsin modulates rhodopsin-transducin interaction. J. Biol. Chem. 275, 1930-1936 (2000).
58. Ernst, O. P., Meyer, C. K., Marin, E. P., Henklein, P., Fu, W. Y., Sakmar, T. P., Hofmann, K. P. Mutation of the fourth cytoplasmic loop of rhodopsin affects binding of transducin and peptides derived from the carboxyl-terminal sequences of transducin alpha and gamma subunits. J. Biol. Chem. 275, 1937-1943 (2000).
59. Hubbard, S. J. Thornton, J. M. ‘NACCESS’, computer program. Depertment of Biochemistry Molecular Biology, University College London (1993).
60. Downs M. A., Arimoto, R., Marshall, G. R., Kisselev, O. G. G-protein alpha and beta-gamma subunits interact with conformationally distinct signaling states of rhodopsin. Vision res., 46, 4442-4448 (2006).
61. Maggio, R., Novi, F., Scarselli, M., and Corsini, G. U. FEBS Journal 272, 2939-2946 (2005).
62. Prinster, S. C., Hague, C., and Hall, R. A. Pharmacol Rev 57, 289-298 (2005).
63. Fotiadis, D., Liang, Y., Filipek, S., Saperstein, D. A., Engel, A., and Palczewski, K. Nature 421, 127-128 (2003).
64. Liang, Y., Fotiadis, D., Filipek, S., Saperstein, D. A., Palczewski, K., and Engel, A. J. Biol. Chem. 278, 21655-21662 (2003).
65. Park, P. S. H. and Wells, J. W. J Neurochem 90, 537-548 (2004).
66. Philip, F., Sengupta, P., and Scarlata, S. J. Biol. Chem. 282, 19203-19216 (2007).
67. Breitwieser, G. E. Circ Res 94, 17-27 (2004).
68. Lee, S. P., So, C. H., Rashid, A. J., Varghese, G., Cheng, R., Lanca, A. J., O'Dowd, B. F., and George, S. R. J. Biol. Chem. 279, 35671-35678 (2004).
69. Rashid, A. J., So, C. H., Kong, M. M. C., Furtak, T., El Ghundi, M., Cheng, R., O'Dowd, B. F., and George, S. R. PNAS 104, 654-659 (2007).
70. O'Dowd, B. F., Ji, X., Alijaniaram, M., Rajaram, R. D., Kong, M. M. C., Rashid, A., Nguyen, T., and George, S. R. J. Biol. Chem. 280, 37225-37235 (2005).
71. Dziedzicka-Wasylewska, M., Faron-Gorecka, A., Andrecka, J., Polit, A., Kusmider, M., and Wasylewski, Z. Biochemistry 45, 8751-8759 (2006).
72. So, C. H., Varghese, G., Curley, K. J., Kong, M. M. C., Alijaniaram, M., Ji, X., Nguyen, T., O'Dowd, B. F., and George, S. R. Mol Pharmacol 68, 568-578 (2005).
73. So, C. H., Verma, V., O'Dowd, B. F., and George, S. R. Mol Pharmacol 72, 450-462 (2007).
74. Kong, M. M. C., Fan, T., Varghese, G., O'Dowd, B. F., and George, S. R. Mol Pharmacol 70, 78-89 (2006).
75. Milligan, G. Drug Discovery Today 11, 541-549 (2006).
76. Mannoury la Cour, C., Vidal, S., Pasteau, V., Cussac, D., and Millan, M. J. Neuropharmacology 52, 1003-1014 (2007).
77. Jin, L. Q., Wang, H. Y., and Friedman, E. J Neurochem 78, 981-990 (2001).
78. Ming, Y. L., Zhang, H., Long, L. H., Wang, F., Chen, J. G., and Zhen, X. C. J Neurochem 98, 1316-1323 (2006).
79. Ali, M. K. and Bergson, C. J. Biol. Chem. 278, 51654-51663 (2003).
80. Lidow, M. S., Roberts, A., Zhang, L., Koh, P. O., Lezcano, N., and Bergson, C. European Journal of Pharmacology 427, 187-193 (2001).
81. Maggio, R., Scarselli, M., Novi, F., Millan, M. J., and Corsini, G. U. J Neurochem 87, 631-641 (2003).
82. Novi, F., Millan, M. J., Corsini, G. U., and Maggio, R. J Neurochem 102, 1410-1424 (2007).
83. Scarselli, M., Novi, F., Schallmach, E., Lin, R., Baragli, A., Colzi, A., Griffon, N., Corsini, G. U., Sokoloff, P., Levenson, R., Vogel, Z., and Maggio, R. J. Biol. Chem. 276, 30308-30314 (2001).
84. Bolan, E. A., Kivell, B., Jaligam, V., Oz, M., Jayanthi, L. D., Han, Y., Sen, N., Urizar, E., Gomes, I., Devi, L. A., Ramamoorthy, S., Javitch, J. A., Zapata, A., and Shippenberg, T. S. Mol Pharmacol mol (2007).
85. Lee, F. J. S., Pei, L., Moszczynska, A., Vukusic, B., Fletcher, P. J., and Liu, F. Embo Journal 26, 2127-2136 (2007).
86. Rocheville, M., Lange, D. C., Kumar, U., Patel, S. C., Patel, R. C., and Patel, Y. C. Science 288, 154-157 (2000).
87. Hillion, J., Canals, M., Torvinen, M., Casado, V., Scott, R., Terasmaa, A., Hansson, A., Watson, S., Olah, M. E., Mallol, J., Canela, E. I., Zoli, M., Agnati, L. F., Ibanez, C. F., Lluis, C., Franco, R., Ferre, S., and Fuxe, K. J. Biol. Chem. 277, 18091-18097 (2002).
88. Kamiya, T., Saitoh, O., Yoshioka, K., and Nakata, H. Biochemical and Biophysical Research Communications 306, 544-549 (2003).
89. Kearn, C. S., Blake-Palmer, K., Daniel, E., Mackie, K., and Glass, M. Mol Pharmacol 67, 1697-1704 (2005).
90. Hlavackova, V., Goudet, C., Kniazeff, J., Zikova, A., Maurel, D., Vol, C., Trojanova, J., Prezeau, L., Pin, J. P., and Blahos, J. Embo Journal 24, 499-509 (2005).
91. Kniazeff, J., Bessis, A. S., Maurel, D., Ansanay, H., Prezeau, L., and Pin, J. P. Nature Structural & Molecular Biology 11, 706-713 (2004).
92. Chandrashekar, J., Hoon, M. A., Ryba, N. J. P., and Zuker, C. S. Nature 444, 288-294 (2006).
93. Springael, J. Y., Urizar, E., Costagliola, S., Vassart, G., and Parmentier, M. Pharmacology & Therapeutics 115, 410-418 (2007).
94. El Asmar, L., Springael, J. Y., Ballet, S., Andrieu, E. U., Vassart, G., and Parmentier, M. Mol Pharmacol 67, 460-469 (2005).
95. Sohy, D., Parmentier, M., and Springael, J. Y. J. Biol. Chem. 282, 30062-30069 (2007).
96. Springael, J. Y., Le Minh, P. N., Urizar, E., Costagliola, S., Vassart, G., and Parmentier, M. Mol Pharmacol 69, 1652-1661. (2006)
97. Waldhoer, M., Fong, J., Jones, R. M., Lunzer, M. M., Sharma, S. K., Kostenis, E., Portoghese, P. S., and Whistler, J. L. Proceedings of the National Academy of Sciences of the United States of America 102, 9050-9055 (2005).
98. Daniels, D. J., Lenard, N. R., Etienne, C. L., Law, P. Y., Roerig, S. C., and Portoghese, P. S. PNAS 102, 19208-19213 (2005).
99. Breit, A., Gagnidze, K., Devi, L. A., Lagace, M., and Bouvier, M. Mol Pharmacol 70, 686-696 (2006).
100. Park, P. S. H. and Palczewski, K. PNAS 102, 8793-8794 (2005).
101. Maggio, R., Vogel, Z., and Wess, J. Proceedings of the National Academy of Sciences of the United States of America 90, 3103-3107 (1993).
102. Hadac, E. M., Ji, Z. S., Pinon, D. I., Henne, R. M., Lybrand, T. P., and Miller, L. J. Journal of Medicinal Chemistry 42, 2105-2111 (1999).
103. Osuga, Y., Hayashi, M., Kudo, M., Conti, M., Kobilka, B., and Hsuch, A. J. W. J. Biol. Chem. 272, 25006-25012 (1997).
104. Urizar, E., Montanelli, L., Loy, T., Bonomi, M., Swillens, S., Gales, C., Bouvier, M., Smits, G., Vassar(G., and Costagliola, S. Embo Journal 24, 1954-1964 (2005).
105. Ji, I., Lee, C., Jeoung, M., Koo, Y., Sievert, G. A., and Ji, T. H. Mol Endocrinol 18, 968-978 (2004).
106. Yao, L., Fan, P., Jiang, Z., Mailliard, W. S., Gordon, A. S., and Diamond, I. PNAS 100, 14379-14384 (2003).
107. Chneiweiss, H., Glowinski, J., and Premont, J. J. Neurosci. 8, 3376-3382 (1988).
108. Arenas, E., Alberch, J., and Marsal, J. Neuroscience 42, 707-714 (1991).
109. Ambrose, L. M., Gallagher, S. M., Unterwald, E. M., and Van Bockstaele, E. J. Neuroscience Letters 399, 191-196 (2006).
110. Wang, H. and Pickel, V. M. J. Neurosci. 21, 3242-3250 (2001).
111. Hamm, H. E. Proc. Natl. Acad. Sci. USA 98, pp. 4819-4821 (2001).
112. Papac D I et al., J Biol Chem. 267(24):16889-94 (1992).
113. Baneres J-L et al., J. Mol. Biol. 329: 801-814 (2003).
114. Javitch, J. A. et al. The fourth transmembrane segment of the dopamine D2 receptor: Accessibility in the binding-site crevice and position in the transmembrane bundle. Biochemistry 39, 12190-12199 (2000).
115. Damian, M., Mary, S., Martin, A., Pin, J. P. & Baneres, J. L. G protein activation by the leukotriene B4 receptor dimer: Evidence for an absence of trans-activation. J. Biol. Chem. 283, 21084-21092 (2008).
116. Costagliola, S. et al. Structure-function relationships of two loss-of-function mutations of the thyrotropin receptor gene. Thyroid 9, 995-1000 (1999).
117. Guo, W. et al. Dopamine D2 receptors form higher order oligomers at physiological expression levels. EMBO J. (2008).
118. Scheerer, P. et al. Crystal structure of opsin in its G-protein-interacting conformation. Nature 455, 497-502 (2008).
119. Goudet, C. et al. Asymmetric Functioning of Dimeric Metabotropic Glutamate Receptors Disclosed by Positive Allosteric Modulators. J. Biol. Chem. 280, 24380-24385 (2005).
120. Kaupmann, K. et al. GABAB-receptor subtypes assemble into functional heteromeric complexes. Nature 396, 683-687 (1998).
121. Urban, J. D. et al. Functional selectivity and classical concepts of quantitative pharmacology. J Pharmacol Exp Ther. 320, 1-13 (2007).
122. Barnes, W. G., Reiter, E., Violin, J. D., Ren, X. R., Milligan, G., and Lefkowitz, R. J. (2005) beta-Arrestin 1 and Galphaq/11 coordinately activate RhoA and stress fiber formation following receptor stimulation. J. Biol. Chem. 280, 8041-8050.
123. Atkinson, P. J., Young, K. W., Ennion, S. J., Kew, J. N., Nahorski, S. R., and Challiss, R. A. (2006) Altered expression of G(q/11alpha) protein shapes mGlu1 and mGlu5 receptor-mediated single cell inositol 1,4,5-trisphosphate and Ca²⁺ signaling. Mol. Pharmacol. 69, 174-184.

Although illustrative embodiments of the present invention have been described herein, it should be understood that the invention is not limited to those described, and that various other changes or modifications may be made by one skilled in the art without departing from the scope or spirit of the invention.

1. A biological reagent comprising a complex having
(a) a first G-protein coupled receptor (GPCR); and
(b) a second GPCR linked to a G-protein, the linkage between the second GPCR and the G-protein being of a length, which prevents productive interaction between the G-protein and the second GPCR,
wherein the first GPCR and the second GPCR linked to the G-protein alone are incapable of producing a signal when presented with a ligand.

2. A biological reagent according to claim 1, wherein the complex is present in a cell membrane.

3. A biological reagent according to claim 2, wherein the cell membrane is part of an intact cell.

4. A biological reagent according to claim 1, wherein the second GPCR and the G-protein are linked as a fusion protein.

5. A biological reagent according to claim 4, wherein the second GPCR is linked directly to the G-protein.

6. A biological reagent according to claim 4, wherein the second GPCR is linked to the G-protein through a linker.

7. A biological reagent according to claim 6, wherein the linker is from 1 to 3 amino acids in length.

8. A biological reagent according to claim 1, wherein the first and/or second GPCRs are class A GPCRs.

9. A biological reagent according to claim 8, wherein the class A GPCR are selected from the group consisting of 5-Hydroxytryptamine 1A (5HT1A) receptor, 5-Hydroxytryptamine 1B (5HT1 B) receptor, 5-Hydroxytryptamine 1D (5HT1D) receptor, 5-Hydroxytryptamine 2A (5HT2A) receptor, 5-Hydroxytryptamine 2C (5HT2C) receptor, 5-Hydroxytryptamine 4 (5HT4) receptor, 5-Hydroxytryptamine 5A (5HT5A) receptor, 5-Hydroxytryptamine 6 (5HT6) receptor, α1A adrenergic receptor, al b adrenergic receptor, α2a adrenergic receptor, α2b adrenergic receptor, β1 adrenergic receptor, β2 adrenergic receptor, β3 adrenergic receptor, A1 adenosine receptor, A2 adenosine receptor, A3 adenosine receptor, muscarinic acetylcholine 1 (M1) receptor, muscarinic acetylcholine 2 (M2) receptor, muscarinic acetylcholine 3 (M3) receptor, muscarinic acetylcholine 4 (M4) receptor, Melanocortin2 receptor, angiotensin AT1A receptor, angiotensin AT1B receptor, B2 bradykinin receptor, CXCR3, CXCR4, D1 dopamine receptor, D2 dopamine receptor (D2R), D3 dopamine receptor, D4 dopamine receptor, follicle-stimulating hormone receptor (FSHR), gonadotropin-releasing hormone receptor (GRHR), histamine H1 receptor, histamine H2 receptor, lutropin-choriogonadotropic hormone receptor (LSHR), δ opioid receptor 1, κ opioid receptor 1, μ opioid receptor 1, rhodopsin, Oxytocin receptor, P2U purinoreceptor 1, Prostaglandin D2 receptor, Prostaglandin E2 receptor (EP1 subtype), Somatostatin receptor 2, Somatostatin receptor 5 (SSTR5), thyrotropin releasing hormone (TRH) receptor, Vasopressin 1A receptor, chemokine (C-C motif) receptor 5 (CCR5), and cannabinoid receptor 1.

10. A biological reagent according to claim 9, wherein the GPCR is a D2R.

11. A biological reagent according to claim 9, wherein the first GPCR is a SSTR5, and the second GPCR is a D2R.

12. A biological reagent according to claim 1, wherein the first GPCR and/or the second GPCR are Gi/o-coupled GPCRs.

13. A biological reagent according to claim 1, wherein the first GPCR and/or the second GPCR are Gq/11-coupled GPCRs.

14. A biological reagent according to claim 1, wherein the G-protein is a Gqi.

15. A biological reagent according to claim 14, wherein the G-protein is Gqi5.

16. A biological reagent according to claim 1, wherein the G-protein is a Gq/11 subfamily protein.

17. A biological reagent according to claim 1, wherein the second GPCR comprises a cysteine amino acid toward the terminal end of domain H8, which cysteine is palmitylated.

18. A biological reagent according to claim 17, wherein the G-protein is fused directly to the cysteine amino acid toward the terminal end of H8, which is palmitylated.

19. A biological reagent according to claim 1, wherein the G-protein is fused to an amino acid that corresponds to a position selected from the group consisting of position 410, 411, 412, 413, 414, 415, 416, 417, and 418 of the human wild type D2R (SEQ ID NO: 61) and isoforms, homologs, and orthologs thereof.

20. A biological reagent according to claim 19, wherein the G-protein is fused to an amino acid that corresponds to a position selected from the group consisting of position 413, 414, 415, 416, and 417 of SEQ ID NO: 61 and isoforms, homologs, and orthologs thereof.

21. A biological reagent according to claim 19, wherein the G-protein is fused to an amino acid that corresponds to position 414 of SEQ ID NO: 61 and isoforms, homologs, and orthologs thereof.

22. A biological reagent according to claim 19, wherein the amino acid is cysteine.

23. A biological reagent according to claim 19, wherein if the amino acid is not cysteine, it is modified to be cysteine prior to fusion of the G-protein.

24. A biological reagent according to claim 1, wherein the first GPCR comprises a mutation.

25. A biological reagent according to claim 1, wherein the second GPCR comprises a mutation.

26. A biological reagent according to claim 1, wherein both the first and second GPCRs comprise a mutation.

27. A biological reagent according to claim 24, wherein the mutation is from 1 to 3 single amino acid substitutions.

28. A biological reagent according to claim 27, wherein the mutation creates a mutant D2R.

29. A biological reagent according to claim 24, wherein the mutant D2R is selected from the group consisting of SFD80AGqi5, SFD80A/CAMGqi5, sMycD80A, SFD114AGqi5, SFD114A/CAMGqi5, sMycD114A, SFR132AGqi5, SF132A/CAMGqi5, sMycR132A, SFV136DM140EGqi5, SFV136DM140E/CAMGqi5, sMycV136DM140E, SFA213-219Gqi5, SFA 213-219/CAMGqi5, sMycΔ 213-219, SFAAAA(219-222RRKR) Gqi5, SFD2S AAAA(219-222RRKR)/CAMGqi5, sMycAAAA(219-222RRKR), SFAAAA(IYIV212-215)Gqi5, sMycAAAA(IVIY212-215), SFN393AGqi5, SFN393A/CAMGqi5, sMycN393A, SFD24LGqi5, SFD24L/CAMGqi5, sMycD24L, SFCAMGqi5, sMycD24short, SFD131A/R132A Gqi5, sMycCAM, SFD2/D4short Gqi5, sMycD131N, sMycD131A/R132A, sMycD2S D114A/CAM, sMycD2S D114A/D131N, sMycD2S D114A/R132A, sMycD2S D114A/V136D/M140E, and sMycD2S Y397F (Y7.53F).

30. A biological reagent according to claim 1, wherein the complex is capable of producing a signal when presented with a ligand.

31. A method of producing a biological reagent comprising the steps of:
(a) expressing a first nucleic acid in a cell, the nucleic acid encoding a first GPCR;
(b) expressing a second nucleic acid in the cell, the second nucleic acid encoding a fusion protein comprising a second GPCR fused to a G-protein, the G-protein being fused to the second GPCR in such a manner so that it cannot participate in a productive interaction with the second GPCR; and
(c) allowing the expressed proteins from steps (a) and (b) to assemble into a complex in the cell membrane, wherein the expressed proteins from steps (a) and (b) alone are incapable of producing a signal when presented with a ligand.

32. A method of producing a biological reagent according to claim 31 further comprising, prior to step (a), producing a construct comprising the first nucleic acid encoding the first GPCR and the second nucleic acid encoding the fusion protein of the second GPCR and the G-protein, the G-protein being fused to the second GPCR.

33. A method of producing a biological reagent according to claim 31 further comprising prior to step (a):
(i) producing a first construct comprising the first nucleic acid encoding the first GPCR;
(ii) producing a second construct comprising the second nucleic acid encoding the fusion protein of the second GPCR and the G-protein.

34. A method according to claim 31 further comprising isolating a part of the cell membrane comprising the complex.

35. A method of determining whether a first and second GPCR have affinity for each other such that they form a functional GPCR oligomer comprising:
(a) producing or providing a first nucleic acid construct encoding a first GPCR;
(b) producing or providing a second nucleic acid construct encoding a second GPCR and its associated G-protein as a fusion protein, the G-protein being fused to the second GPCR in such a manner so that it cannot participate in a productive interaction with the second GPCR, wherein the first GPCR and the second GPCR and its associated G-protein alone are incapable of producing a signal when presented with a ligand;
(c) co-expressing the first and second nucleic acid constructs in a cell; and
(d) determining the presence of a complex comprising the first and second GPCRs.

36. A method according to claim 35, wherein the presence of a complex is determined by contacting the cell with a ligand that binds the first GPCR and determining whether the G-protein is activated.

37. A method according to claim 35, wherein the cell expresses aequorin (AEQ).

38. A method of determining an effect a compound has on a GPCR oligomer comprising:
(a) contacting a compound with a first cell expressing a GPCR oligomer having:
(i) a first GPCR; and
(ii) a second GPCR fused to a G-protein, wherein the G-protein is fused to the second GPCR in such a manner so that it cannot participate in a productive interaction with the second GPCR, and the first GPCR and the second GPCR fused to the G-protein alone are incapable of producing a signal when presented with a ligand;
(b) detecting the presence of a cellular signal resulting from contact between the compound and the GPCR oligomer; and
(c) determining an effect the compound has on the GPCR oligomer.

39. A method according to claim 38 further comprising comparing the effect with that resulting from contact between the compound and a mutant of the first GPCR and/or with that resulting from contact between the compound and a mutant of the second GPCR and/or G-protein.

40. A method according to claim 38, wherein the method is adapted to be a high throughput screen.

41. A method of identifying a compound capable of interacting with a GPCR oligomer comprising:
(a) providing a cell expressing the biological reagent according to claim 1;
(b) contacting the biological reagent with the compound; and
(c) determining whether the compound interacts with the GPCR oligomer.

42. A method according to claim 41, wherein interaction between the compound and the GPCR oligomer is determined by detecting a change in a cellular signal resulting from the interaction.

43. A method according to claim 42, wherein the cellular signal is selected from the group consisting of Ca²⁺ flux, cAMP levels, inositol 1,4,5 triphosphate levels, protein kinase C activation, and MAP kinase activation.

44. A method according to claim 41, wherein the cellular signal is determined using a reporter assay.

45. A method according to claim 41, wherein the cell further comprises a plasmid encoding apoaequorin and the cellular signal is determined by a change in the luminescence of the cell.

46. A method according to claim 45, wherein the cell is a Flp-in T-rex 293 cell.

47. A method according to claim 41, wherein the compound interacts with the GPCR oligomer as an agonist, antagonist, an inverse agonist, or inverse antagonist.

48. A method according to claim 41, wherein the first GPCR has a modified amino acid sequence compared to the wild-type GPCR sequence so as to render it non-functional.

49. A method according to claim 41, wherein the second GPCR is a hD2R and the first GPCR is selected from the group consisting of hD1, hD3, hCCR5, hSSTR5, hDOR, hTSHR, hGluR1, hGluR5, hCB1, hA2a, hM4, and h5HT1b.

50. A method according to claim 41, wherein the second GPCR is a mutant D2R selected from the group consisting of SFD80AGqi5, SFD80A/CAMGqi5, sMycD80A, SFD114AGqi5, SFD114A/CAMGqi5, sMycD114A, SFR132AGqi5, SF132A/CAMGqi5, sMycR132A, SFV136DM140EGqi5, SFV136DM140E/CAMGqi5, sMycV136DM140E, SFA213-219Gqi5, SFΔ 213-219/CAMGqi5, sMycΔ 213-219, SFAAAA(219-222RRKR) Gqi5, SFD2S AAAA(219-222RRKR)/CAMGqi5, sMycAAAA(219-222RRKR), SFAAAA(IYIV212-215)Gqi5, sMycAAAA(IVIY212-215), SFN393AGqi5, SFN393A/CAMGqi5, sMycN393A, SFD24LGqi5, SFD24UCAMGqi5, sMycD24L, SFCAMGqi5, sMycD24short, SFD131A/R132A Gqi5, sMycCAM, SFD2/D4short Gqi5, sMycD131N, sMycD131A/R132A, sMycD2S D114A/CAM, sMycD2S D114A/D131N, sMycD2S D114A/R132A, sMycD2S D114A/V136D/M140E, and sMycD2S Y397F (Y7.53F).

51. A method according to claim 41, wherein one of the GPCRs is selected from the group consisting of 3HA-human D1, 3HAD1-linker-Gqi5, 3HA-human 5HT1b, 3HA-human A2a, 3HA-human CB1, mGluR1a (rat), mGluR5a (rat), SF-human D3, SFD3Gqi5, SFD3-linker-Gqi5, SF-human SSTR5, smyc-human SSTR5, 3HA-M4-linker-Gqi5, 3HA-M4Gqi5a, human CCR5, CCR5 Gqi5, smycDOR, and TSHr Gqi5.

52. A method according to claim 41, wherein the first GPCR is a wild type D2R and the second GPCR fused to a G protein is D2R-Gqi5.

53. A method according to claim 41, wherein the method is adapted to be a high throughput screen.

54. A method of identifying a compound having the ability to modulate binding between a GPCR oligomer and its ligand comprising:
(a) providing a cell expressing a GPCR oligomer comprising:
(i) a first GPCR; and
(ii) a second GPCR linked to a G-protein, the linkage between the second GPCR and the G-protein being of a length, which prevents productive interaction between the G-protein and the second GPCR, wherein the first GPCR and the second GPCR linked to the G-protein alone are incapable of producing a signal when presented with a ligand;
(b) contacting the cell with a test compound in the presence of the ligand; and
(c) comparing the ability of the ligand to bind to the GPCR oligomer with the ability of the ligand to bind to the GPCR oligomer under comparable conditions but in the absence of the compound.

55. A method according to claim 54, wherein the compound is a protein or a peptide.

56. A method according to claim 55, wherein the protein is a third GPCR.

57. A method according to claim 54, wherein the ligand binds to a new or altered ligand binding site determined to be present on the oligomer.

58. A method according to claim 54, wherein the first GPCR, the second GPCR, and/or the G-protein has a modified amino acid sequence compared to a wild-type sequence.

59. A method for evaluating differential G-protein coupling comprising:
(a) providing a first cell expressing a first GPCR oligomer comprising:
(i) a first wild type GPCR;
(ii) a second wild type GPCR linked to a G-protein, the linkage between the second GPCR and the G-protein being of a length, which prevents productive interaction between the G-protein and the second GPCR, wherein the first GPCR and second GPCR linked to the G-protein alone are incapable of producing a signal when presented with a ligand;
(b) providing a second cell expressing a second GPCR oligomer comprising:
(i) the first GPCR comprising a mutation;
(ii) the second GPCR linked to a G-protein, the linkage between the second GPCR and the G-protein being of a length, which prevents productive interaction between the G-protein and the second GPCR, wherein the mutant first GPCR and the second GPCR linked to the G-protein alone are incapable of producing a signal when presented with a ligand;
(c) providing a third cell expressing a third GPCR oligomer comprising:
(i) the first GPCR;
(ii) the second GPCR, which comprises a mutation and is linked to a G-protein, the linkage between the second mutant GPCR and the G-protein being of a length, which prevents productive interaction between the G-protein and the second mutant GPCR, wherein the first GPCR and the second mutant GPCR linked to the G-protein alone are incapable of producing a signal when presented with a ligand;
(d) contacting the first, second and third cells with a compound capable of binding to the ligand binding site present on the first and/or the second GPCR;
(e) repeating steps (a) to (d) with a different G-protein; and
(f) evaluating differential G-protein coupling.

60. A method according to claim 59, wherein the G-protein is Gqi.

61. A method according to claim 59, wherein the G-protein modulates an intracellular signal selected from the group consisting of Ca²⁺ level, cAMP level, cGMP level, inositol 1, 4, 5 triphosphate level, diacylglycerol level, protein kinase C activity, and MAP kinase activity.

62. A method according to claim 59, wherein the first, second, and third cell each express aequorin and the evaluation step comprises detecting luminescence.

63. A method of identifying a compound having the ability to modulate the activity of a GPCR oligomer comprising:
(a) providing a cell expressing a GPCR oligomer comprising:
(i) a first GPCR; and
(ii) a second GPCR linked to a G-protein, the linkage between the second GPCR and the G-protein being of a length, which prevents productive interaction between the G-protein and the second GPCR, wherein the first GPCR and the second GPCR linked to the G-protein alone are incapable of producing a signal when presented with a ligand;
(b) contacting the cell with a test compound in the presence of a ligand of the first GPCR or of the second GPCR; and
(c) comparing the activity of the GPCR oligomer with the activity of the GPCR oligomer under comparable conditions but in the absence of the compound.

64. The method according to claim 63, wherein the compound binds to the first GPCR but not the second GPCR.

65. The method according to claim 63, wherein the compound binds to the second GPCR but not the first GPCR.

66. The method according to claim 63, wherein the second GPCR is D2R.

67. The method according to claim 66, wherein the first GPCR is selected from the group consisting of D2R, SSTR5, and DOR.

68. A method for evaluating differential effects of a compound on the activity of a GPCR oligomer comprising:
(a) providing a first cell expressing a first GPCR oligomer comprising:
(i) a first GPCR;
(ii) a second GPCR linked to a G-protein, the linkage between the second GPCR and the G-protein being of a length, which prevents productive interaction between the G-protein and the second GPCR, wherein the first GPCR and second GPCR linked to the G-protein alone are incapable of producing a signal when presented with a first ligand;
(b) providing a second cell expressing a second GPCR oligomer comprising:
(i) a third GPCR;
(ii) a fourth GPCR linked to a G-protein, the linkage between the fourth GPCR and the G-protein being of a length, which prevents productive interaction between the G-protein and the fourth GPCR, wherein the third GPCR and the fourth GPCR linked to the G-protein alone are incapable of producing a signal when presented with a second ligand;
(c) contacting the first and second cells with a compound capable of binding to the first, the second, the third, and/or the fourth GPCR; and
(d) evaluating the differential activity, if any, of each GPCR oligomer under comparable conditions but in the absence of the compound.

69. The method according to claim 68, wherein the first, the second, and the fourth GPCRs are the same.

70. The method according to claim 69, wherein the first, the second, and the fourth GPCRs are D2R.

71. The method according to claim 70, wherein the third GPCR is SSTR5.

72. A biological reagent according to claim 20, wherein the amino acid is cysteine.

73. A biological reagent according to claim 21, wherein the amino acid is cysteine.

74. A biological reagent according to claim 20, wherein if the amino acid is not cysteine, it is modified to be cysteine prior to fusion of the G-protein.

75. A biological reagent according to claim 21, wherein if the amino acid is not cysteine, it is modified to be cysteine prior to fusion of the G-protein.

76. A biological reagent according to claim 25, wherein the mutation is from 1 to 3 single amino acid substitutions.

77. A biological reagent according to claim 26, wherein the mutation is from 1 to 3 single amino acid substitutions.

78. A biological reagent according to claim 76, wherein the mutation creates a mutant D2R.

79. A biological reagent according to claim 77, wherein the mutation creates a mutant D2R.

80. A biological reagent according to claim 25, wherein the mutant D2R is selected from the group consisting of SFD80AGqi5, SFD80A/CAMGqi5, sMycD80A, SFD114AGqi5, SFD114A/CAMGqi5, sMycD114A, SFR132AGqi5, SF132A/CAMGqi5, sMycR132A, SFV136DM140EGqi5, SFV136DM140E/CAMGqi5, sMycV136DM140E, SFA213-219Gqi5, SFA 213-219/CAMGqi5, sMycΔ 213-219, SFAAAA(219-222RRKR) Gqi5, SFD2S AAAA(219-222RRKR)/CAMGqi5, sMycAAAA(219-222RRKR), SFAAAA(IYIV212-215)Gqi5, sMycAAAA(IVIY212-215), SFN393AGqi5, SFN393A/CAMGqi5, sMycN393A, SFD24LGqi5, SFD24L/CAMGqi5, sMycD24L, SFCAMGqi5, sMycD24short, SFD131A/R132A Gqi5, sMycCAM, SFD2/D4short Gqi5, sMycD131N, sMycD131A/R132A, sMycD2S D114A/CAM, sMycD2S D114A/D131N, sMycD2S D114A/R132A, sMycD2S D114A/V136D/M140E, and sMycD2S Y397F (Y7.53F).

81. A biological reagent according to claim 26, wherein the mutant D2R is selected from the group consisting of SFD80AGqi5, SFD80A/CAMGqi5, sMycD80A, SFD114AGqi5, SFD114A/CAMGqi5, sMycD114A, SFR132AGqi5, SF132A/CAMGqi5, sMycR132A, SFV136DM140EGqi5, SFV136DM140E/CAMGqi5, sMycV136DM140E, SFA213-219Gqi5, SFA 213-219/CAMGqi5, sMycΔ 213-219, SFAAAA(219-222RRKR) Gqi5, SFD2S AAAA(219-222RRKR)/CAMGqi5, sMycAAAA(219-222RRKR), SFAAAA(IYIV212-215)Gqi5, sMycAAAA(IVIY212-215), SFN393AGqi5, SFN393A/CAMGqi5, sMycN393A, SFD24LGqi5, SFD24L/CAMGqi5, sMycD24L, SFCAMGqi5, sMycD24short, SFD131A/R132A Gqi5, sMycCAM, SFD2/D4short Gqi5, sMycD131N, sMycD131A/R132A, sMycD2S D114A/CAM, sMycD2S D114A/D131N, sMycD2S D114A/R132A, sMycD2S D114A/V136D/M140E, and sMycD2S Y397F (Y7.53F).
